(19) United States
(12) Patent Application Publication
(10) Pub. No.: US 2024/0327401 A1
(43) Pub. Date: Oct. 3, 2024

(54) FUSED RING COMPOUND, PHARMACEUTICAL COMPOSITION, AND APPLICATION THEREOF

(71) Applicant: EVOPOINT BIOSCIENCES CO., LTD., Jiangsu (CN)

(72) Inventors: Dongdong WU, Jiangsu (CN); Xin LI, Jiangsu (CN); Jiajing XU, Jiangsu (CN); Linfeng XU, Jiangsu (CN); Yuchuan WU, Jiangsu (CN); Yonghan HU, Jiangsu (CN)

(73) Assignee: EVOPOINT BIOSCIENCES CO., LTD., Jiangsu (CN)

(21) Appl. No.: 18/293,764

(22) PCT Filed: Aug. 3, 2022
(86) PCT No.: PCT/CN2022/110019

§ 371 Date: Jan. 30, 2024

(30) Foreign Application Priority Data
Aug. 3, 2021 (CN) 202110884595.1
Sep. 10, 2021 (CN) 202111063419.8
Nov. 4, 2021 (CN) 202111300099.3
Nov. 25, 2021 (CN) 202111415010.8
Jan. 20, 2022 (CN) 202210068494.1

(51) Int. Cl. C07D 471/04; A61K 31/519

(52) U.S. Cl. CPC C07D 471/04; A61K 31/519

(57) ABSTRACT

A fused ring compound shown in formula I or a pharmaceutically acceptable salt has a novel structure and good SOS1 inhibitory activity.

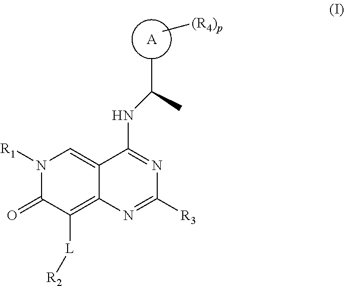

The present application claims priority to Chinese Patent Application No. 202110884595.1 filed on Aug. 3, 2021, Chinese Patent Application No. 202111063419.8 filed on Sep. 10, 2021, Chinese Patent Application No. 202111300099.3 filed on Nov. 4, 2021, Chinese Patent Application No. 202111415010.8 filed on Nov. 25, 2021, and Chinese Patent Application No. 2022100684941 filed on Jan. 20, 2022, which are incorporated herein by reference in their entireties.

### TECHNICAL FIELD

The present invention relates to a fused ring compound, a pharmaceutical composition, and use thereof.

### BACKGROUND

SOS (son of sevenless) protein was first discovered in 1992 by Bonfini et al. in their study of Drosophila eyes. SOS proteins are products of SOS genes encoding guanyl-releasing proteins and play an important role in the growth and development of Drosophila, nematodes, mice, and humans. SOS1 (son of sevenless homolog 1) is a guanine nucleotide exchange factor (GEF) of Ras and Rac, which can play an important role in Ras and Rac signaling pathways by regulating the guanosine diphosphate (GDP)/guanosine triphosphate (GTP) exchange of G proteins. SOS1 was identified to be involved in the Ras signaling pathway and in the tumorigenic process of many tumors.

There are two SOS homologues in humans, namely hSOS1 and hSOS2. The hSOS1 protein has a size of 150 kDa and consists of 1300 amino acid residues. The C-terminus of hSOS1 contains a proline-rich (PxxP) domain that can interact with the SH3 (Src homology 3) domain of proteins such as growth factor receptor-bound protein 2 (Grb2) in the Ras pathway, E3B1 in the Rac pathway, and the like. Ras-GTP is a more effective SOS allosteric activator, and single-molecule sequencing results show that Ras-GTP can bind to an allosteric binding site of SOS1 to cause a change in the structure of SOS1, which prevents the PxxP domain of SOS1 from binding to Grb2 to generate activity, thus exerting an inhibitory effect.

SOS1 can cause Ras-GTP to be converted to activate Ras. The activation of Ras plays an important role in cell growth and differentiation, and can further cause the activation of the Raf-mitogen-activated protein kinase (MAPK)-extracellular signal-regulated protein kinase (ERK) signaling pathway, thereby having a decisive role in cell proliferation, transformation and migration. Excessive activation of this signaling pathway can cause the development of cancer.

### BRIEF SUMMARY OF THE INVENTION

The present invention aims to solve the technical problem that existing SOS1 inhibitors have fewer structural varieties, and therefore, the present invention provides a fused ring compound, a pharmaceutical composition, and use thereof. The compound has a novel structure and good SOS1 inhibitory activity.

The present invention solves the above technical problem by means of the following technical solutions.

The present invention provides a fused ring compound represented by formula I or a pharmaceutically acceptable salt thereof:

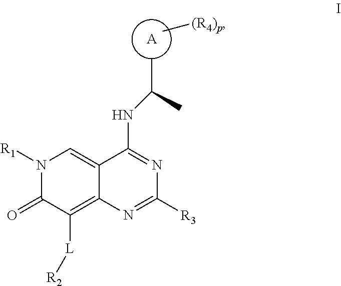

wherein,
R₁ is C₁₋₆ alkyl, C₁₋₆ alkyl substituted with one or more R₁₋₁ₐ, C₃₋₁₀ cycloalkyl, C₃₋₁₀ cycloalkyl substituted with one or more R_{1-1b}, “3- to 10-membered heterocyclyl having 1, 2 or 3 heteroatoms selected from 1, 2 or 3 of N, O and S”, “3- to 10-membered heterocyclyl having 1, 2 or 3 heteroatoms selected from 1, 2 or 3 of N, O and S” substituted with one or more R_{1-1c}, C₆₋₁₀ aryl, C₆₋₁₀ aryl substituted with one or more R_{1-1d}, “5- to 10-membered heteroaryl having 1, 2 or 3 heteroatoms selected from 1, 2 or 3 of N, O and S”, or “5- to 10-membered heteroaryl having 1, 2 or 3 heteroatoms selected from 1, 2 or 3 of N, O and S” substituted with one or more R₁₋₁ₑ;
R₁₋₁ₐ, R_{1-1b}, R_{1-1c}, R_{1-1d}, and R₁₋₁ₑ are each independently —OR₁₋₂ₐ, —NR_{1-2b}R_{1-2c}, halogen, —CN, —C(O)R_{1-2d}, —C(O)OR₁₋₂ₑ, —C(O)NR_{1-2f}R_{1-2g}, C₁₋₆ alkyl, C₁₋₆ alkyl substituted with one or more R₁₋₃ₐ, C₃₋₁₀ cycloalkyl, or “C₃₋₁₀ cycloalkyl substituted with one or more R_{1-3b}”;
R₁₋₂ₐ, R_{1-2b}, R_{1-2c}, R_{1-2d}, R₁₋₂ₑ, R_{1-2f}, and R_{1-2g} are each independently H, C₁₋₆ alkyl, C₁₋₆ alkyl substituted with one or more R_{1-3c}, C₃₋₁₀ cycloalkyl, C₃₋₁₀ cycloalkyl substituted with one or more R_{1-3d}, “3- to 10-membered heterocyclyl having 1, 2 or 3 heteroatoms selected from 1, 2 or 3 of N, O and S”, “3- to 10-membered heterocyclyl having 1, 2 or 3 heteroatoms selected from 1, 2 or 3 of N, O and S” substituted with one or more R₁₋₃ₑ, C₆₋₁₀ aryl, C₆₋₁₀ aryl substituted with one or more R_{1-3f}, “5- to 10-membered heteroaryl having 1, 2 or 3 heteroatoms selected from 1, 2 or 3 of N, O and S”, or “5- to 10-membered heteroaryl having 1, 2 or 3 heteroatoms selected from 1, 2 or 3 of N, O and S” substituted with one or more R_{1-3g};
R₁₋₃ₐ, R_{1-3b}, R_{1-3c}, R_{1-3d}, R₁₋₃ₑ, R_{1-3f}, and R_{1-3g} are each independently —OR₁₋₄ₐ, —NR_{1-4b}R_{1-4c}, halogen, C₁₋₆ alkyl substituted with one or more halogens, C₃₋₁₀ cycloalkyl, “3- to 10-membered heterocyclyl having 1, 2 or 3 heteroatoms selected from 1, 2 or 3 of N, O and S”, C₆₋₁₀ aryl, or “5- to 10-membered heteroaryl having 1, 2 or 3 heteroatoms selected from 1, 2 or 3 of N, O and S”;
R₁₋₄ₐ, R_{1-4b}, and R_{1-4c} are each independently hydrogen, C₁₋₆ alkyl, C₁₋₆ alkyl substituted with one or more halogens, C₃₋₁₀ cycloalkyl, “3- to 10-membered heterocyclyl having 1, 2 or 3 heteroatoms selected from 1, 2 or 3 of N, O and S”, C₆₋₁₀ aryl, or “5- to 10-membered heteroaryl having 1, 2 or 3 heteroatoms selected from 1, 2 or 3 of N, O and S”;
L is —O—, —S—, —SO₂—, or —NR_{L-1}; R_{L-1} is hydrogen or C₁₋₆ alkyl;
R₂ is selected from hydrogen, C₁₋₆ alkyl, C₁₋₆ alkyl substituted with one or more R₂₋₁, C₃₋₁₀ cycloalkyl, or “3- to 10-membered heterocyclyl having 1, 2 or 3 heteroatoms selected from 1, 2 or 3 of N, O and S”;
each R₂₋₁ is independently deuterium, halogen, C₃₋₁₀ cycloalkyl, or hydroxy;
R₃ is hydrogen, C₁₋₆ alkyl, or C₁₋₆ alkyl substituted with one or more halogens;
each R₄ is independently halogen, —NH₂, C₁₋₆ alkyl, C₁₋₆ alkyl substituted with one or more R₄₋₁, “3- to 10-membered heterocyclyl having 1, 2 or 3 heteroatoms selected from 1, 2 or 3 of N, O and S”, C₃₋₆ cycloalkyl, C₆₋₁₀ aryl, or C₆₋₁₀ aryl substituted with one or more —(CH₂)ₘNR₄₋₂R₄₋₃, wherein m is 0, 1, 2 or 3;
each R₄₋₁ is independently halogen or hydroxy;
R₄₋₂ and R₄₋₃ are each independently C₁₋₆ alkyl;
ring A is C₆₋₁₀ aryl, “5- to 10-membered heterocyclyl having 1, 2 or 3 heteroatoms selected from 1, 2 or 3 of N, O and S”, or “5- to 10-membered heteroaryl having 1, 2 or 3 heteroatoms selected from 1, 2 or 3 of N, O and S”; and
p is 1, 2 or 3.

In certain preferred embodiments of the present invention, certain groups in the fused ring compound represented by formula I or the pharmaceutically acceptable salt thereof are defined below, and groups not mentioned are as described in any of the embodiments of the present invention (referred to as “in some embodiments”).

In some embodiments, each R₄ may also be independently —CN.

In some embodiments, R₁ is C₃₋₁₀ cycloalkyl, C₃₋₁₀ cycloalkyl substituted with one or more R_{1-1b}, “3- to 10-membered heterocyclyl having 1, 2 or 3 heteroatoms selected from 1, 2 or 3 of N, O and S”, or “3- to 10-membered heterocyclyl having 1, 2 or 3 heteroatoms selected from 1, 2 or 3 of N, O and S” substituted with one or more R_{1-1c}, preferably C₃₋₁₀ cycloalkyl substituted with one or more R_{1-1b}, “3- to 10-membered heterocyclyl having 1, 2 or 3 heteroatoms selected from 1, 2 or 3 of N, O and S”, or “3- to 10-membered heterocyclyl having 1, 2 or 3 heteroatoms selected from 1, 2 or 3 of N, O and S” substituted with one or more R₁₋₁c.

In some embodiments, R₁₋₁ₐ, R_{1-1b}, R_{1-1c}, R_{1-1d}, and R₁₋₁ₑ are each independently halogen, —CN, —C(O)R_{1-2d}, C₁₋₆ alkyl, or C₁₋₆ alkyl substituted with one or more R₁₋₃ₐ, preferably C₁₋₆ alkyl or C₁₋₆ alkyl substituted with one or more R₁₋₃ₐ.

In some embodiments, R₁₋₂ₐ, R_{1-2b}, R_{1-2c}, R_{1-2d}, R₁₋₂ₑ, R_{1-2f}, and R_{1-2g} are each independently C₃₋₁₀ cycloalkyl.

In some embodiments, R₁₋₃ₐ, R_{1-3b}, R_{1-3c}, R_{1-3d}, R₁₋₃ₑ, R_{1-3f}, and R_{1-3g} are each independently —OR₁₋₄ₐ or halogen, preferably halogen.

In some embodiments, R₁₋₄ₐ, R_{1-4b}, and R_{1-4c} are each independently hydrogen or C₁₋₆ alkyl.

In some embodiments, R₃ is C₁₋₆ alkyl.

In some embodiments, each R₄ is independently halogen, —NH₂, C₁₋₆ alkyl, C₁₋₆ alkyl substituted with one or more R₄₋₁, or C₆₋₁₀ aryl substituted with one or more —(CH₂)ₘNR₄₋₂R₄₋₃.

In some embodiments, L is —O— or —S—.

In some embodiments, R₂ is C₁₋₆ alkyl, C₁₋₆ alkyl substituted with one or more R₂₋₁, C₃₋₁₀ cycloalkyl, or “3- to 10-membered heterocyclyl having 1, 2 or 3 heteroatoms selected from 1, 2 or 3 of N, O and S”, preferably C₁₋₆ alkyl or C₁₋₆ alkyl substituted with one or more R₂₋₁. In some embodiments, each R₄ is independently halogen, —NH₂, C₁₋₆ alkyl, or C₁₋₆ alkyl substituted with one or more R₄₋₁.

In some embodiments, each R₄ is independently —CN.

In some embodiments, R₁ is C₃₋₁₀ cycloalkyl substituted with one or more R_{1-1b}, “3- to 10-membered heterocyclyl having 1, 2 or 3 heteroatoms selected from 1, 2 or 3 of N, O and S”, or “3- to 10-membered heterocyclyl having 1, 2 or 3 heteroatoms selected from 1, 2 or 3 of N, O and S” substituted with one or more R_{1-1c};
each R_{1-1b} is independently C₁₋₆ alkyl substituted with one or more R₁₋₃a;
each R_{1-1c} is independently C₁₋₆ alkyl;
each R₁₋₃ₐ is independently halogen;
L is —O—;
R₂ is C₁₋₆ alkyl or C₁₋₆ alkyl substituted with one or more R₂₋₁;
R₂₋₁ is deuterium;
R₃ is C₁₋₆ alkyl;
ring A is C₆₋₁₀ aryl, “5- to 10-membered heterocyclyl having 1, 2 or 3 heteroatoms selected from 1, 2 or 3 of N, O and S”, or “5- to 10-membered heteroaryl having 1, 2 or 3 heteroatoms selected from 1, 2 or 3 of N, O and S”;
each R₄ is independently halogen, —NH₂, C₁₋₆ alkyl, C₁₋₆ alkyl substituted with one or more R₄₋₁, or C₆₋₁₀ aryl substituted with one or more —(CH₂)ₘNR₄₋₂R₄₋₃; and
each R₄₋₁ is independently halogen or hydroxy.

In some embodiments, R₁ is C₃₋₁₀ cycloalkyl substituted with one or more R_{1-1b}, “3- to 10-membered heterocyclyl having 1, 2 or 3 heteroatoms selected from 1, 2 or 3 of N, O and S”, or “3- to 10-membered heterocyclyl having 1, 2 or 3 heteroatoms selected from 1, 2 or 3 of N, O and S” substituted with one or more R_{1-1c};
each R_{1-1b} is independently C₁₋₆ alkyl substituted with one or more R₁₋₃a;
each R_{1-1c} is independently C₁₋₆ alkyl;
each R₁₋₃ₐ is independently halogen;
L is —O—;
R₂ is C₁₋₆ alkyl or C₁₋₆ alkyl substituted with one or more R₂₋₁;
R₂₋₁ is deuterium;
R₃ is C₁₋₆ alkyl;
ring A is C₆₋₁₀ aryl, “5- to 10-membered heterocyclyl having 1, 2 or 3 heteroatoms selected from 1, 2 or 3 of N, O and S”, or “5- to 10-membered heteroaryl having 1, 2 or 3 heteroatoms selected from 1, 2 or 3 of N, O and S”;
each R₄ is independently halogen, —NH₂, —CN, C₁₋₆ alkyl, C₁₋₆ alkyl substituted with one or more R₄₋₁, or C₆₋₁₀ aryl substituted with one or more —(CH₂)ₘNR₄₋₂R₄₋₃; and
each R₄₋₁ is independently halogen or hydroxy.

In some embodiments, in R₁, R₁₋₁ₐ, R_{1-1b}, R_{1-1c}, R_{1-1d}, R₁₋₁ₑ, R₁₋₂ₐ, R_{1-2b}, R_{1-2c}, R_{1-2d}, R₁₋₂ₑ, R_{1-2f}, R_{1-2g}, R₁₋₃ₐ, R_{1-3b}, R_{1-3c}, R_{1-3d}, R₁₋₃ₑ, R_{1-3f}, R_{1-3g}, R₁₋₄ₐ, R_{1-4b}, R_{1-4c}, R_{L-1}, R₂, R₃, R₄, R₄₋₂, and R₄₋₃, the “C₁₋₆ alkyl” in each of the “C₁₋₆ alkyl substituted with one or more R₁₋₁ₐ”, the “C₁₋₆ alkyl substituted with one or more R₁₋₃ₐ”, the “C₁₋₆ alkyl substituted with one or more R_{1-3c}”, the “C₁₋₆ alkyl substituted with one or more halogens”, the “C₁₋₆ alkyl”, the “C₁₋₆ alkyl substituted with one or more R₂₋₁”, and the “C₁₋₆ alkyl substituted with one or more R₄₋₁” is independently methyl, ethyl, n-propyl, isopropyl, n-butyl, isobutyl, sec-butyl, or tert-butyl, preferably methyl, ethyl, isobutyl, or isopropyl.

In some embodiments, in R₁, R₁₋₁ₐ, R_{1-1b}, R_{1-1c}, R_{1-1d}, R₁₋₁ₑ, R₁₋₂ₐ, R_{1-2b}, R_{1-2c}, R_{1-2d}, R₁₋₂ₑ, R_{1-2f}, R_{1-2g}, R₁₋₃ₐ, R_{1-3b}, R_{1-3c}, R_{1-3d}, R₁₋₃ₑ, R_{1-3f}, R_{1-3g}, R₁₋₄ₐ, R_{1-4b}, R_{1-4c}, R₂, and R₂₋₁, the “C₃₋₁₀ cycloalkyl” in each of the “C₃₋₁₀ cycloalkyl”, the “C₃₋₁₀ cycloalkyl substituted with one or more R_{1-1b}”, the “C₃₋₁₀ cycloalkyl substituted with one or more R_{1-3b}”, and the “C₃₋₁₀ cycloalkyl substituted with one or more R_{1-3d}” is independently C₃₋₆ cycloalkyl, such as cyclopropyl, cyclobutyl, or cyclopentyl

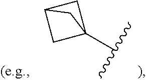 [SMILES: *C12CC(C1)C2]

preferably

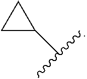 [SMILES: *C1CC1]

In some embodiments, in R₁, R₁₋₂ₐ, R_{1-2b}, R_{1-2c}, R_{1-2d}, R₁₋₂ₑ, R_{1-2f}, R_{1-2g}, R₁₋₃ₐ, R_{1-3b}, R_{1-3c}, R_{1-3d}, R₁₋₃ₑ, R_{1-3f}, R_{1-3g}, R₁₋₄ₐ, R_{1-4b}, R_{1-4c}, R₂, and R₄, the “3- to 10-membered heterocyclyl” in each of the “3- to 10-membered heterocyclyl having 1, 2 or 3 heteroatoms selected from 1, 2 or 3 of N, O and S”, the “3- to 10-membered heterocyclyl having 1, 2 or 3 heteroatoms selected from 1, 2 or 3 of N, O and S′ substituted with one or more R_{1-1c}”, and the “3- to 10-membered heterocyclyl having 1, 2 or 3 heteroatoms selected from 1, 2 or 3 of N, O and S′ substituted with one or more R₁₋₃ₑ” is independently 5- to 6-membered heterocyclyl, preferably saturated 5- to 6-membered heterocyclyl, and further preferably

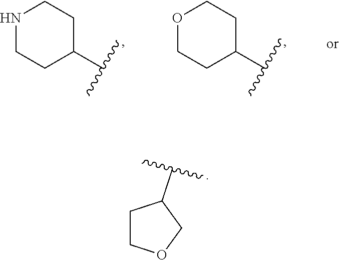 [SMILES: *C1CCNCC1 ; *C1CCOCC1 ; *C1CCOC1]

In some embodiments, in R₁, R₁₋₂ₐ, R_{1-2b}, R_{1-2c}, R_{1-2d}, R₁₋₂ₑ, R_{1-2f}, R_{1-2g}, R₁₋₃ₐ, R_{1-3b}, R_{1-3c}, R_{1-3d}, R₁₋₃ₑ, R_{1-3f}, R_{1-3g}, R₁₋₄ₐ, R_{1-4b}, R_{1-4c}, R₄, and ring A, the “C₆₋₁₀ aryl” in each of the “C₆₋₁₀ aryl”, the “C₆₋₁₀ aryl substituted with one or more R_{1-1d}”, the “C₆₋₁₀ aryl substituted with one or more R_{1-3f}”, and the “C₆₋₁₀ aryl substituted with one or more —(CH₂)ₘNR₄₋₂R₄₋₃” is independently phenyl.

In some embodiments, in R₁, R₁₋₂ₐ, R_{1-2b}, R_{1-2c}, R_{1-2d}, R₁₋₂ₑ, R_{1-2f}, R_{1-2g}, R₁₋₃ₐ, R_{1-3b}, R_{1-3c}, R_{1-3d}, R₁₋₃ₑ, R_{1-3f}, R_{1-3g}, R₁₋₄ₐ, R_{1-4b}, R_{1-4c}, and ring A, the “5- to 10-membered heteroaryl” in each of the “5- to 10-membered heteroaryl having 1, 2 or 3 heteroatoms selected from 1, 2 or 3 of N, O and S”, the “5- to 10-membered heteroaryl having 1, 2 or 3 heteroatoms selected from 1, 2 or 3 of N, O and S′ substituted with one or more R₁₋₁ₑ”, and the “5- to 10-membered heteroaryl having 1, 2 or 3 heteroatoms selected from 1, 2 or 3 of N, O and S′ substituted with one or more R_{1-3g}” is independently “9- to 10-membered heteroaryl”, such as benzofuranyl, preferably

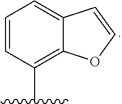 [SMILES: *c1cccc2ccoc12]

In some embodiments, in ring A, the “5- to 10-membered heterocyclyl” in the “5- to 10-membered heterocyclyl having 1, 2 or 3 heteroatoms selected from 1, 2 or 3 of N, O and S” is “9- to 10-membered heterocyclyl”, such as 2,3-dihydrobenzofuranyl, preferably

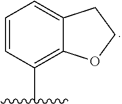 [SMILES: *c1cccc2c1OCC2]

In some embodiments, in R₁₋₁ₐ, R_{1-1b}, R_{1-1c}, R_{1-1d}, R₁₋₁ₑ, R₁₋₃ₐ, R_{1-3b}, R_{1-3c}, R_{1-3d}, R₁₋₃ₑ, R_{1-3f}, R_{1-3g}, R₂₋₁, R₃, R₄, and R₄₋₁, the “halogen” in each of the “halogen” and the “C₁₋₆ alkyl substituted with one or more halogens” is independently fluorine, chlorine, bromine, or iodine, preferably fluorine.

In some embodiments, R₁ is

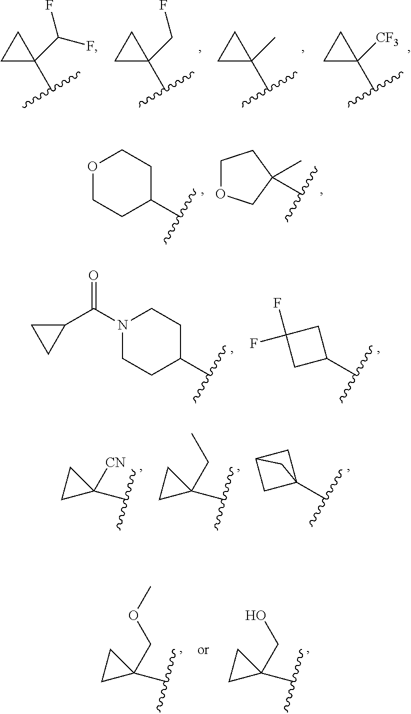

preferably

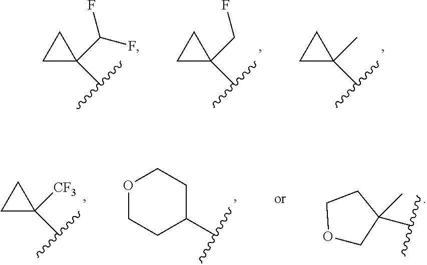

In some embodiments, R₂ is hydrogen, methyl, ethyl, —CD₃, isopropyl, —CH₂CF₃,

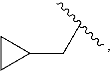 [SMILES: *CC1CC1]

cyclopropyl,

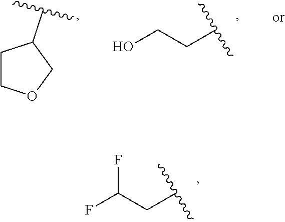

preferably methyl, ethyl, or —CD₃.

In some embodiments, -L-R₂ is —OCH₃, —OCH₂CH₃, —OCD₃, —SO₂CH₃, —OCH₂CF₃,

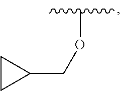 [SMILES: *OCC1CC1]

—SCH₃, —N(CH₃)(CH₃), —NH₂,

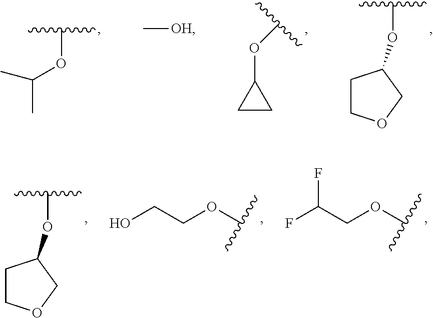

or —SO₂CH₂CH₃, preferably —OCH₃, —OCH₂CH₃, —OCD₃, or —SCH₃.

In some embodiments, R₃ is methyl.

In some embodiments, R₄ is

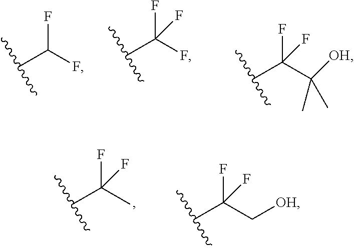

—F, —CH₃, —NH₂,

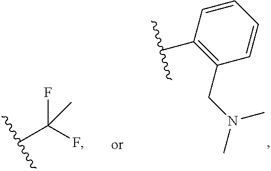

preferably

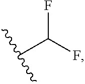 [SMILES: *C(F)F]

### —F, —CH₃,

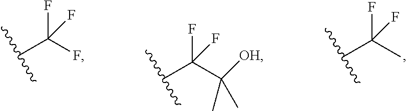

### —NH₂,

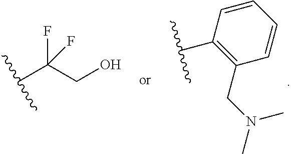 [SMILES: *C(F)(F)CO ; "or" ; *c1ccccc1CN(C)C]

In some embodiments, ring A is phenyl, benzofuranyl, 2,3-dihydrobenzofuranyl, or thienyl, preferably phenyl,

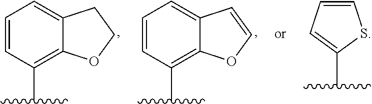

In some embodiments,

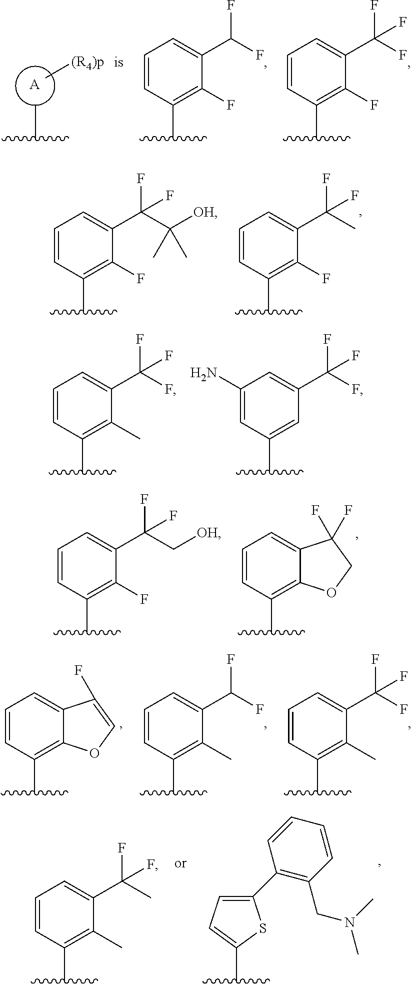

preferably

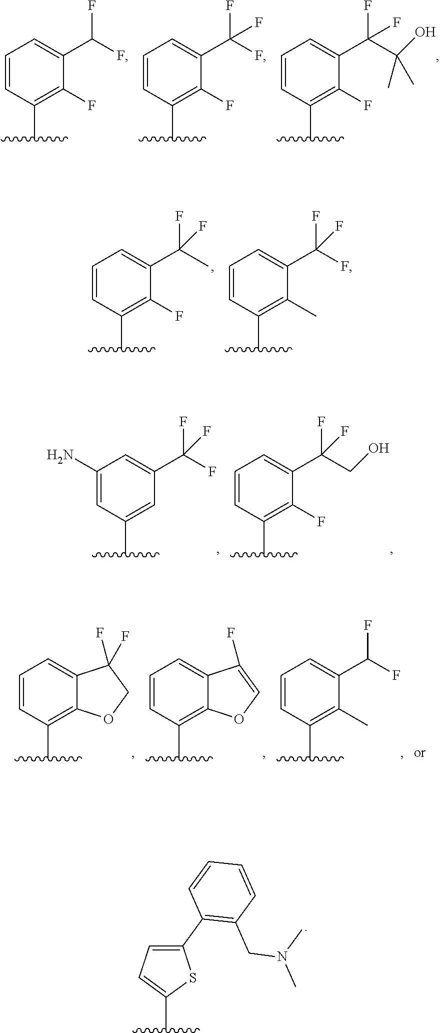

In some embodiments,

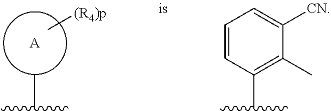

In some embodiments, the fused ring compound represented by formula I is any one of the following compounds:

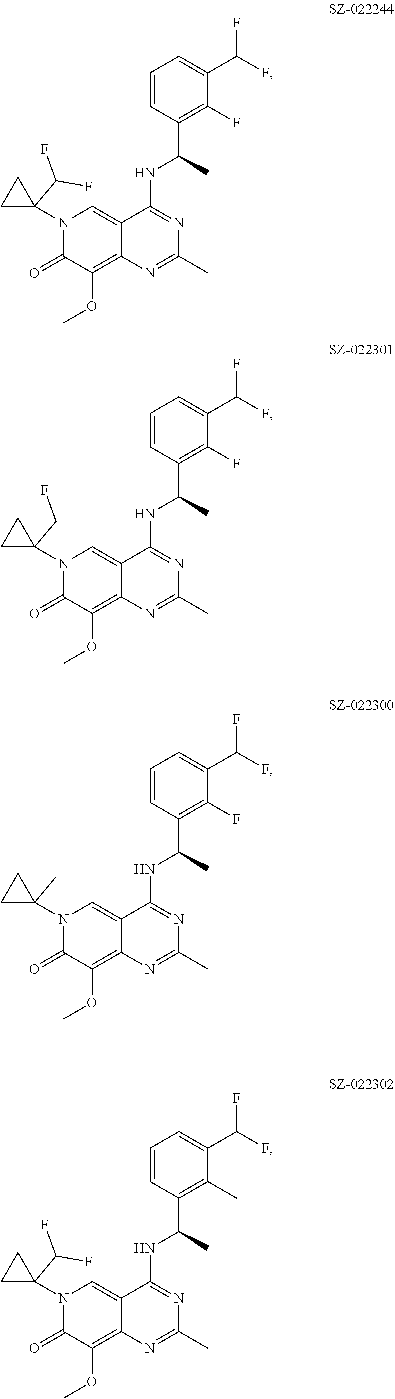 [SMILES: COc1c(=O)n(C2(C(F)F)CC2)cc2c(N[C@H](C)c3cccc(C(F)F)c3F)nc(C)nc12 ; COc1c(=O)n(C2(CF)CC2)cc2c(N[C@H](C)c3cccc(C(F)F)c3F)nc(C)nc12 ; COc1c(=O)n(C2(C)CC2)cc2c(N[C@H](C)c3cccc(C(F)F)c3F)nc(C)nc12 ; COc1c(=O)n(C2(C(F)F)CC2)cc2c(N[C@H](C)c3cccc(C(F)F)c3C)nc(C)nc12]

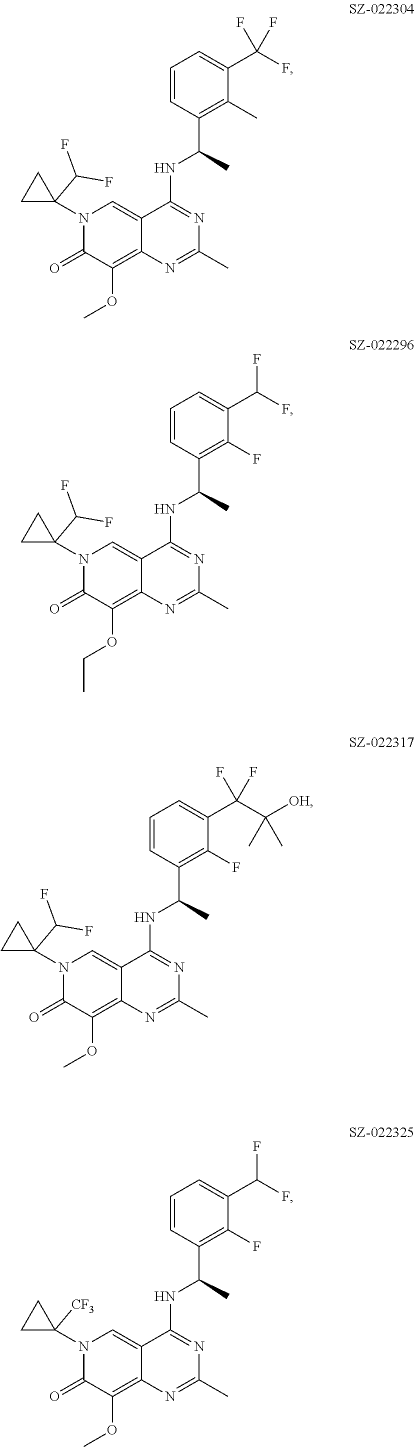 [SMILES: COc1c(=O)n(C2(C(F)F)CC2)cc2c(N[C@H](C)c3cccc(C(F)(F)F)c3C)nc(C)nc12 ; CCOc1c(=O)n(C2(C(F)F)CC2)cc2c(N[C@H](C)c3cccc(C(F)F)c3F)nc(C)nc12 ; COc1c(=O)n(C2(C(F)F)CC2)cc2c(N[C@H](C)c3cccc(C(F)(F)C(C)(C)O)c3F)nc(C)nc12 ; COc1c(=O)n(C2(C(F)(F)F)CC2)cc2c(N[C@H](C)c3cccc(C(F)F)c3F)nc(C)nc12]

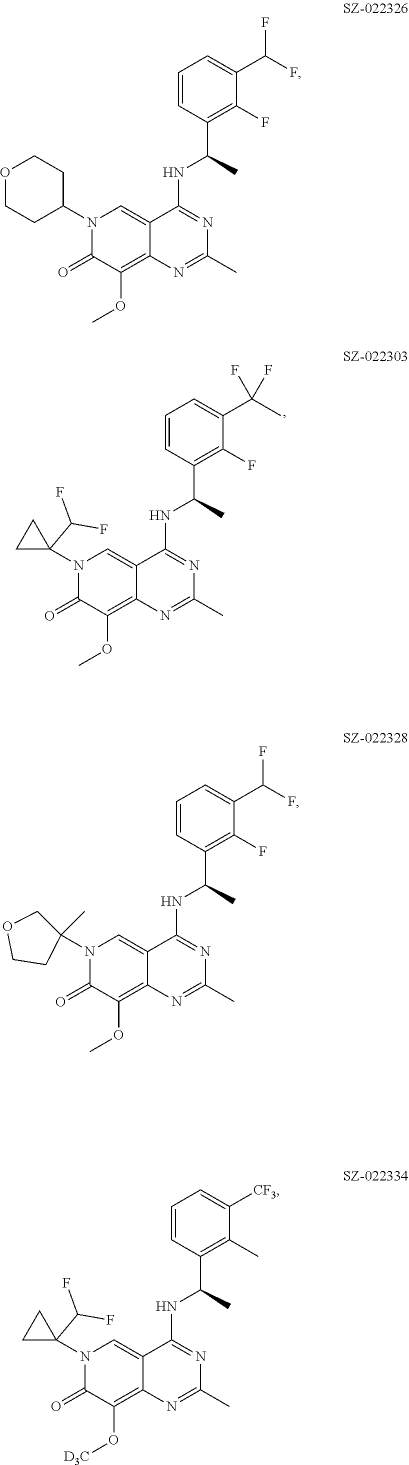 [SMILES: COc1c(=O)n(C2CCOCC2)cc2c(N[C@H](C)c3cccc(C(F)F)c3F)nc(C)nc12 ; COc1c(=O)n(C2(C(F)F)CC2)cc2c(N[C@H](C)c3cccc(C(C)(F)F)c3F)nc(C)nc12 ; COc1c(=O)n(C2(C)CCOC2)cc2c(N[C@H](C)c3cccc(C(F)F)c3F)nc(C)nc12 ; [2H]C([2H])([2H])Oc1c(=O)n(C2(C(F)F)CC2)cc2c(N[C@H](C)c3cccc(C(F)(F)F)c3C)nc(C)nc12]

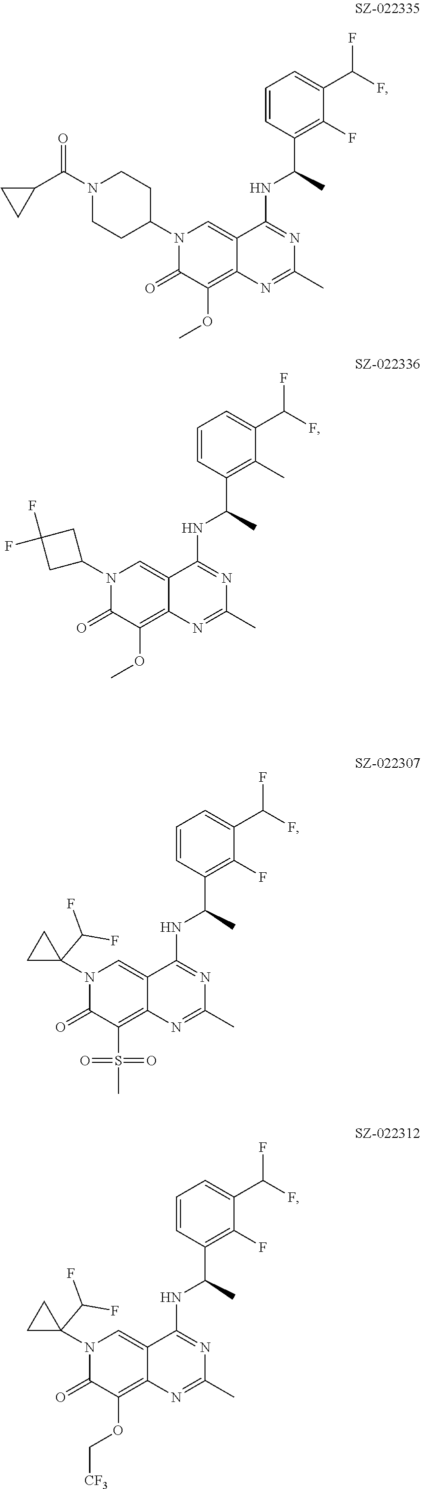 [SMILES: COc1c(=O)n(C2CCN(C(=O)C3CC3)CC2)cc2c(N[C@H](C)c3cccc(C(F)F)c3F)nc(C)nc12 ; COc1c(=O)n(C2CC(F)(F)C2)cc2c(N[C@H](C)c3cccc(C(F)F)c3C)nc(C)nc12 ; Cc1nc(N[C@H](C)c2cccc(C(F)F)c2F)c2cn(C3(C(F)F)CC3)c(=O)c(S(C)(=O)=O)c2n1 ; Cc1nc(N[C@H](C)c2cccc(C(F)F)c2F)c2cn(C3(C(F)F)CC3)c(=O)c(OCC(F)(F)F)c2n1]

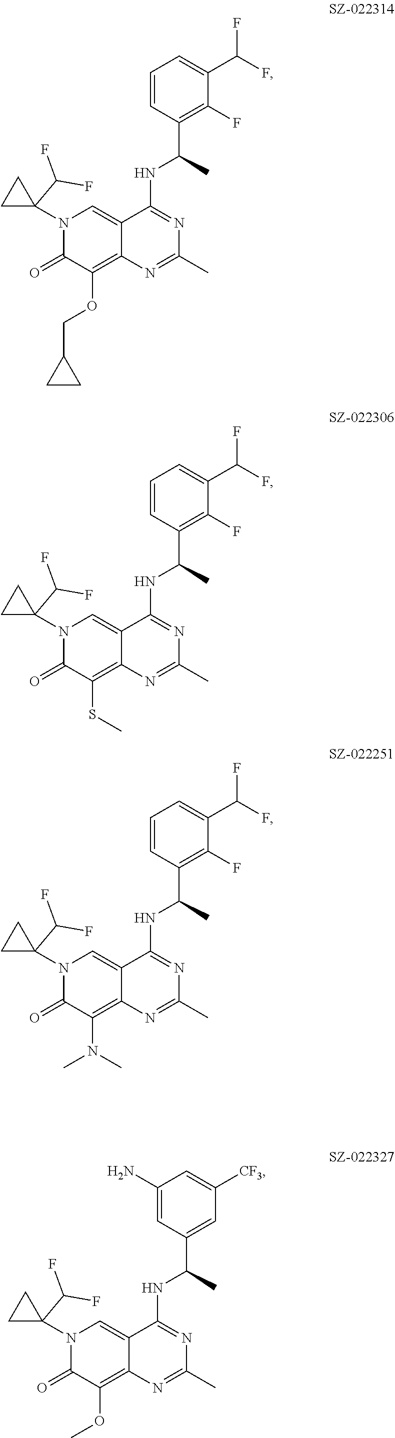 [SMILES: Cc1nc(N[C@H](C)c2cccc(C(F)F)c2F)c2cn(C3(C(F)F)CC3)c(=O)c(OCC3CC3)c2n1 ; CSc1c(=O)n(C2(C(F)F)CC2)cc2c(N[C@H](C)c3cccc(C(F)F)c3F)nc(C)nc12 ; Cc1nc(N[C@H](C)c2cccc(C(F)F)c2F)c2cn(C3(C(F)F)CC3)c(=O)c(N(C)C)c2n1 ; COc1c(=O)n(C2(C(F)F)CC2)cc2c(N[C@H](C)c3cc(N)cc(C(F)(F)F)c3)nc(C)nc12]

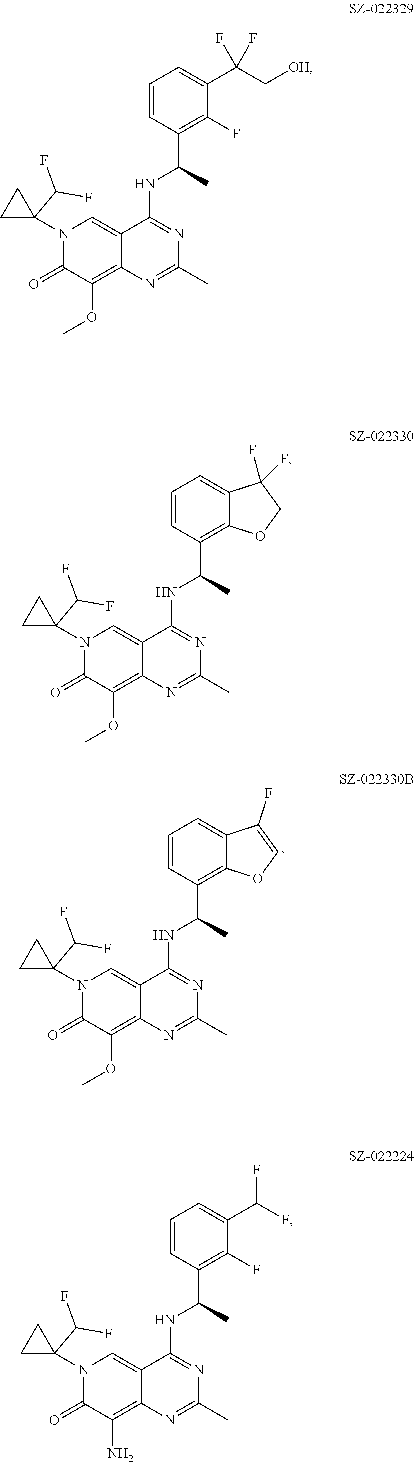 [SMILES: COc1c(=O)n(C2(C(F)F)CC2)cc2c(N[C@H](C)c3cccc(C(F)(F)CO)c3F)nc(C)nc12 ; COc1c(=O)n(C2(C(F)F)CC2)cc2c(N[C@H](C)c3cccc4c3OCC4(F)F)nc(C)nc12 ; COc1c(=O)n(C2(C(F)F)CC2)cc2c(N[C@H](C)c3cccc4c(F)coc34)nc(C)nc12 ; Cc1nc(N[C@H](C)c2cccc(C(F)F)c2F)c2cn(C3(C(F)F)CC3)c(=O)c(N)c2n1]

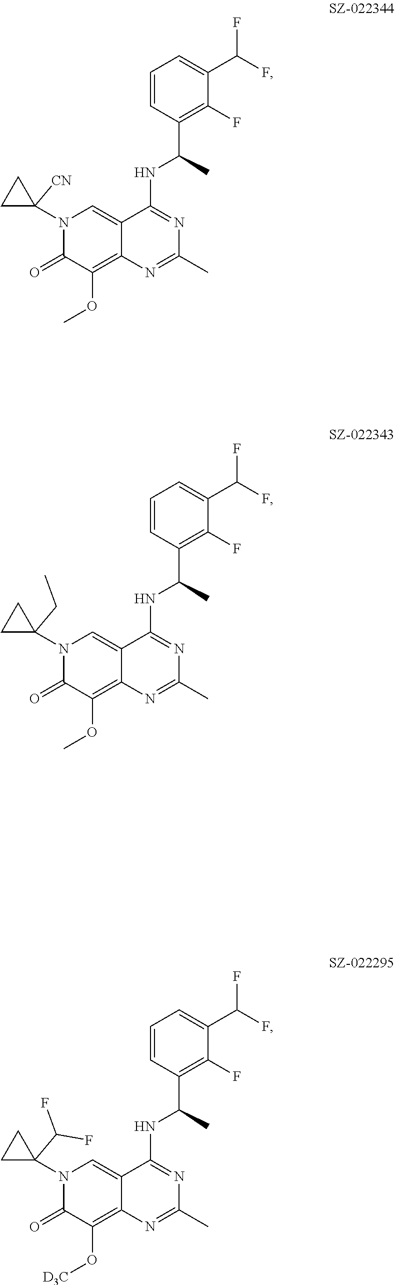 [SMILES: COc1c(=O)n(C2(C#N)CC2)cc2c(N[C@H](C)c3cccc(C(F)F)c3F)nc(C)nc12 ; CCC1(n2cc3c(N[C@H](C)c4cccc(C(F)F)c4F)nc(C)nc3c(OC)c2=O)CC1 ; [2H]C([2H])([2H])Oc1c(=O)n(C2(C(F)F)CC2)cc2c(N[C@H](C)c3cccc(C(F)F)c3F)nc(C)nc12]

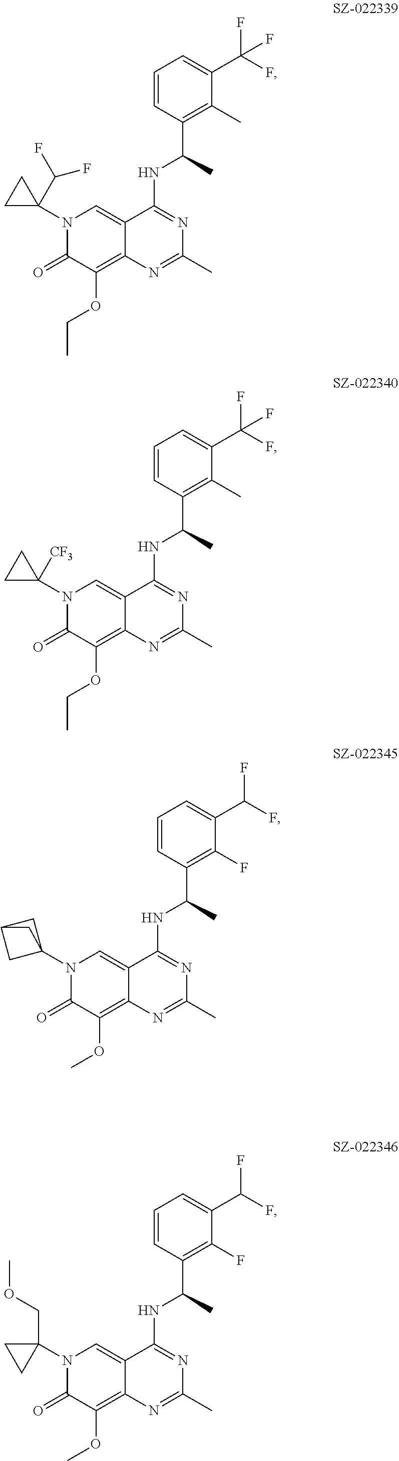 [SMILES: CCOc1c(=O)n(C2(C(F)F)CC2)cc2c(N[C@H](C)c3cccc(C(F)(F)F)c3C)nc(C)nc12 ; CCOc1c(=O)n(C2(C(F)(F)F)CC2)cc2c(N[C@H](C)c3cccc(C(F)(F)F)c3C)nc(C)nc12 ; COc1c(=O)n(C23CC(C2)C3)cc2c(N[C@H](C)c3cccc(C(F)F)c3F)nc(C)nc12 ; COCC1(n2cc3c(N[C@H](C)c4cccc(C(F)F)c4F)nc(C)nc3c(OC)c2=O)CC1]

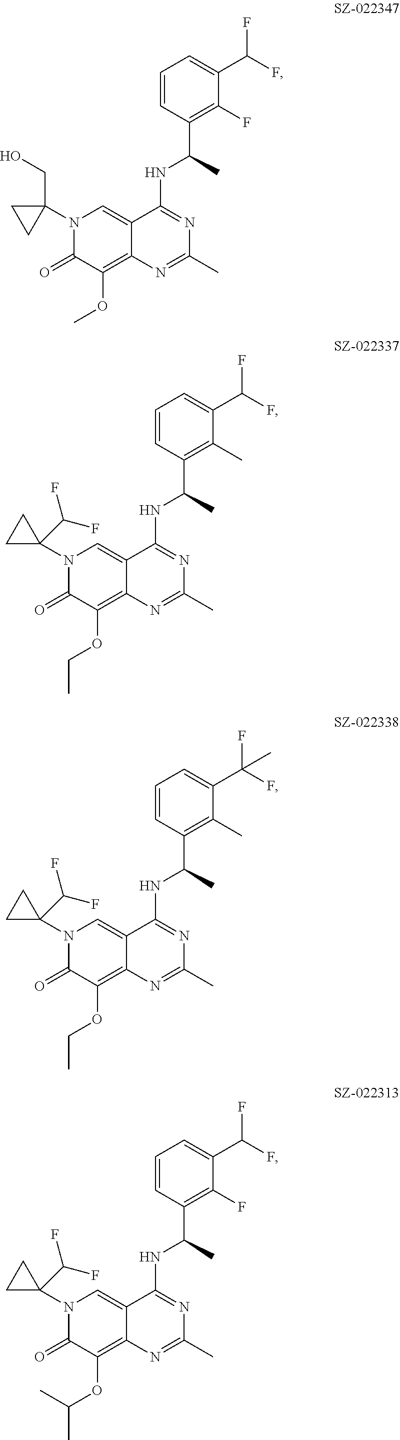 [SMILES: COc1c(=O)n(C2(CO)CC2)cc2c(N[C@H](C)c3cccc(C(F)F)c3F)nc(C)nc12 ; CCOc1c(=O)n(C2(C(F)F)CC2)cc2c(N[C@H](C)c3cccc(C(F)F)c3C)nc(C)nc12 ; CCOc1c(=O)n(C2(C(F)F)CC2)cc2c(N[C@H](C)c3cccc(C(C)(F)F)c3C)nc(C)nc12 ; Cc1nc(N[C@H](C)c2cccc(C(F)F)c2F)c2cn(C3(C(F)F)CC3)c(=O)c(OC(C)C)c2n1]

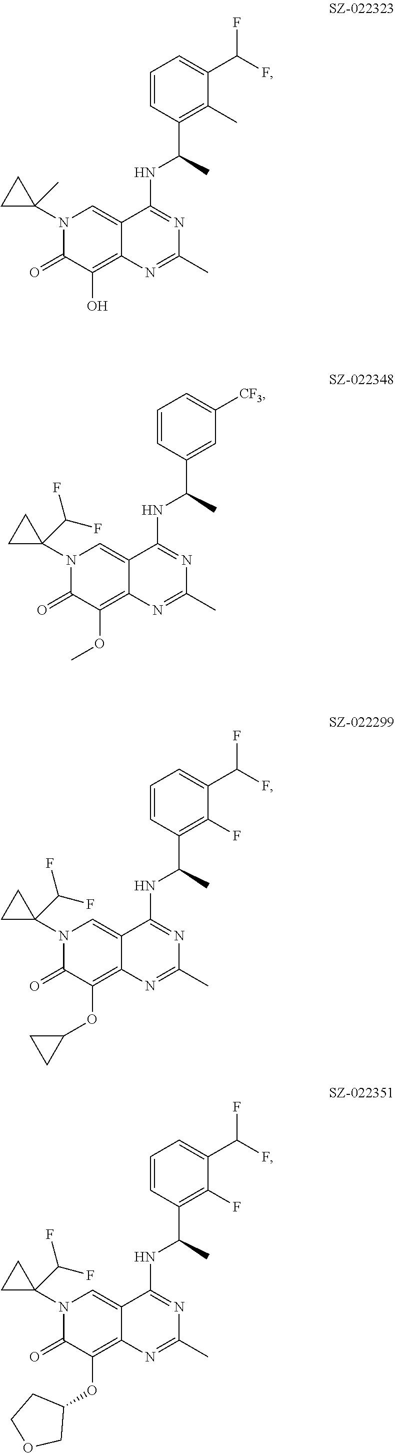 [SMILES: Cc1nc(N[C@H](C)c2cccc(C(F)F)c2C)c2cn(C3(C)CC3)c(=O)c(O)c2n1 ; COc1c(=O)n(C2(C(F)F)CC2)cc2c(N[C@H](C)c3cccc(C(F)(F)F)c3)nc(C)nc12 ; Cc1nc(N[C@H](C)c2cccc(C(F)F)c2F)c2cn(C3(C(F)F)CC3)c(=O)c(OC3CC3)c2n1 ; Cc1nc(N[C@H](C)c2cccc(C(F)F)c2F)c2cn(C3(C(F)F)CC3)c(=O)c(O[C@H]3CCOC3)c2n1]

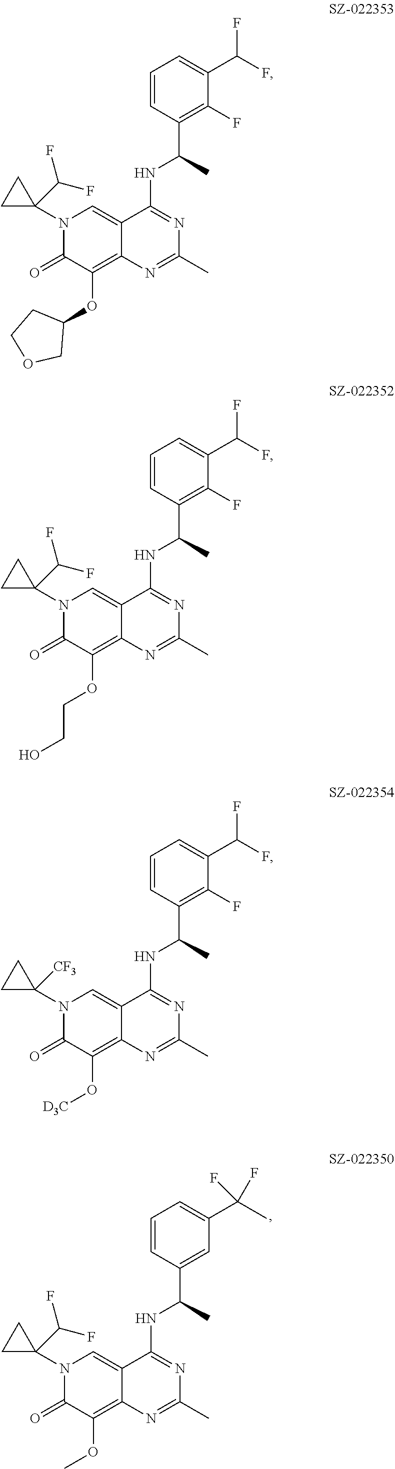 [SMILES: Cc1nc(N[C@H](C)c2cccc(C(F)F)c2F)c2cn(C3(C(F)F)CC3)c(=O)c(O[C@@H]3CCOC3)c2n1 ; Cc1nc(N[C@H](C)c2cccc(C(F)F)c2F)c2cn(C3(C(F)F)CC3)c(=O)c(OCCO)c2n1 ; [2H]C([2H])([2H])Oc1c(=O)n(C2(C(F)(F)F)CC2)cc2c(N[C@H](C)c3cccc(C(F)F)c3F)nc(C)nc12 ; COc1c(=O)n(C2(C(F)F)CC2)cc2c(N[C@H](C)c3cccc(C(C)(F)F)c3)nc(C)nc12]

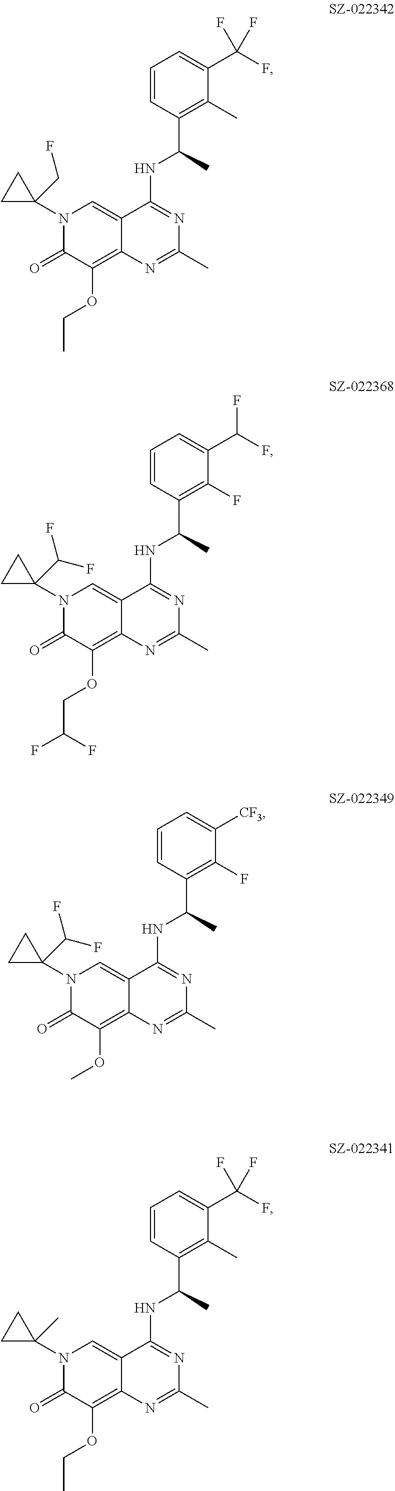 [SMILES: CCOc1c(=O)n(C2(CF)CC2)cc2c(N[C@H](C)c3cccc(C(F)(F)F)c3C)nc(C)nc12 ; Cc1nc(N[C@H](C)c2cccc(C(F)F)c2F)c2cn(C3(C(F)F)CC3)c(=O)c(OCC(F)F)c2n1 ; COc1c(=O)n(C2(C(F)F)CC2)cc2c(N[C@H](C)c3cccc(C(F)(F)F)c3F)nc(C)nc12 ; CCOc1c(=O)n(C2(C)CC2)cc2c(N[C@H](C)c3cccc(C(F)(F)F)c3C)nc(C)nc12]

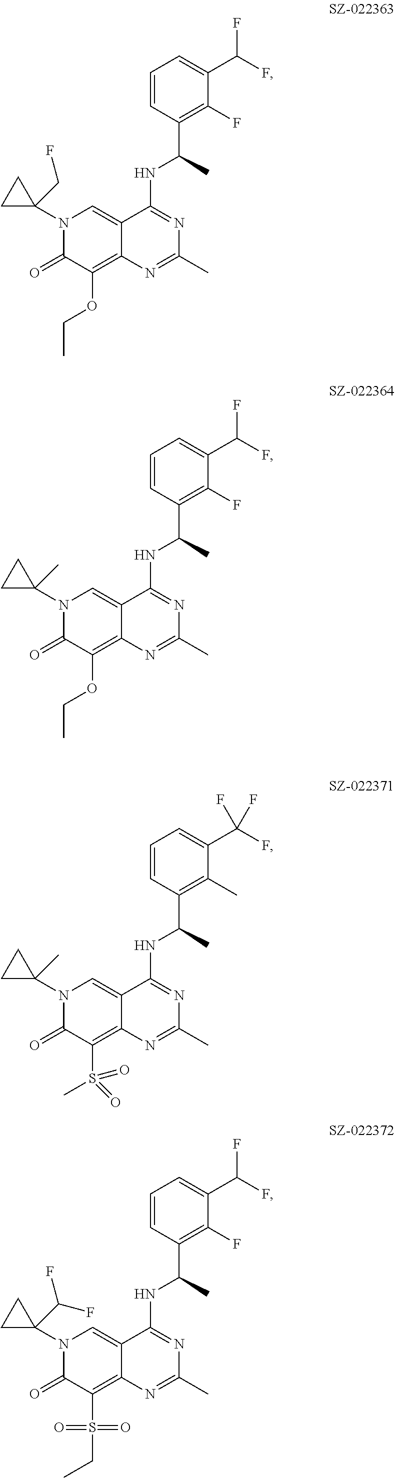 [SMILES: CCOc1c(=O)n(C2(CF)CC2)cc2c(N[C@H](C)c3cccc(C(F)F)c3F)nc(C)nc12 ; CCOc1c(=O)n(C2(C)CC2)cc2c(N[C@H](C)c3cccc(C(F)F)c3F)nc(C)nc12 ; Cc1nc(N[C@H](C)c2cccc(C(F)(F)F)c2C)c2cn(C3(C)CC3)c(=O)c(S(C)(=O)=O)c2n1 ; CCS(=O)(=O)c1c(=O)n(C2(C(F)F)CC2)cc2c(N[C@H](C)c3cccc(C(F)F)c3F)nc(C)nc12]

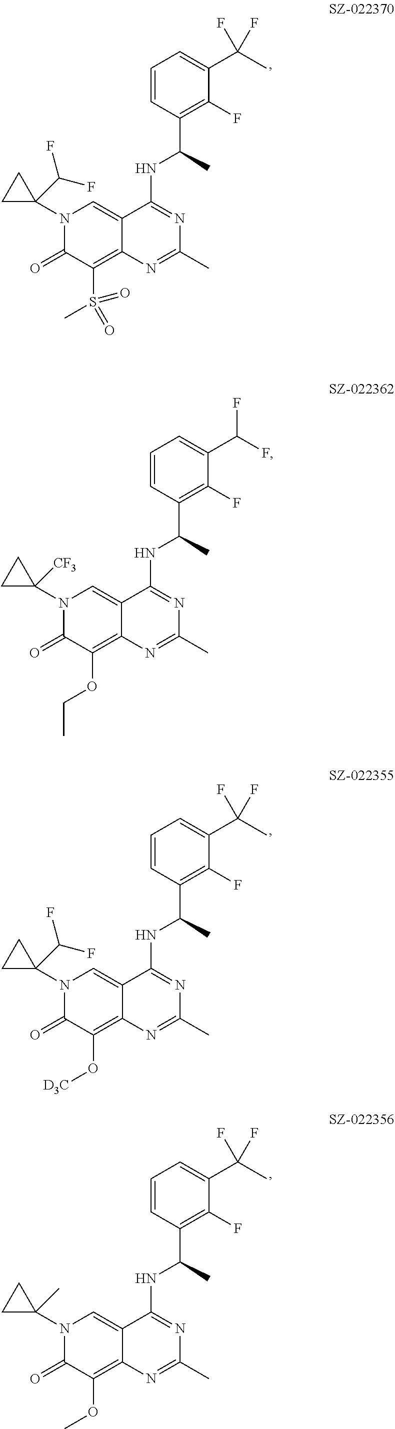 [SMILES: Cc1nc(N[C@H](C)c2cccc(C(C)(F)F)c2F)c2cn(C3(C(F)F)CC3)c(=O)c(S(C)(=O)=O)c2n1 ; CCOc1c(=O)n(C2(C(F)(F)F)CC2)cc2c(N[C@H](C)c3cccc(C(F)F)c3F)nc(C)nc12 ; [2H]C([2H])([2H])Oc1c(=O)n(C2(C(F)F)CC2)cc2c(N[C@H](C)c3cccc(C(C)(F)F)c3F)nc(C)nc12 ; COc1c(=O)n(C2(C)CC2)cc2c(N[C@H](C)c3cccc(C(C)(F)F)c3F)nc(C)nc12]

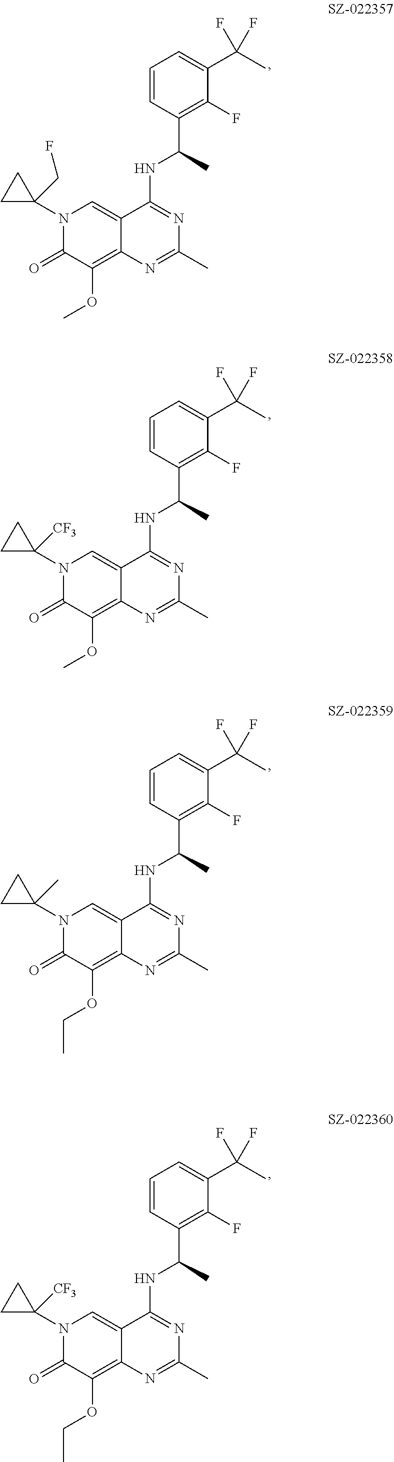 [SMILES: COc1c(=O)n(C2(CF)CC2)cc2c(N[C@H](C)c3cccc(C(C)(F)F)c3F)nc(C)nc12 ; COc1c(=O)n(C2(C(F)(F)F)CC2)cc2c(N[C@H](C)c3cccc(C(C)(F)F)c3F)nc(C)nc12 ; CCOc1c(=O)n(C2(C)CC2)cc2c(N[C@H](C)c3cccc(C(C)(F)F)c3F)nc(C)nc12 ; CCOc1c(=O)n(C2(C(F)(F)F)CC2)cc2c(N[C@H](C)c3cccc(C(C)(F)F)c3F)nc(C)nc12]

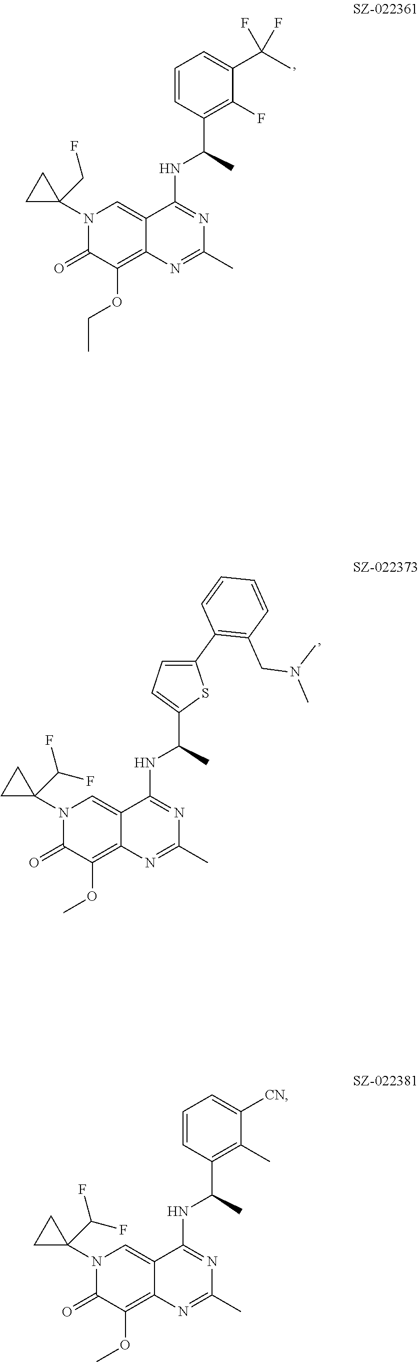 [SMILES: CCOc1c(=O)n(C2(CF)CC2)cc2c(N[C@H](C)c3cccc(C(C)(F)F)c3F)nc(C)nc12 ; COc1c(=O)n(C2(C(F)F)CC2)cc2c(N[C@H](C)c3ccc(-c4ccccc4CN(C)C)s3)nc(C)nc12 ; COc1c(=O)n(C2(C(F)F)CC2)cc2c(N[C@H](C)c3cccc(C#N)c3C)nc(C)nc12]

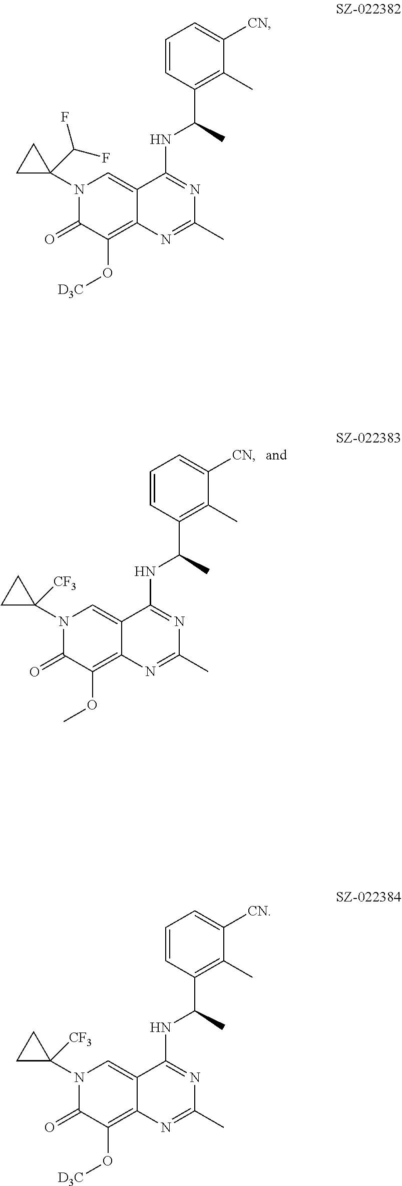 [SMILES: [2H]C([2H])([2H])Oc1c(=O)n(C2(C(F)F)CC2)cc2c(N[C@H](C)c3cccc(C#N)c3C)nc(C)nc12 ; COc1c(=O)n(C2(C(F)(F)F)CC2)cc2c(N[C@H](C)c3cccc(C#N)c3C)nc(C)nc12 ; [2H]C([2H])([2H])Oc1c(=O)n(C2(C(F)(F)F)CC2)cc2c(N[C@H](C)c3cccc(C#N)c3C)nc(C)nc12]

The present invention provides a pharmaceutical composition, comprising:
(1) the fused ring compound represented by formula I or the pharmaceutically acceptable salt thereof as described above, and
(2) a pharmaceutically acceptable auxiliary material.

The present invention further provides use of the fused ring compound represented by formula I or the pharmaceutically acceptable salt as described above or the pharmaceutical composition as described above in the preparation of an SOS1 inhibitor.

In the use, a disease mediated by the SOS1 is lung cancer, pancreatic cancer, pancreatic ductal carcinoma, colon cancer, rectal cancer, appendiceal cancer, esophageal squamous carcinoma, head and neck squamous carcinoma, breast cancer, or other solid tumors.

In the use, the SOS1 inhibitor can be used in a mammalian organism; the SOS1 inhibitor can also be used in vitro, mainly for experimental purposes, for example, used as a standard sample or a control sample to provide comparison, or prepared into a kit according to a conventional method in the art to provide rapid detection for the SOS1 inhibitory effect.

The present invention further provides use of the fused ring compound represented by formula I or the pharmaceutically acceptable salt as described above or the pharmaceutical composition as described above in the preparation of a medicament, wherein the medicament is used for preventing and/or treating one or more of the following diseases:

lung cancer, pancreatic cancer, pancreatic ductal carcinoma, colon cancer, rectal cancer, appendiceal cancer, esophageal squamous carcinoma, head and neck squamous carcinoma, breast cancer, and other solid tumors.

### Definitions and Description

Unless otherwise stated, the following terms and phrases used herein are intended to have the following meanings. A particular term or phrase, unless otherwise specifically defined, should not be considered as indefinite or unclear, but construed according to its common meaning. When referring to a trade name, it is intended to refer to its corresponding commercial product or its active ingredient.

As will be understood by those skilled in the art,

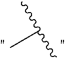

used in the structural formulas describing groups herein means that the corresponding groups are connected to other fragments and groups in the compound through this site, according to conventions used in the art.

As used herein, a substituent may be preceded by a single dash “-” indicating that the named substituent is connected to a parent moiety by a single bond.

In the present invention, the term “pharmaceutically acceptable salt” refers to a salt prepared with the compound of the present invention and a relatively non-toxic, pharmaceutically acceptable acid or base.

In the present invention, the term “pharmaceutical auxiliary material” refers to excipients and additives used in the manufacture of a pharmaceutical product and in the formulation of pharmaceutical formulas, and refers to all substances, other than the active ingredient, contained in a pharmaceutical preparation. See Volume 4 of Pharmacopoeia of The People's Republic of China (2015 Edition), or Handbook of Pharmaceutical Excipients (Raymond C Rowe, 2009 Sixth Edition).

In the present invention, the term “substituted” or “substituent” refers to the replacement of a hydrogen atom in a group by the designated group. When the substitution position is not specified, the substitution can be at any position, but is permissible only if it results in the formation of a stable or chemically feasible chemical. An example is given as follows: structure

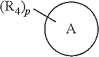

indicates that the hydrogen atom on ring A is replaced by p R₄.

When any variable (e.g., R) occurs more than once in the constitution or structure of a compound, the variable is independently defined in each case. Thus, for example, if a group is substituted with one or more R, the group can optionally be substituted with at least one R, and the definition of R in each case is independent. Furthermore, a combination of the substituent and/or the variant thereof is permissible only if the combination can result in a stable compound.

The term “more” refers to 2, 3, 4, or 5, preferably 2 or 3.

In the present invention, the term “alkyl” refers to a saturated linear or branched chain monovalent hydrocarbon group. C₁-C₆ alkyl refers to an alkyl group having 1-6 carbon atoms, preferably an alkyl group having 1-4 carbon atoms, specifically methyl, ethyl, n-propyl, isopropyl, n-butyl, isobutyl, sec-butyl, or tert-butyl.

In the present invention, the term “cycloalkyl” refers to a saturated monocyclic, bridged or spiro cyclic group having a specified number of ring carbon atoms (e.g., C₃₋₁₀), wherein the ring atoms are composed only of carbon atoms. The C₃₋₁₀ cycloalkyl can specifically be 3-, 4-, 5-, 6-, 7-, 8-, 9-, or 10-membered cycloalkyl, including cyclopropyl, cyclobutyl, cyclopentyl, cyclohexyl, and the like. Specific examples of the cycloalkyl include, but are not limited to, cyclopropyl, cyclobutyl, cyclopentyl, cyclohexyl, and

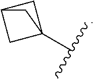 [SMILES: *C12CC(C1)C2]

In the present invention, the term “aryl” refers to an aromatic group in which at least one ring is aromatic, such as a benzene ring.

In the present invention, the term “heteroaryl” refers to an aromatic group containing a heteroatom, preferably an aromatic 5- to 6-membered monocyclic or 9- to 10-membered bicyclic ring containing 1, 2 or 3 heteroatoms independently selected from nitrogen, oxygen and sulfur (when the heteroaryl is bicyclic, each ring is aromatic), such as thienyl, furanyl, pyridinyl, benzofuranyl and the like.

In the present invention, the term “heterocyclyl” refers to a saturated or unsaturated, non-aromatic, monocyclic or polycyclic (e.g., fused, spiro, or bridged) cyclic group formed from carbon atoms and at least one heteroatom independently selected from N, O and S. Examples of the heterocyclyl include, but are not limited to

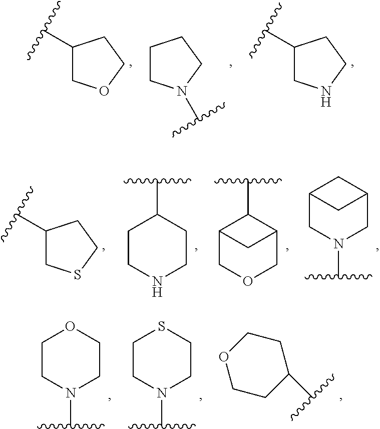

or 2,3-dihydrobenzofuranyl. 5- to 10-membered heterocyclyl may specifically be 5-, 6-, 7-, 8-, 9- or 10-membered heterocyclyl. 3- to 10-membered heterocyclyl may specifically be 3-, 4-, 5-, 6-, 7-, 8-, 9- or 10-membered heterocyclyl.

In the present invention, the term “halogen” refers to fluorine, chlorine, bromine, or iodine.

In the present invention, the term “C₁₋₆ alkyl substituted with one or more halogens” refers to a group formed by substituting one or more (e.g., 2, 3, 4, 5, or 6) hydrogen atoms in an alkyl group with a halogen, wherein each halogen is independently F, Cl, Br, or I.

The above preferred conditions may be combined arbitrarily to obtain preferred embodiments of the present invention without departing from the general knowledge in the art.

The reagents and starting materials used in the present invention are commercially available.

The positive and progressive effects of the present invention are as follows: the compound has a novel structure and good SOS1 inhibitory activity.

### DETAILED DESCRIPTION

The present invention is further illustrated by the following examples, which are not intended to limit the present invention. Experimental procedures without specified conditions in the following examples were performed in accordance with conventional procedures and conditions, or in accordance with instructions.

### Example 1: SZ-022244

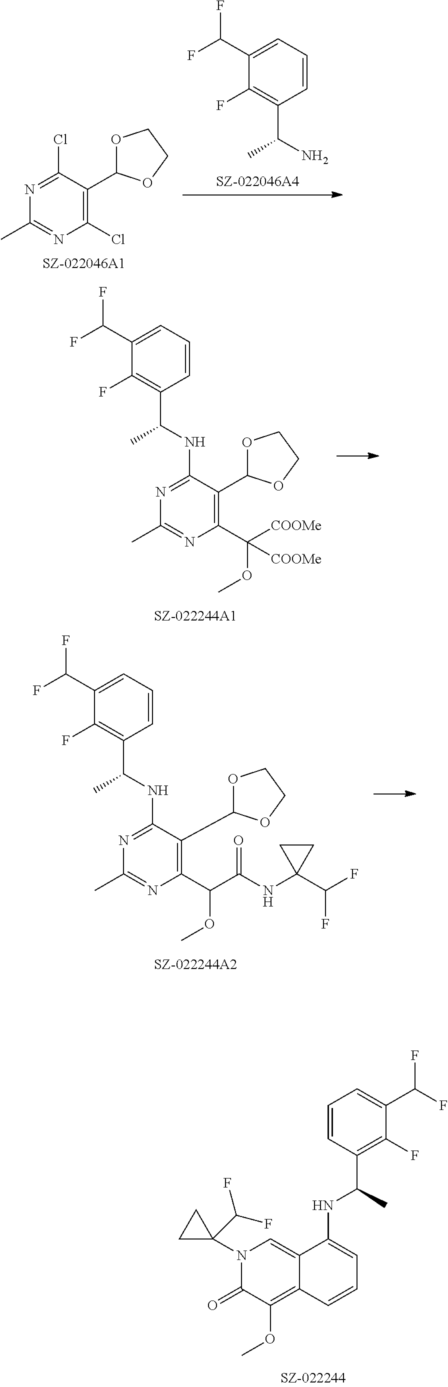 [SMILES: Cc1nc(Cl)c(C2OCCO2)c(Cl)n1 ; C[C@@H](N)c1cccc(C(F)F)c1F ; COC(=O)C(OC)(C(=O)OC)c1nc(C)nc(N[C@H](C)c2cccc(C(F)F)c2F)c1C1OCCO1 ; COC(C(=O)NC1(C(F)F)CC1)c1nc(C)nc(N[C@H](C)c2cccc(C(F)F)c2F)c1C1OCCO1 ; COc1c(=O)n(C2(C(F)F)CC2)cc2c(N[C@H](C)c3cccc(C(F)F)c3F)cccc12]

### Step 1:

Compound SZ-022046A1 (800 mg, 3.48 mmol, synthesized according to WO2019122129), dimethyl methoxymalonate (560 mg, 3.48 mmol), and potassium carbonate (960 mg, 6.96 mmol) were added to dimethyl sulfoxide (10 mL), and the mixture was heated to 100° C. for reaction for 1 h. After the starting materials were consumed as monitored by LC-MS, the reaction mixture was cooled to 60° C. SZ-022046A4 (1.18 g, 5.22 mmol, synthesized according to WO2019122129) and triethylamine (528 mg, 5.22 mmol) were added, and the resulting mixture was allowed to react at 60° C. for 3 h. After the starting materials were consumed as monitored by LC-MS, the reaction mixture was cooled to room temperature. Water (50 mL) and ethyl acetate (50 mL) were added to the reaction mixture for extraction, and the organic layer was separated, dried, and concentrated by rotary evaporation to give a crude product. The crude product was purified by flash silica gel column chromatography (PE:EA=3:1; Rf=0.4) to give product SZ-022244A1 as a yellow oil (170 mg). LC-MS: [M+H]⁺ 514.15.

### Step 2:

SZ-022244A1 (170 mg, 0.33 mmol) was added to dimethyl sulfoxide (2 mL), and then a 20% sodium hydroxide solution (150 μL) was added. The mixture was allowed to react at 40° C. for 2 h. 1-(Difluoromethyl)cyclopropan-1-amine hydrochloride (62 mg, 0.43 mmol), HATU (189 mg, 0.50 mmol), and triethylamine (67 mg, 0.66 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022244A2 as a yellow viscous substance (170 mg). LC-MS: [M+H]⁺ 531.19.

### Step 3:

SZ-022244A2 (170 mg, 0.32 mmol) was added to isopropanol (2 mL), and then a hydrochloric acid solution (2 M, 0.8 mL) was added. The mixture was allowed to react at 50° C. for 4 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to reversed-phase column chromatography (acetonitrile/water with 0.1% formic acid) and then lyophilized to give SZ-022244 (49.1 mg). LC-MS: [M+H]⁺ 469.13.

SZ-022244: ¹H NMR (400 MHz, DMSO-d₆) δ 8.92 (s, 1H), 8.79 (d, J=6.8 Hz, 1H), 7.64 (t, J=7.6 Hz, 1H), 7.52 (t, J=7.2 Hz, 1H), 7.32 (t, J=8 Hz, 1H), 7.23 (t, J=56 Hz, 1H), 6.34 (t, J=56 Hz, 1H), 5.80-5.70 (m, 1H), 3.78 (s, 3H), 2.21 (s, 3H), 1.58 (d, J=7.2 Hz, 3H), 1.53-1.46 (m, 2H), 1.45-1.33 (m, 2H).

### Example 2: SZ-022300

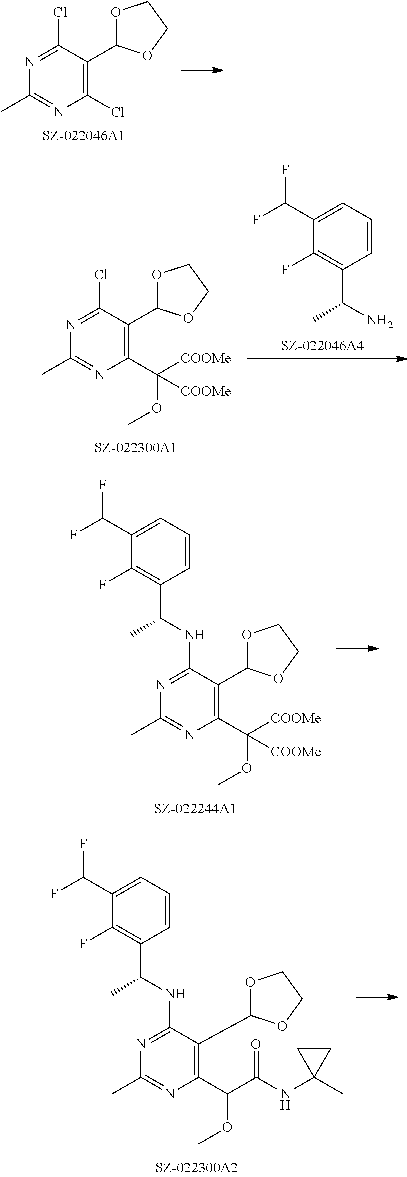 [SMILES: Cc1nc(Cl)c(C2OCCO2)c(Cl)n1 ; COC(=O)C(OC)(C(=O)OC)c1nc(C)nc(Cl)c1C1OCCO1 ; C[C@@H](N)c1cccc(C(F)F)c1F ; COC(=O)C(OC)(C(=O)OC)c1nc(C)nc(N[C@H](C)c2cccc(C(F)F)c2F)c1C1OCCO1 ; COC(C(=O)NC1(C)CC1)c1nc(C)nc(N[C@H](C)c2cccc(C(F)F)c2F)c1C1OCCO1]

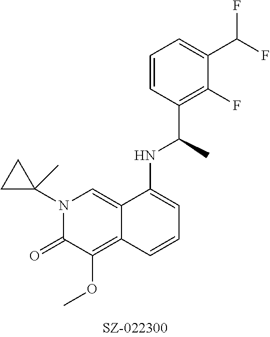 [SMILES: COc1c(=O)n(C2(C)CC2)cc2c(N[C@H](C)c3cccc(C(F)F)c3F)cccc12]

### Step 1:

SZ-022046A1 (3.5 g, 15 mmol, synthesized according to WO2019122129) was added to dimethyl sulfoxide (30 mL), and the mixture was heated to 100° C. Potassium carbonate (4.14 g, 30 mmol) and dimethyl methoxymalonate (3 g, 18.5 mmol) were then added, and the mixture was allowed to react for 2 h with the temperature maintained at 100° C. After the starting materials were consumed as monitored by LC-MS, water (100 mL) and ethyl acetate (100 mL) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA=3:1; Rf=0.4) to give product SZ-022300A1 as a yellow oil (3 g). LC-MS: [M+H]⁺ 361.11, 363.11.

### Step 2:

SZ-022300A1 (670 mg, 1.86 mmol), SZ-022046A4 (462 mg, 2 mmol, synthesized according to WO2019122129), and triethylamine (566 mg, 5.56 mmol) were added to dimethyl sulfoxide (5 mL), and the mixture was heated to 60° C. for reaction for 8 h. After the starting materials were consumed as monitored by LC-MS, water (100 mL) and ethyl acetate (100 mL) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA=3:1; Rf=0.35) to give product SZ-022244A1 as a yellow oil (540 mg). LC-MS: [M+H]⁺ 514.19.

### Step 3:

SZ-022244A1 (150 mg, 0.29 mmol) was added to dimethyl sulfoxide (2 mL), and then a 20% sodium hydroxide solution (150 μL) was added. The mixture was allowed to react at 40° C. for 1 h. 1-Methylcyclopropanamine hydrochloride (41 mg, 0.38 mmol), HATU (167 mg, 0.44 mmol), and triethylamine (59 mg, 0.58 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022300A2 as a yellow viscous substance (150 mg). LC-MS: [M+H]⁺ 494.89.

### Step 4:

SZ-022300A2 (150 mg, 0.30 mmol) was added to isopropanol (2 mL), and then a hydrochloric acid solution (2 M, 0.75 mL) was added. The mixture was allowed to react at 50° C. for 4 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022300 (66 mg). LC-MS: [M+H]⁺ 432.94.

SZ-022300: ¹H NMR (400 MHz, DMSO-d₆) δ 8.99 (s, 1H), 8.69 (d, J=7.2 Hz, 1H), 7.66 (t, J=7.6 Hz, 1H), 7.52 (t, J=7.2 Hz, 1H), 7.31 (t, J=7.6 Hz, 1H), 7.23 (t, J=54.4 Hz, 1H), 5.80-5.70 (m, 1H), 3.75 (s, 3H), 2.20 (s, 3H), 1.58 (d, J=7.2 Hz, 3H), 1.52 (s, 3H), 1.17-1.09 (m, 2H), 1.09-1.00 (m, 2H).

### Example 3: SZ-022296

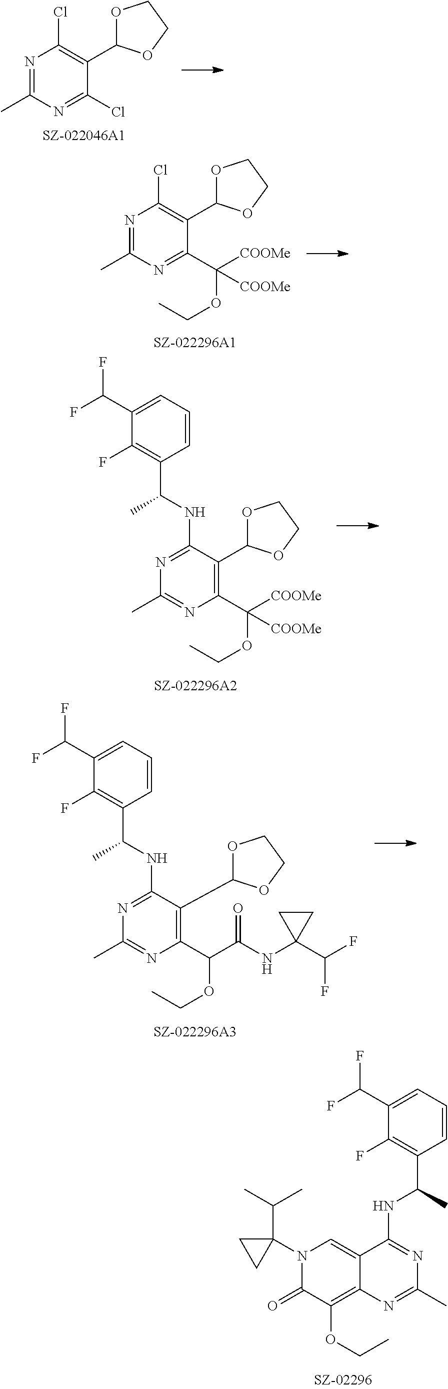 [SMILES: Cc1nc(Cl)c(C2OCCO2)c(Cl)n1 ; CCOC(C(=O)OC)(C(=O)OC)c1nc(C)nc(Cl)c1C1OCCO1 ; CCOC(C(=O)OC)(C(=O)OC)c1nc(C)nc(N[C@H](C)c2cccc(C(F)F)c2F)c1C1OCCO1 ; CCOC(C(=O)NC1(C(F)F)CC1)c1nc(C)nc(N[C@H](C)c2cccc(C(F)F)c2F)c1C1OCCO1 ; CCOc1c(=O)n(C2(C(C)C)CC2)cc2c(N[C@H](C)c3cccc(C(F)F)c3F)nc(C)nc12]

### Step 1:

Compound SZ-022046A1 (500 mg, 2.12 mmol, synthesized according to WO2019122129), diethyl ethoxymalonate (502 mg, 2.55 mmol), and potassium carbonate (560 mg, 4.25 mmol) were added to dimethyl sulfoxide (5 mL), and the mixture was heated to 100° C. for reaction for 1 h. After the starting materials were consumed as monitored by LC-MS, the reaction mixture was cooled to room temperature. Water (50 mL) and ethyl acetate (50 mL) were added to the reaction mixture for extraction, and the organic layer was separated, dried, and concentrated by rotary evaporation to give a crude product. The crude product was purified by flash silica gel column chromatography (PE:EA=5:1; Rf=0.4) to give product SZ-022296A1 as a colorless oil (310 mg). LC-MS: [M+H]⁺ 402.8.

### Step 2:

Compound SZ-022296A1 (310 mg, 0.77 mmol), (1R)-1-(3-(difluoromethyl)-2-fluorophenyl)ethylamine hydrochloride (208 mg, 0.92 mmol, synthesized according to WO2019122129), and triethylamine (234 mg, 2.31 mmol) were allowed to react at 60° C. for 3 h. After the starting materials were consumed as monitored by LC-MS, the reaction mixture was cooled to room temperature. Water (50 mL) and ethyl acetate (50 mL) were added to the reaction mixture for extraction, and the organic layer was separated, dried, and concentrated by rotary evaporation to give a crude product. The crude product was purified by flash silica gel column chromatography (PE:EA=3:1; Rf=0.3) to give product SZ-022296A2 as a light yellow oil (110 mg). LC-MS: [M+H]⁺ 556.09.

### Step 3:

SZ-022296A2 (110 mg, 0.20 mmol) was added to dimethyl sulfoxide (2 mL), and then a 20% sodium hydroxide solution (100 μL) was added. The mixture was allowed to react at 35° C. for 2 h. 1-(Difluoromethyl)cyclopropan-1-amine hydrochloride (62 mg, 0.43 mmol), HATU (189 mg, 0.50 mmol), and triethylamine (37 mg, 0.26 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022296A3 as a yellow viscous substance (120 mg). LC-MS: [M+H]⁺ 545.04.

### Step 4:

SZ-022296A3 (120 mg, 0.20 mmol) was added to isopropanol (2 mL), and then a hydrochloric acid solution (2 M, 0.5 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022296 (43 mg). LC-MS: [M+H]⁺ 482.91.

SZ-022296: ¹H NMR (400 MHz, DMSO-d₆) δ 8.91 (s, 1H), 8.80 (d, J=7.6 Hz, 1H), 7.64 (t, J=7.2 Hz, 1H), 7.52 (t, J=6.8 Hz, 1H), 7.32 (t, J=7.6 Hz, 1H), 7.23 (t, J=54.4 Hz, 1H), 6.34 (t, J=56.8 Hz, 1H), 5.81-5.71 (m, 1H), 4.08 (q, J=7.2 Hz, 2H), 2.21 (s, 3H), 1.58 (d, J=7.2 Hz, 3H), 1.52-1.45 (m, 2H), 1.44-1.32 (m, 2H), 1.22 (t, J=7.20 Hz, 3H).

### Example 4: SZ-022301

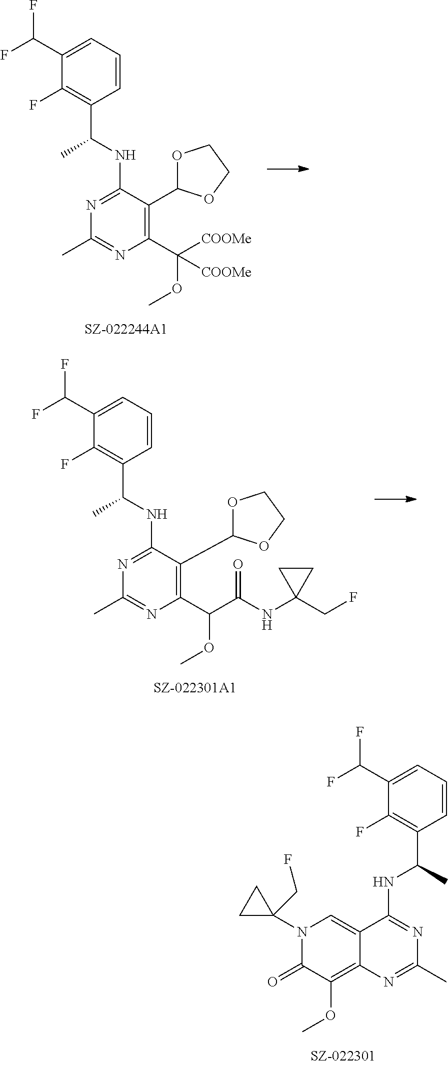 [SMILES: COC(=O)C(OC)(C(=O)OC)c1nc(C)nc(N[C@H](C)c2cccc(C(F)F)c2F)c1C1OCCO1 ; COC(C(=O)NC1(CF)CC1)c1nc(C)nc(N[C@H](C)c2cccc(C(F)F)c2F)c1C1OCCO1 ; COc1c(=O)n(C2(CF)CC2)cc2c(N[C@H](C)c3cccc(C(F)F)c3F)nc(C)nc12]

### Step 1:

SZ-022244A1 (190 mg, 0.37 mmol) was added to dimethyl sulfoxide (2 mL), and then a 20% sodium hydroxide solution (150 μL) was added. The mixture was allowed to react at 40° C. for 2 h. 1-(Monofluoromethyl)cyclopropanamine hydrochloride (61 mg, 0.48 mmol), HATU (212 mg, 0.56 mmol), and triethylamine (75 mg, 0.74 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022301A1 as a yellow viscous substance (190 mg). LC-MS: [M+H]⁺ 513.22.

### Step 2:

SZ-022301A1 (190 mg, 0.37 mmol) was added to isopropanol (2 mL), and then a hydrochloric acid solution (2 M, 0.93 mL) was added. The mixture was allowed to react at 50° C. for 4 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022301 (53 mg). LC-MS: [M+H]⁺ 450.92.

SZ-022301: ¹H NMR (400 MHz, DMSO-d₆) δ 8.96 (s, 1H), 8.90-8.805 (br, 1H), 7.64 (t, J=7.6 Hz, 1H), 7.52 (t, J=7.2 Hz, 1H), 7.32 (t, J=7.6 Hz, 1H), 7.23 (t, J=54.4 Hz, 1H), 5.81-5.71 (m, 1H), 4.80-4.47 (m, 2H), 3.77 (s, 3H), 2.22 (s, 3H), 1.58 (d, J=7.2 Hz, 3H), 1.38-1.22 (m, 4H).

### Example 5: SZ-022302

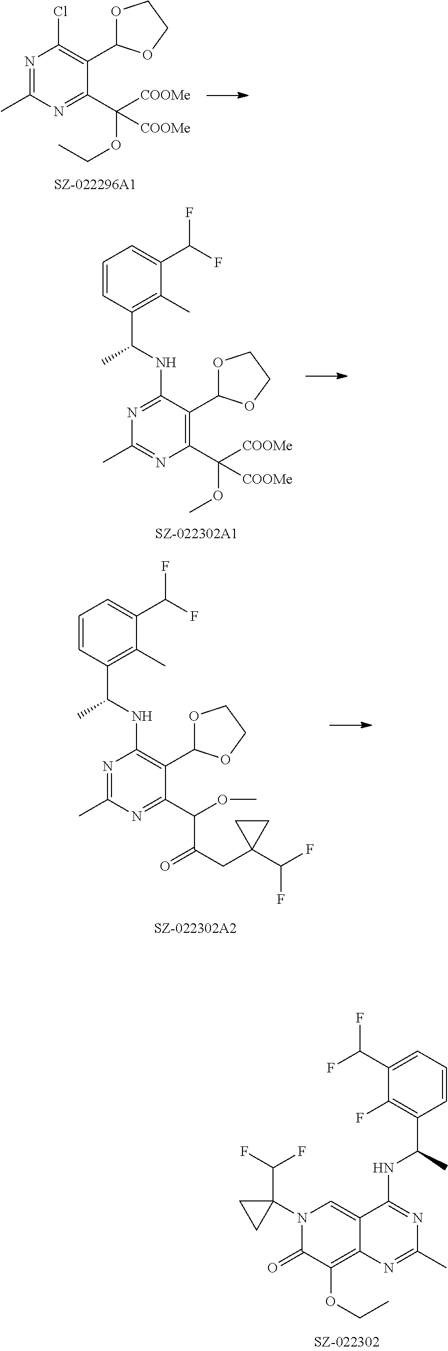 [SMILES: CCOC(C(=O)OC)(C(=O)OC)c1nc(C)nc(Cl)c1C1OCCO1 ; COC(=O)C(OC)(C(=O)OC)c1nc(C)nc(N[C@H](C)c2cccc(C(F)F)c2C)c1C1OCCO1 ; COC(C(=O)CC1(C(F)F)CC1)c1nc(C)nc(N[C@H](C)c2cccc(C(F)F)c2C)c1C1OCCO1 ; CCOc1c(=O)n(C2(C(F)F)CC2)cc2c(N[C@H](C)c3cccc(C(F)F)c3F)nc(C)nc12]

### Step 1:

Compound SZ-022300A1 (200 mg, 0.55 mmol), (1R)-1-(3-(difluoromethyl)-2-methylphenyl)ethylamine hydrochloride (147 mg, 0.67 mmol, synthesized according to WO2019122129), and triethylamine (112 mg, 1.11 mmol) were allowed to react at 60° C. for 3 h. After the starting materials were consumed as monitored by LC-MS, the reaction mixture was cooled to room temperature. Water (50 mL) and ethyl acetate (50 mL) were added to the reaction mixture for extraction, and the organic layer was separated, dried, and concentrated by rotary evaporation to give a crude product. The crude product was purified by flash silica gel column chromatography (PE:EA=3:1; Rf=0.2) to give product SZ-022302A1 as a colorless oil (100 mg). LC-MS: [M+H]⁺ 510.92.

### Step 2:

SZ-022302A1 (100 mg, 0.19 mmol) was added to dimethyl sulfoxide (2 mL), and then a 20% sodium hydroxide solution (100 μL) was added. The mixture was allowed to react at 35° C. for 2 h. 1-(Difluoromethyl)cyclopropan-1-amine hydrochloride (37 mg, 0.26 mmol), HATU (111 mg, 0.29 mmol), and triethylamine (40 mg, 0.39 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022302A2 as a yellow viscous substance (180 mg). LC-MS: [M+H]⁺ 527.11.

### Step 3:

SZ-022302A2 (180 mg, 0.19 mmol) was added to isopropanol (2 mL), and then a hydrochloric acid solution (2 M, 0.5 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022302 (45 mg). LC-MS: [M+H]⁺+465.17.

SZ-022302: ¹H NMR (400 MHz, DMSO-d₆) δ 8.91 (s, 1H), 8.83 (d, J=7.2 Hz, 1H), 7.60 (d, J=8.0 Hz, 1H), 7.42 (d, J=8.0 Hz, 1H), 7.34 (d, J=8.0 Hz, 1H), 7.21 (t, J=44.8 Hz, 1H), 6.34 (t, J=57.2 Hz, 1H), 5.78-5.68 (m, 1H), 3.77 (s, 3H), 2.51 (s, 3H), 2.24 (s, 3H), 1.53 (d, J=7.2 Hz, 3H), 1.51-1.45 (m, 2H), 1.44-1.32 (m, 2H).

### Example 6: SZ-022304

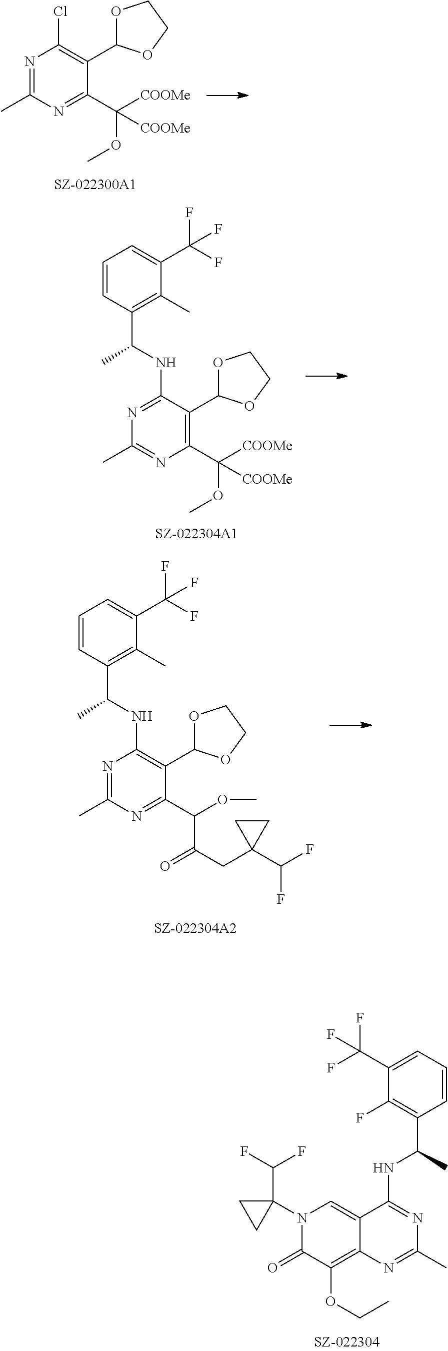 [SMILES: COC(=O)C(OC)(C(=O)OC)c1nc(C)nc(Cl)c1C1OCCO1 ; COC(=O)C(OC)(C(=O)OC)c1nc(C)nc(N[C@H](C)c2cccc(C(F)(F)F)c2C)c1C1OCCO1 ; COC(C(=O)CC1(C(F)F)CC1)c1nc(C)nc(N[C@H](C)c2cccc(C(F)(F)F)c2C)c1C1OCCO1 ; CCOc1c(=O)n(C2(C(F)F)CC2)cc2c(N[C@H](C)c3cccc(C(F)(F)F)c3F)nc(C)nc12]

### Step 1:

Compound SZ-022300A1 (200 mg, 0.55 mmol), (1R)-1-(3-(trifluoromethyl)-2-methylphenyl)ethylamine hydrochloride (170 mg, 0.71 mmol, synthesized according to WO2019122129), and triethylamine (112 mg, 1.11 mmol) were allowed to react at 60° C. for 3 h. After the starting materials were consumed as monitored by LC-MS, the reaction mixture was cooled to room temperature. Water (50 mL) and ethyl acetate (50 mL) were added to the reaction mixture for extraction, and the organic layer was separated, dried, and concentrated by rotary evaporation to give a crude product. The crude product was purified by flash silica gel column chromatography (PE:EA=3:1; Rf=0.3) to give product SZ-022304A1 as a colorless oil (150 mg). LC-MS: [M+H]⁺ 528.22.

### Step 2:

SZ-022304A1 (150 mg, 0.28 mmol) was added to dimethyl sulfoxide (2 mL), and then a 20% sodium hydroxide solution (100 μL) was added. The mixture was allowed to react at 35° C. for 2 h. 1-(Difluoromethyl)cyclopropan-1-amine hydrochloride (53 mg, 0.37 mmol), HATU (162 mg, 0.43 mmol), and triethylamine (58 mg, 0.57 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022304A2 as a light yellow viscous substance (150 mg). LC-MS: [M+H]⁺ 545.08.

### Step 3:

SZ-022304A2 (150 mg, 0.28 mmol) was added to isopropanol (2 mL), and then a hydrochloric acid solution (2 M, 0.5 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022304 (65 mg). LC-MS: [M+H]⁺ 483.13.

SZ-022304: ¹H NMR (400 MHz, DMSO-d₆) δ 9.56 (s, 1H), 9.03 (s, 1H), 7.72 (d, J=7.6 Hz, 1H), 7.60 (d, J=7.6 Hz, 1H), 7.42 (t, J=8.0 Hz, 1H), 6.32 (t, J=56.4 Hz, 1H), 5.83-5.73 (m, 1H), 3.81 (s, 3H), 2.56 (s, 3H), 2.33 (s, 3H), 1.58 (d, J=6.8 Hz, 3H), 1.55-1.49 (m, 2H), 1.47-1.35 (m, 2H).

### Example 7: SZ-022317

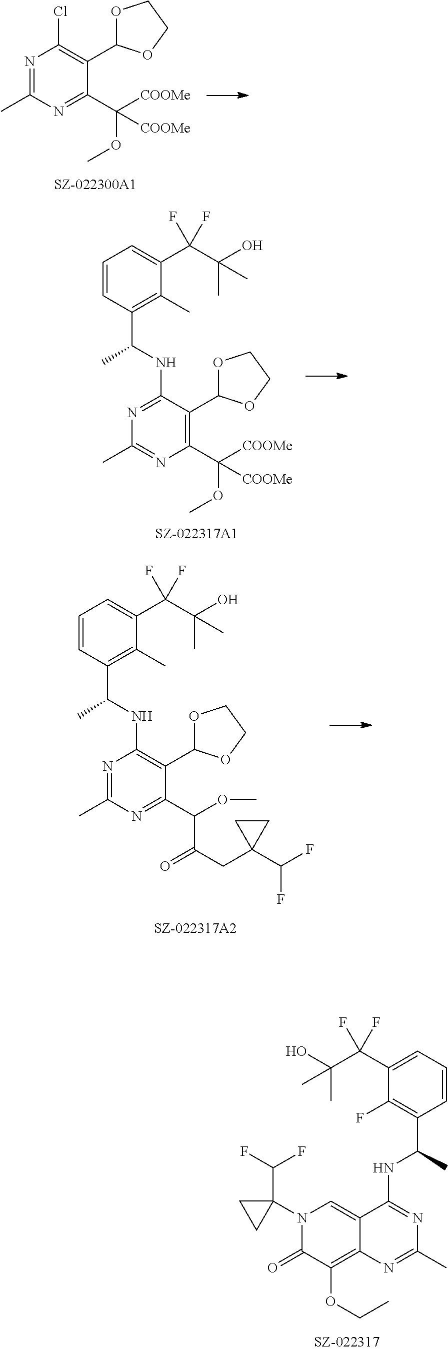 [SMILES: COC(=O)C(OC)(C(=O)OC)c1nc(C)nc(Cl)c1C1OCCO1 ; COC(=O)C(OC)(C(=O)OC)c1nc(C)nc(N[C@H](C)c2cccc(C(F)(F)C(C)(C)O)c2C)c1C1OCCO1 ; COC(C(=O)CC1(C(F)F)CC1)c1nc(C)nc(N[C@H](C)c2cccc(C(F)(F)C(C)(C)O)c2C)c1C1OCCO1 ; CCOc1c(=O)n(C2(C(F)F)CC2)cc2c(N[C@H](C)c3cccc(C(F)(F)C(C)(C)O)c3F)nc(C)nc12]

### Step 1:

Compound SZ-022300A1 (200 mg, 0.55 mmol), (1R)-1-(3-(1,1-difluoro-2-hydroxy-isobutyl)-2-fluorophenyl)ethylamine (135 mg, 0.55 mmol, synthesized according to WO2019122129), and triethylamine (112 mg, 1.11 mmol) were allowed to react at 60° C. for 3 h. After the starting materials were consumed as monitored by LC-MS, the reaction mixture was cooled to room temperature. Water (50 mL) and ethyl acetate (50 mL) were added to the reaction mixture for extraction, and the organic layer was separated, dried, and concentrated by rotary evaporation to give a crude product. The crude product was purified by flash silica gel column chromatography (PE:EA=3:1; Rf=0.3) to give product SZ-022317A1 as a colorless oil (100 mg). LC-MS: [M+H]⁺ 572.06.

### Step 2:

SZ-022317A1 (100 mg, 0.17 mmol) was added to dimethyl sulfoxide (2 mL), and then a 20% sodium hydroxide solution (100 μL) was added. The mixture was allowed to react at 35° C. for 2 h. 1-(Difluoromethyl)cyclopropan-1-amine hydrochloride (33 mg, 0.23 mmol), N,N,N,N-tetramethyl-O-(7-azabenzotriazol-1-yl)uronium hexafluorophosphate (135 mg, 0.36 mmol), and triethylamine (42 mg, 0.40 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022317A2 as a white viscous substance (95 mg). LC-MS: [M+H]⁺ 588.98.

### Step 3:

SZ-022317A2 (95 mg, 0.16 mmol) was added to isopropanol (2 mL), and then a hydrochloric acid solution (2 M, 0.5 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022317 (48 mg). LC-MS: [M+H]⁺ 527.18.

SZ-022317: ¹H NMR (400 MHz, DMSO-d₆) δ 8.93 (s, 1H), 8.79 (d, J=7.2 Hz, 1H), 7.55 (t, J=6.4 Hz, 1H), 7.34 (t, J=6.4 Hz, 1H), 7.24 (t, J=7.6 Hz, 1H), 6.34 (t, J=56.8 Hz, 1H), 5.78-5.71 (m, 1H), 5.32 (s, 1H), 3.77 (s, 3H), 2.21 (s, 3H), 1.56 (d, J=7.2 Hz, 3H), 1.52-1.48 (m, 2H), 1.43-1.35 (m, 2H), 1.22 (s, 3H), 1.20 (s, 3H).

### Example 8: SZ-022325

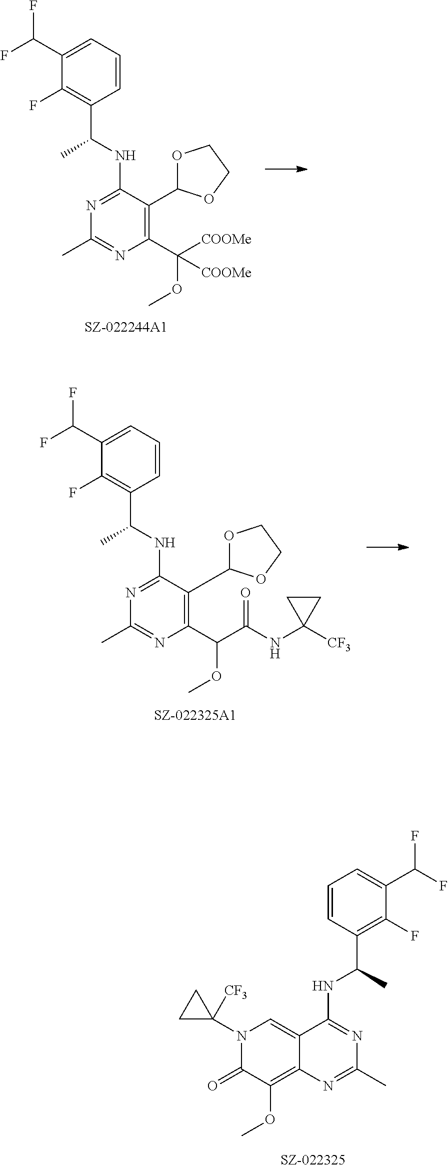 [SMILES: COC(=O)C(OC)(C(=O)OC)c1nc(C)nc(N[C@H](C)c2cccc(C(F)F)c2F)c1C1OCCO1 ; COC(C(=O)NC1(C(F)(F)F)CC1)c1nc(C)nc(N[C@H](C)c2cccc(C(F)F)c2F)c1C1OCCO1 ; COc1c(=O)n(C2(C(F)(F)F)CC2)cc2c(N[C@H](C)c3cccc(C(F)F)c3F)nc(C)nc12]

### Step 1:

SZ-022244A1 (120 mg, 0.23 mmol) was added to dimethyl sulfoxide (2 mL), and then a 20% sodium hydroxide solution (100 μL) was added. The mixture was allowed to react at 40° C. for 2 h. 1-Trifluoromethylcyclopropanamine hydrochloride (57 mg, 0.35 mmol), N,N,N,N-tetramethyl-O-(7-azabenzotriazol-1-yl)uronium hexafluorophosphate (267 mg, 0.70 mmol), and triethylamine (71 mg, 0.70 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022325A1 as a yellow viscous substance (120 mg). LC-MS: [M+H]⁺ 549.02.

### Step 2:

SZ-022325A1 (120 mg, 0.22 mmol) was added to isopropanol (2 mL), and then a hydrochloric acid solution (2 M, 0.50 mL) was added. The mixture was allowed to react at 50° C. for 4 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022325 (53 mg). LC-MS: [M+H]⁺ 487.10.

SZ-022325: ¹H NMR (400 MHz, DMSO-d₆) δ 8.99 (s, 1H), 8.81 (d, J=7.2 Hz, 1H), 7.63 (t, J=7.2 Hz, 1H), 7.52 (t, J=7.2 Hz, 1H), 7.33 (t, J=6.8 Hz, 1H), 7.23 (t, J=54 Hz, 1H), 5.80-5.70 (m, 1H), 3.76 (s, 3H), 2.21 (s, 3H), 1.88-1.48 (m, 4H), 1.68 (d, J=6.8 Hz, 3H).

### Example 9: SZ-022326

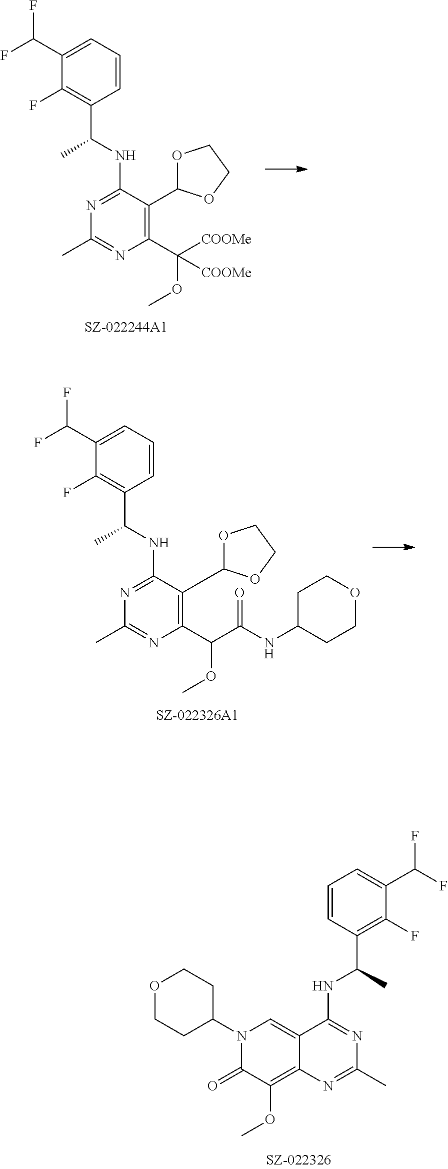 [SMILES: COC(=O)C(OC)(C(=O)OC)c1nc(C)nc(N[C@H](C)c2cccc(C(F)F)c2F)c1C1OCCO1 ; COC(C(=O)NC1CCOCC1)c1nc(C)nc(N[C@H](C)c2cccc(C(F)F)c2F)c1C1OCCO1 ; COc1c(=O)n(C2CCOCC2)cc2c(N[C@H](C)c3cccc(C(F)F)c3F)nc(C)nc12]

### Step 1:

SZ-022244A1 (120 mg, 0.23 mmol) was added to dimethyl sulfoxide (2 mL), and then a 20% sodium hydroxide solution (100 μL) was added. The mixture was allowed to react at 40° C. for 2 h. 4-Aminotetrahydropyran hydrochloride (48 mg, 0.35 mmol), N,N,N,N-tetramethyl-O-(7-azabenzotriazol-1-yl)uronium hexafluorophosphate (267 mg, 0.70 mmol), and triethylamine (71 mg, 0.70 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022326A1 as a yellow viscous substance (120 mg). LC-MS: [M+H]⁺ 525.12.

### Step 2:

SZ-022326A1 (120 mg, 0.23 mmol) was added to isopropanol (2 mL), and then a hydrochloric acid solution (2 M, 0.60 mL) was added. The mixture was allowed to react at 50° C. for 4 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022326 (53 mg). LC-MS: [M+H]⁺ 463.17.

SZ-022326: ¹H NMR (400 MHz, DMSO-d₆) δ 8.90 (s, 1H), 8.71 (d, J=7.2 Hz, 1H), 7.64 (t, J=7.6 Hz, 1H), 7.52 (t, J=7.2 Hz, 1H), 7.32 (t, J=7.6 Hz, 1H), 7.23 (t, J=54 Hz, 1H), 5.78-5.70 (m, 1H), 5.27-5.18 (m, 1H), 4.09-4.03 (m, 2H), 3.76 (s, 3H), 3.58-3.50 (m, 2H), 2.22 (s, 3H), 2.11-2.02 (m, 2H), 1.83-1.77 (m, 2H), 1.59 (d, J=7.2 Hz, 3H).

### Example 10: SZ-022303

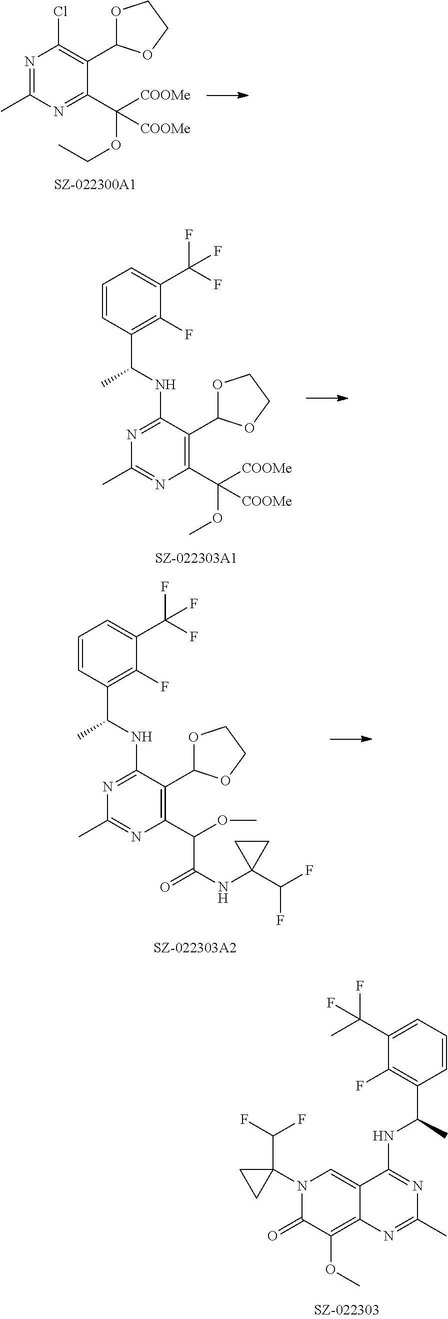 [SMILES: CCOC(C(=O)OC)(C(=O)OC)c1nc(C)nc(Cl)c1C1OCCO1 ; COC(=O)C(OC)(C(=O)OC)c1nc(C)nc(N[C@H](C)c2cccc(C(F)(F)F)c2F)c1C1OCCO1 ; COC(C(=O)NC1(C(F)F)CC1)c1nc(C)nc(N[C@H](C)c2cccc(C(F)(F)F)c2F)c1C1OCCO1 ; COc1c(=O)n(C2(C(F)F)CC2)cc2c(N[C@H](C)c3cccc(C(C)(F)F)c3F)nc(C)nc12]

### Step 1:

Compound SZ-022300A1 (200 mg, 0.55 mmol), (1R)-1-(3-(1,1-difluoroethyl)-2-fluorophenyl)ethylamine hydrochloride (159 mg, 0.67 mmol), and triethylamine (112 mg, 1.11 mmol) were allowed to react at 60° C. for 3 h. After the starting materials were consumed as monitored by LC-MS, the reaction mixture was cooled to room temperature. Water (50 mL) and ethyl acetate (50 mL) were added to the reaction mixture for extraction, and the organic layer was separated, dried, and concentrated by rotary evaporation to give a crude product. The crude product was purified by flash silica gel column chromatography (PE:EA=3:1; Rf=0.3) to give product SZ-022303A1 as a colorless oil (130 mg). LC-MS: [M+H]⁺ 528.06.

### Step 2:

SZ-022303A1 (130 mg, 0.25 mmol) was added to dimethyl sulfoxide (2 mL), and then a 20% sodium hydroxide solution (100 μL) was added. The mixture was allowed to react at 35° C. for 2 h. 1-(Difluoromethyl)cyclopropan-1-amine hydrochloride (46 mg, 0.32 mmol), N,N,N,N-tetramethyl-O-(7-azabenzotriazol-1-yl)uronium hexafluorophosphate (187 mg, 0.49 mmol), and triethylamine (50 mg, 0.49 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022303A2 as a light yellow viscous substance (100 mg). LC-MS: [M+H]⁺ 545.02.

### Step 3:

SZ-022303A2 (100 mg, 0.25 mmol) was added to isopropanol (2 mL), and then a hydrochloric acid solution (2 M, 0.5 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022303 (60 mg). LC-MS: [M+H]⁺ 483.17.

SZ-022303: ¹H NMR (400 MHz, DMSO-d₆) δ 8.93 (s, 1H), 8.80 (d, J=7.2 Hz, 1H), 7.59 (t, J=6.8 Hz, 1H), 7.45 (t, J=6.8 Hz, 1H), 7.28 (t, J=7.6 Hz, 1H), 6.34 (t, J=56.8 Hz, 1H), 5.78-5.71 (m, 1H), 3.77 (s, 3H), 2.21 (s, 3H), 2.02 (t, J=19.2 Hz, 3H), 1.58 (d, J=7.2 Hz, 3H), 1.51-1.48 (m, 2H), 1.44-1.35 (m, 2H).

### Example 11: SZ-022328

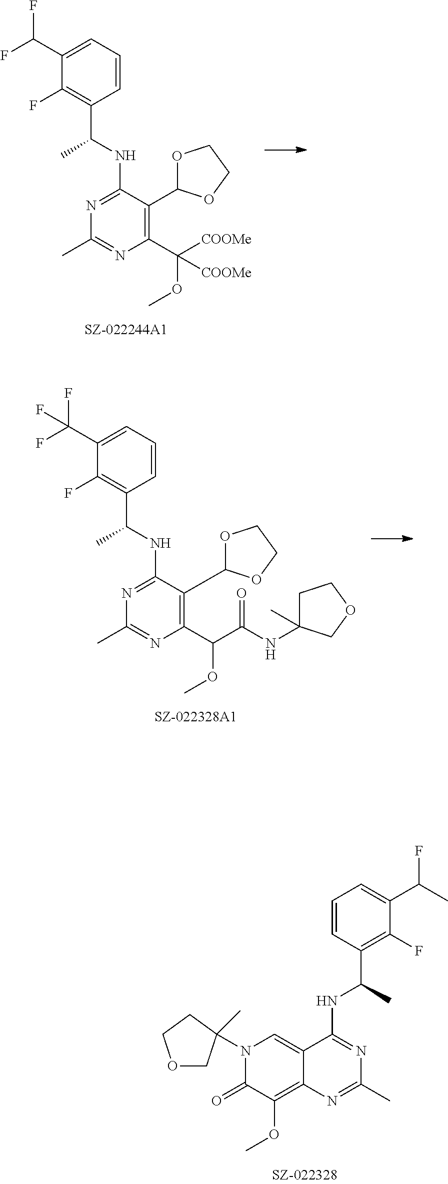 [SMILES: COC(=O)C(OC)(C(=O)OC)c1nc(C)nc(N[C@H](C)c2cccc(C(F)F)c2F)c1C1OCCO1 ; COC(C(=O)NC1(C)CCOC1)c1nc(C)nc(N[C@H](C)c2cccc(C(F)(F)F)c2F)c1C1OCCO1 ; COc1c(=O)n(C2(C)CCOC2)cc2c(N[C@H](C)c3cccc(C(C)F)c3F)nc(C)nc12]

### Step 1:

SZ-022244A1 (120 mg, 0.23 mmol) was added to dimethyl sulfoxide (2 mL), and then a 20% sodium hydroxide solution (120 μL) was added. The mixture was allowed to react at 40° C. for 2 h. 3-Methyloxolan-3-amine (36 mg, 0.35 mmol), N,N,N,N-tetramethyl-O-(7-azabenzotriazol-1-yl)uronium hexafluorophosphate (267 mg, 0.70 mmol), and triethylamine (71 mg, 0.70 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022328A1 as a yellow viscous substance (120 mg). LC-MS: [M+H]⁺ 525.21.

### Step 2:

SZ-022328A1 (120 mg, 0.23 mmol) was added to isopropanol (2 mL), and then a hydrochloric acid solution (2 M, 0.58 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022328 (16.4 mg). LC-MS: [M+H]⁺ 463.16.

SZ-022328: ¹H NMR (400 MHz, DMSO-d₆) δ 8.79-8.72 (m, 1H), 8.64 (d, J=4.8 Hz, 1H), 7.66 (t, J=7.2 Hz, 1H), 7.52 (t, J=7.2 Hz, 1H), 7.31 (td, J=7.7, 2.8 Hz, 1H), 7.23 (t, J=54.4 Hz, 1H), 5.82-5.73 (m, 1H), 4.42 (d, J=9.2 Hz, 1H), 3.98-3.85 (m, 3H), 3.74 (s, 3H), 2.57-2.52 (m, 2H), 2.21 (s, 3H), 1.61 (s, 3H), 1.59 (d, J=6.4 Hz, 3H).

### Example 12: SZ-022334

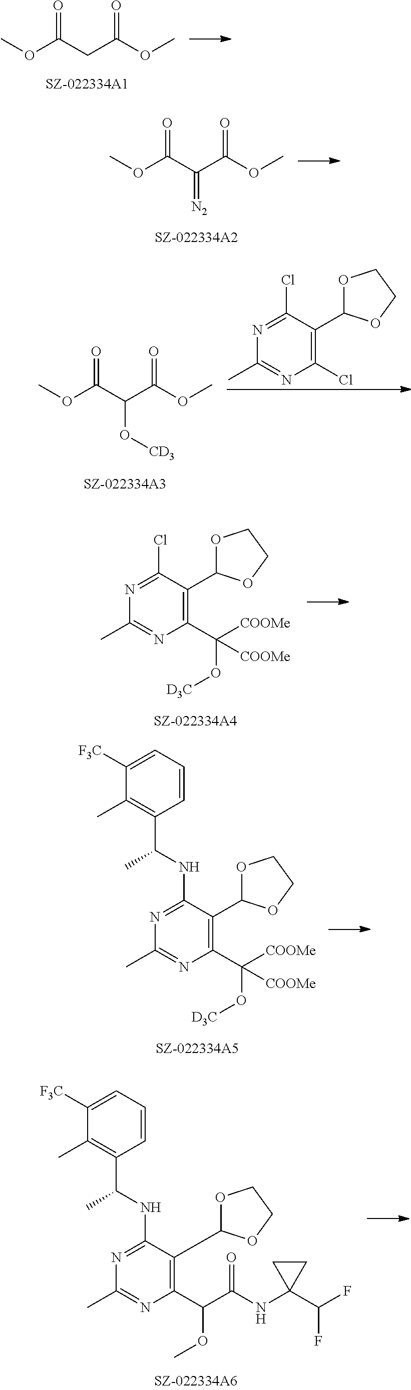

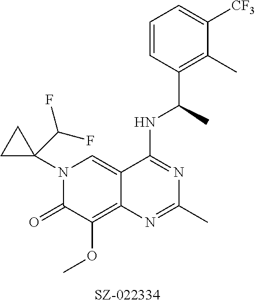 [SMILES: COc1c(=O)n(C2(C(F)F)CC2)cc2c(N[C@H](C)c3cccc(C(F)(F)F)c3C)nc(C)nc12]

### Step 1:

SZ-022334A1 (1 g, 7.57 mmol) was added to acetonitrile (5 mL), and then triethylamine (920 mg, 9.09 mmol) was added. The mixture was cooled to 0° C., and 4-acetamidobenzenesulfonyl azide (2.18 g, 9.09 mmol) was slowly added. The resulting mixture was allowed to react at room temperature overnight. After the starting materials were consumed as monitored by LC-MS, the mixture was filtered under vacuum. The filter cake was washed with ethyl acetate (20 mL), and water (10 mL) was added for extraction. The organic layer was separated, washed with a saturated sodium chloride solution, dried, and concentrated by rotary evaporation to give a crude product. The crude product was purified by flash silica gel column chromatography (PE:EA=5:1; Rf=0.3) to give product SZ-022334A2 as a light yellow oil (1 g).

### Step 2:

SZ-022334A2 (1 g, 6.33 mmol) was added to dichloromethane (10 mL), and then deuterated methanol (250 mg, 6.96 mmol) and rhodium(II) acetate dimer (28 mg, 63 μmol) were added. The mixture was allowed to react for 3 h with the temperature maintained at 70° C. After the starting materials were consumed as monitored by TLC, the reaction mixture was concentrated by rotary evaporation to give a crude product. The crude product was purified by flash silica gel column chromatography (PE:EA=10:1; Rf=0.2) to give product SZ-022334A3 as a colorless oil (840 mg).

### Step 3:

SZ-022046A1 (600 mg, 2.56 mmol, synthesized according to WO2019122129), SZ-022334A3 (840 mg, 5.1 mmol), and potassium carbonate (1.1 g, 7.7 mmol) were added to dimethyl sulfoxide (10 mL), and the mixture was heated to 100° C. for reaction for 5 h. After the starting materials were consumed as monitored by LC-MS, water (100 mL) and ethyl acetate (100 mL) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA=3:1; Rf=0.4) to give product SZ-022334A4 as a yellow oil (220 mg). LC-MS: [M+H]⁺ 364.22, 366.20.

### Step 4:

SZ-022334A4 (220 mg, 0.61 mmol), (1R)-1-(3-(trifluoromethyl)-2-methylphenyl)ethylamine hydrochloride (174 mg, 0.73 mmol, synthesized according to WO2019122129), triethylamine (184 mg, 1.82 mmol), and potassium carbonate (84 mg, 0.61 mmol) were added to dimethyl sulfoxide (5 mL), and the mixture was heated to 60° C. for reaction for 8 h. After the starting materials were consumed as monitored by LC-MS, water (100 mL) and ethyl acetate (100 mL) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA=3:1; Rf=0.35) to give product SZ-022334A5 as a yellow oil (200 mg). LC-MS: [M+H]⁺ 530.99.

### Step 5:

SZ-022334A5 (200 mg, 0.38 mmol) was added to dimethyl sulfoxide (2 mL), and then a 10% sodium hydroxide solution (750 μL) was added. The mixture was allowed to react at 40° C. for 1 h. 1-Difluoromethylcyclopropanamine hydrochloride (71 mg, 0.57 mmol), N,N,N,N-tetramethyl-O-(7-azabenzotriazol-1-yl)uronium hexafluorophosphate (430 mg, 1.13 mmol), and triethylamine (191 mg, 1.89 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022334A6 as a yellow viscous substance (200 mg). LC-MS: [M+H]⁺ 548.31.

### Step 6:

SZ-022334A6 (200 mg, 0.37 mmol) was added to isopropanol (5 mL), and then a hydrochloric acid solution (2 M, 0.9 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022334 (85 mg). LC-MS: [M+H]⁺ 485.88.

SZ-022334: ¹H NMR (400 MHz, DMSO-d₆) δ 8.91 (s, 1H), 8.88 (d, J=6.8 Hz, 1H), 7.70 (d, J=7.6 Hz, 1H), 7.56 (d, J=7.6 Hz, 1H), 7.39 (t, J=7.6 Hz, 1H), 6.34 (t, J=56.8 Hz, 1H), 5.72-5.65 (m, 1H), 2.57 (s, 3H), 2.21 (s, 3H), 1.54 (d, J=7.2 Hz, 3H), 1.51-1.45 (m, 2H), 1.45-1.30 (m, 2H).

### Example 13: SZ-022335

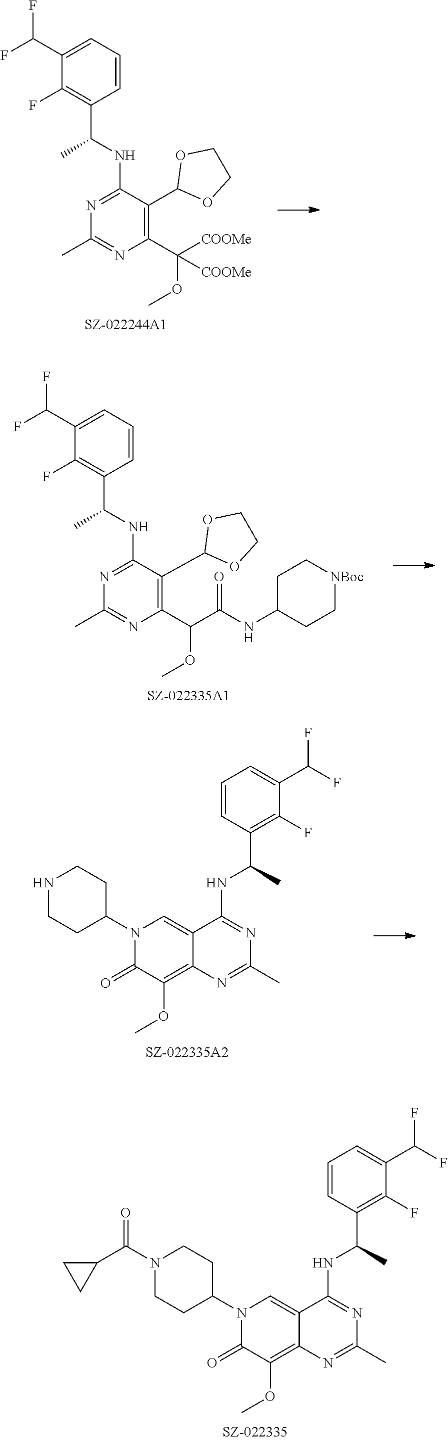 [SMILES: COC(=O)C(OC)(C(=O)OC)c1nc(C)nc(N[C@H](C)c2cccc(C(F)F)c2F)c1C1OCCO1 ; COC(C(=O)NC1CCN(C(=O)OC(C)(C)C)CC1)c1nc(C)nc(N[C@H](C)c2cccc(C(F)F)c2F)c1C1OCCO1 ; COc1c(=O)n(C2CCNCC2)cc2c(N[C@H](C)c3cccc(C(F)F)c3F)nc(C)nc12 ; COc1c(=O)n(C2CCN(C(=O)C3CC3)CC2)cc2c(N[C@H](C)c3cccc(C(F)F)c3F)nc(C)nc12]

### Step 1:

SZ-022244A1 (145 mg, 0.28 mmol) was added to dimethyl sulfoxide (2 mL), and then a 10% sodium hydroxide solution (230 μL) was added. The mixture was allowed to react at 40° C. for 1 h. 4-Amino-1-BOC-piperidine (85 mg, 0.42 mmol), N,N,N,N-tetramethyl-O-(7-azabenzotriazol-1-yl)uronium hexafluorophosphate (322 mg, 0.85 mmol), and triethylamine (86 mg, 0.85 mmol) were then added into the system, and the mixture was allowed to react at room temperature for another 8 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022335A1 as a yellow viscous substance (140 mg). LC-MS: [M+H]⁺ 624.41.

### Step 2:

SZ-022335A1 (140 mg, 0.23 mmol) was added to isopropanol (2 mL), and then a hydrochloric acid solution (2 M, 0.50 mL) was added. The mixture was allowed to react at 50° C. for 8 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative medium-pressure reversed-phase chromatography and then lyophilized to give SZ-022335A2 (150 mg). LC-MS: [M+H]⁺ 461.88.

### Step 3:

SZ-022335A2 (140 mg, 0.30 mmol), cyclopropanecarboxylic acid (26 mg, 0.30 mmol), N,N,N,N-tetramethyl-O-(7-azabenzotriazol-1-yl)uronium hexafluorophosphate (173 mg, 0.45 mmol), and triethylamine (92 mg, 0.90 mmol) were added to N,N-dimethylformamide (5 mL). The mixture was allowed to react at room temperature for 3 h. After the starting materials were consumed as monitored by LC-MS, water (20 mL) and ethyl acetate (20 mL) were added for extraction, and the organic layer was separated and washed with a saturated sodium chloride solution. The solvent was removed by rotary evaporation, and the residue was subjected to preparative high performance liquid chromatography and then lyophilized to give SZ-022335 (42 mg). LC-MS: [M+H]⁺ 530.00.

SZ-022335: ¹H NMR (400 MHz, DMSO-d₆) δ 8.80 (s, 1H), 8.70 (d, J=7.2 Hz, 1H), 7.64 (t, J=7.2 Hz, 1H), 7.52 (t, J=7.2 Hz, 1H), 7.32 (t, J=7.6 Hz, 1H), 7.23 (t, J=54.4 Hz, 1H), 5.78-5.71 (m, 1H), 5.28-5.18 (m, 1H), 4.74-4.45 (m, 2H), 3.77 (s, 3H), 2.82-2.68 (m, 1H), 2.22 (s, 3H), 2.11-2.05 (m, 1H), 2.03-1.82 (m, 4H), 1.59 (d, J=7.2 Hz, 3H), 0.86-0.66 (m, 4H).

### Example 14: SZ-022336

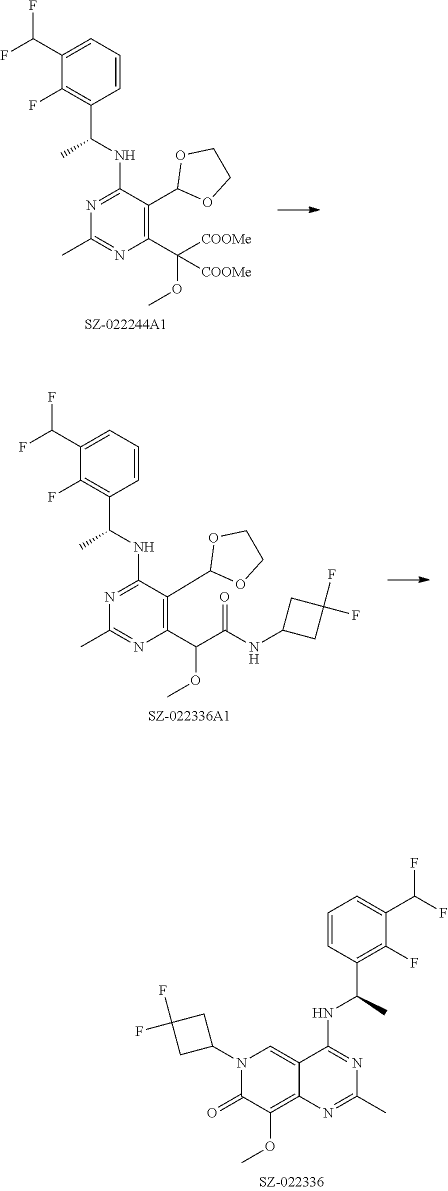 [SMILES: COC(=O)C(OC)(C(=O)OC)c1nc(C)nc(N[C@H](C)c2cccc(C(F)F)c2F)c1C1OCCO1 ; COC(C(=O)NC1CC(F)(F)C1)c1nc(C)nc(N[C@H](C)c2cccc(C(F)F)c2F)c1C1OCCO1 ; COc1c(=O)n(C2CC(F)(F)C2)cc2c(N[C@H](C)c3cccc(C(F)F)c3F)nc(C)nc12]

### Step 1:

SZ-022244A1 (140 mg, 0.28 mmol) was added to dimethyl sulfoxide (2 mL), and then a 10% sodium hydroxide solution (500 μL) was added. The mixture was allowed to react at 40° C. for 1 h. 3,3-Difluorocyclobutanamine hydrochloride (60 mg, 0.42 mmol), N,N,N,N-tetramethyl-O-(7-azabenzotriazol-1-yl)uronium hexafluorophosphate (320 mg, 0.84 mmol), and triethylamine (85 mg, 0.84 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022336A1 as a yellow viscous substance (140 mg). LC-MS: [M+H]⁺ 531.23.

### Step 2:

SZ-022336A1 (140 mg, 0.26 mmol) was added to isopropanol (2 mL), and then a hydrochloric acid solution (2 M, 0.6 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022336 (44 mg). LC-MS: [M+H]⁺ 468.9.

SZ-022336: ¹H NMR (400 MHz, DMSO-d₆) δ 8.76 (s, 1H), 8.71 (d, J=7.2 Hz, 1H), 7.67 (t, J=7.2 Hz, 1H), 7.53 (t, J=7.2 Hz, 1H), 7.32 (t, J=7.6 Hz, 1H), 7.24 (t, J=54.4 Hz, 1H), 5.75-5.82 (m, 1H), 4.97-4.86 (m, 1H), 3.77 (s, 3H), 3.29-3.10 (m, 4H), 2.23 (s, 3H), 1.60 (d, J=7.2 Hz, 3H).

### Example 15: SZ-022307

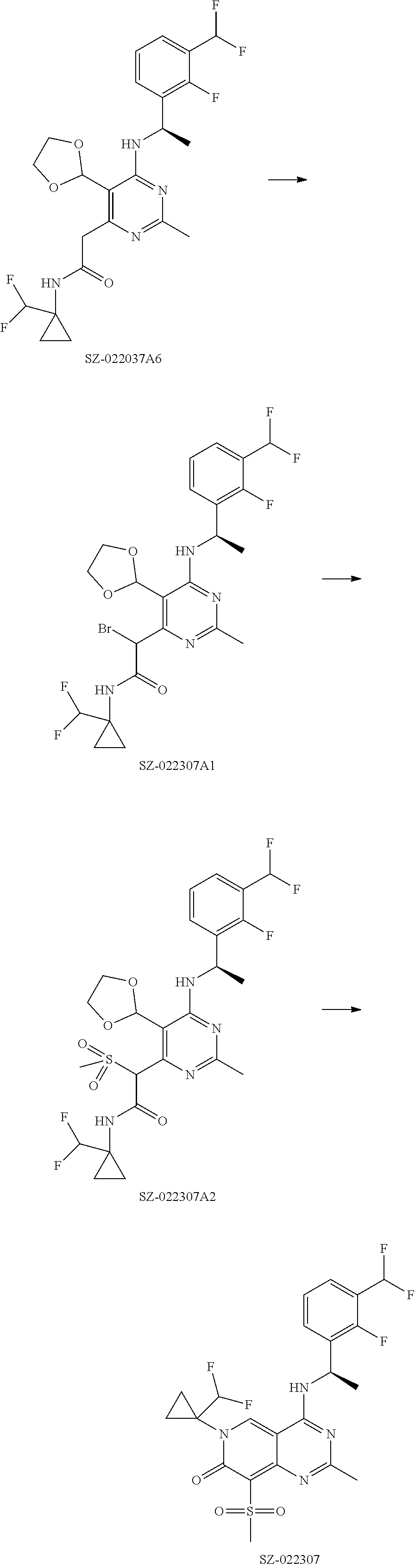 [SMILES: Cc1nc(CC(=O)NC2(C(F)F)CC2)c(C2OCCO2)c(N[C@H](C)c2cccc(C(F)F)c2F)n1 ; Cc1nc(N[C@H](C)c2cccc(C(F)F)c2F)c(C2OCCO2)c(C(Br)C(=O)NC2(C(F)F)CC2)n1 ; Cc1nc(N[C@H](C)c2cccc(C(F)F)c2F)c(C2OCCO2)c(C(C(=O)NC2(C(F)F)CC2)S(C)(=O)=O)n1 ; Cc1nc(N[C@H](C)c2cccc(C(F)F)c2F)c2cn(C3(C(F)F)CC3)c(=O)c(S(C)(=O)=O)c2n1]

### Step 1:

SZ-022037A6 (650 mg, 1.30 mmol, synthesized according to WO2019122129), N-bromosuccinimide (231 mg, 1.30 mmol), and azobisisobutyronitrile (21.3 mg, 0.13 mmol) were suspended in carbon tetrachloride (15 mL) under argon atmosphere, and the mixture was stirred at 60° C. for 1 h. The reaction mixture was then concentrated to give a crude product. The crude product was purified by column chromatography (petroleum ether:ethyl acetate=1:1, Rf=0.5) to give SZ-022307A1 as a white solid (500 mg). LCMS: [M+H]⁺ 578.84, 580.76.

### Step 2:

SZ-022307A1 (100 mg, 0.17 mmol) and sodium methanesulfinate (21 mg, 0.20 mmol) were mixed in dimethyl sulfoxide (10 mL) under argon atmosphere, and then the mixture was stirred at room temperature for 2 h. After the reaction was completed, 100 mL of water was added to the reaction mixture. The resulting mixture was extracted with 50 mL of ethyl acetate, and the organic phase was dried and concentrated to give SZ-022307A2 as a yellow liquid (100 mg). LCMS: [M+H]⁺ 578.92.

### Step 3:

SZ-022307A2 (100 mg, 0.17 mmol) and hydrochloric acid (0.2 mL, 5 M) were mixed in isopropanol (2 mL) under argon atmosphere, and then the mixture was stirred at 40° C. for 2 h. After the reaction was completed, the reaction mixture was diluted with 10 mL of water. The dilution was extracted with 20 mL of ethyl acetate, and the organic phase was dried and concentrated to give a crude product. The crude product was purified by preparative reversed-phase chromatography and lyophilized to give SZ-022307 as a yellow solid (13.91 mg). LCMS: [M+H]⁺ 516.86.

SZ-022307: ¹H NMR (400 MHz, DMSO-d₆) δ 9.46 (s, 1H), 9.18 (d, J=4.0 Hz, 1H), 7.66 (t, J=8.0 Hz, 1H), 7.54 (t, J=8.0 Hz, 1H), 7.34 (t, J=8.0 Hz, 1H), 7.23 (t, J=52.0 Hz, 1H), 6.36 (t, J=56.0 Hz, 1H), 5.81-5.76 (m, 1H), 3.33 (s, 3H), 2.28 (s, 3H), 1.60 (d, J=4.0 Hz, 3H), 1.52-1.43 (m, 4H).

### Example 16: SZ-022312

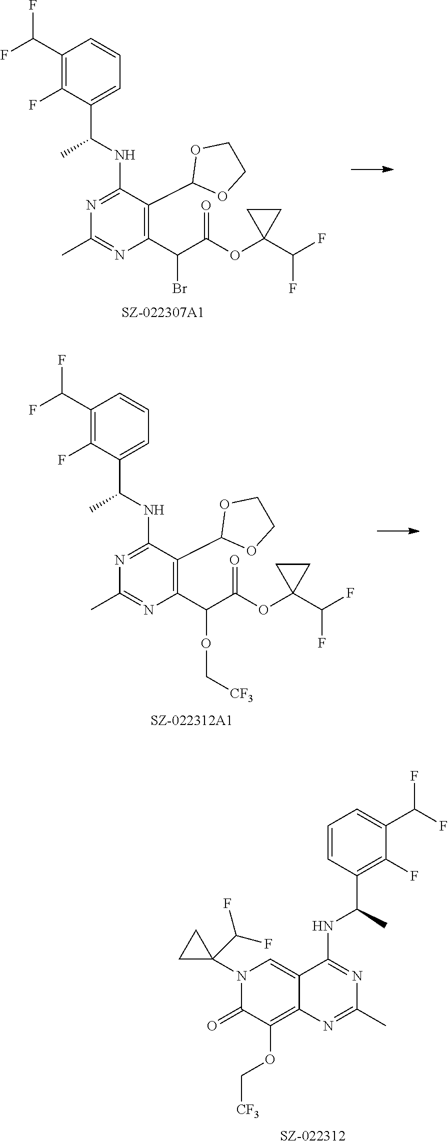 [SMILES: Cc1nc(N[C@H](C)c2cccc(C(F)F)c2F)c(C2OCCO2)c(C(Br)C(=O)OC2(C(F)F)CC2)n1 ; Cc1nc(N[C@H](C)c2cccc(C(F)F)c2F)c(C2OCCO2)c(C(OCC(F)(F)F)C(=O)OC2(C(F)F)CC2)n1 ; Cc1nc(N[C@H](C)c2cccc(C(F)F)c2F)c2cn(C3(C(F)F)CC3)c(=O)c(OCC(F)(F)F)c2n1]

### Step 1:

Sodium hydride (14 mg, 0.3 mmol) was added to tetrahydrofuran (5 mL), and then trifluoroethanol (20 mg, 0.2 mmol) was added. The mixture was stirred at room temperature for 0.5 h. A solution of SZ-022307A1 (57 mg, 0.1 mmol) in tetrahydrofuran (1 mL) was added dropwise to the reaction mixture, and the resulting mixture was stirred for another 1.5 h. After the starting materials were consumed as monitored by LC-MS, water (10 mL) and ethyl acetate (10 mL) were added to the reaction mixture for extraction, and the organic layer was separated, washed with a saturated sodium chloride solution, dried, and concentrated by rotary evaporation to give crude product SZ-022312A1 as a yellow viscous substance (55 mg). LC-MS: [M+H]⁺ 598.98.

### Step 2:

SZ-022312A1 (55 mg, 0.1 mmol) was added to isopropanol (1 mL), and then a hydrochloric acid solution (2 M, 0.3 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL) and ethyl acetate (10 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022312 (4 mg). LC-MS: [M+H]⁺ 536.91.

SZ-022312: ¹H NMR (400 MHz, DMSO-d₆) δ 8.94 (s, 1H), 8.88 (d, J=6.8 Hz, 1H), 7.61 (t, J=6.8 Hz, 1H), 7.49 (t, J=6.8 Hz, 1H), 7.29 (t, J=7.6 Hz, 1H), 7.19 (t, J=54.4 Hz, 1H), 6.30 (t, J=56.8 Hz, 1H), 5.74-5.70 (m, 1H), 4.62 (q, J=9.2 Hz, 2H), 2.19 (s, 3H), 1.55 (d, J=6.8 Hz, 4H), 1.47 (s, 3H).

### Example 17: SZ-022314

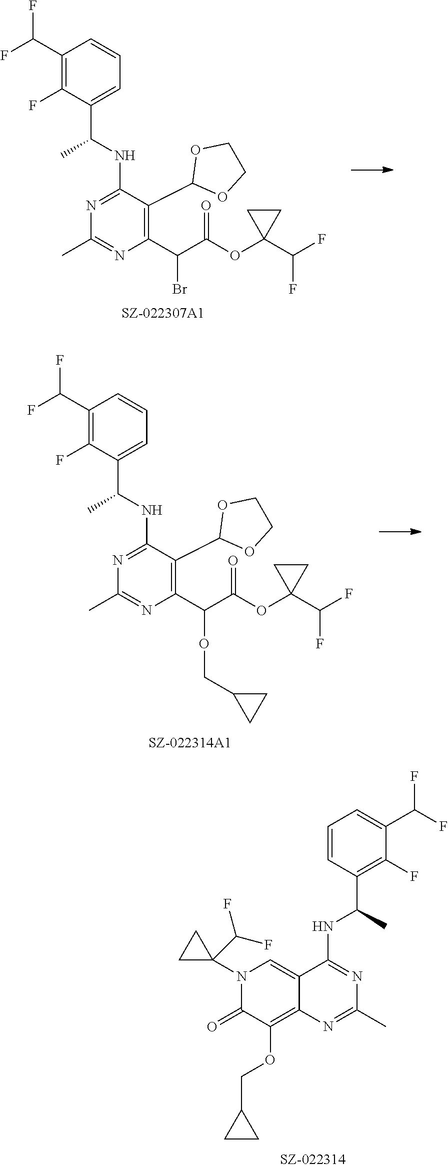 [SMILES: Cc1nc(N[C@H](C)c2cccc(C(F)F)c2F)c(C2OCCO2)c(C(Br)C(=O)OC2(C(F)F)CC2)n1 ; Cc1nc(N[C@H](C)c2cccc(C(F)F)c2F)c(C2OCCO2)c(C(OCC2CC2)C(=O)OC2(C(F)F)CC2)n1 ; Cc1nc(N[C@H](C)c2cccc(C(F)F)c2F)c2cn(C3(C(F)F)CC3)c(=O)c(OCC3CC3)c2n1]

### Step 1:

Sodium hydride (14 mg, 0.3 mmol) was added to tetrahydrofuran (5 mL), and then cyclopropanemethanol (14.4 mg, 0.2 mmol) was added. The mixture was stirred at room temperature for 0.5 h. A solution of SZ-022307A1 (57 mg, 0.1 mmol) in tetrahydrofuran (1 mL) was added dropwise to the reaction mixture, and the resulting mixture was stirred for another 1.5 h. After the starting materials were consumed as monitored by LC-MS, water (10 mL) and ethyl acetate (10 mL) were added to the reaction mixture for extraction, and the organic layer was separated, washed with a saturated sodium chloride solution, dried, and concentrated by rotary evaporation to give crude product SZ-022314A1 as a yellow viscous substance (60 mg). LC-MS: [M+H]⁺ 571.01.

### Step 2:

SZ-022314A1 (60 mg, 0.1 mmol) was added to isopropanol (1 mL), and then a hydrochloric acid solution (2 M, 0.3 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL) and ethyl acetate (10 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022314 (5 mg). LC-MS: [M+H]⁺ 508.86.

SZ-022314: ¹H NMR (400 MHz, DMSO-d₆) δ 8.94 (s, 1H), 8.81 (d, J=6.8 Hz, 1H), 7.66 (t, J=7.6 Hz, 1H), 7.54 (t, J=7.2 Hz, 1H), 7.34 (t, J=7.6 Hz, 1H), 7.25 (t, J=54.4 Hz, 1H), 6.35 (t, J=57.2 Hz, 1H), 5.79-5.76 (m, 1H), 3.89 (d, J=7.2 Hz, 2H), 2.24 (s, 3H), 1.59 (d, J=7.2 Hz, 4H), 1.51 (s, 3H), 1.19-1.13 (m, 1H), 0.47-0.42 (m, J=7.2 Hz, 2H), 0.27-0.23 (m, J=7.2 Hz, 2H).

### Example 18: SZ-022306

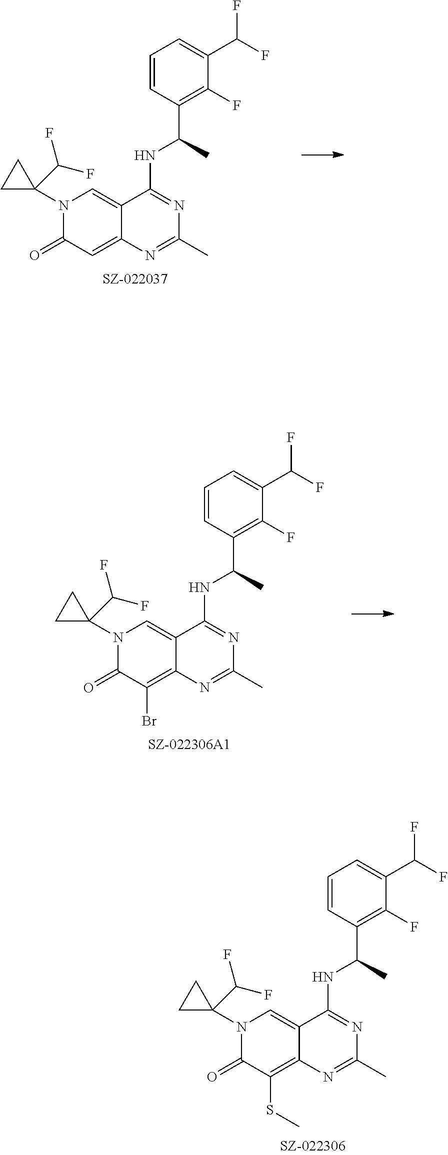 [SMILES: Cc1nc(N[C@H](C)c2cccc(C(F)F)c2F)c2cn(C3(C(F)F)CC3)c(=O)cc2n1 ; Cc1nc(N[C@H](C)c2cccc(C(F)F)c2F)c2cn(C3(C(F)F)CC3)c(=O)c(Br)c2n1 ; CSc1c(=O)n(C2(C(F)F)CC2)cc2c(N[C@H](C)c3cccc(C(F)F)c3F)nc(C)nc12]

### Step 1:

SZ-022037 (200 mg, 0.46 mmol, synthesized according to WO2019122129) and N-bromosuccinimide (96.1 mg, 0.55 mmol) were mixed in acetonitrile (5 mL) under argon atmosphere, and the mixture was stirred at room temperature for 1 h. The reaction mixture was then directly purified by medium-pressure reversed-phase chromatography (acetonitrile/water=40%) to give SZ-022306A1 as a white solid (160 mg). LCMS: [M+H]⁺ 516.82, 518.79.

### Step 2:

SZ-022306A1 (50 mg, 0.10 mmol) and a sodium thiomethoxide solution (0.1 mL, 20% wt) were mixed in N,N-dimethylformamide (3 mL) under argon atmosphere, and then the mixture was stirred for half an hour in an ice-water bath. After the reaction was completed, 30 mL of water was added to the reaction mixture. The resulting mixture was extracted with 50 mL of ethyl acetate, and the organic phase was dried and concentrated to give a crude product. The crude product was purified by preparative high-pressure chromatography and lyophilized to give SZ-022306 as a yellow solid (10 mg). LCMS: [M+H]⁺ 485.12.

SZ-022306: ¹H NMR (400 MHz, DMSO-d₆) δ 9.11 (s, 1H), 8.88 (d, J=8.0 Hz, 1H), 7.64 (t, J=8.0 Hz, 1H), 7.52 (t, J=8.0 Hz, 1H), 7.32 (t, J=8.0 Hz, 1H), 7.23 (t, J=52.0 Hz, 1H), 6.37 (t, J=56.0 Hz, 1H), 5.78-5.72 (m, 1H), 2.39 (s, 3H), 2.24 (s, 3H), 1.58 (d, J=4.0 Hz, 3H), 1.50-1.40 (m, 4H).

### Example 19: Intermediate SZ-022037

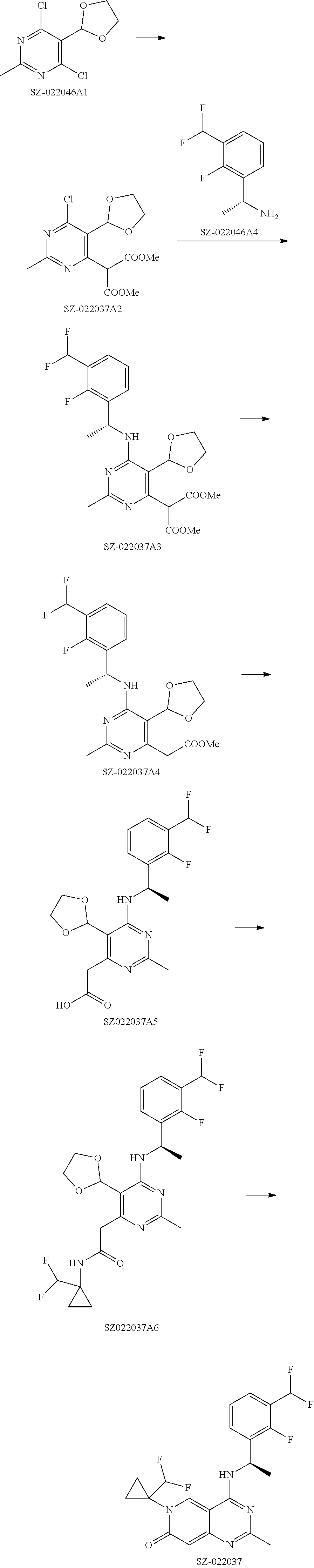

### Step 1:

SZ-022046A1 (38.0 g, 161.7 mmol), dimethyl malonate (21.8 g, 164.9 mmol), and cesium carbonate (105.3 g, 323.3 mmol) were added to dimethyl sulfoxide (300 mL), and the mixture was heated to 100° C. for reaction for 3 h. Water (1 L) and ethyl acetate (1 L) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA=3:1; Rf=0.2) to give product SZ-022037A2 as an off-white solid (31.9 g). LC-MS: [M+H]⁺ 330.69, 332.51.

### Step 2:

SZ-022037A2 (15.0 g, 45.4 mmol), SZ-022046A4 (10.4 g, 46.3 mmol), and N,N-diisopropylethylamine (17.6 g, 136.0 mmol) were added to dimethyl sulfoxide (150 mL), and the mixture was heated to 80° C. for reaction for 15 h. After the starting materials were consumed as monitored by LC-MS, water (1 L) and ethyl acetate (1 L) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA=1:1; Rf=0.2) to give product SZ-022037A3 as a brown-yellow oil (17.3 g). LC-MS: [M+H]⁺ 483.93, 484.88.

### Step 3:

SZ-022037A3 (5.4 g, 11.2 mmol) and lithium chloride (1.9 g, 44.7 mmol) were added to dimethyl sulfoxide (190 mL), and the mixture was heated to 120° C. for reaction for 15 h. After the starting materials were consumed as monitored by LC-MS, water (1.5 L) and ethyl acetate (1.5 L) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA:DCM=1:1:0.1; Rf=0.2) to give product SZ-022037A4 as a brown-yellow oil (5.3 g). LC-MS: [M+H]⁺ 426.98.

### Step 4:

SZ-022037A4 (2.9 g, 6.8 mmol) was dissolved in a mixed solvent of acetonitrile (30 mL) and dimethyl sulfoxide (45 mL), and then a 10% sodium hydroxide solution (11 mL, 27.3 mmol) was added. The mixture was allowed to react at 25° C. for 1.5 h. After the starting materials were consumed as monitored by LC-MS, the reaction mixture was used directly in the next step without work-up. LC-MS: [M+H]⁺ 411.92.

### Step 5:

1-(Difluoromethyl)cyclopropanamine hydrochloride (1.3 g, 9.4 mmol), N,N,N,N-tetramethyl-O-(7-azabenzotriazol-1-yl)uronium hexafluorophosphate (7.6 g, 20.4 mmol), and triethylamine (2.1 g, 20.4 mmol) were added to the reaction system in the previous step, and the mixture was allowed to react at 25° C. for 1 h. After the starting materials were consumed as monitored by LC-MS, water (500 mL) and ethyl acetate (500 mL) were added to the reaction mixture for extraction, and the organic layer was separated, washed with a saturated sodium chloride solution, dried, and concentrated to dryness under reduced pressure to give crude product SZ-022037A6 as a light yellow solid (4.0 g). LC-MS: [M+H]⁺ 501.3.

### Step 6:

Crude product SZ-022037A6 (4.0 g, 6.8 mmol) was added to isopropanol (50 mL), and then a hydrochloric acid solution (2 M, 17 mL) was added. The mixture was allowed to react at 50° C. for 3 h. After the starting materials were consumed as monitored by LC-MS, the reaction mixture was directly concentrated to dryness under reduced pressure. A saturated sodium bicarbonate solution (50 mL) and ethyl acetate (50 mL) were then added for extraction, and the organic layer was separated, washed with a saturated sodium chloride solution, dried, and concentrated under reduced pressure to give a crude product. The crude product was separated by preparative high performance liquid chromatography and then lyophilized to give SZ-022037 as a yellow solid (1.8 g). LC-MS: [M+H]⁺ 438.83.

SZ-022037: ¹H NMR (400 MHz, DMSO-d₆) δ 9.16 (s, 1H), 8.89 (d, J=6.8 Hz, 1H), 7.65 (t, J=7.6 Hz, 1H), 7.53 (t, J=7.2 Hz, 1H), 7.33 (t, J=7.6 Hz, 1H), 7.24 (t, J=54.4 Hz, 1H), 6.34 (t, J=57.2 Hz, 1H), 6.09 (s, 1H), 5.78-5.71 (m, 1H), 2.18 (s, 3H), 1.58 (d, J=7.2 Hz, 3H), 1.50-1.46 (m, 2H), 1.45-1.36 (m, 2H).

### Example 20: SZ-022251

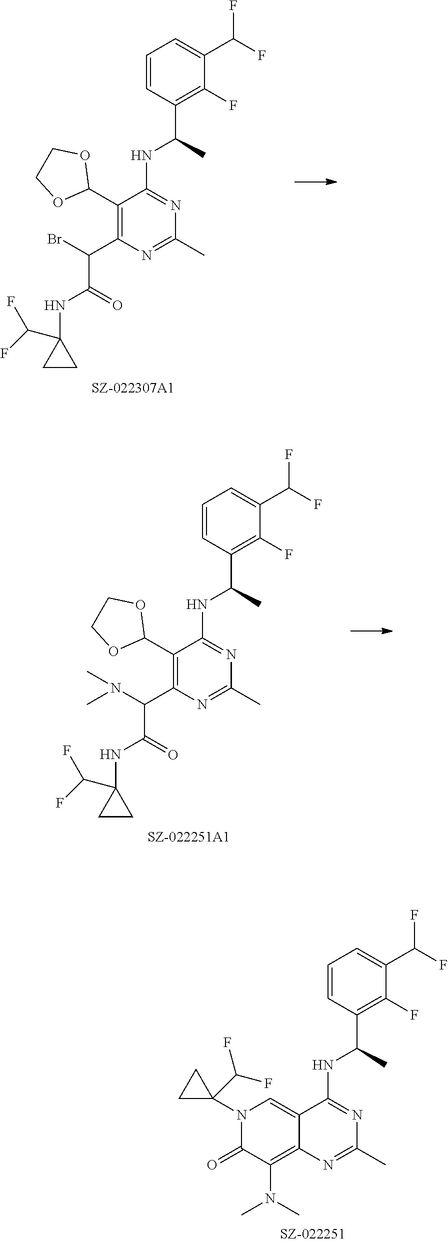 [SMILES: Cc1nc(N[C@H](C)c2cccc(C(F)F)c2F)c(C2OCCO2)c(C(Br)C(=O)NC2(C(F)F)CC2)n1 ; Cc1nc(N[C@H](C)c2cccc(C(F)F)c2F)c(C2OCCO2)c(C(C(=O)NC2(C(F)F)CC2)N(C)C)n1 ; Cc1nc(N[C@H](C)c2cccc(C(F)F)c2F)c2cn(C3(C(F)F)CC3)c(=O)c(N(C)C)c2n1]

### Step 1:

SZ-022307A1 (100 mg, 0.17 mmol), a solution of dimethylamine in tetrahydrofuran (0.10 mL, 2 M), and sodium carbonate (18.4 mg, 0.17 mmol) were mixed in tetrahydrofuran (5 mL) under argon atmosphere, and then the mixture was stirred at room temperature for 1 h. The reaction mixture was filtered and concentrated to give SZ-022251A1 as a yellow liquid (60 mg). LCMS: [M+H]⁺ 543.98.

### Step 2:

SZ-022251A1 (60 mg, 0.11 mmol) and hydrochloric acid (0.08 mL, 2 M) were mixed in isopropanol (3 mL) under argon atmosphere, and then the mixture was stirred at 40° C. for 1.5 h. After the reaction was completed, the reaction mixture was diluted with 10 mL of water. The dilution was extracted with 20 mL of ethyl acetate, and the organic phase was dried and concentrated to give a crude product. The crude product was purified by preparative reversed-phase chromatography and lyophilized to give SZ-022251 as a yellow solid (2.07 mg). LCMS: [M+H]⁺ 482.18.

SZ-022251: ¹H NMR (400 MHz, CD₃OD) δ 8.95 (s, 1H), 7.61 (t, J=8.0 Hz, 1H), 7.53 (t, J=8.0 Hz, 1H), 7.29 (t, J=8.0 Hz, 1H), 7.04 (t, J=56.0 Hz, 1H), 6.32 (t, J=56.0 Hz, 1H), 5.87-5.82 (m, 1H), 2.89 (s, 6H), 2.37 (s, 3H), 1.69 (d, J=4.0 Hz, 3H), 1.64-1.33 (m, 4H).

### Example 21: SZ-022327

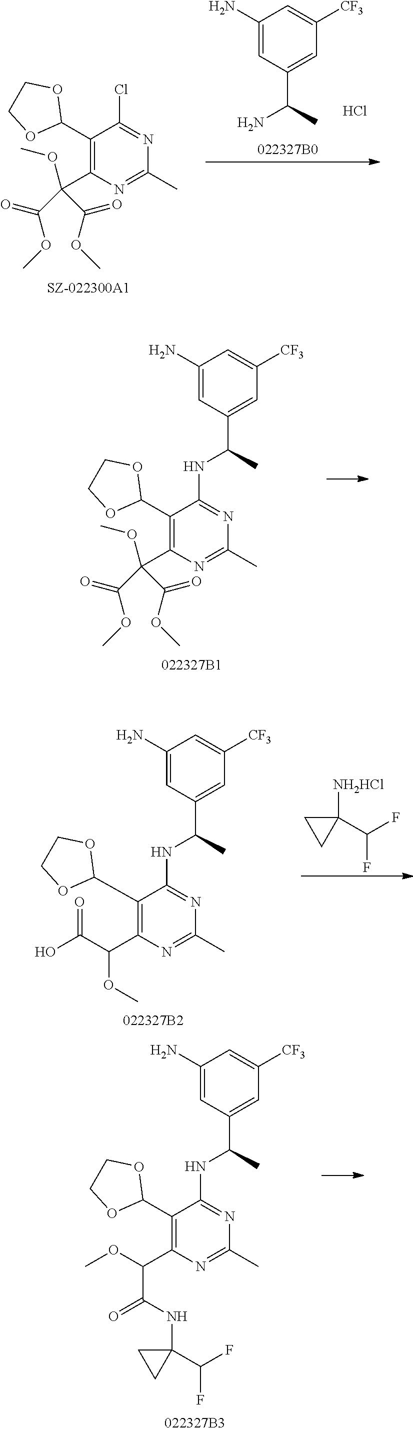

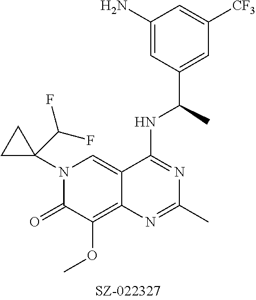 [SMILES: COc1c(=O)n(C2(C(F)F)CC2)cc2c(N[C@H](C)c3cc(N)cc(C(F)(F)F)c3)nc(C)nc12]

### Step 1:

SZ-022300A1 (270 mg, 0.75 mmol), 022327B0 (200 mg, 0.83 mmol), and triethylamine (400 mg, 3.75 mmol) were added to tetrahydrofuran (10 mL), and the mixture was heated to 80° C. for reaction for 16 h. After the starting materials were consumed as monitored by LC-MS, water (20 mL) and ethyl acetate (50 mL) were added to the reaction mixture for extraction, and the organic layer was separated, washed with a saturated sodium chloride solution, dried, and concentrated to dryness under reduced pressure to give a crude product. The crude product was purified by flash silica gel column chromatography (PE:EA=1:1) to give 022327B1 as a light yellow oil (270 mg). LC-MS: [M+H]⁺ 529.25.

### Step 2:

022327B1 (270 mg, 0.51 mmol) was added to dimethyl sulfoxide (4 mL), and then a 20% sodium hydroxide solution (500 μL) was added. The mixture was allowed to react at room temperature for 1 h. After the starting materials were consumed as monitored by LC-MS, the reaction mixture was used directly in the next step without work-up. LC-MS: [M+H]⁺ 457.19.

### Step 3:

(Difluoromethyl)cyclopropyl-1-amine hydrochloride (97 mg, 0.76 mmol), HATU (580 mg, 1.53 mmol), and triethylamine (260 mg, 2.55 mmol) were added to the reaction system in the previous step, and the mixture was allowed to react at room temperature for another 16 h. After the starting materials were consumed as monitored by LC-MS, water (20 mL) and ethyl acetate (50 mL) were added to the reaction mixture for extraction, and the organic layer was separated, washed with a saturated sodium chloride solution, dried, and concentrated to dryness under reduced pressure to give crude product 022327B3 as a light yellow oil (100 mg). LC-MS: [M+H]⁺ 546.26.

### Step 4:

022327B3 (50 mg, 0.09 mmol) was added to isopropanol (3 mL), and then a hydrochloric acid solution (2 M, 0.52 mL) was added. The mixture was allowed to react at 50° C. for 4 h. After the starting materials were consumed as monitored by LC-MS, the reaction mixture was directly concentrated to dryness under reduced pressure. A saturated sodium bicarbonate solution (5 mL), water (10 mL), and ethyl acetate (20 mL) were then added for extraction, and the organic layer was separated, washed with a saturated sodium chloride solution, dried, and concentrated to dryness under reduced pressure to give a crude product. The crude product was separated by preparative high performance liquid chromatography and then lyophilized to give SZ-022327 as a yellow solid (13.0 mg). LC-MS: [M+H]⁺ 483.98.

SZ-022327: ¹H NMR (400 MHz, CDCl₃) δ 8.31 (s, 1H), 7.05 (s, 1H), 6.91 (s, 1H), 6.81 (s, 2H), 6.31 (t, J=58.4 Hz, 1H), 5.75-5.70 (m, 1H), 4.06 (s, 3H), 3.92 (s, 2H), 2.47 (s, 3H), 1.64 (d, J=6.8 Hz, 3H), 1.56 (br, 2H), 1.26 (br, 2H).

### Example 22: SZ-022329

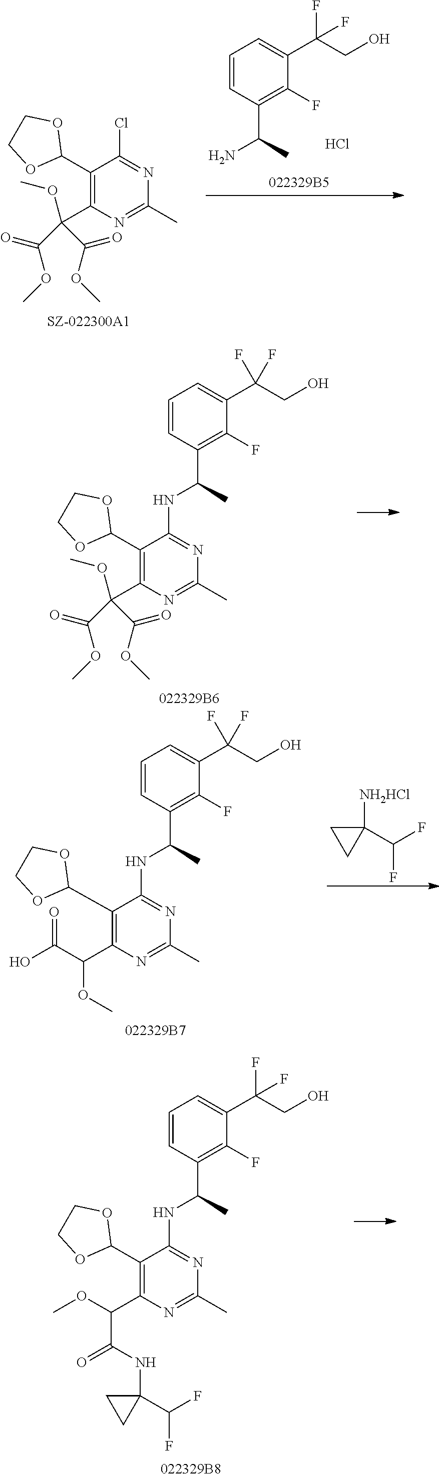

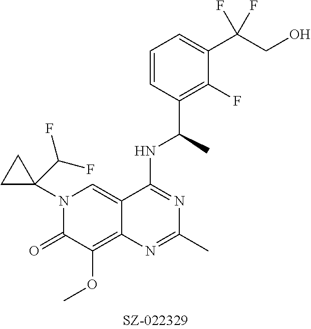 [SMILES: COc1c(=O)n(C2(C(F)F)CC2)cc2c(N[C@H](C)c3cccc(C(F)(F)CO)c3F)nc(C)nc12]

### Step 1:

SZ-022300A1 (180 mg, 0.50 mmol), 022329B5 (153 mg, 0.60 mmol, prepared by reference to the patent method), and triethylamine (253 mg, 2.50 mmol) were added to dimethyl sulfoxide (5 mL), and the mixture was heated to 70° C. for reaction for 16 h. After the starting materials were consumed as monitored by LC-MS, water (50 mL) and ethyl acetate (50 mL) were added to the reaction mixture for extraction, and the organic layer was separated, washed with a saturated sodium chloride solution, dried, and concentrated to dryness under reduced pressure to give a crude product. The crude product was purified by flash silica gel column chromatography (PE:EA=1:2) to give compound 022329B6 as a yellow solid (70 mg). LC-MS: [M+H]⁺ 544.23.

### Step 2:

022329B6 (70 mg, 0.13 mmol) was added to dimethyl sulfoxide (2 mL), and then a 20% sodium hydroxide solution (150 μL) was added. The mixture was allowed to react at room temperature for 3 h. After the starting materials were consumed as monitored by LC-MS, the reaction mixture was used directly in the next step without work-up. LC-MS: [M+H]⁺ 472.17.

### Step 3:

(Difluoromethyl)cyclopropyl-1-amine hydrochloride (28 mg, 0.20 mmol), HATU (148 mg, 0.39 mmol), and triethylamine (66 mg, 0.65 mmol) were added to the reaction system in the previous step, and the mixture was allowed to react at room temperature for 16 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and then concentrated to dryness under reduced pressure to give a crude product. The crude product was purified by flash silica gel column chromatography (DCM:MeOH=20:1) to give 022329B8 as a light yellow oil (50 mg). LC-MS: [M+H]⁺ 561.29.

### Step 4:

022329B8 (50 mg, 0.09 mmol) was added to isopropanol (2 mL), and then a hydrochloric acid solution (2 M, 0.44 mL) was added. The mixture was allowed to react at 50° C. for 4 h. After the starting materials were consumed as monitored by LC-MS, the reaction mixture was directly concentrated to dryness under reduced pressure. A saturated sodium bicarbonate solution (5 mL), water (10 mL), and ethyl acetate (20 mL) were then added for extraction, and the organic layer was separated, washed with a saturated sodium chloride solution, dried, and then concentrated to dryness under reduced pressure to give a crude product. The crude product was subjected to preparative high performance liquid chromatography and then lyophilized to give SZ-022329 as a yellow solid (30 mg). LC-MS: [M+H]⁺ 499.16.

SZ-022329: ¹H NMR (400 MHz, DMSO-d₆) δ 8.92 (s, 1H), 8.78 (d, J=7.2 Hz, 1H), 7.59 (t, J=7.2 Hz, 1H), 7.43 (t, J=7.2 Hz, 1H), 7.28 (t, J=7.6 Hz, 1H), 6.34 (t, J=57.2 Hz, 1H), 5.77-5.68 (m, 2H), 3.97-3.88 (m, 2H), 3.77 (s, 3H), 2.22 (s, 3H), 1.57 (d, J=6.8 Hz, 3H), 1.49-1.39 (m, 4H).

### Example 23 and Example 24: SZ-022330 and SZ-022330B

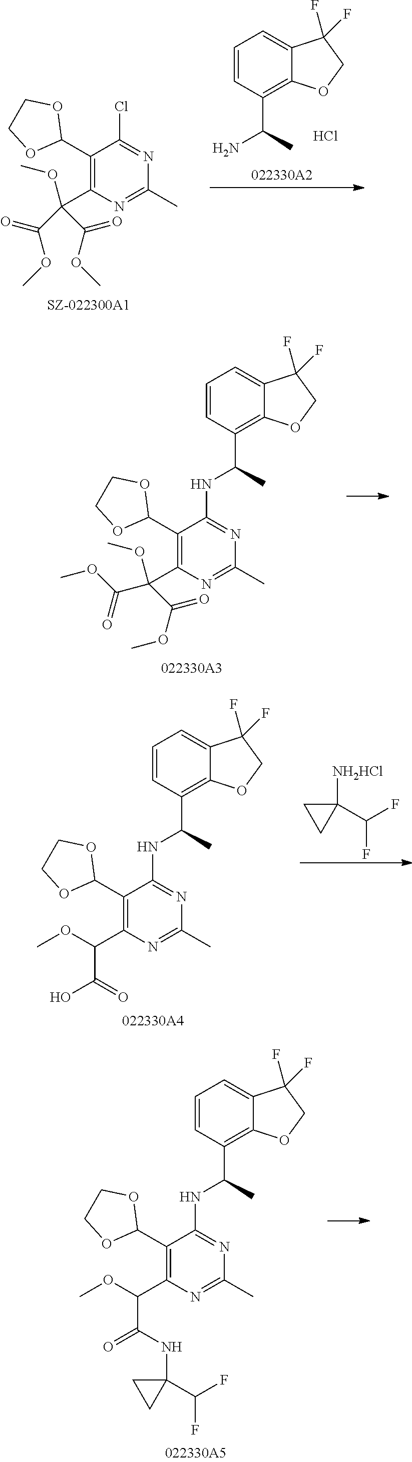

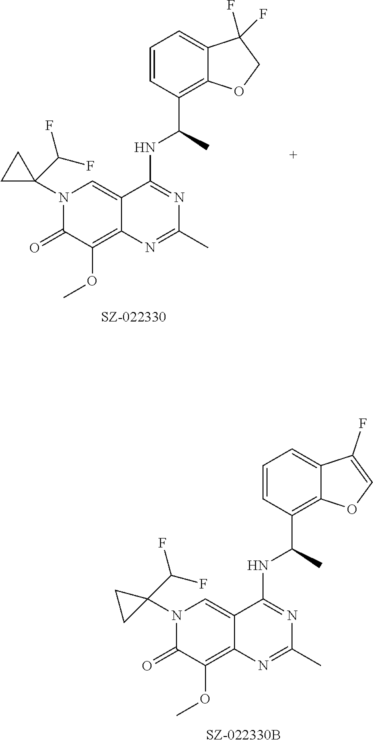 [SMILES: COc1c(=O)n(C2(C(F)F)CC2)cc2c(N[C@H](C)c3cccc4c3OCC4(F)F)nc(C)nc12 ; COc1c(=O)n(C2(C(F)F)CC2)cc2c(N[C@H](C)c3cccc4c(F)coc34)nc(C)nc12]

### Step 1:

SZ-022300A1 (90 mg, 0.25 mmol), 022330A2 (71 mg, 0.30 mmol, prepared by reference to the patent method), and triethylamine (126 mg, 1.25 mmol) were added to tetrahydrofuran (5 mL), and the mixture was heated to 80° C. for reaction for 16 h. After the starting materials were consumed as monitored by LC-MS, water (20 mL) and ethyl acetate (50 mL) were added to the reaction mixture for extraction, and the organic layer was separated, washed with a saturated sodium chloride solution, dried, and concentrated to dryness under reduced pressure to give a crude product. The crude product was purified by flash silica gel column chromatography (PE:EA=1:1) to give 022330A3 as a light yellow oil (70 mg). LC-MS: [M+H]⁺ 524.26.

### Step 2:

022330A3 (70 mg, 0.13 mmol) was added to dimethyl sulfoxide (2 mL), and then a 20% sodium hydroxide solution (150 μL) was added. The mixture was allowed to react at room temperature for 3 h. After the starting materials were consumed as monitored by LC-MS, the reaction mixture was used directly in the next step without work-up. LC-MS: [M+H]⁺ 432.28, 452.28.

### Step 3:

(Difluoromethyl)cyclopropyl-1-amine hydrochloride (28 mg, 0.20 mmol), HATU (148 mg, 0.39 mmol), and triethylamine (66 mg, 0.65 mmol) were added to the reaction system in the previous step, and the mixture was allowed to react at room temperature for another 16 h. After the starting materials were consumed as monitored by LC-MS, water (20 mL) and ethyl acetate (30 mL) were added to the reaction mixture for extraction, and the organic layer was separated, washed with a saturated sodium chloride solution, dried, and concentrated to dryness under reduced pressure to give crude product 022330A5 as a light yellow oil (70 mg). LC-MS: [M+H]⁺ 521.23, 541.22.

### Step 4:

022330A5 (70 mg, 0.13 mmol) was added to isopropanol (3 mL), and then a hydrochloric acid solution (2 M, 0.52 mL) was added. The mixture was allowed to react at 50° C. for 4 h. After the starting materials were consumed as monitored by LC-MS, the reaction mixture was directly concentrated to dryness under reduced pressure. A saturated sodium bicarbonate solution (5 mL), water (10 mL), and ethyl acetate (20 mL) were then added for extraction, and the organic layer was separated, washed with a saturated sodium chloride solution, dried, and concentrated to dryness under reduced pressure to give a crude product. The crude product was separated by preparative high performance liquid chromatography and then lyophilized to give SZ-022330 as a yellow solid (14.8 mg) (LC-MS: [M+H]⁺ 478.90) and SZ-022330B as a yellow solid (12.4 mg). LC-MS: [M+H]⁺ 458.86.

SZ-022330: ¹H NMR (400 MHz, CDCl₃) δ 7.97 (br, 1H), 7.48-7.44 (m, 2H), 7.05 (t, J=7.6 Hz, 1H), 6.30 (t, J=58.4 Hz, 1H), 5.75-5.70 (m, 1H), 4.77-4.58 (m, 2H), 4.06 (s, 3H), 2.47 (s, 3H), 1.66 (d, J=6.8 Hz, 3H), 1.63-1.61 (m, 2H), 1.31-1.24 (m, 2H).

SZ-022330B: ¹H NMR (400 MHz, CDCl₃) δ 8.05 (br, 1H), 7.62 (d, J=4.4 Hz, 1H), 7.53 (d, J=8.8 Hz, 1H), 7.38 (d, J=7.2 Hz, 1H), 7.26-7.23 (m, 2H), 6.30 (t, J=58.4 Hz, 1H), 6.01-5.96 (m, 1H), 4.04 (s, 3H), 2.46 (s, 3H), 1.78 (d, J=7.2 Hz, 3H), 1.63-1.59 (m, 2H), 1.29-1.24 (m, 2H).

### Example 25: SZ-022224

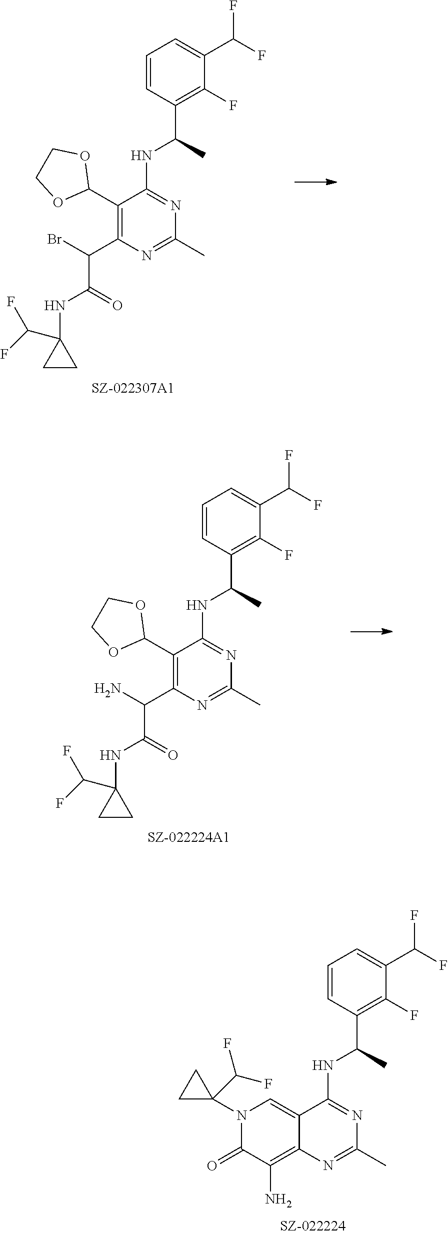 [SMILES: Cc1nc(N[C@H](C)c2cccc(C(F)F)c2F)c(C2OCCO2)c(C(Br)C(=O)NC2(C(F)F)CC2)n1 ; Cc1nc(N[C@H](C)c2cccc(C(F)F)c2F)c(C2OCCO2)c(C(N)C(=O)NC2(C(F)F)CC2)n1 ; Cc1nc(N[C@H](C)c2cccc(C(F)F)c2F)c2cn(C3(C(F)F)CC3)c(=O)c(N)c2n1]

### Step 1:

SZ-022307A1 (80 mg, 0.14 mmol) was dissolved in a solution of amine in methanol (5.00 mL, 7 M) under argon atmosphere, and then the mixture was microwaved at 130° C. for reaction for half an hour. The reaction mixture was concentrated to give SZ-022224A1 as a yellow liquid (90 mg). LCMS: [M+H]⁺ 515.95.

### Step 2:

SZ-022224A1 (90 mg, 0.17 mmol) and hydrochloric acid (0.2 mL, 5 M) were mixed in isopropanol (3 mL) under argon atmosphere, and then the mixture was stirred at 40° C. for half an hour. After the reaction was completed, the reaction mixture was diluted with 10 mL of water. The dilution was extracted with 20 mL of ethyl acetate, and the organic phase was dried and concentrated to give a crude product. The crude product was purified by preparative reversed-phase chromatography and lyophilized to give SZ-022224 as a yellow solid (1.73 mg). LCMS: [M+H]⁺ 453.91.

SZ-022224: ¹H NMR (400 MHz, CD₃OD) δ 7.73 (s, 1H), 7.62 (t, J=8.0 Hz, 1H), 7.51 (t, J=8.0 Hz, 1H), 7.27 (t, J=8.0 Hz, 1H), 7.05 (t, J=56.0 Hz, 1H), 6.06 (t, J=56.0 Hz, 1H), 5.92-5.85 (m, 1H), 2.44 (s, 3H), 1.68 (d, J=4.0 Hz, 3H), 1.38-1.08 (m, 4H).

### Example 26: SZ-022344

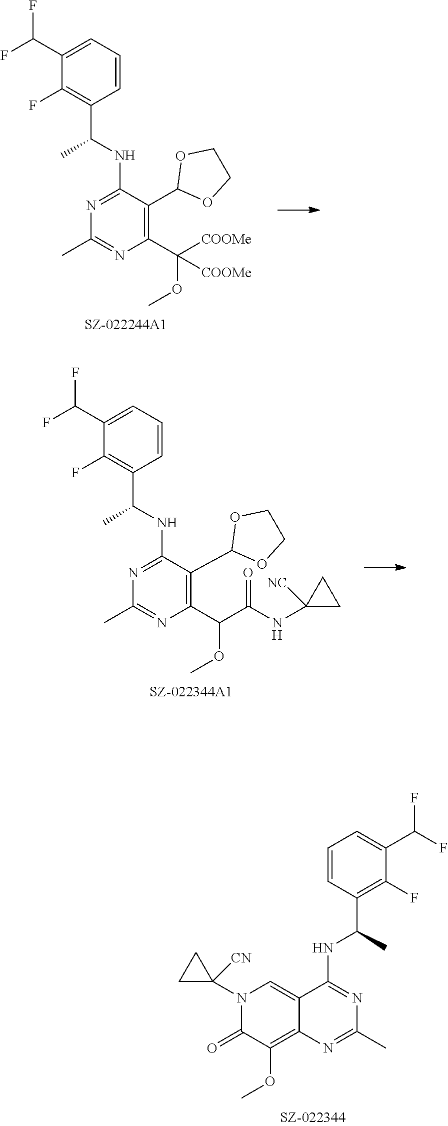 [SMILES: COC(=O)C(OC)(C(=O)OC)c1nc(C)nc(N[C@H](C)c2cccc(C(F)F)c2F)c1C1OCCO1 ; COC(C(=O)NC1(C#N)CC1)c1nc(C)nc(N[C@H](C)c2cccc(C(F)F)c2F)c1C1OCCO1 ; COc1c(=O)n(C2(C#N)CC2)cc2c(N[C@H](C)c3cccc(C(F)F)c3F)nc(C)nc12]

### Step 1:

SZ-022244A1 (100 mg, 0.19 mmol) was added to dimethyl sulfoxide (2 mL), and then a 10% sodium hydroxide solution (380 μL) was added. The mixture was allowed to react at 40° C. for 1 h. 1-Amino-1-cyclopropanecarbonitrile hydrochloride (34 mg, 0.28 mmol), N,N,N,N-tetramethyl-O-(7-azabenzotriazol-1-yl)uronium hexafluorophosphate (216 mg, 0.57 mmol), and triethylamine (58 mg, 0.57 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022344A1 as a yellow viscous substance (100 mg). LC-MS: [M+H]⁺ 506.08.

### Step 2:

SZ-022344A1 (100 mg, 0.20 mmol) was added to isopropanol (2 mL), and then a hydrochloric acid solution (4 M, 0.25 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022344 (19.4 mg). LC-MS: [M+H]⁺ 444.14.

SZ-022344: ¹H NMR (400 MHz, DMSO-d₆) δ 8.97 (s, 1H), 8.75 (d, J=5.2 Hz, 1H) 7.68 (t, J=7.2 Hz, 1H), 7.53 (t, J=7.2 Hz, 1H), 7.32 (t, J=7.6 Hz, 1H), 5.78-5.71 (m, 1H), 3.80 (s, 3H), 2.22 (s, 3H), 2.05-1.97 (m, 2H), 1.82-1.74 (m, 2H), 1.58 (d, J=7.2 Hz, 3H).

### Example 27: SZ-022343

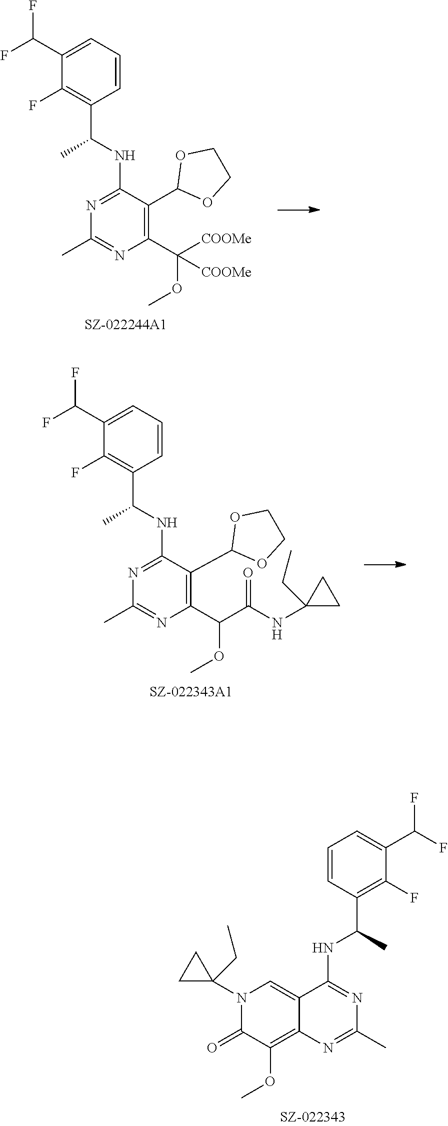 [SMILES: COC(=O)C(OC)(C(=O)OC)c1nc(C)nc(N[C@H](C)c2cccc(C(F)F)c2F)c1C1OCCO1 ; CCC1(NC(=O)C(OC)c2nc(C)nc(N[C@H](C)c3cccc(C(F)F)c3F)c2C2OCCO2)CC1 ; CCC1(n2cc3c(N[C@H](C)c4cccc(C(F)F)c4F)nc(C)nc3c(OC)c2=O)CC1]

### Step 1:

SZ-022244A1 (110 mg, 0.21 mmol) was added to dimethyl sulfoxide (2 mL), and then a 10% sodium hydroxide solution (400 μL) was added. The mixture was allowed to react at 40° C. for 1 h. 1-Ethylcyclopropanamine hydrochloride (35 mg, 0.29 mmol), N,N,N,N-tetramethyl-O-(7-azabenzotriazol-1-yl)uronium hexafluorophosphate (223 mg, 0.59 mmol), and triethylamine (59 mg, 0.59 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022343A1 as a yellow viscous substance (110 mg). LC-MS: [M+H]⁺ 509.29.

### Step 2:

SZ-022343A1 (110 mg, 0.22 mmol) was added to isopropanol (2 mL), and then a hydrochloric acid solution (4 M, 0.25 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022343 (71 mg). LC-MS: [M+H]⁺ 447.18.

SZ-022343: ¹H NMR (400 MHz, DMSO-d₆) δ 8.91 (s, 1H), 8.73 (d, J=7.2 Hz, 1H), 7.68-7.60 (m, 1H), 7.52 (t, J=7.2 Hz, 1H), 7.31 (t, J=7.6 Hz, 1H), 7.24 (t, J=54.4 Hz, 1H), 5.79-5.71 (m, 1H), 3.74 (s, 3H), 2.20 (s, 3H), 1.58 (d, J=7.2 Hz, 3H), 1.10 (br, 4H), 0.81 (t, J=7.2 Hz, 3H).

### Example 28: SZ-022295

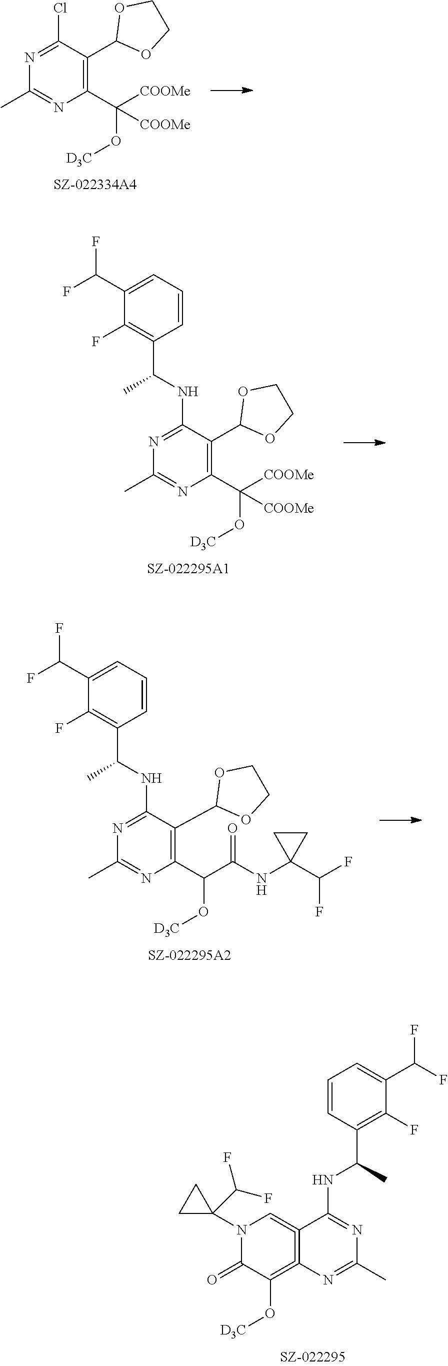

### Step 1:

SZ-022334A4 (120 mg, 0.33 mmol), (1R)-1-(2-fluoro-3-difluoromethylphenyl)ethylamine hydrochloride (112 mg, 0.49 mmol, synthesized according to WO2019122129), triethylamine (100 mg, 0.98 mmol), and potassium carbonate (46 mg, 0.33 mmol) were added to dimethyl sulfoxide (5 mL), and the mixture was heated to 60° C. for reaction for 8 h. After the starting materials were consumed as monitored by LC-MS, water (100 mL) and ethyl acetate (100 mL) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA=3:1; Rf=0.3) to give product SZ-022295A1 as a yellow oil (110 mg). LC-MS: [M+H]⁺ 516.90.

### Step 2:

SZ-022295A1 (110 mg, 0.21 mmol) was added to dimethyl sulfoxide (2 mL), and then a 10% sodium hydroxide solution (430 μL) was added. The mixture was allowed to react at 40° C. for 1 h. 1-Difluoromethylcyclopropanamine hydrochloride (40 mg, 0.32 mmol), N,N,N,N-tetramethyl-O-(7-azabenzotriazol-1-yl)uronium hexafluorophosphate (243 mg, 0.64 mmol), and triethylamine (65 mg, 0.64 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022295A2 as a yellow viscous substance (110 mg). LC-MS: [M+H]⁺ 534.27.

### Step 3:

SZ-022295A2 (110 mg, 0.25 mmol) was added to isopropanol (5 mL), and then a hydrochloric acid solution (2 M, 0.31 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022295 (47 mg). LC-MS: [M+H]⁺ 472.20.

SZ-022295: ¹H NMR (400 MHz, DMSO-d₆) δ 8.93 (s, 1H), 8.81 (d, J=7.2 Hz, 1H), 7.63 (t, J=7.2 Hz, 1H), 7.52 (t, J=7.2 Hz, 1H), 7.32 (t, J=7.6 Hz, 1H), 7.23 (t, J=54.4 Hz, 1H), 6.34 (t, J=57.2 Hz, 1H), 5.78-5.71 (m, 1H), 2.21 (s, 3H), 1.58 (d, J=7.2 Hz, 3H), 1.52-1.45 (m, 2H), 1.45-1.32 (m, 2H).

### Example 29: SZ-022339

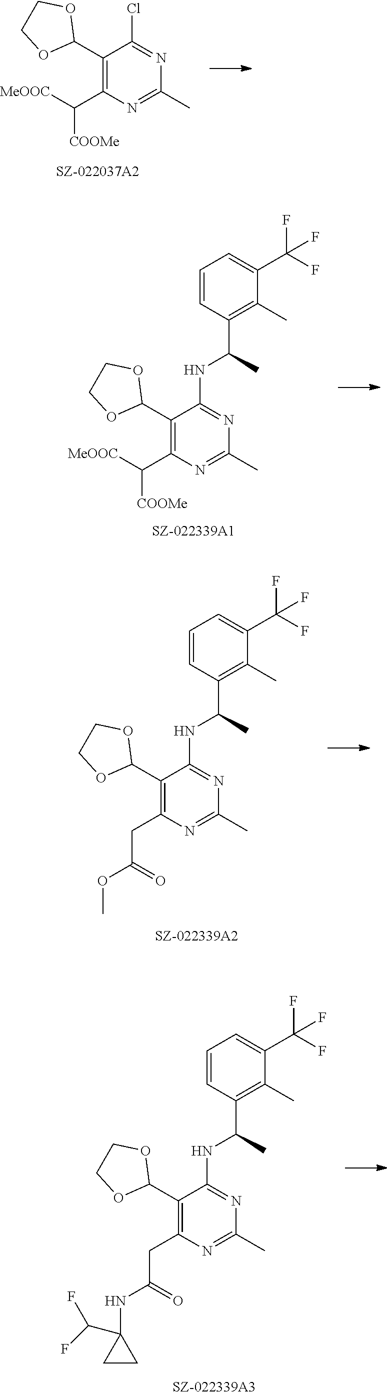 [SMILES: COC(=O)C(C(=O)OC)c1nc(C)nc(Cl)c1C1OCCO1 ; COC(=O)C(C(=O)OC)c1nc(C)nc(N[C@H](C)c2cccc(C(F)(F)F)c2C)c1C1OCCO1 ; COC(=O)Cc1nc(C)nc(N[C@H](C)c2cccc(C(F)(F)F)c2C)c1C1OCCO1 ; Cc1nc(CC(=O)NC2(C(F)F)CC2)c(C2OCCO2)c(N[C@H](C)c2cccc(C(F)(F)F)c2C)n1]

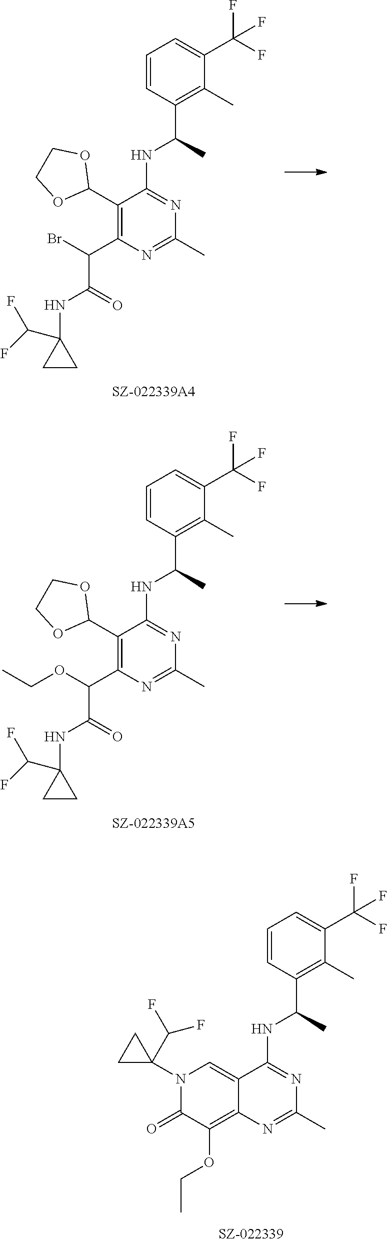 [SMILES: Cc1nc(N[C@H](C)c2cccc(C(F)(F)F)c2C)c(C2OCCO2)c(C(Br)C(=O)NC2(C(F)F)CC2)n1 ; CCOC(C(=O)NC1(C(F)F)CC1)c1nc(C)nc(N[C@H](C)c2cccc(C(F)(F)F)c2C)c1C1OCCO1 ; CCOc1c(=O)n(C2(C(F)F)CC2)cc2c(N[C@H](C)c3cccc(C(F)(F)F)c3C)nc(C)nc12]

### Step 1:

SZ-022037A2 (7.8 g, 23.6 mmol, synthesized according to WO2019122129), (1R)-1-(2-methyl-3-trifluoromethylphenyl)ethylamine hydrochloride (5.7 g, 23.6 mmol, synthesized according to WO2019122129), and diisopropylethylamine (9.2 g, 70.8 mmol) were added to N,N-dimethylformamide (80 mL), and the mixture was heated to 80° C. for reaction for 18 h. After the starting materials were consumed as monitored by LC-MS, water (500 mL) and ethyl acetate (250 mL) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA=1:1; Rf=0.3) to give product SZ-022339A1 as a yellow oil (8.7 g). LC-MS: [M+H]⁺ 498.07.

### Step 2:

SZ-022339A1 (8.7 g, 17.5 mmol) and lithium chloride (2.97 g, 70.0 mmol) were added to dimethyl sulfoxide (300 mL), and the mixture was heated to 120° C. for reaction for 16 h. After the starting materials were consumed as monitored by LC-MS, water (1500 mL) and ethyl acetate (500 mL) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA=3:7; Rf=0.4) to give product SZ-022339A2 as a yellow oil (3.6 g). LC-MS: [M+H]⁺ 440.17.

### Step 3:

SZ-022339A2 (500 mg, 1.14 mmol) and sodium hydroxide (182 mg, 4.56 mmol) were added to dimethyl sulfoxide (5 mL), and the mixture was allowed to react at room temperature for 1 h. After the starting materials were consumed as monitored by LC-MS, triethylamine (346 mg, 3.42 mmol), 1-(difluoromethylcyclopropyl)amine hydrochloride (245 mg, 1.71 mmol), and N,N,N,N-tetramethyl-O-(7-azabenzotriazol-1-yl)uronium hexafluorophosphate (1300 mg, 3.42 mmol) were added to the reaction mixture, and the resulting mixture was allowed to react at room temperature for 2 h. Water (50 mL) and ethyl acetate (50 mL) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA=0:1; Rf=0.5) to give SZ-022339A3 as a white solid (500 mg). LC-MS: [M+H]⁺ 515.21.

### Step 4:

SZ-022339A3 (500 mg, 0.97 mmol), N-bromosuccinimide (173 mg, 0.97 mmol), and azobisisobutyronitrile (16 mg, 0.097 mmol) were added to carbon tetrachloride (15 mL), and the mixture was heated to 60° C. for reaction for 1 h. After the starting materials were consumed as monitored by LC-MS, the reaction mixture was concentrated by rotary evaporation to give a crude product. The crude product was purified by flash silica gel column chromatography (PE:EA=3:7; Rf=0.5) to give product SZ-022339A4 as a yellow oil (360 mg). LC-MS: [M+H]⁺ 592.88, 594.79.

### Step 5:

Ethanol (340 mg, 7.40 mmol) was dissolved in tetrahydrofuran (10 mL), and sodium hydride (88 mg, 2.22 mmol) was slowly added under argon atmosphere in an ice-water bath. The reaction mixture was stirred for half an hour, and a solution of SZ-022339A4 (360 mg, 0.61 mmol) in tetrahydrofuran (1 mL) was slowly added dropwise into the above system. The resulting mixture was warmed to room temperature and stirred for 2 h. After the starting materials were consumed as monitored by LC-MS, a saturated ammonium chloride solution (50 mL) and ethyl acetate (100 mL) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA=0:1; Rf=0.5) to give product SZ-022339A5 as a yellow oil (210 mg). LC-MS: [M+H]⁺ 559.23.

### Step 6:

SZ-022339A5 (210 mg, 0.38 mmol) and hydrochloric acid (0.2 mL, 5 M) were added to isopropanol (2 mL), and the mixture was heated to 50° C. for reaction for 6 h. After the starting materials were consumed as monitored by LC-MS, water (50 mL) and ethyl acetate (50 mL) were added to the reaction mixture for extraction, and the organic layer was separated, washed with a saturated sodium chloride solution, dried, and concentrated by rotary evaporation to give a crude product. The crude product was subjected to preparative high performance liquid chromatography and then lyophilized to give SZ-022339 (28.49 mg). LC-MS: [M+H]⁺ 497.2.

SZ-022339: ¹H NMR (400 MHz, DMSO-d₆) δ 8.90 (s, 1H), 8.87 (d, J=8.0 Hz, 1H), 7.70 (d, J=8.0 Hz, 1H), 7.56 (d, J=8.0 Hz, 1H), 7.39 (t, J=8.0 Hz, 1H), 6.34 (t, J=56.0 Hz, 1H), 5.70-5.67 (m, 1H), 4.06 (q, J=8.0 Hz, 2H), 2.57 (s, 3H), 2.20 (s, 3H), 1.53 (d, J=8.0 Hz, 3H), 1.50-1.35 (m, 4H), 1.21 (t, J=8.0 Hz, 3H).

### Example 30: SZ-022340

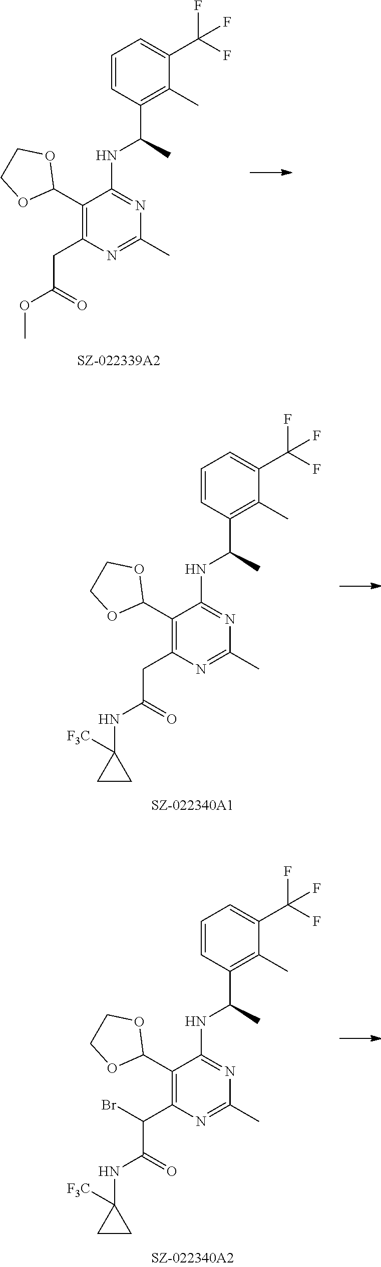 [SMILES: COC(=O)Cc1nc(C)nc(N[C@H](C)c2cccc(C(F)(F)F)c2C)c1C1OCCO1 ; Cc1nc(CC(=O)NC2(C(F)(F)F)CC2)c(C2OCCO2)c(N[C@H](C)c2cccc(C(F)(F)F)c2C)n1 ; Cc1nc(N[C@H](C)c2cccc(C(F)(F)F)c2C)c(C2OCCO2)c(C(Br)C(=O)NC2(C(F)(F)F)CC2)n1]

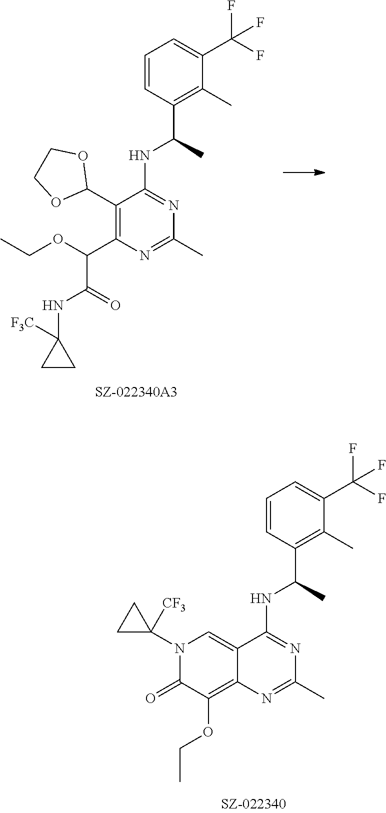 [SMILES: CCOC(C(=O)NC1(C(F)(F)F)CC1)c1nc(C)nc(N[C@H](C)c2cccc(C(F)(F)F)c2C)c1C1OCCO1 ; CCOc1c(=O)n(C2(C(F)(F)F)CC2)cc2c(N[C@H](C)c3cccc(C(F)(F)F)c3C)nc(C)nc12]

### Step 1:

SZ-022339A2 (500 mg, 1.14 mmol) and sodium hydroxide (182 mg, 4.56 mmol) were added to dimethyl sulfoxide (5 mL), and the mixture was allowed to react at room temperature for 1 h. After the starting materials were consumed as monitored by LC-MS, triethylamine (346 mg, 3.42 mmol), 1-(trifluoromethylcyclopropyl)amine hydrochloride (276 mg, 1.71 mmol), and N,N,N,N-tetramethyl-O-(7-azabenzotriazol-1-yl)uronium hexafluorophosphate (1300 mg, 3.42 mmol) were added to the reaction mixture, and the resulting mixture was allowed to react at room temperature for 2 h. Water (50 mL) and ethyl acetate (50 mL) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA=0:1; Rf=0.5) to give SZ-022340A1 as a yellow solid (440 mg). LC-MS: [M+H]⁺ 533.12.

### Step 2:

SZ-022340A1 (440 mg, 0.83 mmol), N-bromosuccinimide (147 mg, 0.83 mmol), and azobisisobutyronitrile (14 mg, 0.083 mmol) were added to carbon tetrachloride (15 mL), and the mixture was heated to 60° C. for reaction for 1 h. After the starting materials were consumed as monitored by LC-MS, the reaction mixture was concentrated by rotary evaporation to give a crude product. The crude product was purified by flash silica gel column chromatography (PE:EA=3:7; Rf=0.5) to give SZ-022340A2 as a white solid (280 mg). LC-MS: [M+H]⁺ 610.90, 612.83.

### Step 3:

Ethanol (210 mg, 4.60 mmol) was dissolved in tetrahydrofuran (10 mL), and sodium hydride (56 mg, 1.38 mmol) was slowly added under argon atmosphere in an ice-water bath. The reaction mixture was stirred for half an hour, and a solution of SZ-022340A2 (280 mg, 0.46 mmol) in tetrahydrofuran (1 mL) was slowly added dropwise into the above system. The resulting mixture was warmed to room temperature and stirred for 2 h. After the starting materials were consumed as monitored by LC-MS, a saturated ammonium chloride solution (50 mL) and ethyl acetate (100 mL) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA=0:1; Rf=0.5) to give product SZ-022340A3 as a yellow oil (70 mg). LC-MS: [M+H]⁺ 577.20.

### Step 4:

SZ-022340A3 (70 mg, 0.12 mmol) and hydrochloric acid (0.1 mL, 5 M) were added to isopropanol (2 mL), and the mixture was heated to 50° C. for reaction for 6 h. After the starting materials were consumed as monitored by LC-MS, water (50 mL) and ethyl acetate (50 mL) were added to the reaction mixture for extraction, and the organic layer was separated, washed with a saturated sodium chloride solution, dried, and concentrated by rotary evaporation to give a crude product. The crude product was subjected to preparative high performance liquid chromatography and then lyophilized to give SZ-022340 (13.02 mg). LC-MS: [M+H]⁺ 515.18.

SZ-022340: ¹H NMR (400 MHz, DMSO-d₆) δ 8.96 (s, 1H), 8.87 (d, J=8.0 Hz, 1H), 7.70 (d, J=8.0 Hz, 1H), 7.56 (d, J=8.0 Hz, 1H), 7.39 (t, J=8.0 Hz, 1H), 5.70-5.66 (m, 1H), 4.05 (q, J=8.0 Hz, 2H), 2.57 (s, 3H), 2.20 (s, 3H), 1.81-1.65 (m, 4H), 1.54 (d, J=8.0 Hz, 3H), 1.20 (t, J=8.0 Hz, 3H).

### Example 31: SZ-022345

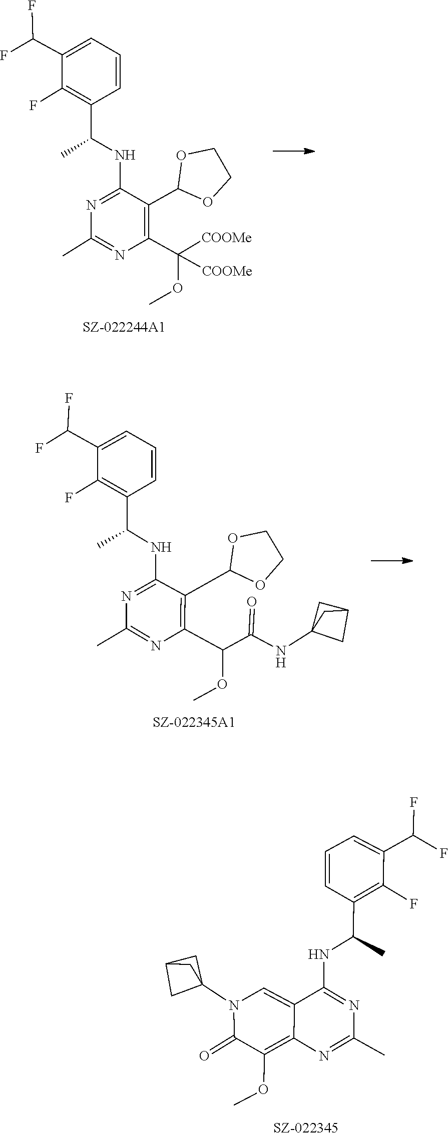 [SMILES: COC(=O)C(OC)(C(=O)OC)c1nc(C)nc(N[C@H](C)c2cccc(C(F)F)c2F)c1C1OCCO1 ; COC(C(=O)NC12CC(C1)C2)c1nc(C)nc(N[C@H](C)c2cccc(C(F)F)c2F)c1C1OCCO1 ; COc1c(=O)n(C23CC(C2)C3)cc2c(N[C@H](C)c3cccc(C(F)F)c3F)nc(C)nc12]

### Step 1:

SZ-022244A1 (100 mg, 0.19 mmol) was added to dimethyl sulfoxide (2 mL), and then a 10% sodium hydroxide solution (500 μL) was added. The mixture was allowed to react at 40° C. for 1 h. 1-Bicyclo[1,1,1]pentylamine hydrochloride (34 mg, 0.28 mmol), N,N,N,N-tetramethyl-O-(7-azabenzotriazol-1-yl)uronium hexafluorophosphate (216 mg, 0.57 mmol), and triethylamine (58 mg, 0.57 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022345A1 as a yellow viscous substance (210 mg). LC-MS: [M+H]⁺ 507.13.

### Step 2:

SZ-022345A1 (210 mg, 0.19 mmol) was added to isopropanol (2 mL), and then a hydrochloric acid solution (2 M, 0.6 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022345 (29 mg). LC-MS: [M+H]⁺ 444.89.

SZ-022345: ¹H NMR (400 MHz, DMSO-d₆) δ 8.77 (d, J=7.2 Hz, 1H), 8.60 (s, 1H), 7.65 (t, J=7.2 Hz, 1H), 7.52 (t, J=6.8 Hz, 1H), 7.32 (t, J=8.0 Hz, 1H), 7.24 (t, J=54.4 Hz, 1H), 5.81-5.74 (m, 1H), 3.75 (s, 3H), 2.72 (s, 1H), 2.21 (s, 3H), 1.59 (d, J=7.2 Hz, 3H).

### Example 32: SZ-022346

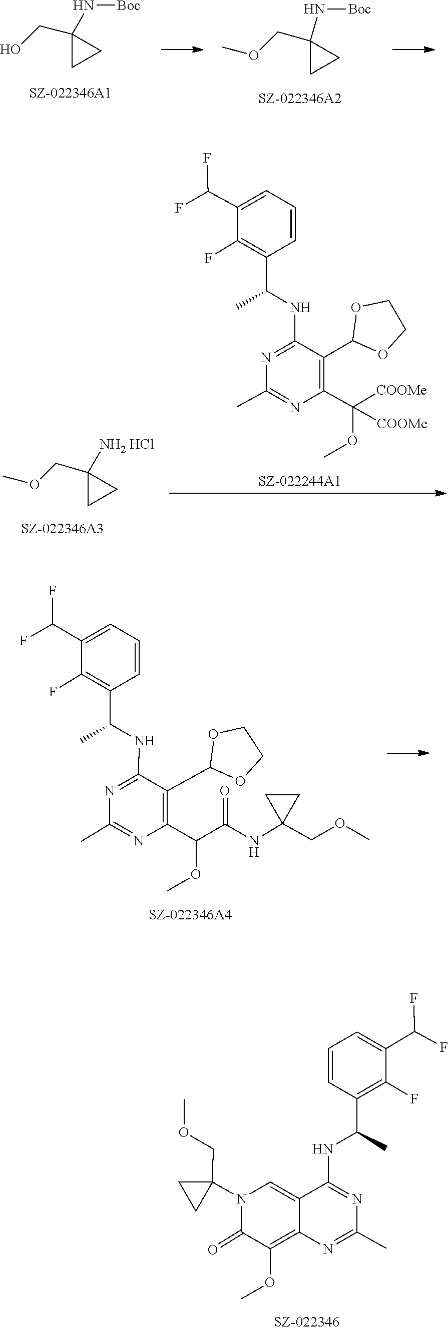

### Step 1:

SZ-022346A1 (500 mg, 2.67 mmol) was added to N,N-dimethylformamide (5 mL), and the mixture was cooled to 0° C. under argon atmosphere. Sodium hydride (128 mg, 3.20 mmol) was added, and the mixture was stirred at 0° C. for 0.5 h. Methyl iodide (417 mg, 2.94 mmol) was then added dropwise, and the resulting mixture was naturally warmed to room temperature and stirred for 15 h. After the starting materials were consumed as monitored by TLC, water (50 mL) and ethyl acetate (50 mL) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA=10:1; Rf=0.5) to give product SZ-022346A2 as a colorless oil (220 mg).

SZ-022346A2: ¹H NMR (400 MHz, CDCl₃) δ 5.03 (s, 1H), 4.14-4.09 (m, 1H), 3.38 (s, 2H), 3.36 (s, 3H), 1.44 (s, 9H), 0.83-0.72 (m, 4H).

### Step 2:

SZ-022346A2 (220 mg, 0.99 mmol) was added to a solution of hydrogen chloride in 1,4-dioxane (4 M, 5 mL), and the mixture was allowed to react at room temperature for 1 h. After the starting materials were consumed as monitored by TLC, the reaction mixture was concentrated by rotary evaporation to give SZ-022346A3 as an off-white solid (150 mg). The crude product was used directly in the next step.

### Step 3:

SZ-022244A1 (100 mg, 0.19 mmol) was added to dimethyl sulfoxide (5 mL), and then a 10% sodium hydroxide solution (500 μL) was added. The mixture was allowed to react at 40° C. for 1 h. SZ-022346A3 (150 mg, 0.99 mmol), N,N,N,N-tetramethyl-O-(7-azabenzotriazol-1-yl)uronium hexafluorophosphate (216 mg, 0.57 mmol), and triethylamine (58 mg, 0.57 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022346A4 as a light yellow viscous substance (180 mg). LC-MS: [M+H]⁺ 525.12.

### Step 4:

SZ-022346A4 (180 mg, 0.19 mmol) was added to isopropanol (2 mL), and then a hydrochloric acid solution (2 M, 0.5 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022346 (60 mg). LC-MS: [M+H]⁺ 462.96.

SZ-022346: ¹H NMR (400 MHz, DMSO-d₆) δ 8.94 (s, 1H), 8.84 (d, J=6.8 Hz, 1H), 7.66 (t, J=7.2 Hz, 1H), 7.55 (t, J=7.2 Hz, 1H), 7.35 (t, J=8.0 Hz, 1H), 7.26 (t, J=54.4 Hz, 1H), 5.82-5.75 (m, 1H), 3.78 (s, 3H), 3.24 (s, 3H), 2.24 (s, 3H), 1.61 (d, J=7.2 Hz, 3H), 1.20 (s, 4H).

### Example 33: SZ-022347

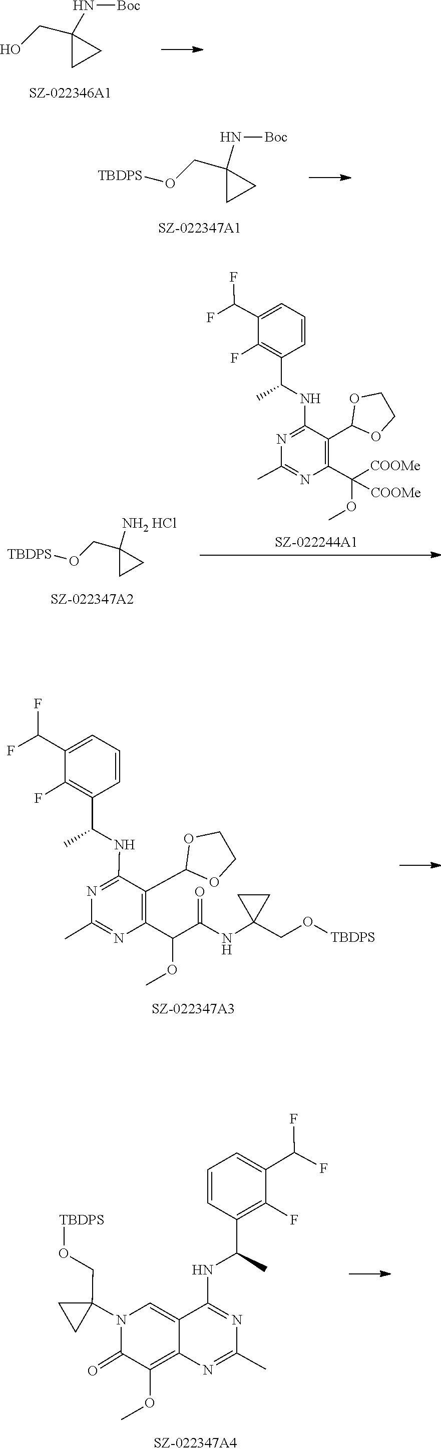

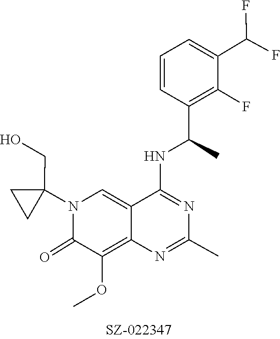 [SMILES: COc1c(=O)n(C2(CO)CC2)cc2c(N[C@H](C)c3cccc(C(F)F)c3F)nc(C)nc12]

### Step 1:

SZ-022346A1 (500 mg, 2.67 mmol), tert-butyldiphenylchlorosilane (771 mg, 2.80 mmol), and imidazole (382 mg, 5.61 mmol) were added to dichloromethane (5 mL), and the mixture was stirred at room temperature for 15 h. After the starting materials were consumed as monitored by TLC, water (50 mL) and dichloromethane (50 mL) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA=10:1; Rf=0.5) to give product SZ-022347A1 as a colorless oil (1.1 g).

SZ-022347A1: ¹H NMR (400 MHz, DMSO-d₆) δ 7.62-7.59 (m, 4H), 7.46-7.39 (m, 6H), 7.20 (s, 1H), 3.65 (s, 2H), 1.35 (s, 9H), 0.99 (s, 9H), 0.78-0.65 (m, 2H), 0.62-0.59 (m, 2H).

### Step 2:

SZ-022347A1 (1.1 g, 2.58 mmol) was added to a solution of hydrogen chloride in 1,4-dioxane (4 M, 5 mL), and the mixture was allowed to react at room temperature for 1 h. After the starting materials were consumed as monitored by TLC, the reaction mixture was concentrated by rotary evaporation to give SZ-022347A2 as an off-white solid (800 mg). The crude product was used directly in the next step.

### Step 3:

SZ-022244A1 (100 mg, 0.19 mmol) was added to dimethyl sulfoxide (5 mL), and then a 10% sodium hydroxide solution (500 μL) was added. The mixture was allowed to react at 40° C. for 1 h. SZ-022347A2 (800 mg, 2.21 mmol), N,N,N,N-tetramethyl-O-(7-azabenzotriazol-1-yl)uronium hexafluorophosphate (216 mg, 0.57 mmol), and triethylamine (58 mg, 0.57 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA=1:1; Rf=0.4) to give product SZ-022347A3 as a light yellow oil (200 mg). LC-MS: [M+H]⁺ 749.97.

### Step 4:

SZ-022347A3 (200 mg, 0.26 mmol) was added to isopropanol (2 mL), and then a hydrochloric acid solution (2 M, 0.5 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation to give SZ-022347A4 as a light yellow oil (160 mg). LC-MS: [M+H]⁺ 687.87.

### Step 5:

SZ-022347A4 (160 mg, 0.23 mmol) was added to a solution of tetrabutylammonium fluoride in tetrahydrofuran (1 M, 4 mL), and the mixture was allowed to react at room temperature for 3 h. After the starting materials were consumed as monitored by LC-MS, the reaction mixture was directly concentrated by rotary evaporation. Water (20 mL) and ethyl acetate (20 mL) were then added for extraction, and the organic layer was separated, washed with a saturated sodium chloride solution, dried, and concentrated by rotary evaporation. The residue was subjected to preparative high performance liquid chromatography and then lyophilized to give SZ-022347 (24 mg). LC-MS: [M+H]⁺.

SZ-022347: ¹H NMR (400 MHz, DMSO-d₆) δ 8.90 (s, 1H), 8.79 (d, J=6.8 Hz, 1H), 7.66 (t, J=7.2 Hz, 1H), 7.52 (t, J=6.8 Hz, 1H), 7.32 (t, J=7.6 Hz, 1H), 7.23 (t, J=54.4 Hz, 1H), 5.81-5.74 (m, 1H), 5.01 (t, J=6.0 Hz, 1H), 3.75 (s, 3H), 3.21 (s, 3H), 1.57 (d, J=6.8 Hz, 3H), 1.22-1.10 (m, 4H).

### Example 34: SZ-022337

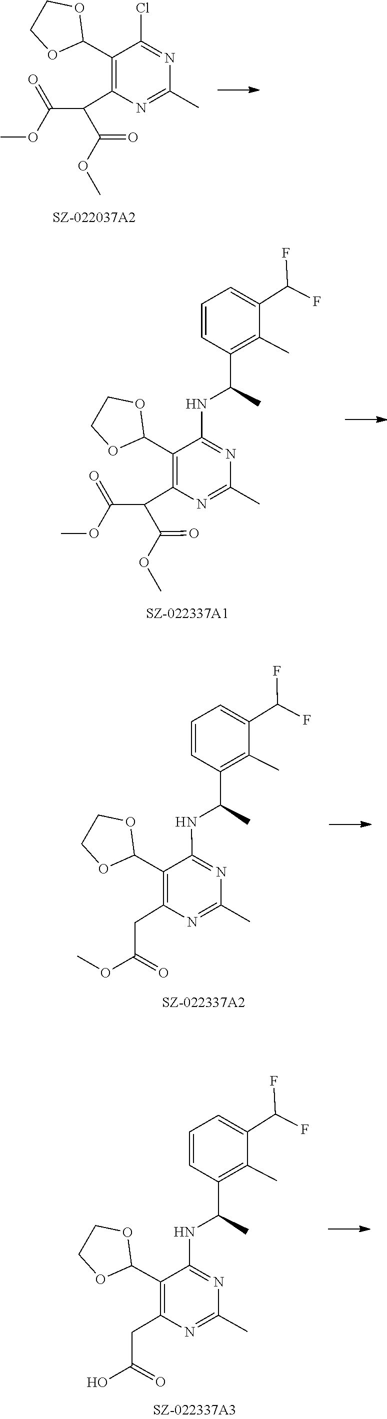

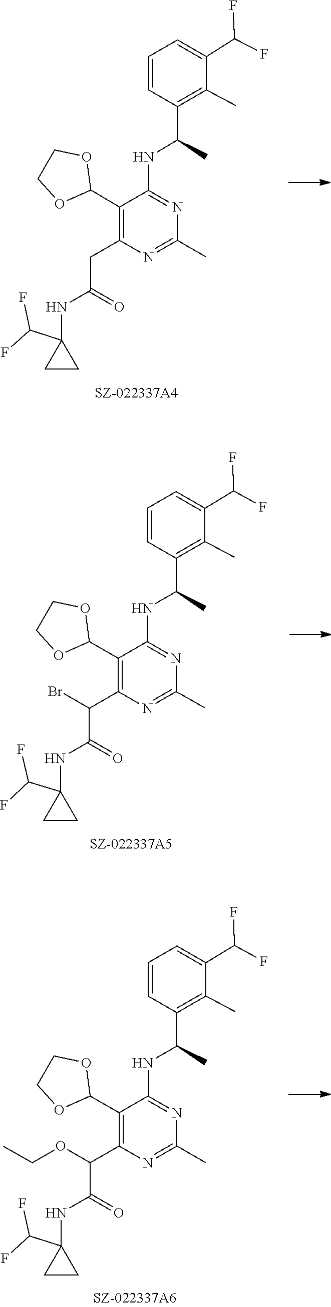 [SMILES: Cc1nc(CC(=O)NC2(C(F)F)CC2)c(C2OCCO2)c(N[C@H](C)c2cccc(C(F)F)c2C)n1 ; Cc1nc(N[C@H](C)c2cccc(C(F)F)c2C)c(C2OCCO2)c(C(Br)C(=O)NC2(C(F)F)CC2)n1 ; CCOC(C(=O)NC1(C(F)F)CC1)c1nc(C)nc(N[C@H](C)c2cccc(C(F)F)c2C)c1C1OCCO1]

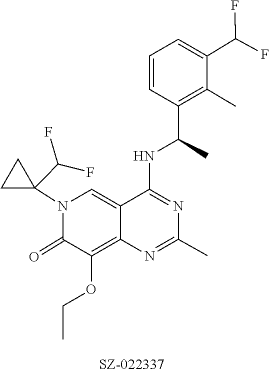 [SMILES: CCOc1c(=O)n(C2(C(F)F)CC2)cc2c(N[C@H](C)c3cccc(C(F)F)c3C)nc(C)nc12]

### Step 1:

SZ-022037A2 (1 g, 3.02 mmol, synthesized according to WO2019122129) and (1R)-1-(3-(difluoromethyl)-2-methylphenyl)ethylamine hydrochloride (670 mg, 3.02 mmol, synthesized according to WO2019122129) were added to N,N-dimethylformamide (10 mL), and then diisopropylethylamine (1.2 g, 9.07 mmol) was added. The mixture was heated to 80° C. for reaction for 8 h. After the starting materials were consumed as monitored by LC-MS, water (50 mL) and ethyl acetate (50 mL) were added to the reaction mixture for extraction, and the organic layer was separated, washed with a saturated sodium chloride solution, dried, and concentrated to dryness under reduced pressure to give a crude product. The crude product was purified by flash silica gel column chromatography (PE:EA=3:2) to give SZ-022337A1 as a light yellow oil (1.2 g). LC-MS: [M+H]⁺ 480.04.

### Step 2:

SZ-022337A1 (1.2 g, 2.50 mmol) was added to dimethyl sulfoxide (10 mL), and then anhydrous lithium chloride (425 mg, 10.0 mL) was added. The mixture was heated to 120° C. for reaction for 16 h. After the starting materials were consumed as monitored by LC-MS, water (40 mL) and ethyl acetate (40 mL) were added to the reaction mixture for extraction, and the organic layer was separated, washed with a saturated sodium chloride solution, dried, and concentrated to dryness under reduced pressure to give a crude product. The crude product was purified by flash silica gel column chromatography (PE:EA=2:3) to give SZ-022337A2 as a light yellow oil (320 mg). LC-MS: [M+H]⁺ 421.94.

### Step 3:

SZ-022337A2 (320 mg, 0.76 mmol) was added to dimethyl sulfoxide (3 mL), and then a 10% sodium hydroxide solution (1.2 mL, 3.04 mmol) was added. The mixture was allowed to react at room temperature for 1 h. After the starting materials were consumed as monitored by LC-MS, the reaction mixture was used directly in the next step without work-up. LC-MS: [M+H]⁺ 407.91.

### Step 4:

1-Difluoromethylcyclopropanamine hydrochloride (78 mg, 0.55 mmol), N,N,N,N-tetramethyl-O-(7-azabenzotriazol-1-yl)uronium hexafluorophosphate (415 mg, 1.09 mmol), and triethylamine (110 mg, 1.09 mmol) were added to the reaction system in the previous step, and the mixture was allowed to react at room temperature for 1 h. After the starting materials were consumed as monitored by LC-MS, water (20 mL) and ethyl acetate (30 mL) were added to the reaction mixture for extraction, and the organic layer was separated, washed with a saturated sodium chloride solution, dried, and concentrated to dryness under reduced pressure to give a crude product. The crude product was purified by flash silica gel column chromatography (PE:EA=2:3) to give SZ-022337A4 as a light yellow oil (220 mg). LC-MS: [M+H]⁺ 497.07.

### Step 5:

SZ-022337A4 (220 mg, 0.44 mmol) was added to carbon tetrachloride (4 mL), and then N-bromosuccinimide (80 mg, 0.44 mmol) and azobisisobutyronitrile (7 mg, 0.044 mmol) were added. The mixture was heated to 60° C. for reaction for 1 h. After the starting materials were consumed as monitored by LC-MS, the reaction mixture was directly purified by flash silica gel column chromatography (PE:EA=4:1) to give SZ-022337A5 as a light yellow oil (120 mg). LC-MS: [M+H]⁺ 574.74.

### Step 6:

Sodium hydride (25 mg, 0.63 mmol) was added to tetrahydrofuran (3 mL), and then ethanol (19 mg, 0.42 mmol) was added. The mixture was stirred at 0° C. for 0.5 h. A solution of SZ-022337A5 (120 mg, 0.21 mmol) in tetrahydrofuran (1.5 mL) was added dropwise to the reaction mixture, and the resulting mixture was stirred for another 2.5 h. After the starting materials were consumed as monitored by LC-MS, water (10 mL) and ethyl acetate (10 mL) were added to the reaction mixture for extraction, and the organic layer was separated, washed with a saturated sodium chloride solution, dried, and concentrated by rotary evaporation to give crude product SZ-022337A6 as a yellow oil (85 mg). LC-MS: [M+H]⁺ 541.12.

### Step 7:

SZ-022337A6 (40 mg, 0.07 mmol) was added to isopropanol (3 mL), and then a hydrochloric acid solution (2 M, 1.5 mL) was added. The mixture was allowed to react at 50° C. for 5 h. After the starting materials were consumed as monitored by LC-MS, the reaction mixture was directly concentrated by rotary evaporation. A saturated sodium bicarbonate solution (5 mL) and ethyl acetate (10 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022337 (50 mg). LC-MS: [M+H]⁺ 478.94.

SZ-022337: ¹H NMR (400 MHz, CD₃OD) δ 8.92 (s, 1H), 7.56 (d, J=7.6 Hz, 1H), 7.42 (d, J=7.6 Hz, 1H), 7.29 (t, J=7.6 Hz, 1H), 6.95 (t, J=55.2 Hz, 1H), 6.25 (t, J=57.6 Hz, 1H), 5.87-5.82 (m, 1H), 4.16 (q, J=7.2 Hz, 2H), 2.54 (s, 3H), 2.35 (s, 3H), 1.61-1.59 (m, 5H), 1.42 (br, 2H), 1.36 (t, J=6.8 Hz, 3H).

### Example 35: SZ-022338

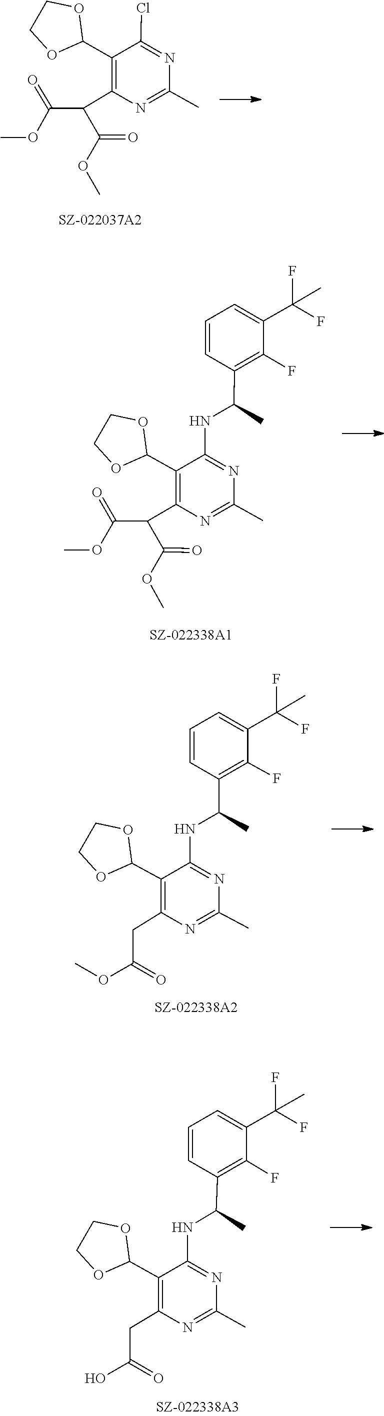

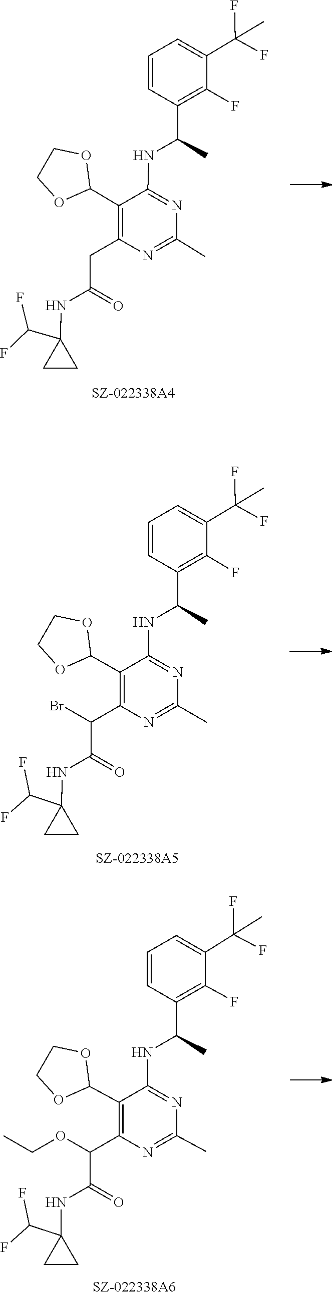 [SMILES: Cc1nc(CC(=O)NC2(C(F)F)CC2)c(C2OCCO2)c(N[C@H](C)c2cccc(C(C)(F)F)c2F)n1 ; Cc1nc(N[C@H](C)c2cccc(C(C)(F)F)c2F)c(C2OCCO2)c(C(Br)C(=O)NC2(C(F)F)CC2)n1 ; CCOC(C(=O)NC1(C(F)F)CC1)c1nc(C)nc(N[C@H](C)c2cccc(C(C)(F)F)c2F)c1C1OCCO1]

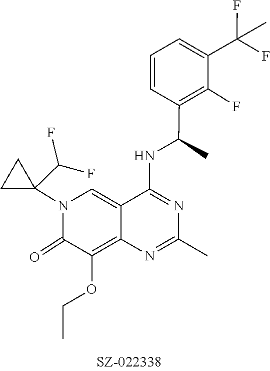 [SMILES: CCOc1c(=O)n(C2(C(F)F)CC2)cc2c(N[C@H](C)c3cccc(C(C)(F)F)c3F)nc(C)nc12]

### Step 1:

SZ-022037A2 (690 mg, 1.73 mmol, synthesized according to WO2019122129) and (1R)-1-(3-(1,1-difluoroethyl)-2-fluorophenyl)ethylamine hydrochloride (494 mg, 1.73 mmol, synthesized according to WO2019122129) were added to N,N-dimethylformamide (6 mL), and then diisopropylethylamine (810 mg, 6.26 mmol) was added. The mixture was heated to 80° C. for reaction for 8 h. After the starting materials were consumed as monitored by LC-MS, water (30 mL) and ethyl acetate (30 mL) were added to the reaction mixture for extraction, and the organic layer was separated, washed with a saturated sodium chloride solution, dried, and concentrated to dryness under reduced pressure to give a crude product. The crude product was purified by flash silica gel column chromatography (PE:EA=3:2) to give SZ-022338A1 as a light yellow oil (600 mg). LC-MS: [M+H]⁺ 498.20.

### Step 2:

SZ-022338A1 (600 mg, 1.21 mmol) was added to dimethyl sulfoxide (5 mL), and then anhydrous lithium chloride (205 mg, 4.83 mL) was added. The mixture was heated to 120° C. for reaction for 16 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated to dryness under reduced pressure to give a crude product. The crude product was purified by flash silica gel column chromatography (PE:EA=3:2) to give SZ-022338A2 as a light yellow oil (160 mg). LC-MS: [M+H]⁺ 439.94.

### Step 3:

SZ-022338A2 (160 mg, 0.36 mmol) was added to dimethyl sulfoxide (2 mL), and then a 10% sodium hydroxide solution (0.58 mL, 1.46 mmol) was added. The mixture was allowed to react at room temperature for 1 h. After the starting materials were consumed as monitored by LC-MS, the reaction mixture was used directly in the next step without work-up. LC-MS: [M+H]⁺ 426.11.

### Step 4:

(Difluoromethyl)cyclopropyl-1-amine hydrochloride (78 mg, 0.55 mmol), N,N,N,N-tetramethyl-O-(7-azabenzotriazol-1-yl)uronium hexafluorophosphate (415 mg, 1.09 mmol), and triethylamine (110 mg, 1.09 mmol) were added to the reaction system in the previous step, and the mixture was allowed to react at room temperature for 1 h. After the starting materials were consumed as monitored by LC-MS, water (20 mL) and ethyl acetate (30 mL) were added to the reaction mixture for extraction, and the organic layer was separated, washed with a saturated sodium chloride solution, dried, and concentrated to dryness under reduced pressure to give a crude product. The crude product was purified by flash silica gel column chromatography (PE:EA=2:3) to give SZ-022338A4 as a light yellow oil (120 mg). LC-MS: [M+H]⁺ 515.22.

### Step 5:

SZ-022338A4 (120 mg, 0.23 mmol) was added to carbon tetrachloride (3 mL), and then N-bromosuccinimide (42 mg, 0.23 mmol) and azobisisobutyronitrile (4 mg, 0.023 mmol) were added. The mixture was heated to 120° C. for reaction for 1 h. After the starting materials were consumed as monitored by LC-MS, the reaction mixture was directly purified by flash silica gel column chromatography (PE:EA=4:1) to give SZ-022338A5 as a light yellow oil (90 mg). LC-MS: [M+H]⁺ 592.73.

### Step 6:

Sodium hydride (18 mg, 0.45 mmol) was added to tetrahydrofuran (2.5 mL), and then ethanol (13.8 mg, 0.3 mmol) was added. The mixture was stirred at 0° C. for 0.5 h. A solution of SZ-022338A5 (90 mg, 0.15 mmol) in tetrahydrofuran (1 mL) was added dropwise to the reaction mixture, and the resulting mixture was stirred for another 2.5 h. After the starting materials were consumed as monitored by LC-MS, water (10 mL) and ethyl acetate (10 mL) were added to the reaction mixture for extraction, and the organic layer was separated, washed with a saturated sodium chloride solution, dried, and concentrated by rotary evaporation to give crude product SZ-022338A6 as a yellow oil (40 mg). LC-MS: [M+H]⁺ 559.05.

### Step 7:

SZ-022338A6 (40 mg, 0.07 mmol) was added to isopropanol (2 mL), and then a hydrochloric acid solution (2 M, 1 mL) was added. The mixture was allowed to react at 50° C. for 5 h. After the starting materials were consumed as monitored by LC-MS, the reaction mixture was directly concentrated by rotary evaporation. A saturated sodium bicarbonate solution (5 mL) and ethyl acetate (10 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022338 (18 mg). LC-MS: [M+H]⁺ 496.99.

SZ-022338: ¹H NMR (400 MHz, DMSO-d₆) δ 8.94 (s, 1H), 8.89 (d, J=6.4 Hz, 1H), 7.59 (t, J=7.2 Hz, 1H), 7.42 (t, J=7.2 Hz, 1H), 7.24 (t, J=7.6 Hz, 1H), 6.31 (t, J=57.2 Hz, 1H), 5.75-5.68 (m, 1H), 4.04 (q, J=6.8 Hz, 2H), 2.18 (s, 3H), 1.99 (t, J=19.2 Hz, 3H), 1.55 (d, J=7.2 Hz, 3H), 1.40 (br, 4H), 1.19 (t, J=7.2 Hz, 3H).

### Example 36: SZ-022313

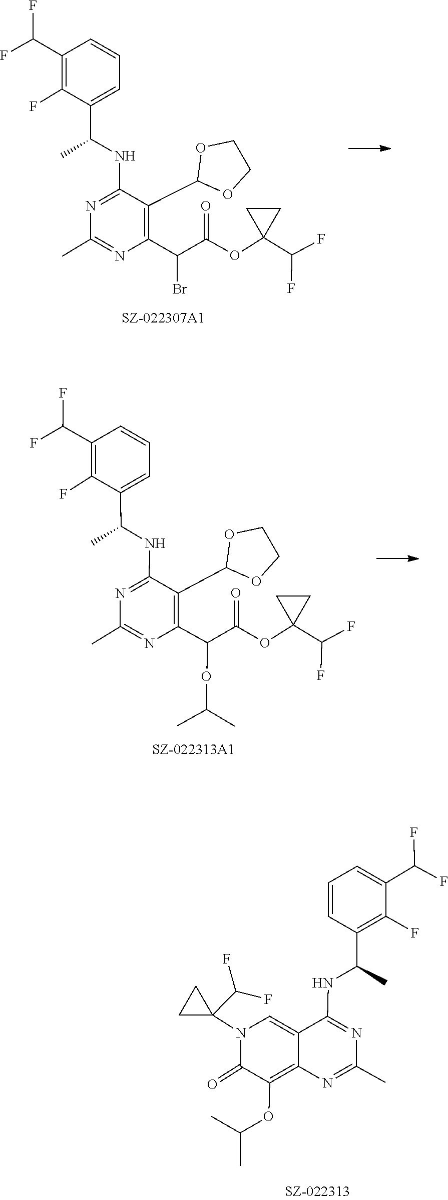 [SMILES: Cc1nc(N[C@H](C)c2cccc(C(F)F)c2F)c(C2OCCO2)c(C(Br)C(=O)OC2(C(F)F)CC2)n1 ; Cc1nc(N[C@H](C)c2cccc(C(F)F)c2F)c(C2OCCO2)c(C(OC(C)C)C(=O)OC2(C(F)F)CC2)n1 ; Cc1nc(N[C@H](C)c2cccc(C(F)F)c2F)c2cn(C3(C(F)F)CC3)c(=O)c(OC(C)C)c2n1]

### Step 1:

Sodium hydride (14 mg, 0.3 mmol) was added to tetrahydrofuran (2 mL), and then isopropanol (12 mg, 0.2 mmol) was added. The mixture was stirred at room temperature for 0.5 h. A solution of SZ-022307A1 (60 mg, 0.1 mmol) in tetrahydrofuran (1 mL) was added dropwise to the reaction mixture, and the resulting mixture was stirred for another 1.5 h. After the starting materials were consumed as monitored by LC-MS, water (10 mL) and ethyl acetate (10 mL) were added to the reaction mixture for extraction, and the organic layer was separated, washed with a saturated sodium chloride solution, dried, and concentrated by rotary evaporation to give crude product SZ-022313A1 as a yellow viscous substance (29 mg). LC-MS: [M+H]⁺ 559.22.

### Step 2:

SZ-022313A1 (29 mg, 0.1 mmol) was added to isopropanol (2 mL), and then a hydrochloric acid solution (2 M, 0.5 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL) and ethyl acetate (10 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022313 (11 mg). LC-MS: [M+H]⁺ 496.90.

SZ-022313: ¹H NMR (400 MHz, DMSO-d₆) δ 8.92 (s, 1H), 8.82 (d, J=7.2 Hz, 1H), 7.66 (t, J=7.6 Hz, 1H), 7.51 (t, J=7.2 Hz, 1H), 7.31 (t, J=7.6 Hz, 1H), 7.22 (t, J=54.4 Hz, 1H), 6.33 (t, J=57.2 Hz, 1H), 5.76-5.73 (m, 1H), 4.61-4.64 (m, 1H), 2.19 (s, 3H), 1.57 (d, J=6.8 Hz, 3H), 1.47 (br, 4H), 1.16 (dd, J₁=4.0 Hz, J₂=6.4 Hz, 6H).

### Example 37: SZ-022323

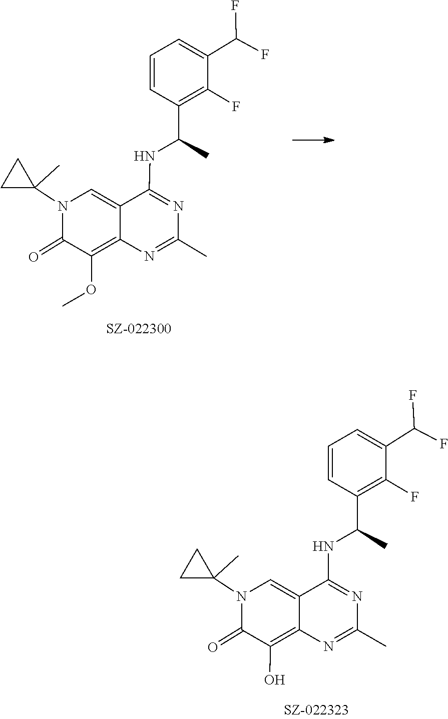 [SMILES: COc1c(=O)n(C2(C)CC2)cc2c(N[C@H](C)c3cccc(C(F)F)c3F)nc(C)nc12 ; Cc1nc(N[C@H](C)c2cccc(C(F)F)c2F)c2cn(C3(C)CC3)c(=O)c(O)c2n1]

### Step 1:

SZ-022300 (38 mg, 88 μmol) was added to pyridine (1 mL), and then pyridine hydrochloride (102 mg, 0.88 mmol) was added. The mixture was purged with argon for 1 min and then microwaved at 150° C. for reaction for 2 h. After the starting materials were consumed as monitored by LC-MS, the reaction mixture was poured into water (10 mL), and the solution was adjusted to pH 6-7 with 1 M diluted hydrochloric acid. Ethyl acetate (20 mL) was then added for extraction, and the organic layer was separated and washed with a saturated sodium chloride solution. The solvent was removed by rotary evaporation, and the residue was subjected to preparative high performance liquid chromatography and then lyophilized to give SZ-022323 (8 mg). LC-MS: [M+H]⁺ 419.10.

SZ-022323: ¹H NMR (400 MHz, DMSO-d₆) δ 8.66 (s, 1H), 8.56 (d, J=7.2 Hz, 1H), 7.66 (t, J=7.2 Hz, 1H), 7.51 (t, J=7.2 Hz, 1H), 7.31 (t, J=7.6 Hz, 1H), 7.23 (t, J=54 Hz, 1H), 5.79-5.72 (m, 1H), 2.19 (s, 3H), 1.57 (d, J=7.2 Hz, 3H), 1.52 (s, 3H), 1.16-1.10 (m, 2H), 1.08-1.01 (m, 2H).

### Example 38: SZ-022299

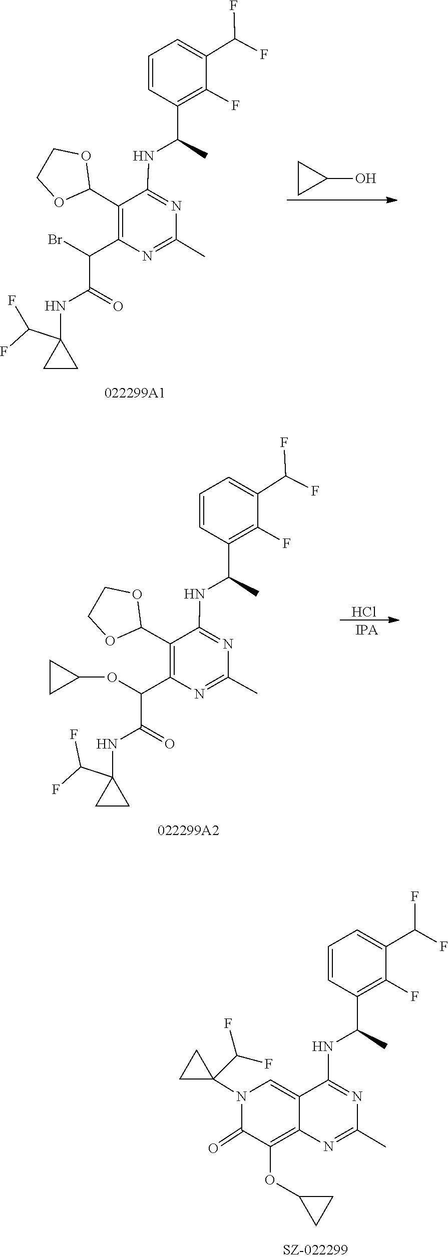

### Step 1:

60% NaH (55 mg, 1.381 mmol, 10 eq) was added to a three-necked flask, and under argon atmosphere, anhydrous tetrahydrofuran (5 mL) was added via a syringe. Cyclopropanol (88 mg, 1.519 mmol, 11 eq) was then added dropwise at room temperature, and the mixture was stirred at room temperature for 30 min. The reaction mixture was then stirred in an ice-water bath, and a solution of 022299A1 (80 mg, 0.138 mmol, 1 eq) and dry tetrahydrofuran (1 mL) was added. The resulting mixture was naturally warmed and stirred for 30 min, and then the reaction was substantially completed as indicated by LC-MS. A saturated ammonium chloride solution (10 mL×1) and water (20 mL×1) were added to the reaction mixture, and the mixture was extracted with ethyl acetate (20 mL×3). The organic phases were combined, washed with saturated brine (20 mL×1), dried over anhydrous sodium sulfate, and filtered. The filtrate was concentrated under reduced pressure, subjected to column chromatography with a 25 g silica gel column, and subjected to gradient elution with 0-65% ethyl acetate/petroleum ether. The fractions were collected and concentrated under reduced pressure to give 022299A2 as a colorless oil (71 mg). LC-MS: [M+H]⁺ 557.05.

### Step 2:

022299A2 (71 mg, 0.128 mmol, 1 eq) and isopropanol (2 mL) were added to a 2 M hydrochloric acid solution (0.3 mL) at room temperature, and under argon atmosphere, the mixture was heated to 50° C. in an oil bath and stirred for 3 h. The reaction was completed as indicated by LC-MS, thus giving a product. The mixture was concentrated under reduced pressure to remove the solvent. Water (30 mL×1) and a saturated sodium bicarbonate solution (5 mL×1) were then added, and the mixture was extracted with ethyl acetate (20 mL×3). The organic phases were combined, washed with saturated brine (20 mL×1), dried over anhydrous sodium sulfate, and filtered. The filtrate was concentrated under reduced pressure to give a yellow oil (73 mg), which was then purified by preparative high-pressure reversed-phase chromatography to give SZ-022299 as a yellow-green solid (25 mg). LC-MS: [M+H]⁺ 494.99, ¹H NMR (400 MHz, DMSO-d₆) δ 8.96 (s, 1H), 8.83 (d, J=7.1 Hz, 1H), 7.65 (t, J=7.5 Hz, 1H), 7.52 (t, J=6.8 Hz, 1H), 7.32 (t, J=7.6 Hz, 1H), 7.23 (t, J=54.0 Hz, 1H), 6.33 (t, J=56.8 Hz, 1H), 5.79-5.71 (m, 1H), 4.39-4.33 (m, 1H), 2.21 (s, 3H), 1.58 (d, J=6.8 Hz, 3H), 1.53-1.29 (m, 4H), 0.81-0.72 (m, 2H), 0.41-0.36 (m, 2H).

### Example 39: SZ-022348

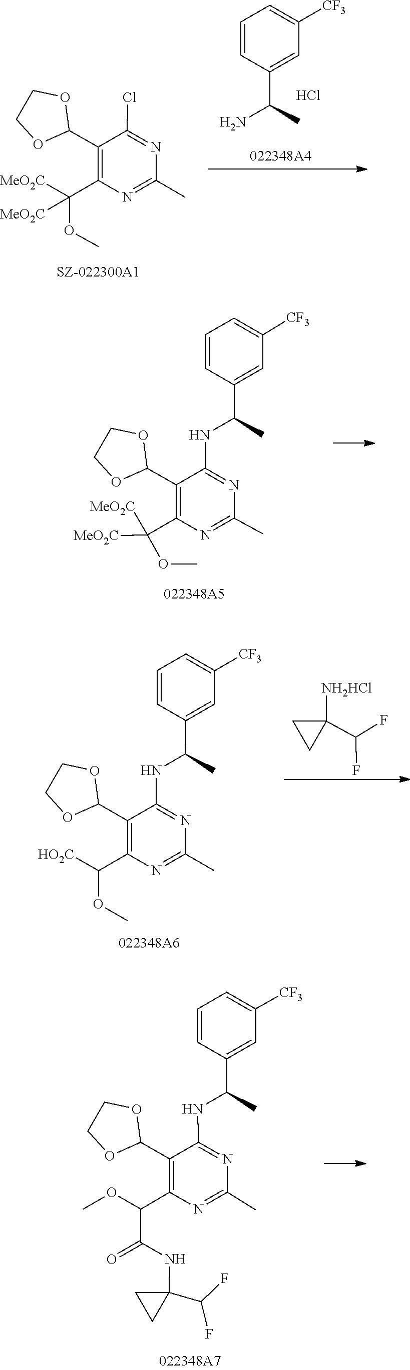

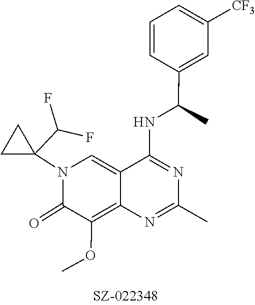 [SMILES: COc1c(=O)n(C2(C(F)F)CC2)cc2c(N[C@H](C)c3cccc(C(F)(F)F)c3)nc(C)nc12]

### Step 1:

SZ-022300A1 (100 mg, 0.27 mmol), 022348A4 (75 mg, 0.33 mmol), and triethylamine (140 mg, 1.39 mmol) were added to tetrahydrofuran (5 mL), and the mixture was heated to 80° C. for reaction for 16 h. After the starting materials were consumed as monitored by LC-MS, water (20 mL) and ethyl acetate (50 mL) were added to the reaction mixture for extraction, and the organic layer was separated, washed with a saturated sodium chloride solution, dried, and concentrated to dryness under reduced pressure to give a crude product. The crude product was purified by flash silica gel column chromatography (PE:EA=1:1) to give 022348A5 as a light yellow oil (90 mg). LC-MS: [M+H]⁺ 514.17.

### Step 2:

022348A5 (90 mg, 0.17 mmol) was added to dimethyl sulfoxide (4 mL), and then a 20% sodium hydroxide solution (0.5 mL) was added. The mixture was allowed to react at room temperature for 1 h. After the starting materials were consumed as monitored by LC-MS, the reaction mixture was used directly in the next step without work-up. LC-MS: [M+H]⁺ 442.14.

### Step 3:

(Difluoromethyl)cyclopropyl-1-amine hydrochloride (36 mg, 0.25 mmol), HATU (194 mg, 0.51 mmol), and triethylamine (86 mg, 0.85 mmol) were added to the reaction system in the previous step, and the mixture was allowed to react at room temperature for another 16 h. After the starting materials were consumed as monitored by LC-MS, water (20 mL) and ethyl acetate (50 mL) were added to the reaction mixture for extraction, and the organic layer was separated, washed with a saturated sodium chloride solution, dried, and concentrated to dryness under reduced pressure to give crude product 022348A7 as a light yellow oil (90 mg). LC-MS: [M+H]⁺ 531.26.

### Step 4:

022348A7 (90 mg, 0.17 mmol) was added to isopropanol (3 mL), and then a hydrochloric acid solution (2 M, 0.52 mL) was added. The mixture was allowed to react at 50° C. for 4 h. After the starting materials were consumed as monitored by LC-MS, the reaction mixture was directly concentrated to dryness under reduced pressure. A saturated sodium bicarbonate solution (5 mL), water (10 mL), and ethyl acetate (20 mL) were then added for extraction, and the organic layer was separated, washed with a saturated sodium chloride solution, dried, and concentrated to dryness under reduced pressure to give a crude product. The crude product was separated by preparative high performance liquid chromatography and then lyophilized to give SZ-022348 as a yellow solid (30 mg). LC-MS: [M+H]⁺ 469.14.

SZ-022348: ¹H NMR (400 MHz, DMSO-d₆) δ 8.88 (s, 1H), 8.80 (d, J=7.6 Hz, 1H), 7.77 (s, 1H), 7.72-7.70 (m, 1H), 7.62-7.56 (m, 2H), 6.33 (t, J=56.8 Hz, 1H), 5.68-5.61 (m, 1H), 3.77 (s, 3H), 2.25 (s, 3H), 1.59 (d, J=7.2 Hz, 3H), 1.49-1.47 (m, 2H), 1.42-1.33 (m, 2H).

### Example 40: SZ-022351

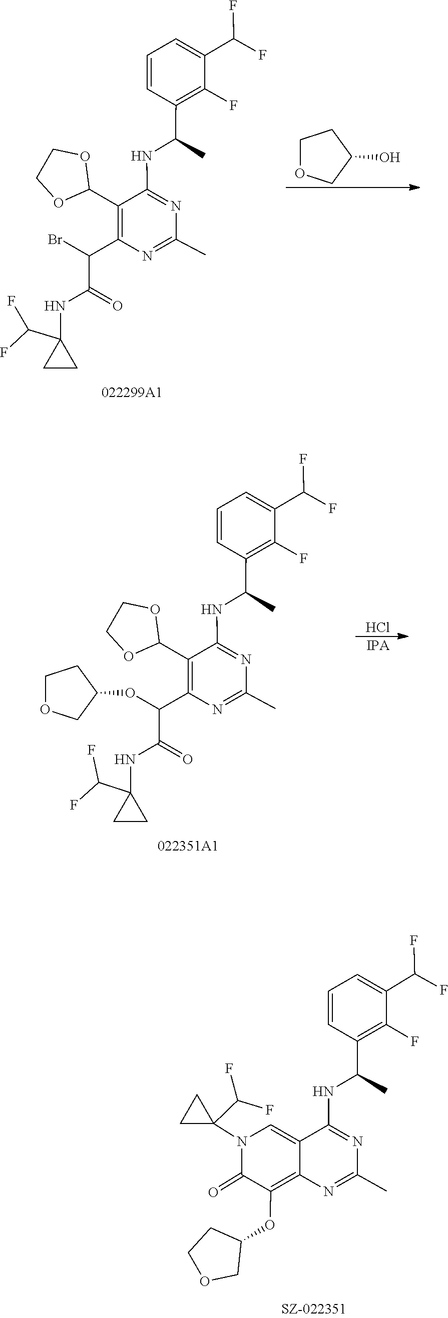

### Step 1:

60% NaH (55 mg, 1.381 mmol, 10 eq) was added to a three-necked flask, and under argon atmosphere, anhydrous tetrahydrofuran (5 mL) was added via a syringe. (S)-3-Hydroxytetrahydrofuran (134 mg, 1.519 mmol, 11 eq) was then added dropwise at room temperature, and the mixture was stirred at room temperature for 30 min. The reaction mixture was then stirred in an ice-water bath, and a solution of 022299A1 (80 mg, 0.138 mmol, 1 eq) and dry tetrahydrofuran (1 mL) was added. The resulting mixture was naturally warmed and stirred for 2.5 h, and then the reaction was substantially completed as indicated by LC-MS. A saturated ammonium chloride solution (10 mL×1) and water (20 mL×1) were added to the reaction mixture, and the mixture was extracted with ethyl acetate (20 mL×3). The organic phases were combined, washed with saturated brine (20 mL×1), dried over anhydrous sodium sulfate, and filtered. The filtrate was concentrated under reduced pressure, subjected to column chromatography with a 25 g silica gel column, and subjected to gradient elution with 0-100% ethyl acetate/petroleum ether. The fractions were collected and concentrated under reduced pressure to give 022351A1 as a colorless oil (51 mg), LC-MS: [M+H]⁺ 587.02.

### Step 2:

022351A1 (51 mg, 0.087 mmol, 1 eq) and isopropanol (2 mL) were added to a 2 M hydrochloric acid solution (0.3 mL) at room temperature, and under argon atmosphere, the mixture was heated to 50° C. in an oil bath and stirred for 3 h. The reaction was completed as indicated by LC-MS, thus giving a product. The mixture was concentrated under reduced pressure to remove the solvent. Water (30 mL×1) and a saturated sodium bicarbonate solution (5 mL×1) were then added, and the mixture was extracted with ethyl acetate (20 mL×3). The organic phases were combined, washed with saturated brine (20 mL×1), dried over anhydrous sodium sulfate, and filtered. The filtrate was concentrated under reduced pressure to give a yellow oil (51 mg), which was then purified by preparative high-pressure reversed-phase chromatography to give SZ-022351 as a yellow-green solid (21 mg). LC-MS: [M+H]⁺ 524.95, ¹H NMR (400 MHz, DMSO-d₆) δ 8.95 (s, 1H), 8.82 (d, J=7.2 Hz, 1H), 7.65 (t, J=7.2 Hz, 1H), 7.52 (t, J=7.2 Hz, 1H), 7.32 (t, J=8.0 Hz, 1H), 7.23 (t, J=54.4 Hz, 1H), 6.34 (t, J=56.8 Hz, 1H), 5.79-5.72 (m, 1H), 5.16-5.14 (m, 1H), 4.00-3.85 (m, 2H), 3.78-3.66 (m, 2H), 2.21 (s, 3H), 2.03-1.97 (m, 1H), 1.75-1.85 (m, 1H), 1.58 (d, J=7.2 Hz, 3H), 1.53-1.30 (m, 4H).

### Example 41: SZ-022352

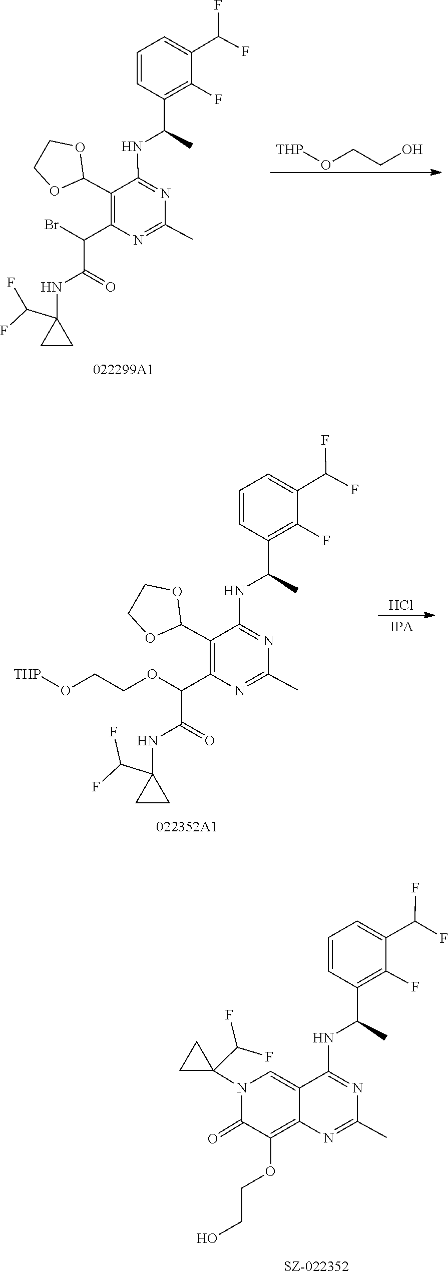

### Step 1:

60% NaH (55 mg, 1.381 mmol, 10 eq) was added to a three-necked flask, and under argon atmosphere, anhydrous tetrahydrofuran (5 mL) was added via a syringe. 2-(Tetrahydro-2H-pyran-2-oxy)ethanol was then added dropwise at room temperature, and the mixture was stirred at room temperature for 50 min. The reaction mixture was then stirred in an ice-water bath, and a solution of 022299A1 (80 mg, 0.138 mmol, 1 eq) and dry tetrahydrofuran (1 mL) was added. The resulting mixture was naturally warmed and stirred for 1 h, and then the reaction was substantially completed as indicated by LC-MS. A saturated ammonium chloride solution (10 mL×1) and water (20 mL×1) were added to the reaction mixture, and the mixture was extracted with ethyl acetate (20 mL×3). The organic phases were combined, washed with saturated brine (20 mL×1), dried over anhydrous sodium sulfate, and filtered. The filtrate was concentrated under reduced pressure, subjected to column chromatography with a 25 g silica gel column, and subjected to gradient elution with 0-100% ethyl acetate/petroleum ether. The fractions were collected and concentrated under reduced pressure to give 022352A1 as a colorless oil (87 mg), LC-MS: [M+H]⁺ 644.98.

### Step 2:

022352A1 (87 mg, 0.135 mmol, 1 eq) and isopropanol (2 mL) were added to a 2 M hydrochloric acid solution (0.5 mL) at room temperature, and under argon atmosphere, the mixture was heated to 50° C. in an oil bath and stirred for 3 h. The reaction was completed as indicated by LC-MS, thus giving a product. The mixture was concentrated under reduced pressure to remove the solvent, and water (30 mL×1) was added. The mixture was adjusted to about pH 7-8 by dropwise addition of a saturated sodium bicarbonate solution and extracted with ethyl acetate (20 mL×3). The organic phases were combined, washed with saturated brine (20 mL×1), dried over anhydrous sodium sulfate, and filtered. The filtrate was concentrated under reduced pressure to give a yellow oil (80 mg), which was then purified by preparative high-pressure reversed-phase chromatography to give SZ-022352 as a yellow-green solid (37 mg). LC-MS: [M+H]⁺ 498.99, ¹H NMR (400 MHz, DMSO-d₆) δ 9.02-8.94 (m, 2H), 7.65 (t, J=7.6 Hz, 1H), 7.53 (t, J=7.2 Hz, 1H), 7.33 (t, J=7.6 Hz, 1H), 7.23 (t, J=54.0 Hz, 1H), 6.33 (t, J=56.8 Hz, 1H), 5.80-5.73 (m, 1H), 5.55 (br s, 1H), 4.06-3.98 (m, 2H), 3.48 (t, J=5.2 Hz, 2H), 2.22 (s, 3H), 1.59 (d, J=7.2 Hz, 3H), 1.54-1.32 (m, 4H).

### Example 42: SZ-022353

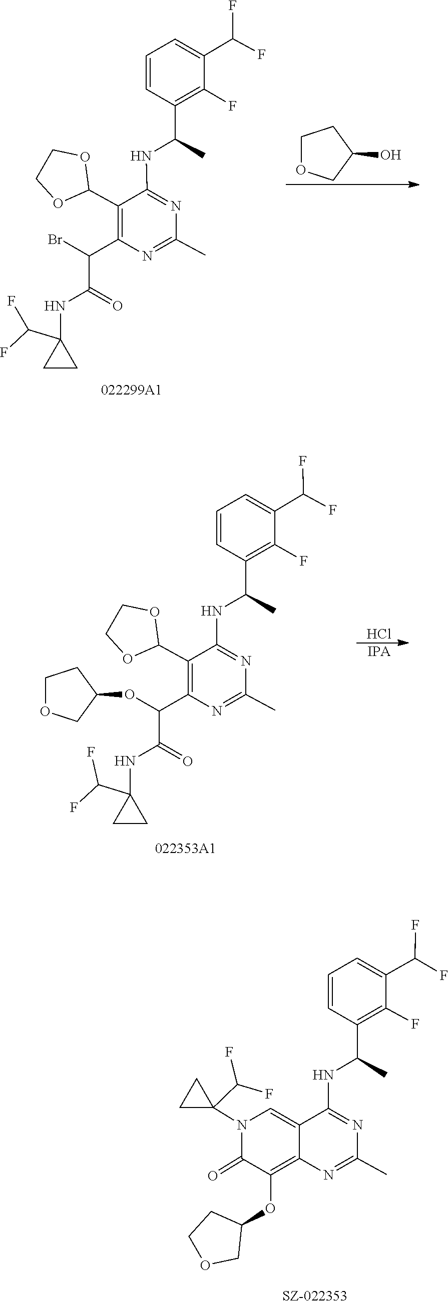

By referring to Example SZ-022351, SZ-022353 as a yellow-green solid (20 mg) was obtained. LC-MS: [M+H]⁺ 525.00, ¹H NMR (400 MHz, DMSO-d₆) δ 8.94 (s, 1H), 8.82 (d, J=6.8 Hz, 1H), 7.65 (t, J=7.6 Hz, 1H), 7.52 (t, J=7.6 Hz, 1H), 7.32 (t, J=7.6 Hz, 1H), 7.23 (t, J=54.4 Hz, 1H), 6.34 (t, J=56.8 Hz, 1H), 5.79-5.72 (m, 1H), 5.18-5.11 (m, 1H), 4.01-3.85 (m, 2H), 3.79-3.67 (m, 2H), 2.21 (s, 3H), 2.06-1.97 (m, 1H), 1.87-1.74 (m, 1H), 1.58 (d, J=6.8 Hz, 3H), 1.54-1.31 (m, 4H).

### Example 43: SZ-022354

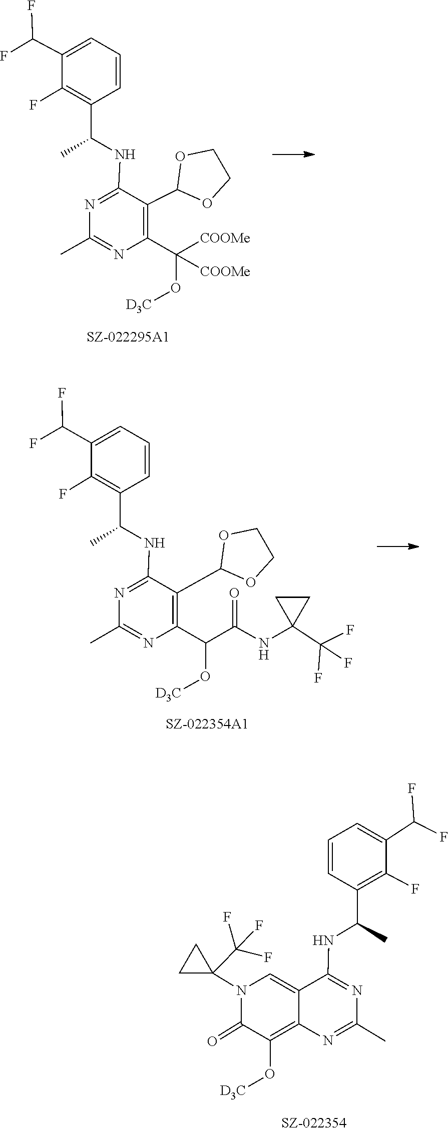 [SMILES: [2H]C([2H])([2H])OC(C(=O)OC)(C(=O)OC)c1nc(C)nc(N[C@H](C)c2cccc(C(F)F)c2F)c1C1OCCO1 ; [2H]C([2H])([2H])OC(C(=O)NC1(C(F)(F)F)CC1)c1nc(C)nc(N[C@H](C)c2cccc(C(F)F)c2F)c1C1OCCO1 ; [2H]C([2H])([2H])Oc1c(=O)n(C2(C(F)(F)F)CC2)cc2c(N[C@H](C)c3cccc(C(F)F)c3F)nc(C)nc12]

### Step 1:

SZ-022295A1 (280 mg, 0.54 mmol) was added to dimethyl sulfoxide (5 mL), and then a 10% sodium hydroxide solution (870 μL) was added. The mixture was allowed to react at room temperature for 1 h. 1-Trifluoromethylcyclopropanamine hydrochloride (132 mg, 0.81 mmol), N,N,N,N-tetramethyl-O-(7-azabenzotriazol-1-yl)uronium hexafluorophosphate (619 mg, 1.63 mmol), and triethylamine (165 mg, 1.63 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022354A1 as a yellow viscous substance (280 mg). LC-MS: [M+H]⁺ 551.99.

### Step 2:

SZ-022354A1 (280 mg, 0.51 mmol) was added to isopropanol (5 mL), and then a hydrochloric acid solution (4 M, 0.64 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022354 (110 mg). LC-MS: [M+H]⁺ 490.16.

SZ-022354: ¹H NMR (400 MHz, DMSO-d₆) δ 9.00 (s, 1H), 8.83 (d, J=7.2 Hz, 1H), 7.63 (t, J=7.6 Hz, 1H), 7.53 (t, J=7.2 Hz, 1H), 7.33 (d, J=8.0 Hz, 1H), 7.24 (t, J=54.4 Hz, 1H), 5.80-5.67 (m, 1H), 2.21 (s, 3H), 1.87-1.49 (m, 4H), 1.59 (d, J=7.2 Hz, 3H).

### Example 44: SZ-022373

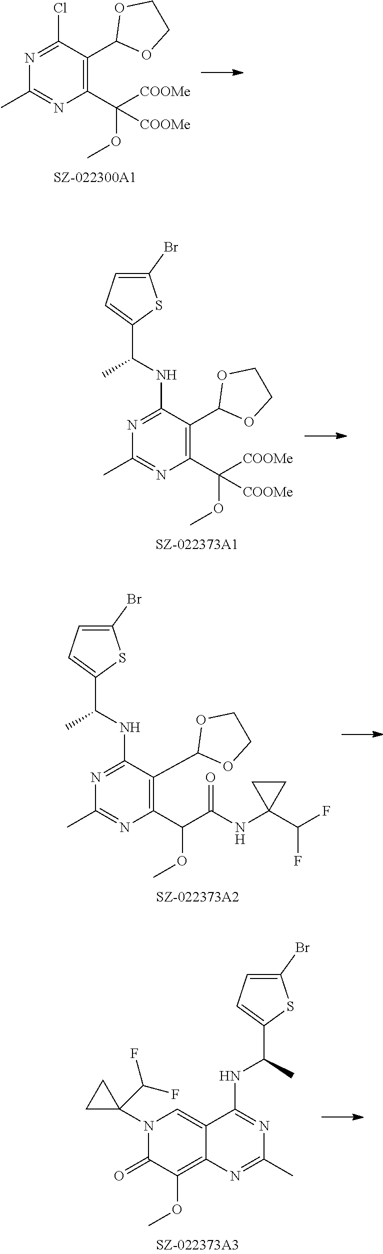 [SMILES: COC(=O)C(OC)(C(=O)OC)c1nc(C)nc(Cl)c1C1OCCO1 ; COC(=O)C(OC)(C(=O)OC)c1nc(C)nc(N[C@H](C)c2ccc(Br)s2)c1C1OCCO1 ; COC(C(=O)NC1(C(F)F)CC1)c1nc(C)nc(N[C@H](C)c2ccc(Br)s2)c1C1OCCO1 ; COc1c(=O)n(C2(C(F)F)CC2)cc2c(N[C@H](C)c3ccc(Br)s3)nc(C)nc12]

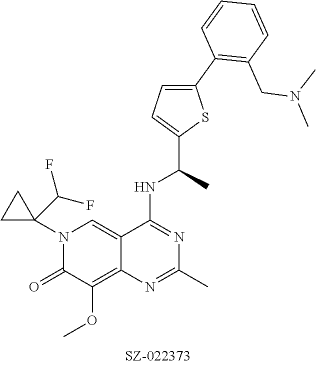 [SMILES: COc1c(=O)n(C2(C(F)F)CC2)cc2c(N[C@H](C)c3ccc(-c4ccccc4CN(C)C)s3)nc(C)nc12]

### Step 1:

Compound SZ-022300A1 (500 mg, 1.39 mmol), (1R)-1-(5-bromothiophen-2-yl)ethylamine hydrochloride (405 mg, 1.67 mmol, synthesized according to WO2019122129), and N,N-diisopropylethylamine (900 mg, 6.94 mmol) were allowed to react at 85° C. overnight. After the starting materials were consumed as monitored by LC-MS, the reaction mixture was cooled to room temperature. Water (50 mL) and ethyl acetate (50 mL) were added to the reaction mixture for extraction, and the organic layer was separated, dried, and concentrated by rotary evaporation to give a crude product. The crude product was purified by flash silica gel column chromatography (PE:EA=3:1; Rf=0.2) to give product SZ-022373A1 as a colorless oil (525 mg). LC-MS: [M+H]⁺ 529.67, 531.64.

### Step 2:

SZ-022373A1 (525 mg, 1 mmol) was added to dimethyl sulfoxide (5 mL), and then a 10% sodium hydroxide solution (1.6 mL) was added. The mixture was allowed to react at room temperature for 1 h. 1-Difluoromethylcyclopropanamine hydrochloride (215 mg, 1.5 mmol), N,N,N,N-tetramethyl-O-(7-azabenzotriazol-1-yl)uronium hexafluorophosphate (1.14 g, 3 mmol), and triethylamine (303 mg, 3 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022373A2 as a yellow viscous substance (520 mg). LC-MS: [M+H]⁺ 546.70, 548.63.

### Step 3:

SZ-022373A2 (520 mg, 0.95 mmol) was added to isopropanol (5 mL), and then a hydrochloric acid solution (4 M, 1.2 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to medium-pressure reversed-phase column chromatography (acetonitrile:water=1:1) and then lyophilized to give SZ-022373A3 (350 mg). LC-MS: [M+H]⁺ 485.07, 487.02.

### Step 4:

SZ-022373A3 (100 mg, 0.21 mmol), 2-(N,N-dimethylamino)phenylboronic acid (50 mg, 0.25 mmol), tetrakis(triphenylphosphine)palladium (30 mg, 0.021 mmol), and sodium carbonate (45 mg, 0.42 mmol) were added to 1,4-dioxane (2 mL) and water (0.4 mL), and the mixture was purged with argon for 1 min and then microwaved at 125° C. for reaction for 1.5 h. After the starting materials were consumed as monitored by LC-MS, the reaction mixture was directly concentrated by rotary evaporation. Water (10 mL) and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022373 (45 mg). LC-MS: [M+H]⁺ 539.92.

SZ-022373: ¹H NMR (400 MHz, DMSO-d₆) δ 8.90 (d, J=8.1 Hz, 1H), 8.84 (s, 1H), 7.46-7.28 (m, 4H), 7.19 (d, J=3.6 Hz, 1H), 7.10 (d, J=3.6 Hz, 1H), 6.32 (t, J=57.2 Hz, 1H), 5.96-5.88 (m, 1H), 3.80 (s, 3H), 3.37 (s, 2H), 2.35 (s, 3H), 2.12 (s, 6H), 1.69 (d, J=6.8 Hz, 3H), 1.46-1.35 (m, 4H).

### Example 45: SZ-022356

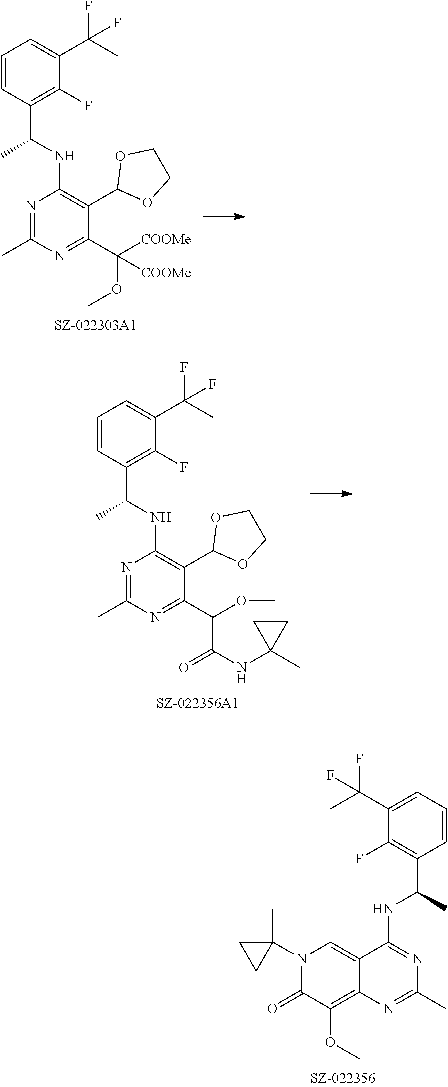 [SMILES: COC(=O)C(OC)(C(=O)OC)c1nc(C)nc(N[C@H](C)c2cccc(C(C)(F)F)c2F)c1C1OCCO1 ; COC(C(=O)NC1(C)CC1)c1nc(C)nc(N[C@H](C)c2cccc(C(C)(F)F)c2F)c1C1OCCO1 ; COc1c(=O)n(C2(C)CC2)cc2c(N[C@H](C)c3cccc(C(C)(F)F)c3F)nc(C)nc12]

### Step 1:

SZ-022303A1 (220 mg, 0.42 mmol) was added to dimethyl sulfoxide (4 mL), and then a 10% sodium hydroxide solution (670 μL) was added. The mixture was allowed to react at room temperature for 1 h. 1-Methylcyclopropanamine hydrochloride (67 mg, 0.63 mmol), N,N,N,N-tetramethyl-O-(7-azabenzotriazol-1-yl)uronium hexafluorophosphate (476 mg, 1.25 mmol), and triethylamine (127 mg, 1.25 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022356A1 as a light yellow viscous substance (220 mg). LC-MS: [M+H]⁺ 509.26.

### Step 2:

SZ-022356A1 (220 mg, 0.43 mmol) was added to isopropanol (5 mL), and then a hydrochloric acid solution (4 M, 0.55 mL) was added. The mixture was allowed to react at 50° C. for 2 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022356 (74 mg). LC-MS: [M+H]⁺ 447.16.

SZ-022356: ¹H NMR (400 MHz, DMSO-d₆) δ 8.99 (s, 1H), 8.69 (d, J=7.2 Hz, 1H), 7.61 (t, J=6.8 Hz, 1H), 7.45 (t, J=6.8 Hz, 1H), 7.27 (t, J=7.6 Hz, 1H), 5.77-5.70 (m, 1H), 3.75 (s, 3H), 2.20 (s, 3H), 2.02 (t, J=19.2 Hz, 3H), 1.57 (d, J=7.2 Hz, 3H), 1.52 (s, 3H), 1.17-1.08 (m, 2H), 1.08-1.00 (m, 2H).

### Example 46: SZ-022357

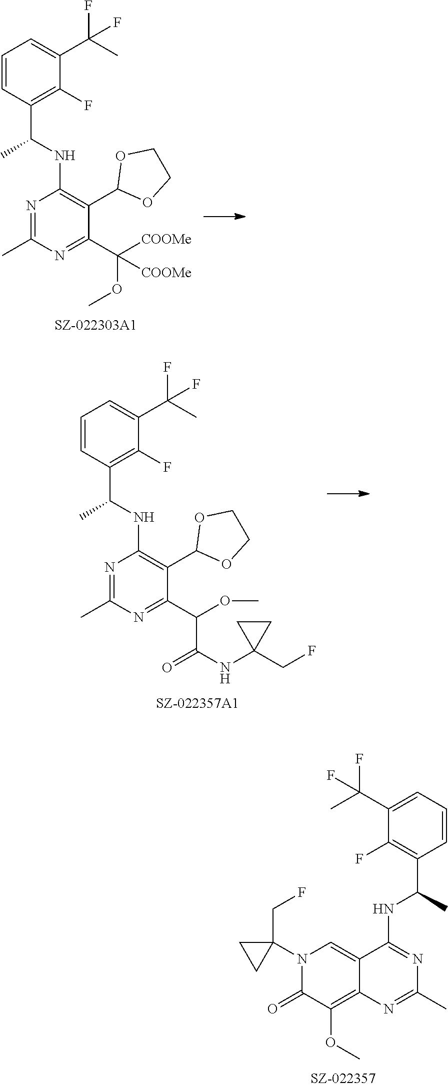 [SMILES: COC(=O)C(OC)(C(=O)OC)c1nc(C)nc(N[C@H](C)c2cccc(C(C)(F)F)c2F)c1C1OCCO1 ; COC(C(=O)NC1(CF)CC1)c1nc(C)nc(N[C@H](C)c2cccc(C(C)(F)F)c2F)c1C1OCCO1 ; COc1c(=O)n(C2(CF)CC2)cc2c(N[C@H](C)c3cccc(C(C)(F)F)c3F)nc(C)nc12]

### Step 1:

SZ-022303A1 (220 mg, 0.41 mmol) was added to dimethyl sulfoxide (4 mL), and then a 10% sodium hydroxide solution (670 μL) was added. The mixture was allowed to react at room temperature for 1 h. 1-(Difluoromethyl)cyclopropanamine hydrochloride (79 mg, 0.63 mmol), N,N,N,N-tetramethyl-O-(7-azabenzotriazol-1-yl)uronium hexafluorophosphate (476 mg, 1.25 mmol), and triethylamine (127 mg, 1.25 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022357A1 as a light yellow viscous substance (220 mg). LC-MS: [M+H]⁺ 527.08.

### Step 2:

SZ-022357A1 (220 mg, 0.41 mmol) was added to isopropanol (5 mL), and then a hydrochloric acid solution (4 M, 0.52 mL) was added. The mixture was allowed to react at 50° C. for 2 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022357 (59 mg). LC-MS: [M+H]⁺ 465.18.

SZ-022357: ¹H NMR (400 MHz, DMSO-d₆) δ 8.95 (s, 1H), 8.81 (d, J=7.2 Hz, 1H), 7.59 (t, J=6.8 Hz, 1H), 7.45 (t, J=7.6 Hz, 1H), 7.27 (t, J=7.6 Hz, 1H), 5.78-5.71 (m, 1H), 4.79-4.49 (m, 2H), 3.76 (s, 3H), 2.21 (s, 3H), 2.02 (t, J=19.2 Hz, 3H), 1.57 (d, J=7.2 Hz, 3H), 1.38-1.25 (m, 4H).

### Example 47: SZ-022358

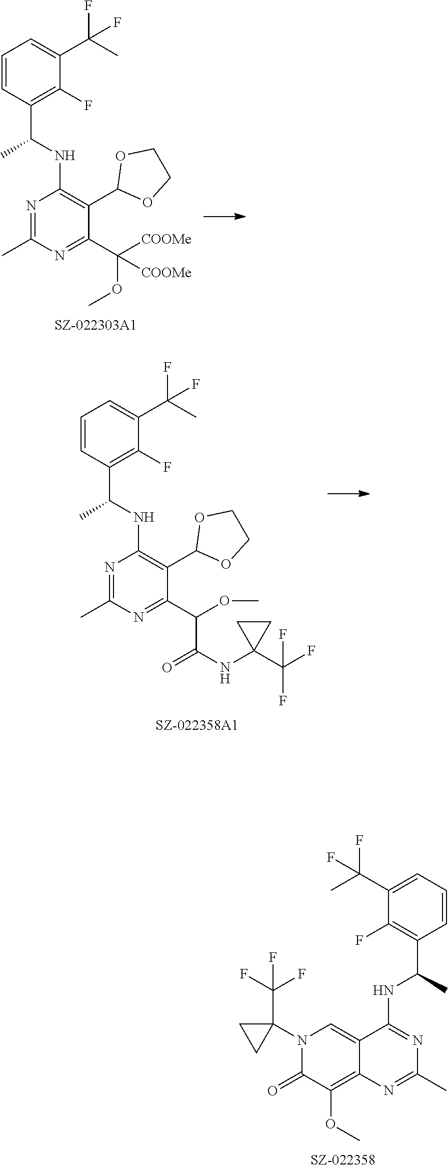 [SMILES: COC(=O)C(OC)(C(=O)OC)c1nc(C)nc(N[C@H](C)c2cccc(C(C)(F)F)c2F)c1C1OCCO1 ; COC(C(=O)NC1(C(F)(F)F)CC1)c1nc(C)nc(N[C@H](C)c2cccc(C(C)(F)F)c2F)c1C1OCCO1 ; COc1c(=O)n(C2(C(F)(F)F)CC2)cc2c(N[C@H](C)c3cccc(C(C)(F)F)c3F)nc(C)nc12]

### Step 1:

SZ-022303A1 (220 mg, 0.41 mmol) was added to dimethyl sulfoxide (2 mL), and then a 10% sodium hydroxide solution (670 μL) was added. The mixture was allowed to react at room temperature for 1 h. 1-(Trifluoromethyl)cyclopropanamine hydrochloride (101 mg, 0.62 mmol), N,N,N,N-tetramethyl-O-(7-azabenzotriazol-1-yl)uronium hexafluorophosphate (476 mg, 1.25 mmol), and triethylamine (127 mg, 1.25 mmol) were then added into the system, and the mixture was allowed to react at room temperature for another 3 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022358A1 as a light yellow viscous substance (220 mg). LC-MS: [M+H]⁺ 563.01.

### Step 2:

SZ-022358A1 (220 mg, 0.39 mmol) was added to isopropanol (5 mL), and then a hydrochloric acid solution (4 M, 0.49 mL) was added. The mixture was allowed to react at 50° C. for 2 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022358 (61 mg). LC-MS: [M+H]⁺ 501.16.

SZ-022358: ¹H NMR (400 MHz, DMSO-d₆) δ 9.00 (s, 1H), 8.83 (d, J=6.8 Hz, 1H), 7.59 (t, J=7.2 Hz, 1H), 7.45 (t, J=7.2 Hz, 1H), 7.28 (d, J=7.6 Hz, 1H), 5.80-5.69 (m, 1H), 3.76 (s, 3H), 2.21 (s, 3H), 2.02 (t, J=19.2 Hz, 3H), 1.88-1.52 (m, 4H), 1.58 (d, J=7.2 Hz, 3H).

### Example 48: SZ-022341

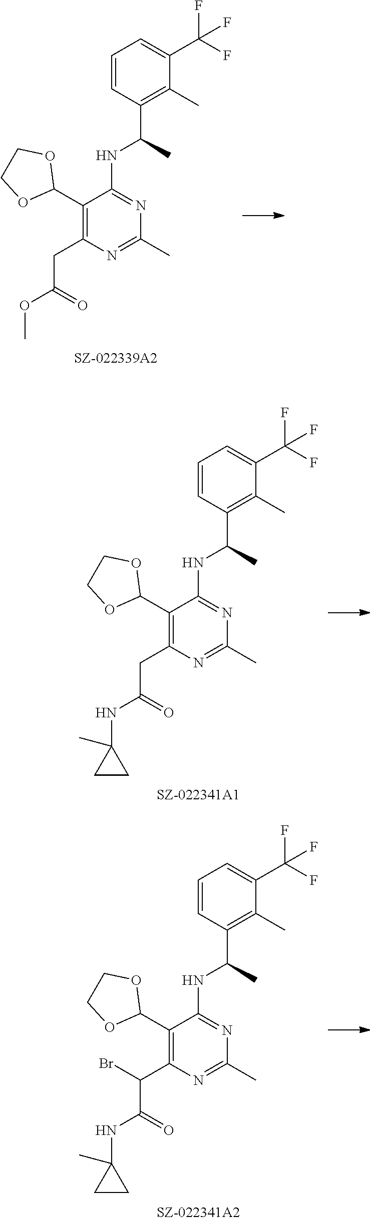 [SMILES: COC(=O)Cc1nc(C)nc(N[C@H](C)c2cccc(C(F)(F)F)c2C)c1C1OCCO1 ; Cc1nc(CC(=O)NC2(C)CC2)c(C2OCCO2)c(N[C@H](C)c2cccc(C(F)(F)F)c2C)n1 ; Cc1nc(N[C@H](C)c2cccc(C(F)(F)F)c2C)c(C2OCCO2)c(C(Br)C(=O)NC2(C)CC2)n1]

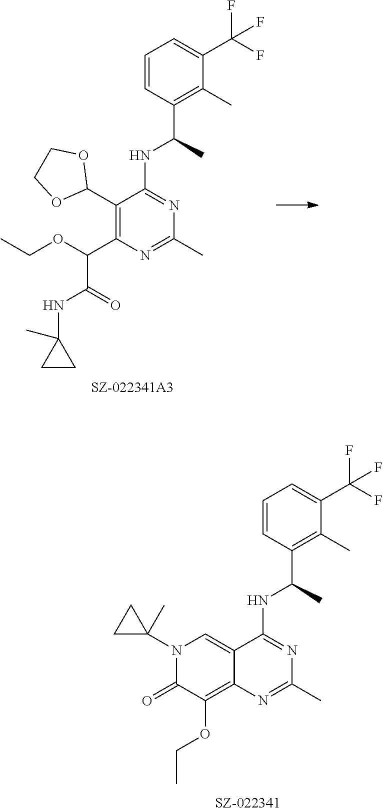 [SMILES: CCOC(C(=O)NC1(C)CC1)c1nc(C)nc(N[C@H](C)c2cccc(C(F)(F)F)c2C)c1C1OCCO1 ; CCOc1c(=O)n(C2(C)CC2)cc2c(N[C@H](C)c3cccc(C(F)(F)F)c3C)nc(C)nc12]

### Step 1:

SZ-022339A2 (500 mg, 1.14 mmol) and sodium hydroxide (182 mg, 4.56 mmol) were added to dimethyl sulfoxide (5 mL), and the mixture was allowed to react at room temperature for 1 h. After the starting materials were consumed as monitored by LC-MS, triethylamine (346 mg, 3.42 mmol), 1-(methylcyclopropyl)amine hydrochloride (184 mg, 1.71 mmol), and N,N,N,N-tetramethyl-O-(7-azabenzotriazol-1-yl)uronium hexafluorophosphate (1300 mg, 3.42 mmol) were added to the reaction mixture, and the resulting mixture was allowed to react at room temperature for 2 h. Water (50 mL) and ethyl acetate (50 mL) were added to the reaction mixture for extraction, and the organic layer was separated, washed with a saturated sodium chloride solution, dried, and concentrated by rotary evaporation to give a crude product. The crude product was purified by flash silica gel column chromatography (DCM:MeOH=10:1; Rf=0.5) to give SZ-022341A1 as a yellow solid (490 mg). LC-MS: [M+H]⁺ 479.24.

### Step 2:

SZ-022341A1 (490 mg, 1.03 mmol), N-bromosuccinimide (183 mg, 1.03 mmol), and azobisisobutyronitrile (17 mg, 0.103 mmol) were added to carbon tetrachloride (15 mL), and the mixture was heated to 60° C. for reaction for 1 h. After the starting materials were consumed as monitored by LC-MS, the reaction mixture was concentrated by rotary evaporation to give a crude product. The crude product was purified by flash silica gel column chromatography (PE:EA=3:7; Rf=0.5) to give SZ-022341A2 as a yellow solid (350 mg). LC-MS: [M+H]⁺ 556.76, 558.70.

### Step 3:

SZ-022341A2 (250 mg, 0.45 mmol) was dissolved in ethanol (25 mL), and a solution of sodium ethoxide in ethanol (0.8 mL, 20% wt) was slowly added dropwise to the above system. The mixture was stirred at room temperature for 2 h. After the starting materials were consumed as monitored by LC-MS, the reaction mixture was concentrated. Water (50 mL) and ethyl acetate (50 mL) were then added for extraction, and the organic layer was separated, washed with a saturated sodium chloride solution, dried, and concentrated by rotary evaporation to give SZ-022341A3 as a yellow solid (140 mg). LC-MS: [M+H]⁺ 522.96.

### Step 4:

SZ-022341A3 (140 mg, 0.27 mmol) and hydrochloric acid (0.15 mL, 5 M) were added to isopropanol (2 mL), and the mixture was heated to 50° C. for reaction for 6 h. After the starting materials were consumed as monitored by LC-MS, the reaction mixture was concentrated by rotary evaporation to give a crude product. The crude product was subjected to preparative high performance liquid chromatography and then lyophilized to give SZ-022341 (35.43 mg). LC-MS: [M+H]⁺ 460.89.

SZ-022341: ¹H NMR (400 MHz, DMSO-d₆) δ 8.95 (s, 1H), 8.75 (d, J=6.8 Hz, 1H), 7.73 (d, J=8.0 Hz, 1H), 7.56 (d, J=8.0 Hz, 1H), 7.38 (t, J=8.0 Hz, 1H), 5.71-5.64 (m, 1H), 4.04 (q, J=7.2 Hz, 2H), 2.57 (s, 3H), 2.19 (s, 3H), 1.54-1.51 (m, 6H), 1.20 (t, J=7.2 Hz, 3H), 1.15-1.01 (m, 4H).

### Example 49: SZ-022342

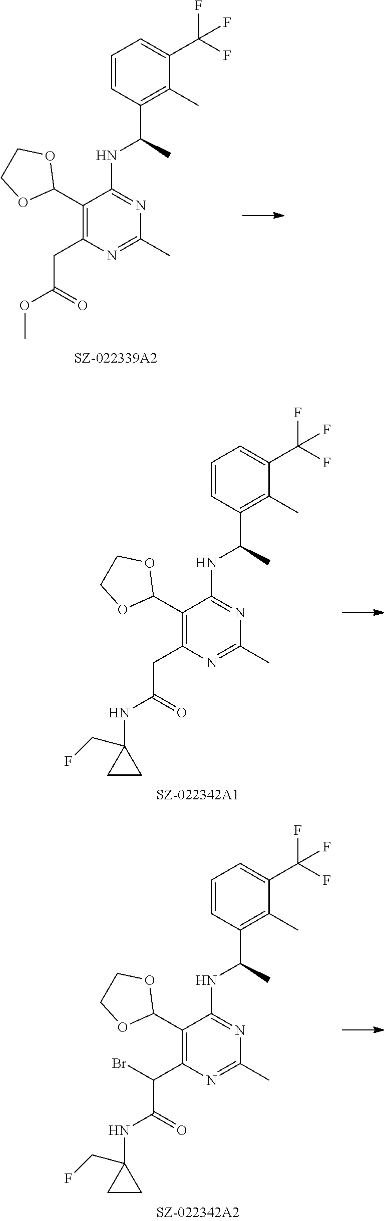 [SMILES: COC(=O)Cc1nc(C)nc(N[C@H](C)c2cccc(C(F)(F)F)c2C)c1C1OCCO1 ; Cc1nc(CC(=O)NC2(CF)CC2)c(C2OCCO2)c(N[C@H](C)c2cccc(C(F)(F)F)c2C)n1 ; Cc1nc(N[C@H](C)c2cccc(C(F)(F)F)c2C)c(C2OCCO2)c(C(Br)C(=O)NC2(CF)CC2)n1]

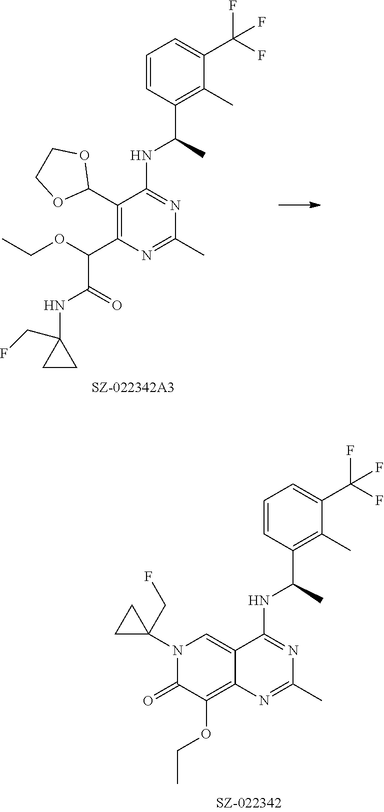 [SMILES: CCOC(C(=O)NC1(CF)CC1)c1nc(C)nc(N[C@H](C)c2cccc(C(F)(F)F)c2C)c1C1OCCO1 ; CCOc1c(=O)n(C2(CF)CC2)cc2c(N[C@H](C)c3cccc(C(F)(F)F)c3C)nc(C)nc12]

### Step 1:

SZ-022339A2 (500 mg, 1.14 mmol) and sodium hydroxide (182 mg, 4.56 mmol) were added to dimethyl sulfoxide (5 mL), and the mixture was allowed to react at room temperature for 1 h. After the starting materials were consumed as monitored by LC-MS, triethylamine (346 mg, 3.42 mmol), 1-(monofluoromethylcyclopropyl)amine hydrochloride (216 mg, 1.71 mmol), and N,N,N,N-tetramethyl-O-(7-azabenzotriazol-1-yl)uronium hexafluorophosphate (1300 mg, 3.42 mmol) were added to the reaction mixture, and the resulting mixture was allowed to react at room temperature for 2 h. Water (50 mL) and ethyl acetate (50 mL) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA=0:1; Rf=0.5) to give SZ-022342A1 as a white solid (490 mg). LC-MS: [M+H]⁺ 496.98.

### Step 2:

SZ-022342A1 (490 mg, 0.99 mmol), N-bromosuccinimide (176 mg, 0.99 mmol), and azobisisobutyronitrile (16 mg, 0.099 mmol) were added to carbon tetrachloride (15 mL), and the mixture was heated to 60° C. for reaction for 1 h. After the starting materials were consumed as monitored by LC-MS, the reaction mixture was concentrated by rotary evaporation to give a crude product. The crude product was purified by flash silica gel column chromatography (PE:EA=1:1; Rf=0.5) to give SZ-022342A2 as a white solid (320 mg). LC-MS: [M+H]⁺ 574.87, 576.77.

### Step 3:

SZ-022342A2 (320 mg, 0.56 mmol) was dissolved in ethanol (5 mL), and a solution of sodium ethoxide in ethanol (1.1 mL, 20% wt) was slowly added dropwise to the above system. The mixture was stirred at room temperature for 2 h. After the starting materials were consumed as monitored by LC-MS, the reaction mixture was concentrated. Water (50 mL) and ethyl acetate (50 mL) were then added for extraction, and the organic layer was separated, washed with a saturated sodium chloride solution, dried, and concentrated by rotary evaporation to give SZ-022342A3 as a yellow solid (270 mg). LC-MS: [M+H]⁺ 541.00.

### Step 4:

SZ-022342A3 (270 mg, 0.50 mmol) and hydrochloric acid (0.3 mL, 5 M) were added to isopropanol (5 mL), and the mixture was heated to 50° C. for reaction for 3 h. After the starting materials were consumed as monitored by LC-MS, water (50 mL) and ethyl acetate (50 mL) were added to the reaction mixture for extraction, and the organic layer was separated, washed with a saturated sodium chloride solution, dried, and concentrated by rotary evaporation to give a crude product. The crude product was subjected to preparative high performance liquid chromatography and then lyophilized to give SZ-022342 (33.99 mg). LC-MS: [M+H]⁺ 479.19.

SZ-022342: ¹H NMR (400 MHz, DMSO-d₆) δ 8.91 (s, 1H), 8.85 (d, J=6.8 Hz, 1H), 7.71 (d, J=8.0 Hz, 1H), 7.56 (d, J=8.0 Hz, 1H), 7.39 (t, J=8.0 Hz, 1H), 5.72-5.67 (m, 1H), 4.73-4.54 (m, 2H), 4.06 (q, J=7.2 Hz, 2H), 2.57 (s, 3H), 2.20 (s, 3H), 1.53 (d, J=7.2 Hz, 3H), 1.33-1.30 (m, 4H), 1.21 (t, J=7.2 Hz, 3H).

### Example 50: SZ-022371

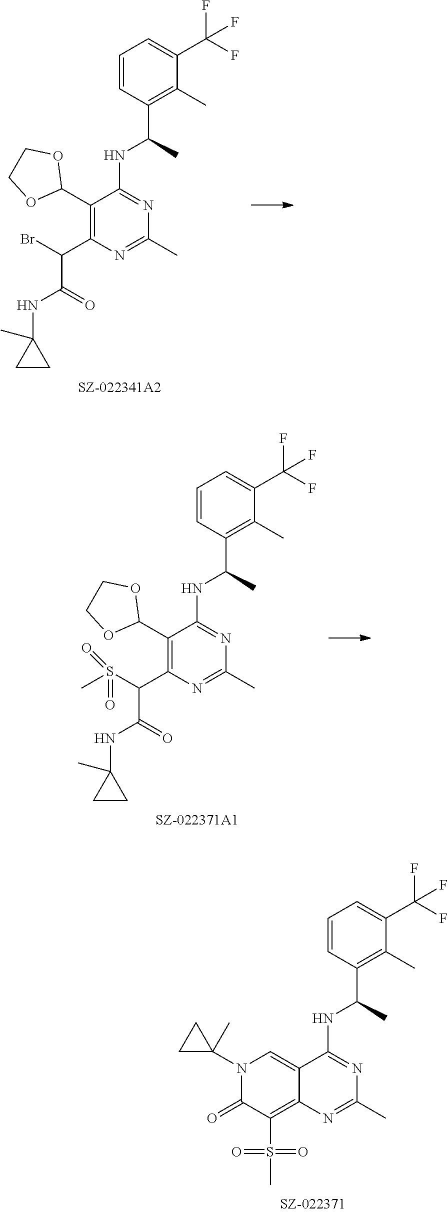 [SMILES: Cc1nc(N[C@H](C)c2cccc(C(F)(F)F)c2C)c(C2OCCO2)c(C(Br)C(=O)NC2(C)CC2)n1 ; Cc1nc(N[C@H](C)c2cccc(C(F)(F)F)c2C)c(C2OCCO2)c(C(C(=O)NC2(C)CC2)S(C)(=O)=O)n1 ; Cc1nc(N[C@H](C)c2cccc(C(F)(F)F)c2C)c2cn(C3(C)CC3)c(=O)c(S(C)(=O)=O)c2n1]

### Step 1:

SZ-022341A2 (100 mg, 0.18 mmol) and sodium methanesulfinate (22 mg, 0.22 mmol) were dissolved in dimethyl sulfoxide (10 mL), and the solution was stirred at room temperature for 2 h. After the starting materials were consumed as monitored by LC-MS, water (100 mL) and ethyl acetate (50 mL) were added to the reaction mixture for extraction, and the organic layer was separated, washed with a saturated sodium chloride solution, dried, and concentrated by rotary evaporation to give SZ-022371A1 as a yellow solid (80 mg). LC-MS: [M+H]⁺ 557.26.

### Step 2:

SZ-022371A1 (80 mg, 0.14 mmol) and hydrochloric acid (0.1 mL, 5 M) were added to isopropanol (2 mL), and the mixture was heated to 50° C. for reaction for 3 h. After the starting materials were consumed as monitored by LC-MS, the reaction mixture was concentrated by rotary evaporation to give a crude product. The crude product was subjected to preparative high performance liquid chromatography and then lyophilized to give SZ-022371 (21.51 mg). LC-MS: [M+H]⁺ 494.84.

SZ-022371: ¹H NMR (400 MHz, DMSO-d₆) δ 9.50 (s, 1H), 9.10 (d, J=6.8 Hz, 1H), 7.74 (d, J=8.0 Hz, 1H), 7.57 (d, J=8.0 Hz, 1H), 7.39 (t, J=8.0 Hz, 1H), 5.73-5.66 (m, 1H), 3.31 (s, 3H), 2.56 (s, 3H), 2.25 (s, 3H), 1.56-1.54 (m, 6H), 1.17-1.05 (m, 4H).

### Example 51: SZ-022372

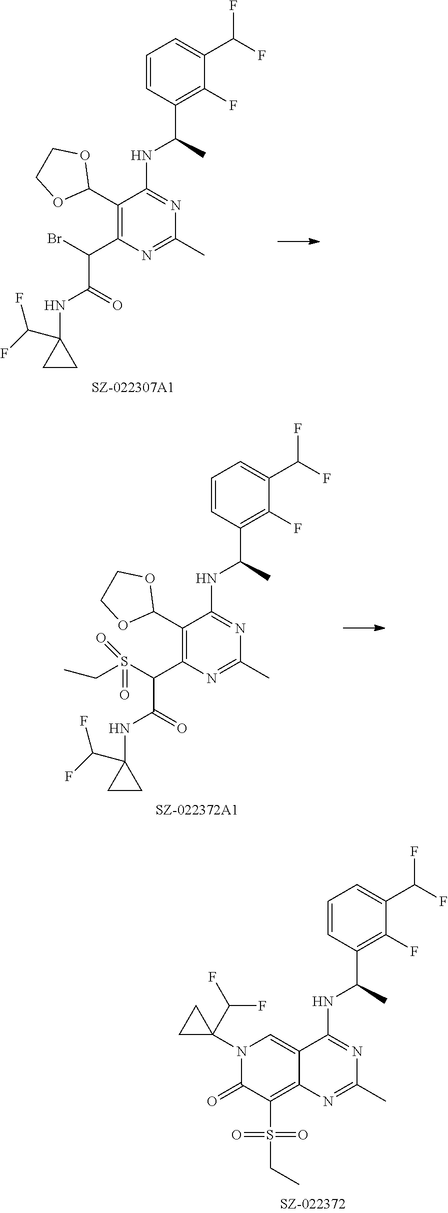 [SMILES: Cc1nc(N[C@H](C)c2cccc(C(F)F)c2F)c(C2OCCO2)c(C(Br)C(=O)NC2(C(F)F)CC2)n1 ; CCS(=O)(=O)C(C(=O)NC1(C(F)F)CC1)c1nc(C)nc(N[C@H](C)c2cccc(C(F)F)c2F)c1C1OCCO1 ; CCS(=O)(=O)c1c(=O)n(C2(C(F)F)CC2)cc2c(N[C@H](C)c3cccc(C(F)F)c3F)nc(C)nc12]

### Step 1:

SZ-022307A1 (90 mg, 0.16 mmol) and sodium ethanesulfinate (22 mg, 0.19 mmol) were dissolved in dimethyl sulfoxide (10 mL), and the solution was stirred at room temperature for 2 h. After the starting materials were consumed as monitored by LC-MS, water (100 mL) and ethyl acetate (50 mL) were added to the reaction mixture for extraction, and the organic layer was separated, washed with a saturated sodium chloride solution, dried, and concentrated by rotary evaporation to give SZ-022372A1 as a yellow solid (145 mg). LC-MS: [M+H]⁺ 592.89.

### Step 2:

SZ-022372A1 (145 mg, 0.24 mmol) and hydrochloric acid (0.15 mL, 5 M) were added to isopropanol (3 mL), and the mixture was heated to 50° C. for reaction for 3 h. After the starting materials were consumed as monitored by LC-MS, the reaction mixture was concentrated by rotary evaporation to give a crude product. The crude product was subjected to preparative high performance liquid chromatography and then lyophilized to give SZ-022372 (21.95 mg). LC-MS: [M+H]⁺ 530.86.

SZ-022372: ¹H NMR (400 MHz, DMSO-d₆) δ 9.47 (s, 1H), 9.20 (d, J=6.8 Hz, 1H), 7.67 (t, J=7.2 Hz, 1H), 7.54 (t, J=7.2 Hz, 1H), 7.34 (t, J=7.6 Hz, 1H), 7.24 (t, J=56.0 Hz, 1H), 6.36 (t, J=56.8 Hz, 1H), 5.80-5.76 (m, 1H), 3.54 (q, J=7.4 Hz, 2H), 2.26 (s, 3H), 1.60 (d, J=7.2 Hz, 3H), 1.51-1.45 (m, 4H), 1.10 (t, J=7.2 Hz, 3H).

### Example 52: SZ-022363

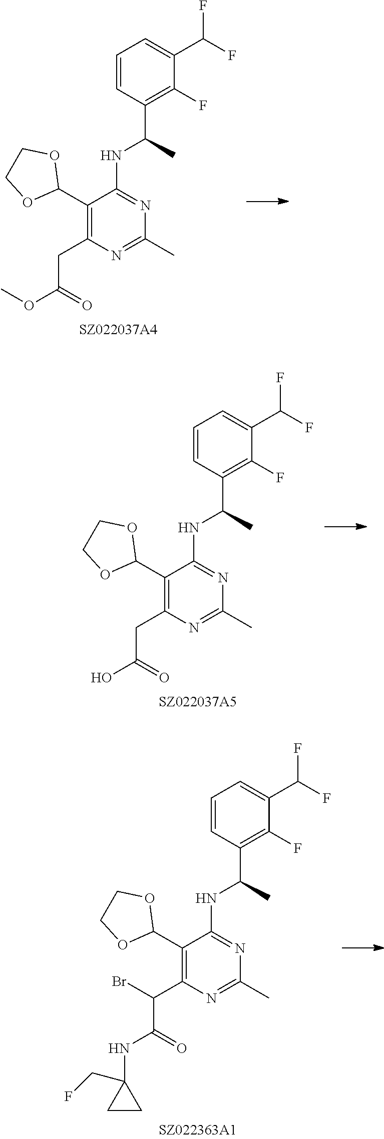 [SMILES: COC(=O)Cc1nc(C)nc(N[C@H](C)c2cccc(C(F)F)c2F)c1C1OCCO1 ; Cc1nc(CC(=O)O)c(C2OCCO2)c(N[C@H](C)c2cccc(C(F)F)c2F)n1 ; Cc1nc(N[C@H](C)c2cccc(C(F)F)c2F)c(C2OCCO2)c(C(Br)C(=O)NC2(CF)CC2)n1]

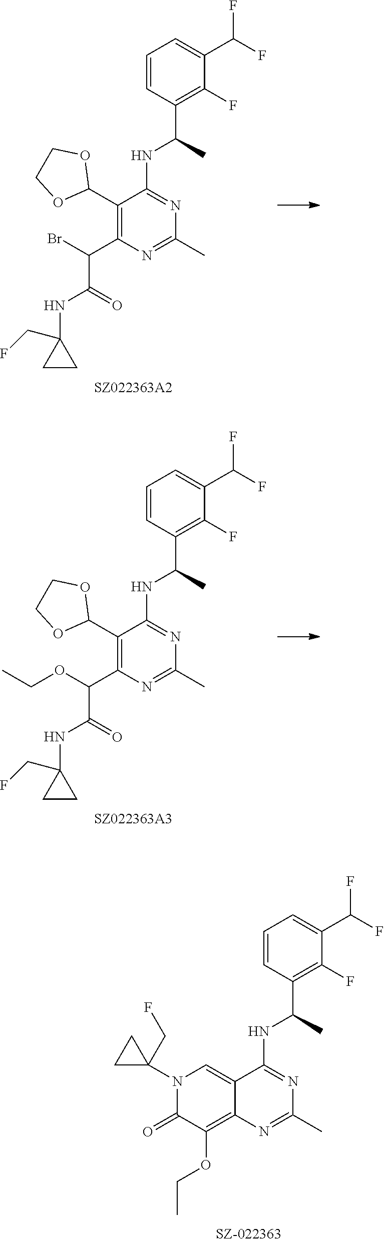 [SMILES: Cc1nc(N[C@H](C)c2cccc(C(F)F)c2F)c(C2OCCO2)c(C(Br)C(=O)NC2(CF)CC2)n1 ; CCOC(C(=O)NC1(CF)CC1)c1nc(C)nc(N[C@H](C)c2cccc(C(F)F)c2F)c1C1OCCO1 ; CCOc1c(=O)n(C2(CF)CC2)cc2c(N[C@H](C)c3cccc(C(F)F)c3F)nc(C)nc12]

### Step 1:

SZ-022037A4 (150 mg, 0.35 mmol) was added to dimethyl sulfoxide (3 mL), and then a 10% sodium hydroxide solution (0.42 mL, 1.06 mmol) was added. The mixture was allowed to react at room temperature for 1 h. After the starting materials were consumed as monitored by LC-MS, the reaction mixture was used directly in the next step without work-up. LC-MS: [M+H]⁺ 411.89.

### Step 2:

1-(Monofluoromethyl)cyclopropanamine hydrochloride (66 mg, 0.53 mmol), N,N,N,N-tetramethyl-O-(7-azabenzotriazol-1-yl)uronium hexafluorophosphate (402 mg, 1.06 mmol), and triethylamine (107 mg, 1.06 mmol) were added to the reaction system in the previous step, and the mixture was allowed to react at room temperature for 1 h. After the starting materials were consumed as monitored by LC-MS, water (20 mL) and ethyl acetate (30 mL) were added to the reaction mixture for extraction, and the organic layer was separated, washed with a saturated sodium chloride solution, dried, and concentrated to dryness under reduced pressure to give a crude product. The crude product was purified by flash silica gel column chromatography (PE:EA=2:3) to give 022363A1 as a white solid (95 mg). LC-MS: [M+H]⁺ 483.16.

### Step 3:

SZ-022363A1 (95 mg, 0.20 mmol) was added to carbon tetrachloride (4 mL), and then N-bromosuccinimide (35 mg, 0.20 mmol) and azobisisobutyronitrile (3.2 mg, 0.019 mmol) were added. The mixture was heated to 60° C. for reaction for 1 h. After the starting materials were consumed as monitored by LC-MS, the reaction mixture was directly purified by flash silica gel column chromatography (PE:EA=4:1) to give 022363A2 as a white solid (100 mg). LC-MS: [M+H]⁺ 560.71.

### Step 4:

Sodium hydride (71 mg, 0.18 mmol) was added to tetrahydrofuran (3 mL), and then ethanol (98 mg, 2.14 mmol) was added. The mixture was stirred at 0° C. for 0.5 h. A solution of SZ-022363A2 (100 mg, 0.18 mmol) in tetrahydrofuran (2 mL) was added dropwise to the reaction mixture, and the resulting mixture was stirred for another 2.5 h. After the starting materials were consumed as monitored by LC-MS, water (10 mL) and ethyl acetate (10 mL) were added to the reaction mixture for extraction, and the organic layer was separated, washed with a saturated sodium chloride solution, dried, and concentrated by rotary evaporation to give crude product SZ-022363A3 as a yellow oil (32 mg). LC-MS: [M+H]⁺ 527.02.

### Step 5:

SZ-022363A3 (32 mg, 0.06 mmol) was added to isopropanol (2.5 mL), and then a hydrochloric acid solution (2 M, 1 mL) was added. The mixture was allowed to react at 50° C. for 5 h. After the starting materials were consumed as monitored by LC-MS, the reaction mixture was directly concentrated by rotary evaporation. A saturated sodium bicarbonate solution (5 mL) and ethyl acetate (10 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022363 (12 mg). LC-MS: [M+H]⁺ 464.88.

SZ-022363: ¹H NMR (400 MHz, MeOH-d₆) δ 8.93 (s, 1H), 8.78 (d, J=7.2 Hz, 1H), 7.63 (t, J=7.6 Hz, 1H), 7.51 (t, J=7.2 Hz, 1H), 7.31 (t, J=7.6 Hz, 1H), 7.22 (t, J=54.4 Hz, 1H), 5.78-5.71 (m, 1H), 4.66-4.59 (m, 2H), 4.05 (q, J=7.2 Hz, 2H), 2.20 (s, 3H), 1.57 (d, J=7.2 Hz, 3H) 1.32 (m, 4H), 1.21 (t, J=7.2 Hz, 3H).

### Example 53: SZ-022364

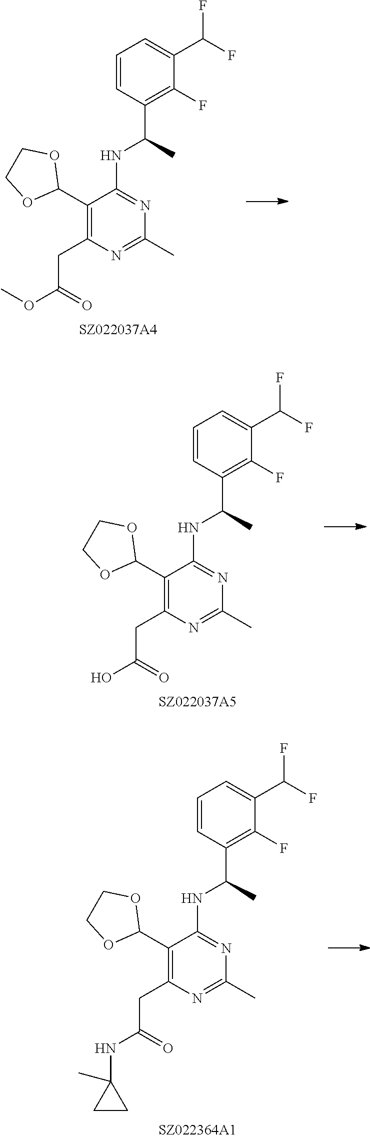 [SMILES: COC(=O)Cc1nc(C)nc(N[C@H](C)c2cccc(C(F)F)c2F)c1C1OCCO1 ; Cc1nc(CC(=O)O)c(C2OCCO2)c(N[C@H](C)c2cccc(C(F)F)c2F)n1 ; Cc1nc(CC(=O)NC2(C)CC2)c(C2OCCO2)c(N[C@H](C)c2cccc(C(F)F)c2F)n1]

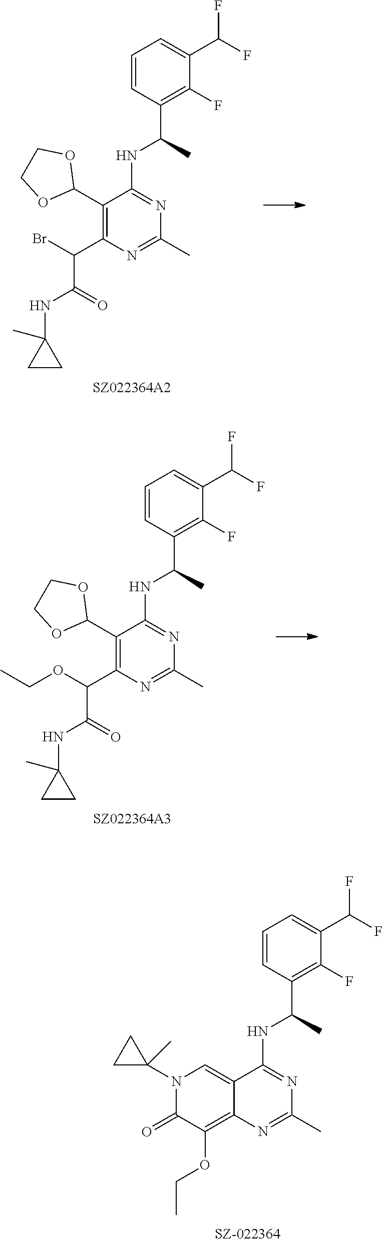 [SMILES: Cc1nc(N[C@H](C)c2cccc(C(F)F)c2F)c(C2OCCO2)c(C(Br)C(=O)NC2(C)CC2)n1 ; CCOC(C(=O)NC1(C)CC1)c1nc(C)nc(N[C@H](C)c2cccc(C(F)F)c2F)c1C1OCCO1 ; CCOc1c(=O)n(C2(C)CC2)cc2c(N[C@H](C)c3cccc(C(F)F)c3F)nc(C)nc12]

### Step 1:

SZ-022037A4 (150 mg, 0.35 mmol) was added to dimethyl sulfoxide (3 mL), and then a 10% sodium hydroxide solution (0.42 mL, 1.06 mmol) was added. The mixture was allowed to react at room temperature for 1 h. After the starting materials were consumed as monitored by LC-MS, the reaction mixture was used directly in the next step without work-up. LC-MS: [M+H]⁺ 411.92.

### Step 2:

1-Methylcyclopropanamine hydrochloride (57 mg, 0.53 mmol), N,N,N,N-tetramethyl-O-(7-azabenzotriazol-1-yl)uronium hexafluorophosphate (402 mg, 1.06 mmol), and triethylamine (107 mg, 1.06 mmol) were added to the reaction system in the previous step, and the mixture was allowed to react at room temperature for 1 h. After the starting materials were consumed as monitored by LC-MS, water (20 mL) and ethyl acetate (30 mL) were added to the reaction mixture for extraction, and the organic layer was separated, washed with a saturated sodium chloride solution, dried, and concentrated to dryness under reduced pressure to give a crude product. The crude product was purified by flash silica gel column chromatography (PE:EA=2:3) to give 022364A1 as a white solid (120 mg). LC-MS: [M+H]⁺ 464.95.

### Step 3:

SZ-022364A1 (120 mg, 0.26 mmol) was added to carbon tetrachloride (3.5 mL), and then N-bromosuccinimide (46 mg, 0.26 mmol) and azobisisobutyronitrile (4.2 mg, 0.026 mmol) were added. The mixture was heated to 60° C. for reaction for 1 h. After the starting materials were consumed as monitored by LC-MS, the reaction mixture was directly purified by flash silica gel column chromatography (PE:EA=4:1) to give 022364A2 as a white solid (80 mg). LC-MS: [M+H]⁺ 542.75.

### Step 4:

Sodium hydride (74 mg, 0.18 mmol) was added to tetrahydrofuran (3 mL), and then ethanol (101 mg, 2.21 mmol) was added. The mixture was stirred at 0° C. for 0.5 h. A solution of SZ-022364A2 (100 mg, 0.18 mmol) in tetrahydrofuran (1.5 mL) was added dropwise to the reaction mixture, and the resulting mixture was stirred for another 2.5 h. After the starting materials were consumed as monitored by LC-MS, water (10 mL) and ethyl acetate (10 mL) were added to the reaction mixture for extraction, and the organic layer was separated, washed with a saturated sodium chloride solution, dried, and concentrated by rotary evaporation to give SZ-022364A3 as a white solid (50 mg). LC-MS: [M+H]⁺ 509.11.

### Step 5:

SZ-022364A3 (50 mg, 0.1 mmol) was added to isopropanol (2 mL), and then a hydrochloric acid solution (2 M, 1 mL) was added. The mixture was allowed to react at 50° C. for 5 h. After the starting materials were consumed as monitored by LC-MS, the reaction mixture was directly concentrated by rotary evaporation. A saturated sodium bicarbonate solution (5 mL) and ethyl acetate (10 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022364 (30 mg). LC-MS: [M+H]⁺ 446.91.

SZ-022364: ¹H NMR (400 MHz, DMSO-d₆) δ 9.13 (s, 1H), 9.05 (s, 1H), 7.73 (t, J=7.6 Hz, 1H), 7.51 (t, J=7.2 Hz, 1H), 7.30 (t, J=7.7 Hz, 1H), 7.23 (t, J=53.6 Hz, 1H), 5.80-5.73 (m, 1H), 4.05 (q, J=7.2 Hz, 2H), 2.20 (s, 3H), 1.58 (d, J=7.2 Hz, 3H), 1.50 (s, 3H), 1.20 (t, J=7.2 Hz, 3H), 1.14-1.01 (m, 4H).

### Example 54: SZ-022355

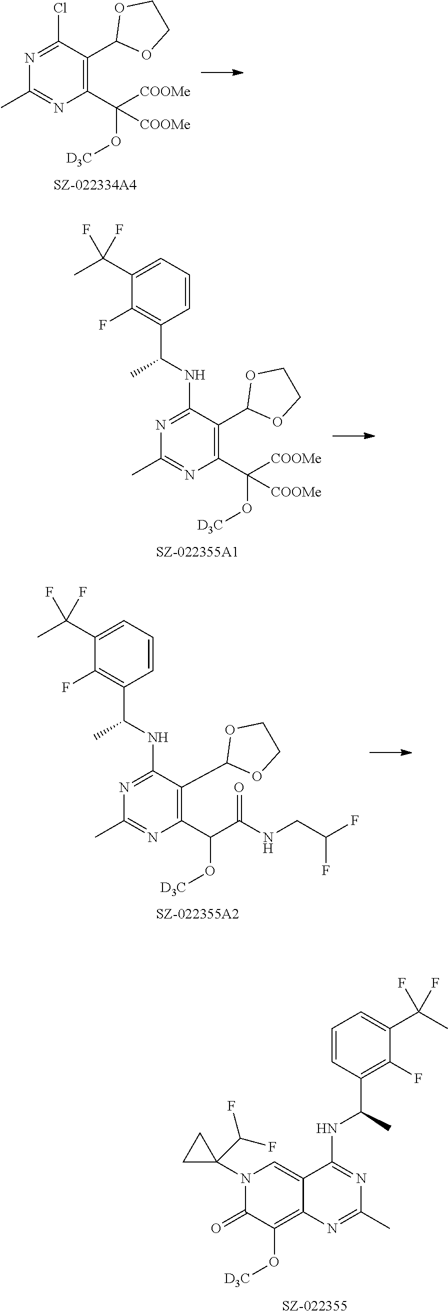 [SMILES: [2H]C([2H])([2H])OC(C(=O)OC)(C(=O)OC)c1nc(C)nc(Cl)c1C1OCCO1 ; [2H]C([2H])([2H])OC(C(=O)OC)(C(=O)OC)c1nc(C)nc(N[C@H](C)c2cccc(C(C)(F)F)c2F)c1C1OCCO1 ; [2H]C([2H])([2H])OC(C(=O)NCC(F)F)c1nc(C)nc(N[C@H](C)c2cccc(C(C)(F)F)c2F)c1C1OCCO1 ; [2H]C([2H])([2H])Oc1c(=O)n(C2(C(F)F)CC2)cc2c(N[C@H](C)c3cccc(C(C)(F)F)c3F)nc(C)nc12]

### Step 1:

SZ-022334A4 (200 mg, 0.55 mmol), (1R)-1-(2-fluoro-3-(1,1-difluoro)ethylphenyl)ethylamine hydrochloride (139 mg, 0.58 mmol), and N,N-diisopropylethylamine (213 mg, 1.65 mmol) were added to dimethyl sulfoxide (2 mL), and the mixture was heated to 80° C. for reaction for 15 h. After the starting materials were consumed as monitored by LC-MS, water (100 mL) and ethyl acetate (100 mL) were added to the reaction mixture for extraction, and the organic layer was separated, washed with a saturated sodium chloride solution, dried, and concentrated by rotary evaporation to give a crude product. The crude product was purified by flash silica gel column chromatography (PE:EA=3:1; Rf=0.3) to give product SZ-022355A1 as a light yellow oil (120 mg). LC-MS: [M+H]⁺ 530.99.

### Step 2:

SZ-022355A1 (120 mg, 0.22 mmol) was added to dimethyl sulfoxide (2 mL), and then a 10% sodium hydroxide solution (500 μL) was added. The mixture was allowed to react at 25° C. for 1 h. 1-Difluoromethylcyclopropanamine hydrochloride (49 mg, 0.34 mmol), N,N,N,N-tetramethyl-O-(7-azabenzotriazol-1-yl)uronium hexafluorophosphate (258 mg, 0.68 mmol), and triethylamine (69 mg, 0.68 mmol) were then added into the system, and the mixture was allowed to react at room temperature for another 2 h. After the starting materials were consumed as monitored by LC-MS, water (20 mL) and ethyl acetate (20 mL) were added to the reaction mixture for extraction, and the organic layer was separated, washed with a saturated sodium chloride solution, dried, and concentrated by rotary evaporation to give crude product SZ-022355A2 as a light yellow oil (190 mg). LC-MS: [M+H]⁺ 548.05.

### Step 3:

SZ-022355A2 (190 mg, 0.22 mmol) was added to isopropanol (2 mL), and then a hydrochloric acid solution (2 M, 0.5 mL) was added. The mixture was allowed to react at 50° C. for 3 h. After the starting materials were consumed as monitored by LC-MS, the reaction mixture was directly concentrated by rotary evaporation. A saturated sodium bicarbonate solution (5 mL), water (10 mL), and ethyl acetate (20 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022355 (60 mg). LC-MS: [M+H]⁺ 486.18.

SZ-022355: ¹H NMR (400 MHz, DMSO-d₆) δ 8.93 (s, 1H), 8.80 (d, J=6.8 Hz, 1H), 7.59 (t, J=6.8 Hz, 1H), 7.45 (t, J=6.8 Hz, 1H), 7.28 (t, J=7.6 Hz, 1H), 6.34 (t, J=57.2 Hz, 1H), 5.78-5.71 (m, 1H), 2.21 (s, 3H), 2.02 (t, J=19.2 Hz, 3H), 1.57 (d, J=6.8 Hz, 3H), 1.51-1.47 (m, 2H), 1.45-1.33 (m, 2H).

### Example 55: SZ-022349

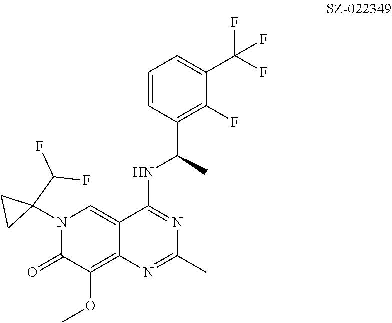 [SMILES: COc1c(=O)n(C2(C(F)F)CC2)cc2c(N[C@H](C)c3cccc(C(F)(F)F)c3F)nc(C)nc12]

By referring to Example SZ-022348 with 022348A4 replaced by (R)-1-(2-fluoro-3-(trifluoromethyl)phenyl)ethyl-1-amine hydrochloride, separation was performed by preparative high performance liquid chromatography, followed by lyophilization to give SZ-022349 as a yellow solid (30 mg). LC-MS: [M+H]⁺ 486.85. ¹H NMR (400 MHz, DMSO-d₆) δ 8.92 (s, 1H), 8.85 (d, J=6.8 Hz, 1H), 7.76 (t, J=7.2 Hz, 1H), 7.66 (t, J=7.6 Hz, 1H), 7.38 (t, J=7.6 Hz, 1H), 6.34 (t, J=56.8 Hz, 1H), 5.74-5.67 (m, 1H), 3.76 (s, 3H), 2.19 (s, 3H), 1.59 (d, J=7.2 Hz, 3H), 1.51-1.47 (m, 2H), 1.42-1.35 (m, 2H).

### Example 56: SZ-022350

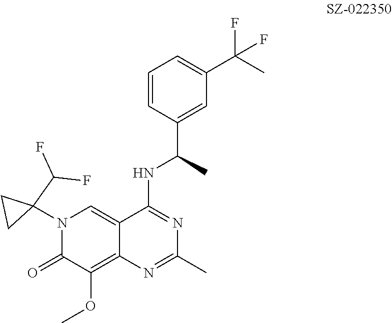 [SMILES: COc1c(=O)n(C2(C(F)F)CC2)cc2c(N[C@H](C)c3cccc(C(C)(F)F)c3)nc(C)nc12]

By referring to Example SZ-022348 with 022348A4 replaced by (R)-1-(3-(1,1-difluoroethyl)phenyl)ethyl-1-amine hydrochloride, separation was performed by preparative high performance liquid chromatography, followed by lyophilization to give SZ-022350 as a yellow solid (20 mg). LC-MS: [M+H]⁺ 465.21. ¹H NMR (400 MHz, CDCl₃) δ 8.22 (s, 1H), 7.60 (s, 1H), 7.51-7.54 (m, 1H), 7.40-7.42 (m, 2H), 6.67 (br, 1H), 6.32 (t, J=58.8 Hz, 1H), 5.78 (br, 1H), 4.06 (s, 3H), 2.486 (s, 3H), 1.94 (t, J=18.0 Hz, 3H), 1.71 (d, J=7.2 Hz, 3H), 1.54-1.56 (m, 2H), 1.25 (br, 2H).

### Example 57: SZ-022368

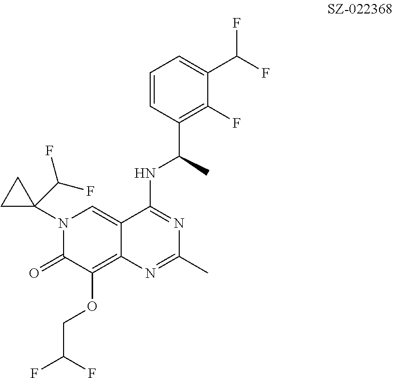 [SMILES: Cc1nc(N[C@H](C)c2cccc(C(F)F)c2F)c2cn(C3(C(F)F)CC3)c(=O)c(OCC(F)F)c2n1]

By referring to Example SZ-022351 with (S)-3-hydroxytetrahydrofuran replaced by 2,2-difluoroethanol, separation was performed by preparative high performance liquid chromatography, followed by lyophilization to give SZ-022368 as a yellow solid (30 mg). LC-MS: [M+H]⁺ 519.17. ¹H NMR (400 MHz, DMSO-d₆) δ 8.98 (s, 1H), 8.88 (d, J=7.1 Hz, 1H), 7.64 (t, J=7.4 Hz, 1H), 7.53 (t, J=7.1 Hz, 1H), 7.33 (t, J=7.7 Hz, 1H), 7.23 (t, J=54.2 Hz, 1H), 6.35 (t, J=56.8 Hz, 1H), 6.32 (tt, J=55.4, 4.0 Hz, 1H), 5.79-5.72 (m, 1H), 4.26 (td, J=14.5, 4.1 Hz, 2H), 2.23 (s, 3H), 1.59 (d, J=7.1 Hz, 3H), 1.53-1.33 (m, 4H).

### Example 58: SZ-022362

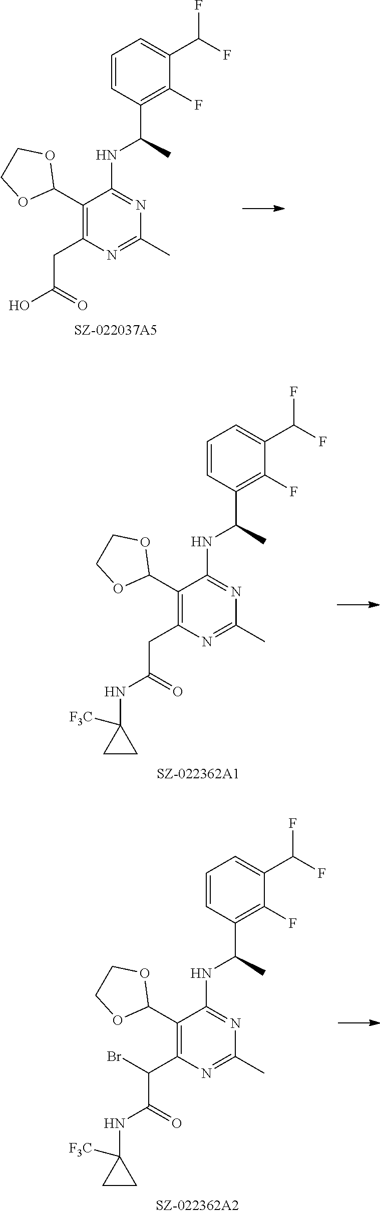 [SMILES: Cc1nc(CC(=O)O)c(C2OCCO2)c(N[C@H](C)c2cccc(C(F)F)c2F)n1 ; Cc1nc(CC(=O)NC2(C(F)(F)F)CC2)c(C2OCCO2)c(N[C@H](C)c2cccc(C(F)F)c2F)n1 ; Cc1nc(N[C@H](C)c2cccc(C(F)F)c2F)c(C2OCCO2)c(C(Br)C(=O)NC2(C(F)(F)F)CC2)n1]

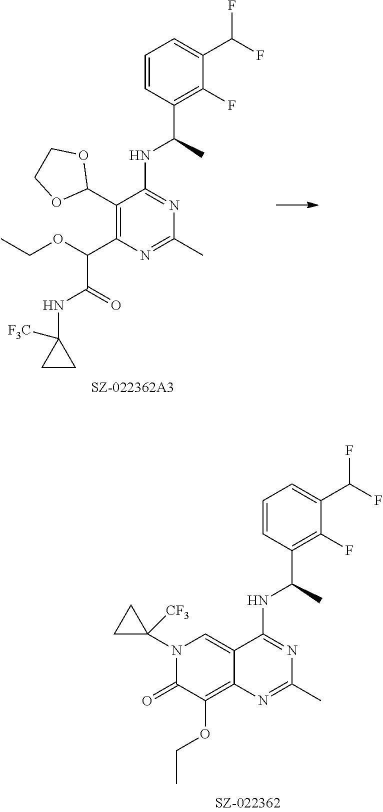 [SMILES: CCOC(C(=O)NC1(C(F)(F)F)CC1)c1nc(C)nc(N[C@H](C)c2cccc(C(F)F)c2F)c1C1OCCO1 ; CCOc1c(=O)n(C2(C(F)(F)F)CC2)cc2c(N[C@H](C)c3cccc(C(F)F)c3F)nc(C)nc12]

### Step 1:

SZ-022037A5 (411 mg, 1 mmol) was added to N,N-dimethylformamide (10 mL), and then 1-trifluoromethylcyclopropanamine hydrochloride (242 mg, 1.5 mmol), N,N,N,N-tetramethyl-O-(7-azabenzotriazol-1-yl)uronium hexafluorophosphate (1.14 mg, 3 mmol), and triethylamine (303 mg, 3 mmol) were added. The mixture was allowed to react at room temperature for 1 h. After the starting materials were consumed as monitored by LC-MS, water (30 mL) and ethyl acetate (50 mL) were added to the reaction mixture for extraction, and the organic layer was separated, washed with a saturated sodium chloride solution, dried, and concentrated to dryness under reduced pressure to give a crude product. The crude product was purified by flash silica gel column chromatography (PE:EA=2:3) to give SZ-022362A1 as a white solid (180 mg). LC-MS: [M+H]⁺ 519.00.

### Step 2:

SZ-022362A1 (180 mg, 0.35 mmol) was added to acetonitrile (7 ml), and then N-bromosuccinimide (62 mg, 0.35 mmol) was added. The mixture was allowed to react at room temperature for 1 h. After the starting materials were consumed as monitored by LC-MS, the reaction mixture was directly purified by flash silica gel column chromatography (PE:EA=4:1) to give SZ-022362A2 as a white solid (110 mg). LC-MS: [M+H]⁺ 596.74.

### Step 3:

Sodium hydride (74 mg, 1.84 mmol) was added to tetrahydrofuran (3 mL), and then ethanol (102 mg, 2.21 mmol) was added. The mixture was stirred at 0° C. for 0.5 h. A solution of SZ-022362A2 (110 mg, 0.18 mmol) in tetrahydrofuran (2 mL) was added dropwise to the reaction mixture, and the resulting mixture was stirred for another 2.5 h. After the starting materials were consumed as monitored by LC-MS, water (15 mL) and ethyl acetate (15 mL) were added to the reaction mixture for extraction, and the organic layer was separated, washed with a saturated sodium chloride solution, dried, and concentrated by rotary evaporation. The residue was purified by flash silica gel column chromatography (PE:EA=3:2) to give SZ-022362A3 as a white solid (20 mg). LC-MS: [M+H]⁺ 562.93.

### Step 4:

SZ-022362A3 (32 mg, 0.06 mmol) was added to isopropanol (2 mL), and then a hydrochloric acid solution (2 M, 1 mL) was added. The mixture was allowed to react at 50° C. for 5 h. After the starting materials were consumed as monitored by LC-MS, the reaction mixture was directly concentrated by rotary evaporation. A saturated sodium bicarbonate solution (5 mL) and ethyl acetate (10 mL) were then added for extraction, and the organic layer was separated and washed with a saturated sodium chloride solution. The solvent was removed by rotary evaporation, and the residue was subjected to preparative high performance liquid chromatography and then lyophilized to give SZ-022362 (7 mg). LC-MS: [M+H]⁺ 500.90.

SZ-022362: ¹H NMR (400 MHz, DMSO-d₆) δ 8.97 (s, 1H), 8.82 (d, J=7.2 Hz, 1H), 7.63 (t, J=7.2 Hz, 1H), 7.52 (t, J=7.2 Hz, 1H), 7.32 (t, J=7.6 Hz, 1H), 7.23 (t, J=54.4 Hz, 1H), 5.76-5.73 (m, 1H), 4.05 (q, J=7.2 Hz, 2H), 2.20 (s, 3H), 1.64-1.53 (m, 4H), 1.57 (d, J=6.8 Hz, 3H), 1.20 (t, J=7.2 Hz, 3H).

### Example 59: SZ-022359

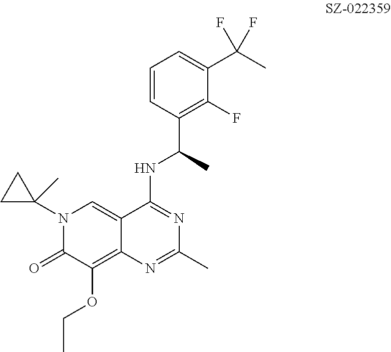 [SMILES: CCOc1c(=O)n(C2(C)CC2)cc2c(N[C@H](C)c3cccc(C(C)(F)F)c3F)nc(C)nc12]

By referring to Example SZ-022338 with 1-difluoromethylcyclopropanamine hydrochloride replaced by 1-methylcyclopropanamine hydrochloride, separation was performed by preparative high performance liquid chromatography, followed by lyophilization to give SZ-022359 as a yellow solid (21 mg). LC-MS: [M+H]⁺ 461.19. ¹H NMR (400 MHz, DMSO-d₆) δ 8.98 (s, 1H), 8.69 (d, J=6.8 Hz, 1H), 7.67-7.56 (m, 1H), 7.43-7.47 (m, 1H), 7.27 (t, J=7.6 Hz, 1H), 5.80-5.68 (m, 1H), 4.05 (q, J=7.2 Hz, 2H), 2.19 (s, 3H), 2.03 (t, J=19.2 Hz, 3H), 1.57 (d, J=7.2 Hz, 3H), 1.51 (s, 3H), 1.21 (t, J=7.2 Hz, 3H), 1.17-0.99 (m, 4H).

### Example 60: SZ-022360

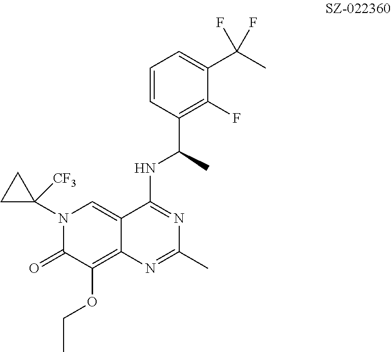 [SMILES: CCOc1c(=O)n(C2(C(F)(F)F)CC2)cc2c(N[C@H](C)c3cccc(C(C)(F)F)c3F)nc(C)nc12]

By referring to Example SZ-022338 with 1-difluoromethylcyclopropanamine hydrochloride replaced by 1-trifluoromethylcyclopropanamine hydrochloride, separation was performed by preparative high performance liquid chromatography, followed by lyophilization to give SZ-022360 as a yellow solid (57 mg). LC-MS: [M+H]⁺ 515.17. ¹H NMR (400 MHz, DMSO-d₆) δ 8.99 (s, 1H), 8.82 (d, J=6.8 Hz, 1H), 7.59 (t, J=6.8 Hz, 1H), 7.46 (t, J=7.2 Hz, 1H), 7.32-7.24 (m, 1H), 5.78-5.70 (m, 1H), 4.06 (q, J=7.2 Hz, 2H), 2.20 (s, 3H), 2.02 (t, J=19.2 Hz, 3H), 1.85-1.77 (m, 1H), 1.73-1.61 (m, 2H), 1.58 (d, J=7.2 Hz, 3H), 1.56-1.49 (m, 1H), 1.21 (t, J=7.2 Hz, 3H).

### Example 61: SZ-022361

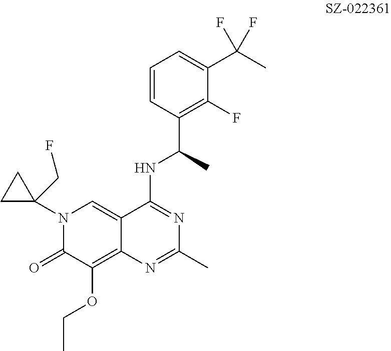 [SMILES: CCOc1c(=O)n(C2(CF)CC2)cc2c(N[C@H](C)c3cccc(C(C)(F)F)c3F)nc(C)nc12]

By referring to Example SZ-022338 with 1-difluoromethylcyclopropanamine hydrochloride replaced by 1-fluoromethylcyclopropanamine hydrochloride, separation was performed by preparative high performance liquid chromatography, followed by lyophilization to give SZ-022361 as a yellow solid (76 mg). LC-MS: [M+H]⁺ 479.20. ¹H NMR (400 MHz, DMSO-d₆) δ 8.94 (s, 1H), 8.84 (s, 1H), 7.59 (t, J=7.6 Hz, 1H), 7.44 (t, J=7.2 Hz, 1H), 7.26 (t, J=8.0 Hz, 1H), 5.77-5.70 (m, 1H), 4.75-4.50 (m, 1H), 4.06 (q, J=7.2 Hz, 2H), 2.20 (s, 3H), 2.01 (t, J=19.2 Hz, 3H), 1.56 (d, J=7.2 Hz, 3H), 1.35-1.25 (m, 4H), 1.21 (t, J=6.8 Hz, 3H).

### Example 62: SZ-022381

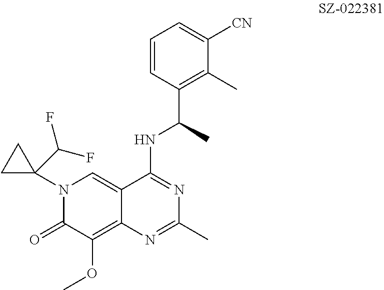 [SMILES: COc1c(=O)n(C2(C(F)F)CC2)cc2c(N[C@H](C)c3cccc(C#N)c3C)nc(C)nc12]

By referring to Example SZ-022304 with (1R)-1-(3-(trifluoromethyl)-2-methylphenyl)ethylamine hydrochloride replaced by (1R)-1-(3-cyano-2-methylphenyl)ethylamine hydrochloride, separation was performed by preparative high performance liquid chromatography, followed by lyophilization to give SZ-022381 as a yellow solid (10 mg). LC-MS: [M+H]⁺ 440.03. ¹H NMR (400 MHz, DMSO-d₆) δ 8.90 (s, 1H), 8.86 (d, J=6.8 Hz, 1H), 7.71 (d, J=7.6 Hz, 1H), 7.66 (d, J=7.6 Hz, 1H), 7.39 (t, J=7.6 Hz, 1H), 6.34 (t, J=56.8 Hz, 1H), 5.65-5.57 (m, 1H), 3.76 (s, 3H), 2.66 (s, 3H), 2.22 (s, 3H), 1.52 (d, J=6.8 Hz, 3H), 1.50-1.35 (m, 4H).

### Example 63: SZ-022382

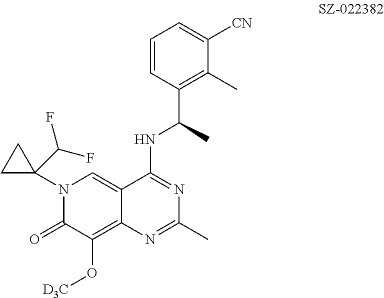 [SMILES: [2H]C([2H])([2H])Oc1c(=O)n(C2(C(F)F)CC2)cc2c(N[C@H](C)c3cccc(C#N)c3C)nc(C)nc12]

By referring to Example SZ-022334 with (1R)-1-(3-(trifluoromethyl)-2-methylphenyl)ethylamine hydrochloride replaced by (1R)-1-(3-cyano-2-methylphenyl)ethylamine hydrochloride, separation was performed by preparative high performance liquid chromatography, followed by lyophilization to give SZ-022382 as a yellow solid (15 mg). LC-MS: [M+H]⁺ 443.34. ¹H NMR (400 MHz, DMSO-d₆) δ 8.89 (s, 1H), 8.86 (d, J=6.8 Hz, 1H), 7.71 (d, J=7.6 Hz, 1H), 7.66 (d, J=7.6 Hz, 1H), 7.39 (t, J=7.6 Hz, 1H), 6.33 (t, J=56.8 Hz, 1H), 5.64-5.68 (m, 1H), 2.66 (s, 3H), 2.22 (s, 3H), 1.52 (d, J=6.8 Hz, 3H), 1.50-1.35 (m, 4H).

### Example 64: SZ-022383

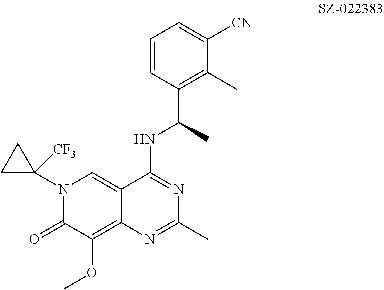 [SMILES: COc1c(=O)n(C2(C(F)(F)F)CC2)cc2c(N[C@H](C)c3cccc(C#N)c3C)nc(C)nc12]

By referring to Example SZ-022304 with (1R)-1-(3-(trifluoromethyl)-2-methylphenyl)ethylamine hydrochloride replaced by (1R)-1-(3-cyano-2-methylphenyl)ethylamine hydrochloride and 1-(difluoromethyl)cyclopropanamine hydrochloride replaced by 1-trifluoromethylcyclopropanamine hydrochloride, separation was performed by preparative high performance liquid chromatography, followed by lyophilization to give SZ-022383 as a yellow solid (78 mg). LC-MS: [M+H]⁺ 458.30. ¹H NMR (400 MHz, DMSO-d₆) δ 8.96 (s, 1H), 8.87 (d, J=6.4 Hz, 1H), 7.71 (d, J=7.6 Hz, 1H), 7.66 (d, J=7.6 Hz, 1H), 7.40 (d, J=6.4 Hz, 1H), 5.66-5.55 (m, 1H), 3.76 (s, 3H), 2.66 (s, 3H), 2.22 (s, 3H), 1.89-1.60 (m, 4H), 1.53 (d, J=6.8 Hz, 3H).

### Example 65: SZ-022384

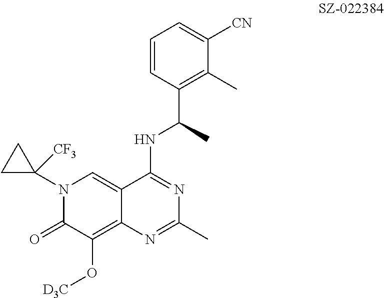 [SMILES: [2H]C([2H])([2H])Oc1c(=O)n(C2(C(F)(F)F)CC2)cc2c(N[C@H](C)c3cccc(C#N)c3C)nc(C)nc12]

By referring to Example SZ-022334 with (1R)-1-(3-(trifluoromethyl)-2-methylphenyl)ethylamine hydrochloride replaced by (1R)-1-(3-cyano-2-methylphenyl)ethylamine hydrochloride and 1-(difluoromethyl)cyclopropanamine hydrochloride replaced by 1-trifluoromethylcyclopropanamine hydrochloride, separation was performed by preparative high performance liquid chromatography, followed by lyophilization to give SZ-022384 as a yellow solid (83 mg). LC-MS: [M+H]⁺ 461.33. ¹H NMR (400 MHz, DMSO-d₆) δ 8.99 (s, 1H), 8.90 (d, J=6.0 Hz, 1H), 7.74 (d, J=7.6 Hz, 1H), 7.69 (d, J=7.6 Hz, 1H), 7.43 (d, J=6.4 Hz, 1H), 5.70-5.58 (m, 1H), 2.69 (s, 3H), 2.25 (s, 3H), 1.90-1.60 (m, 4H), 1.56 (d, J=7.2 Hz, 3H).

### Efficacy Example 1: In-cell Western Blot Assay for ERK Phosphorylation in DLD-1 Cells

DLD-1 cells (ATCC, CCL-221) were thawed and cultured in a culture medium containing RPMI1640 (Gibco, A10491-01), 10% fetal bovine serum (FBS) (Transgene, FS201-02) and 1% antibiotic P/S (Gibco, 15140-122) for 3 days until the cell viability was good. The cells were inoculated into a 384-well plate and incubated at 37° C. with 5% CO₂ overnight. A test compound, a positive control compound, and a negative control were added. The compound was at concentrations of 10000 nM to 1.52 nM (3-fold dilution) and 0.051 nM, for a total of 10 concentrations. The mixture was well mixed and incubated at 37° C. with 5% CO₂. The cells were washed twice with PBS (Solarbio, P1010), and then a blocking buffer was added. After 1 h of blocking at room temperature, a primary antibody mixture (phospho-p44/42 MAPK (T202/Y204) Rabbit mAb (CST, 4 S), GAPDH (D4C6R) Mouse mAb) (CST, 97166S) was added, followed by overnight incubation at 4° C. The cells were washed 3 times with PBST (PBS containing 0.05% Tween-20 (Solarbio, T8220)), and a secondary antibody mixture (goat anti rabbit 800CW (LI-COR, 926-32211), goat anti mouse 680RD (LI-COR, 926-68070)) was added, followed by incubation at room temperature in the dark. The 384-well plate was reversed and centrifuged at 1000 rpm for 1 min, and read on an Odyssey CLx fluorescence imaging system (LI-COR) to obtain fluorescence signal values, which were corrected using DMSO and BI-3406. The specific calculation is as follows, and the results are shown in Table 1:

$$Relative Signal = Signal Value ( total channel 800 ) / Signal Value ( total channel 700 )$$

H=Ave (DMSO)
L=Ave (BI-3406)
SD (H)=STDEV (DMSO)
SD (L)=STDEV (BI-3406)

$$CV \% ( DMSO ) = 100 * ( SD\_H / Ave\_H ) \\ CV \% ( BI - 3406 ) = 100 * SD\_L / Ave\_L \\ {Z}^{′} = 1 - 3 * ( SD\_H + SD\_L ) / ( Ave\_H - Ave\_L ) \\ Relative pERK = ( Sample - Ave\_L ) / ( Ave\_H - Ave\_L ) .$$

The IC₅₀ values of the compounds were calculated using a four-parameter fitting algorithm, specifically as follows:

$$Y = Bottom + ( Top - Bottom ) / ( 1 + 10 \hat{} ( ( Log {IC}_{50} - X ) * HillSlope ) )$$

X: Log of cpd concentration
Y: Ave (relative pERK)
Top and Bottom: Plateaus in same units as Y
log IC₅₀: same log units as X
HillSlope: Slope factor or Hill slope.

TABLE 1

| Compound | DLD-1 p-ERK IC₅₀ (nM) | Compound | DLD-1 p-ERK IC₅₀ (nM) |
|---|---|---|---|
| SZ-022244 | 265 | SZ-022334 | 93 |
| SZ-022300 | 412 | SZ-022327 | 153 |
| SZ-022301 | 262 | SZ-022330 | 117 |
| SZ-022302 | 104 | SZ-022330B | 400 |
| SZ-022304 | 79 | SZ-022295 | 256 |
| SZ-022296 | 143 | SZ-022345 | 885 |
| SZ-022303 | 128 | SZ-022346 | 1016 |
| SZ-022317 | 371 | SZ-022348 | 614 |
| SZ-022325 | 216 | SZ-022337 | 174 |
| SZ-022306 | 213 | SZ-022339 | 186 |
| SZ-022329 | 417 | SZ-022338 | 334 |
| SZ-022328 | 309 | SZ-022340 | 367 |
| SZ-022251 | 146 | SZ-022313 | 1272 |
| SZ-022307 | 253 | SZ-022351 | 555 |
| SZ-022312 | 601 | SZ-022352 | 516 |
| SZ-022314 | 563 | SZ-022353 | 377 |
| SZ-022354 | 138 | SZ-022350 | 211 |
| SZ-022342 | 57 | SZ-022368 | 249 |
| SZ-022349 | 193 | SZ-022355 | 112 |
| SZ-022341 | 161 | SZ-022356 | 218 |
| SZ-022363 | 171 | SZ-022357 | 165 |
| SZ-022364 | 303 | SZ-022358 | 222 |
| SZ-022371 | 274 | SZ-022373 | 42 |
| SZ-022362 | 77 | SZ-022381 | 741 |
| SZ-022359 | 231 | SZ-022382 | 729 |
| SZ-022360 | 221 | SZ-022383 | 985 |
| SZ-022361 | 164 | SZ-022384 | 884 |
| SZ-022299 | 751 | | |

### Efficacy Example 2: Evaluation of Inhibition of SOS1/KRAS G12C Protein Binding

A test compound was dissolved in 100% DMSO to give a solution at a concentration of 10 mM. 10 μL of the solution was taken and 3-fold diluted with 100% DMSO to prepare test compound solutions with concentrations of 10000 nM to 0.017 nM, for a total of 11 concentrations. The compound solutions at different concentrations were then transferred to a 384-well plate at 0.1 μL/well using a sonic droplet device Echo (Labcyte), and 2 replicate wells were set for each concentration.

A 15 nM KRAS G12C (aa 1-169) protein (Pharmaron Beijing Co., Ltd., 20200707) was transferred to the 384-well reaction plate at 5 μL/well using the sonic droplet device Echo (Labcyte), and then the reaction plate was centrifuged in a centrifuge (Eppendorf, 5810R) at 1000 rpm/min for 1 min.

A 2.5 nM His-SOS1 (aa 564-1049) protein (Pharmaron Beijing Co., Ltd., 20200717) was transferred to the 384-well reaction plate at 5 μL/well using the sonic droplet device Echo (Labcyte), and then the reaction plate was centrifuged in a centrifuge (Eppendorf, 5810R) at 1000 rpm/min for 1 min and incubated at 25° C. for 15 min.

A mixed solution of Antibody 1 (MAb Anti-6his-Tb cryptate Gold, Cisbio, 61HI2TLA) and Antibody 2 (MAb Anti-GST-XL665, Cisbio, 61GSTXLA) was transferred to the 384-well reaction plate at 10 μL/well using the sonic droplet device Echo (Labcyte), and then the reaction plate was centrifuged in a centrifuge (Eppendorf, 5810R) at 1000 rpm/min for 1 min and incubated at 25° C. for 120 min.

The 665 nm/615 nm optical density ratio was read using an Envision multifunctional microplate reader (Perkin Elmer, Envision 2104).

### Analysis of Original Data:

The IC₅₀ values of the compounds were fitted by a nonlinear regression equation of Graphpad Prism 8. The results are shown in Table 2:
Negative control: DMSO

The IC₅₀ (half maximal inhibitory concentration) values of the compounds were calculated using the following non-linear fitting equation:

$$Y = Bottom + ( Top - Bottom ) / ( 1 + 10 \hat{} ( ( Log {IC}_{50} - X ) * HillSlope ) )$$

X: Log of cpd concentration
Y: 665 nm/615 nm ratio

TABLE 2

| Compound | KRAS-SOS1 IC₅₀ (nM) | Compound | KRAS-SOS1 IC₅₀ (nM) |
|---|---|---|---|
| SZ-022244 | 17.77 | SZ-022327 | 5.045 |
| SZ-022301 | 6.496 | SZ-022330 | 5.018 |
| SZ-022300 | 12.08 | SZ-022330B | 9.99 |
| SZ-022302 | 3.788 | SZ-022295 | 8.153 |
| SZ-022304 | 6.982 | SZ-022348 | 21.78 |
| SZ-022296 | 9.425 | SZ-022343 | 26.65 |
| SZ-022303 | 9.77 | SZ-022344 | 23.71 |
| SZ-022317 | 4.538 | SZ-022345 | 21.79 |
| SZ-022325 | 15.91 | SZ-022346 | 29.97 |
| SZ-022326 | 10.52 | SZ-022337 | 4.144 |
| SZ-022306 | 12.52 | SZ-022339 | 7.249 |
| SZ-022329 | 2.635 | SZ-022338 | 12.08 |
| SZ-022328 | 9.698 | SZ-022340 | 15.18 |
| SZ-022336 | 36.52 | SZ-022299 | 22.43 |
| SZ-022307 | 25.85 | SZ-022351 | 21.36 |
| SZ-022312 | 17.87 | SZ-022353 | 17.47 |
| SZ-022314 | 17.33 | SZ-022352 | 16 |
| SZ-022334 | 4.178 | SZ-022347 | 60.08 |
| SZ-022335 | 27.87 | SZ-022354 | 21.82 |
| SZ-022350 | 16.73 | SZ-022363 | 7.358 |
| SZ-022342 | 7.147 | SZ-022364 | 22.24 |
| SZ-022368 | 23.45 | SZ-022371 | 21.79 |
| SZ-022349 | 7.234 | SZ-022372 | 36.65 |
| SZ-022341 | 7.755 | SZ-022355 | 2.472 |
| SZ-022356 | 4.358 | SZ-022357 | 3.681 |
| SZ-022358 | 4.793 | SZ-022373 | 1.396 |
| SZ-022362 | 12.44 | SZ-022381 | 12.31 |
| SZ-022359 | 8.568 | SZ-022382 | 8.862 |
| SZ-022360 | 9.461 | SZ-022383 | 18.45 |
| SZ-022361 | 4.618 | SZ-022384 | 15.4 |
| SZ-022313 | 43.97 | | |

1. A fused ring compound represented by formula I or a pharmaceutically acceptable salt thereof: 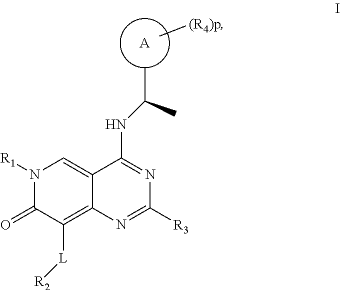
wherein,
R₁ is C₁₋₆ alkyl, C₁₋₆ alkyl substituted with one or more R₁₋₁ₐ, C₃₋₁₀ cycloalkyl, C₃₋₁₀ cycloalkyl substituted with one or more R_{1-1b}, “3- to 10-membered heterocyclyl having 1, 2 or 3 heteroatoms selected from 1, 2 or 3 of N, O and S”, “3- to 10-membered heterocyclyl having 1, 2 or 3 heteroatoms selected from 1, 2 or 3 of N, O and S” substituted with one or more R_{1-1c}, C₆₋₁₀ aryl, C₆₋₁₀ aryl substituted with one or more R_{1-1d}, “5- to 10-membered heteroaryl having 1, 2 or 3 heteroatoms selected from 1, 2 or 3 of N, O and S”, or “5- to 10-membered heteroaryl having 1, 2 or 3 heteroatoms selected from 1, 2 or 3 of N, O and S” substituted with one or more R₁₋₁ₑ;
R₁₋₁ₐ, R_{1-1b}, R_{1-1c}, R_{1-1d}, and R₁₋₁ₑ are each independently —OR₁₋₂ₐ, —NR_{1-2b}R_{1-2c}, halogen, —CN, —C(O)R_{1-2d}, —C(O)OR₁₋₂ₑ, —C(O)NR_{1-2f}R_{1-2g}, C₁₋₆ alkyl, C₁₋₆ alkyl substituted with one or more R₁₋₃ₐ, C₃₋₁₀ cycloalkyl, or “C₃₋₁₀ cycloalkyl substituted with one or more R_{1-3b}”;
R₁₋₂ₐ, R_{1-2b}, R_{1-2c}, R_{1-2d}, R₁₋₂ₑ, R_{1-2f}, and R_{1-2g} are each independently H, C₁₋₆ alkyl, C₁₋₆ alkyl substituted with one or more R_{1-3c}, C₃₋₁₀ cycloalkyl, C₃₋₁₀ cycloalkyl substituted with one or more R_{1-3d}, “3- to 10-membered heterocyclyl having 1, 2 or 3 heteroatoms selected from 1, 2 or 3 of N, O and S”, “3- to 10-membered heterocyclyl having 1, 2 or 3 heteroatoms selected from 1, 2 or 3 of N, O and S” substituted with one or more R₁₋₃ₑ, C₆₋₁₀ aryl, C₆₋₁₀ aryl substituted with one or more R_{1-3f}, “5- to 10-membered heteroaryl having 1, 2 or 3 heteroatoms selected from 1, 2 or 3 of N, O and S”, or “5- to 10-membered heteroaryl having 1, 2 or 3 heteroatoms selected from 1, 2 or 3 of N, O and S” substituted with one or more R_{1-3g};
R₁₋₃ₐ, R_{1-3b}, R_{1-3c}, R_{1-3d}, R₁₋₃ₑ, R_{1-3f}, and R_{1-3g} are each independently —OR₁₋₄ₐ, —NR_{1-4b}R_{1-4c}, halogen, C₁₋₆ alkyl substituted with one or more halogens, C₃₋₁₀ cycloalkyl, “3- to 10-membered heterocyclyl having 1, 2 or 3 heteroatoms selected from 1, 2 or 3 of N, O and S”, C₆₋₁₀ aryl, or “5- to 10-membered heteroaryl having 1, 2 or 3 heteroatoms selected from 1, 2 or 3 of N, O and S”;
R₁₋₄ₐ, R_{1-4b}, and R_{1-4c} are each independently hydrogen, C₁₋₆ alkyl, C₁₋₆ alkyl substituted with one or more halogens, C₃₋₁₀ cycloalkyl, “3- to 10-membered heterocyclyl having 1, 2 or 3 heteroatoms selected from 1, 2 or 3 of N, O and S”, C₆₋₁₀ aryl, or “5- to 10-membered heteroaryl having 1, 2 or 3 heteroatoms selected from 1, 2 or 3 of N, O and S”;
L is —O—, —S—, —SO₂—, or —NR_{L-1}; R_{L-1} is hydrogen or C₁₋₆ alkyl;
R₂ is selected from hydrogen, C₁₋₆ alkyl, C₁₋₆ alkyl substituted with one or more R₂₋₁, C₃₋₁₀ cycloalkyl, or “3- to 10-membered heterocyclyl having 1, 2 or 3 heteroatoms selected from 1, 2 or 3 of N, O and S”;
each R₂₋₁ is independently deuterium, halogen, C₃₋₁₀ cycloalkyl, or hydroxy;
R₃ is hydrogen, C₁₋₆ alkyl, or C₁₋₆ alkyl substituted with one or more halogens;
each R₄ is independently halogen, —NH₂, —CN, C₁₋₆ alkyl, C₁₋₆ alkyl substituted with one or more R₄₋₁, “3- to 10-membered heterocyclyl having 1, 2 or 3 heteroatoms selected from 1, 2 or 3 of N, O and S”, C₃₋₆ cycloalkyl, C₆₋₁₀ aryl, or C₆₋₁₀ aryl substituted with one or more —(CH₂)ₘNR₄₋₂R₄₋₃, wherein m is 0, 1, 2 or 3;
each R₄₋₁ is independently halogen or hydroxy;
R₄₋₂ and R₄₋₃ are each independently C₁₋₆ alkyl;
ring A is C₆₋₁₀ aryl, “5- to 10-membered heterocyclyl having 1, 2 or 3 heteroatoms selected from 1, 2 or 3 of N, O and S”, or “5- to 10-membered heteroaryl having 1, 2 or 3 heteroatoms selected from 1, 2 or 3 of N, O and S”; and
p is 1, 2 or 3.

2. The fused ring compound represented by formula I or the pharmaceutically acceptable salt thereof according to claim 1, wherein,
R₁ is C₁₋₆ alkyl, C₁₋₆ alkyl substituted with one or more R₁₋₁ₐ, C₃₋₁₀ cycloalkyl, C₃₋₁₀ cycloalkyl substituted with one or more R_{1-1b}, “3- to 10-membered heterocyclyl having 1, 2 or 3 heteroatoms selected from 1, 2 or 3 of N, O and S”, “3- to 10-membered heterocyclyl having 1, 2 or 3 heteroatoms selected from 1, 2 or 3 of N, O and S” substituted with one or more R_{1-1c}, C₆₋₁₀ aryl, C₆₋₁₀ aryl substituted with one or more R_{1-1d}, “5- to 10-membered heteroaryl having 1, 2 or 3 heteroatoms selected from 1, 2 or 3 of N, O and S”, or “5- to 10-membered heteroaryl having 1, 2 or 3 heteroatoms selected from 1, 2 or 3 of N, O and S” substituted with one or more R₁₋₁ₑ;
R₁₋₁ₐ, R_{1-1b}, R_{1-1c}, R_{1-1d}, and R₁₋₁ₑ are each independently —OR₁₋₂ₐ, —NR_{1-2b}R_{1-2c}, halogen, —CN, —C(O)R_{1-2d}, —C(O)OR₁₋₂ₑ, —C(O)NR_{1-2f}R_{1-2g}, C₁₋₆ alkyl, C₁₋₆ alkyl substituted with one or more R₁₋₃ₐ, C₃₋₁₀ cycloalkyl, or “C₃₋₁₀ cycloalkyl substituted with one or more R_{1-3b}”;
R₁₋₂ₐ, R_{1-2b}, R_{1-2c}, R_{1-2d}, R₁₋₂ₑ, R_{1-2f}, and R_{1-2g} are each independently H, C₁₋₆ alkyl, C₁₋₆ alkyl substituted with one or more R_{1-3c}, C₃₋₁₀ cycloalkyl, C₃₋₁₀ cycloalkyl substituted with one or more R_{1-3d}, “3- to 10-membered heterocyclyl having 1, 2 or 3 heteroatoms selected from 1, 2 or 3 of N, O and S”, “3- to 10-membered heterocyclyl having 1, 2 or 3 heteroatoms selected from 1, 2 or 3 of N, O and S” substituted with one or more R₁₋₃ₑ, C₆₋₁₀ aryl, C₆₋₁₀ aryl substituted with one or more R_{1-3f}, “5- to 10-membered heteroaryl having 1, 2 or 3 heteroatoms selected from 1, 2 or 3 of N, O and S”, or “5- to 10-membered heteroaryl having 1, 2 or 3 heteroatoms selected from 1, 2 or 3 of N, O and S” substituted with one or more R_{1-3g};
R₁₋₃ₐ, R_{1-3b}, R_{1-3c}, R_{1-3d}, R₁₋₃ₑ, R_{1-3f}, and R_{1-3g} are each independently —OR₁₋₄ₐ, —NR_{1-4b}R_{1-4c}, halogen, C₁₋₆ alkyl substituted with one or more halogens, C₃₋₁₀ cycloalkyl, “3- to 10-membered heterocyclyl having 1, 2 or 3 heteroatoms selected from 1, 2 or 3 of N, O and S”, C₆₋₁₀ aryl, or “5- to 10-membered heteroaryl having 1, 2 or 3 heteroatoms selected from 1, 2 or 3 of N, O and S”;
R₁₋₄ₐ, R_{1-4b}, and R_{1-4c} are each independently hydrogen, C₁₋₆ alkyl, C₁₋₆ alkyl substituted with one or more halogens, C₃₋₁₀ cycloalkyl, “3- to 10-membered heterocyclyl having 1, 2 or 3 heteroatoms selected from 1, 2 or 3 of N, O and S”, C₆₋₁₀ aryl, or “5- to 10-membered heteroaryl having 1, 2 or 3 heteroatoms selected from 1, 2 or 3 of N, O and S”;
L is —O—, —S—, —SO₂—, or —NR_{L-1}; R_{L-1} is hydrogen or C₁₋₆ alkyl;
R₂ is selected from hydrogen, C₁₋₆ alkyl, C₁₋₆ alkyl substituted with one or more R₂₋₁, C₃₋₁₀ cycloalkyl, or “3- to 10-membered heterocyclyl having 1, 2 or 3 heteroatoms selected from 1, 2 or 3 of N, O and S”;
each R₂₋₁ is independently deuterium, halogen, C₃₋₁₀ cycloalkyl, or hydroxy;
R₃ is hydrogen, C₁₋₆ alkyl, or C₁₋₆ alkyl substituted with one or more halogens;
each R₄ is independently halogen, —NH₂, C₁₋₆ alkyl, C₁₋₆ alkyl substituted with one or more R₄₋₁, “3- to 10-membered heterocyclyl having 1, 2 or 3 heteroatoms selected from 1, 2 or 3 of N, O and S”, C₃₋₆ cycloalkyl, C₆₋₁₀ aryl, or C₆₋₁₀ aryl substituted with one or more —(CH₂)ₘNR₄₋₂R₄₋₃, wherein m is 0, 1, 2 or 3;
each R₄₋₁ is independently halogen or hydroxy;
R₄₋₂ and R₄₋₃ are each independently C₁₋₆ alkyl;
ring A is C₆₋₁₀ aryl, “5- to 10-membered heterocyclyl having 1, 2 or 3 heteroatoms selected from 1, 2 or 3 of N, O and S”, or “5- to 10-membered heteroaryl having 1, 2 or 3 heteroatoms selected from 1, 2 or 3 of N, O and S”; and
p is 1, 2 or 3.

3. The fused ring compound represented by formula I or the pharmaceutically acceptable salt thereof according to claim 1, wherein each R₄ is independently —CN.

4. The fused ring compound represented by formula I or the pharmaceutically acceptable salt thereof according to claim 1, wherein the fused ring compound represented by formula I or the pharmaceutically acceptable salt thereof satisfies one or more of the following conditions:
(1) R₁ is C₃₋₁₀ cycloalkyl, C₃₋₁₀ cycloalkyl substituted with one or more R_{1-1b}, “3- to 10-membered heterocyclyl having 1, 2 or 3 heteroatoms selected from 1, 2 or 3 of N, O and S”, or “3- to 10-membered heterocyclyl having 1, 2 or 3 heteroatoms selected from 1, 2 or 3 of N, O and S” substituted with one or more R_{1-1c};
(2) R₁₋₁ₐ, R_{1-1b}, R_{1-1c}, R_{1-1d}, and R₁₋₁ₑ are each independently halogen, —CN, —C(O)R_{1-2d}, C₁₋₆ alkyl, or C₁₋₆ alkyl substituted with one or more R₁₋₃ₐ;
(3) R₁₋₂ₐ, R_{1-2b}, R_{1-2c}, R_{1-2d}, R₁₋₂ₑ, R_{1-2f}, and R_{1-2g} are each independently C₃₋₁₀ cycloalkyl;
(4) R₁₋₃ₐ, R_{1-3b}, R_{1-3c}, R_{1-3d}, R₁₋₃ₑ, R_{1-3f}, and R_{1-3g} are each independently —OR₁₋₄ₐ or halogen;
(5) R₁₋₄ₐ, R_{1-4b}, and R_{1-4c} are each independently hydrogen or C₁₋₆ alkyl;
(6) R₃ is C₁₋₆ alkyl; and
(7) each R₄ is independently halogen, —NH₂, C₁₋₆ alkyl, C₁₋₆ alkyl substituted with one or more R₄₋₁, or C₆₋₁₀ aryl substituted with one or more —(CH₂)ₘNR₄₋₂R₄₋₃.

5. The fused ring compound represented by formula I or the pharmaceutically acceptable salt thereof according to claim 1, wherein the fused ring compound represented by formula I or the pharmaceutically acceptable salt thereof satisfies one or more of the following conditions:
(1) R₁ is C₃₋₁₀ cycloalkyl substituted with one or more R_{1-1b}, “3- to 10-membered heterocyclyl having 1, 2 or 3 heteroatoms selected from 1, 2 or 3 of N, O and S”, or “3- to 10-membered heterocyclyl having 1, 2 or 3 heteroatoms selected from 1, 2 or 3 of N, O and S” substituted with one or more R_{1-1c};
(2) R₁₋₁ₐ, R_{1-1b}, R_{1-1c}, R_{1-1d}, and R₁₋₁ₑ are each independently C₁₋₆ alkyl or C₁₋₆ alkyl substituted with one or more R₁₋₃ₐ;
(3) R₁₋₃ₐ, R_{1-3b}, R_{1-3c}, R_{1-3d}, R₁₋₃ₑ, R_{1-3f}, and R_{1-3g} are each independently halogen;
(4) R₂ is C₁₋₆ alkyl, C₁₋₆ alkyl substituted with one or more R₂₋₁, C₃₋₁₀ cycloalkyl, or “3- to 10-membered heterocyclyl having 1, 2 or 3 heteroatoms selected from 1, 2 or 3 of N, O and S”;
(5) each R₄ is independently halogen, —NH₂, —CN, C₁₋₆ alkyl, or C₁₋₆ alkyl substituted with one or more R₄₋₁; and
(6) L is —O— or —S—.

6. The fused ring compound represented by formula I or the pharmaceutically acceptable salt thereof according to claim 1, wherein,
R₁ is C₃₋₁₀ cycloalkyl substituted with one or more R_{1-1b}, “3- to 10-membered heterocyclyl having 1, 2 or 3 heteroatoms selected from 1, 2 or 3 of N, O and S”, or “3- to 10-membered heterocyclyl having 1, 2 or 3 heteroatoms selected from 1, 2 or 3 of N, O and S” substituted with one or more R_{1-1c};
each R_{1-1b} is independently C₁₋₆ alkyl substituted with one or more R₁₋₃ₐ;
each R_{1-1c} is independently C₁₋₆ alkyl;
each R₁₋₃ₐ is independently halogen;
L is —O—;
R₂ is C₁₋₆ alkyl or C₁₋₆ alkyl substituted with one or more R₂₋₁;
R₂₋₁ is deuterium;
R₃ is C₁₋₆ alkyl;
ring A is C₆₋₁₀ aryl, “5- to 10-membered heterocyclyl having 1, 2 or 3 heteroatoms selected from 1, 2 or 3 of N, O and S”, or “5- to 10-membered heteroaryl having 1, 2 or 3 heteroatoms selected from 1, 2 or 3 of N, O and S”;
each R₄ is independently halogen, —NH₂, C₁₋₆ alkyl, C₁₋₆ alkyl substituted with one or more R₄₋₁, or C₆₋₁₀ aryl substituted with one or more —(CH₂)ₘNR₄₋₂R₄₋₃; and
each R₄₋₁ is independently halogen or hydroxy.

7. The fused ring compound represented by formula I or the pharmaceutically acceptable salt thereof according to claim 1, wherein,
R₁ is C₃₋₁₀ cycloalkyl substituted with one or more R_{1-1b}, “3- to 10-membered heterocyclyl having 1, 2 or 3 heteroatoms selected from 1, 2 or 3 of N, O and S”, or “3- to 10-membered heterocyclyl having 1, 2 or 3 heteroatoms selected from 1, 2 or 3 of N, O and S” substituted with one or more R_{1-1c};
each R_{1-1b} is independently C₁₋₆ alkyl substituted with one or more R₁₋₃ₐ;
each R_{1-1c} is independently C₁₋₆ alkyl;
each R₁₋₃ₐ is independently halogen;
L is —O—;
R₂ is C₁₋₆ alkyl or C₁₋₆ alkyl substituted with one or more R₂₋₁;
R₂₋₁ is deuterium;
R₃ is C₁₋₆ alkyl;
ring A is C₆₋₁₀ aryl, “5- to 10-membered heterocyclyl having 1, 2 or 3 heteroatoms selected from 1, 2 or 3 of N, O and S”, or “5- to 10-membered heteroaryl having 1, 2 or 3 heteroatoms selected from 1, 2 or 3 of N, O and S”;
each R₄ is independently halogen, —NH₂, —CN, C₁₋₆ alkyl, C₁₋₆ alkyl substituted with one or more R₄₋₁, or C₆₋₁₀ aryl substituted with one or more —(CH₂)ₘNR₄₋₂R₄₋₃; and
each R₄₋₁ is independently halogen or hydroxy.

8. The fused ring compound represented by formula I or the pharmaceutically acceptable salt thereof according to claim 1, wherein the fused ring compound represented by formula I or the pharmaceutically acceptable salt thereof satisfies one or more of the following conditions:
(1) in R₁, R₁₋₁ₐ, R_{1-1b}, R_{1-1c}, R_{1-1d}, R₁₋₁ₑ, R₁₋₂ₐ, R_{1-2b}, R_{1-2c}, R_{1-2d}, R₁₋₂ₑ, R_{1-2f}, R_{1-2g}, R₁₋₃ₐ, R_{1-3b}, R_{1-3c}, R_{1-3d}, R₁₋₃ₑ, R_{1-3f}, R_{1-3g}, R₁₋₄ₐ, R_{1-4b}, R_{1-4c}, R_{L-1}, R₂, R₃, R₄, R₄₋₂, and R₄₋₃, the “C₁₋₆ alkyl” in each of the “C₁₋₆ alkyl substituted with one or more R₁₋₁ₐ”, the “C₁₋₆ alkyl substituted with one or more R₁₋₃ₐ”, the “C₁₋₆ alkyl substituted with one or more R_{1-3c}”, the “C₁₋₆ alkyl substituted with one or more halogens”, the “C₁₋₆ alkyl”, the “C₁₋₆ alkyl substituted with one or more R₂₋₁”, and the “C₁₋₆ alkyl substituted with one or more R₄₋₁” is independently methyl, ethyl, n-propyl, isopropyl, n-butyl, isobutyl, sec-butyl, or tert-butyl;
(2) in R₁, R₁₋₁ₐ, R_{1-1b}, R_{1-1c}, R_{1-1d}, R₁₋₁ₑ, R₁₋₂ₐ, R_{1-2b}, R_{1-2c}, R_{1-2d}, R₁₋₂ₑ, R_{1-2f}, R_{1-2g}, R₁₋₃ₐ, R_{1-3b}, R_{1-3c}, R_{1-3d}, R₁₋₃ₑ, R_{1-3f}, R_{1-3g}, R₁₋₄ₐ, R_{1-4b}, R_{1-4c}, R₂, and R₂₋₁, the “C₃₋₁₀ cycloalkyl” in each of the “C₃₋₁₀ cycloalkyl”, the “C₃₋₁₀ cycloalkyl substituted with one or more R_{1-1b}”, the “C₃₋₁₀ cycloalkyl substituted with one or more R_{1-3b}”, and the “C₃₋₁₀ cycloalkyl substituted with one or more R_{1-3d}” is independently C₃₋₆ cycloalkyl;
(3) in R₁, R₁₋₂ₐ, R_{1-2b}, R_{1-2c}, R_{1-2d}, R₁₋₂ₑ, R_{1-2f}, R_{1-2g}, R₁₋₃ₐ, R_{1-3b}, R_{1-3c}, R_{1-3d}, R₁₋₃ₑ, R_{1-3f}, R_{1-3g}, R₁₋₄ₐ, R_{1-4b}, R_{1-4c}, R₂, and R₄, the “3- to 10-membered heterocyclyl” in each of the “3- to 10-membered heterocyclyl having 1, 2 or 3 heteroatoms selected from 1, 2 or 3 of N, O and S”, the “3- to 10-membered heterocyclyl having 1, 2 or 3 heteroatoms selected from 1, 2 or 3 of N, O and S′ substituted with one or more R_{1-1c}”, and the “3- to 10-membered heterocyclyl having 1, 2 or 3 heteroatoms selected from 1, 2 or 3 of N, O and S′ substituted with one or more R₁₋₃ₑ” is independently 5- to 6-membered heterocyclyl;
(4) in R₁, R₁₋₂ₐ, R_{1-2b}, R_{1-2c}, R_{1-2d}, R₁₋₂ₑ, R_{1-2f}, R_{1-2g}, R₁₋₃ₐ, R_{1-3b}, R_{1-3c}, R_{1-3d}, R₁₋₃ₑ, R_{1-3f}, R_{1-3g}, R₁₋₄ₐ, R_{1-4b}, R_{1-4c}, R₄, and ring A, the “C₆₋₁₀ aryl” in each of the “C₆₋₁₀ aryl”, the “C₆₋₁₀ aryl substituted with one or more R_{1-1d}”, the “C₆₋₁₀ aryl substituted with one or more R_{1-3f}”, and the “C₆₋₁₀ aryl substituted with one or more —(CH₂)ₘNR₄₋₂R₄₋₃” is independently phenyl;
(5) in R₁, R₁₋₂ₐ, R_{1-2b}, R_{1-2c}, R_{1-2d}, R₁₋₂ₑ, R_{1-2f}, R_{1-2g}, R₁₋₃ₐ, R_{1-3b}, R_{1-3c}, R_{1-3d}, R₁₋₃ₑ, R_{1-3f}, R_{1-3g}, R₁₋₄ₐ, R_{1-4b}, R_{1-4c}, and ring A, the “5- to 10-membered heteroaryl” in each of the “5- to 10-membered heteroaryl having 1, 2 or 3 heteroatoms selected from 1, 2 or 3 of N, O and S”, the “5- to 10-membered heteroaryl having 1, 2 or 3 heteroatoms selected from 1, 2 or 3 of N, O and S′ substituted with one or more R₁₋₁ₑ”, and the “5- to 10-membered heteroaryl having 1, 2 or 3 heteroatoms selected from 1, 2 or 3 of N, O and S′ substituted with one or more R_{1-3g}” is independently “9- to 10-membered heteroaryl”;
(6) in ring A, the “5- to 10-membered heterocyclyl” in the “5- to 10-membered heterocyclyl having 1, 2 or 3 heteroatoms selected from 1, 2 or 3 of N, O and S” is “9- to 10-membered heterocyclyl”; and
(7) in R₁₋₁ₐ, R_{1-1b}, R_{1-1c}, R_{1-1d}, R₁₋₁ₑ, R₁₋₃ₐ, R_{1-3b}, R_{1-3c}, R_{1-3d}, R₁₋₃ₑ, R_{1-3f}, R_{1-3g}, R₂₋₁, R₃, R₄, and R₄₋₁, the “halogen” in each of the “halogen” and the “C₁₋₆ alkyl substituted with one or more halogens” is independently fluorine, chlorine, bromine, or iodine.

9. The fused ring compound represented by formula I or the pharmaceutically acceptable salt thereof according to claim 8, wherein the fused ring compound represented by formula I or the pharmaceutically acceptable salt thereof satisfies one or more of the following conditions:
(1) in R₁, R₁₋₁ₐ, R_{1-1b}, R_{1-1c}, R_{1-1d}, R₁₋₁ₑ, R₁₋₂ₐ, R_{1-2b}, R_{1-2c}, R_{1-2d}, R₁₋₂ₑ, R_{1-2f}, R_{1-2g}, R₁₋₃ₐ, R_{1-3b}, R_{1-3c}, R_{1-3d}, R₁₋₃ₑ, R_{1-3f}, R_{1-3g}, R₁₋₄ₐ, R_{1-4b}, R_{1-4c}, R_{L-1}, R₂, R₃, R₄, R₄₋₂, and R₄₋₃, the “C₁₋₆ alkyl” in each of the “C₁₋₆ alkyl substituted with one or more R₁₋₁ₐ”, the “C₁₋₆ alkyl substituted with one or more R₁₋₃ₐ”, the “C₁₋₆ alkyl substituted with one or more R_{1-3c}”, the “C₁₋₆ alkyl substituted with one or more halogens”, the “C₁₋₆ alkyl”, the “C₁₋₆ alkyl substituted with one or more R₂₋₁”, and the “C₁₋₆ alkyl substituted with one or more R₄₋₁” is independently methyl, ethyl, n-propyl, or isopropyl;
(2) in R₁, R₁₋₁ₐ, R_{1-1b}, R_{1-1c}, R_{1-1d}, R₁₋₁ₑ, R₁₋₂ₐ, R_{1-2b}, R_{1-2c}, R_{1-2d}, R₁₋₂ₑ, R_{1-2f}, R_{1-2g}, R₁₋₃ₐ, R_{1-3b}, R_{1-3c}, R_{1-3d}, R₁₋₃ₑ, R_{1-3f}, R_{1-3g}, R₁₋₄ₐ, R_{1-4b}, R_{1-4c}, R₂, and R₂₋₁, the “C₃₋₁₀ cycloalkyl” in each of the “C₃₋₁₀ cycloalkyl”, the “C₃₋₁₀ cycloalkyl substituted with one or more R_{1-1b}”, the “C₃₋₁₀ cycloalkyl substituted with one or more R_{1-3b}”, and the “C₃₋₁₀ cycloalkyl substituted with one or more R_{1-3d}” is cyclopropyl, cyclobutyl, or cyclopentyl;
(3) in R₁, R₁₋₂ₐ, R_{1-2b}, R_{1-2c}, R_{1-2d}, R₁₋₂ₑ, R_{1-2f}, R_{1-2g}, R₁₋₃ₐ, R_{1-3b}, R_{1-3c}, R_{1-3d}, R₁₋₃ₑ, R_{1-3f}, R_{1-3g}, R₁₋₄ₐ, R_{1-4b}, R_{1-4c}, R₂, and R₄, the “3- to 10-membered heterocyclyl” in each of the “3- to 10-membered heterocyclyl having 1, 2 or 3 heteroatoms selected from 1, 2 or 3 of N, O and S”, the “3- to 10-membered heterocyclyl having 1, 2 or 3 heteroatoms selected from 1, 2 or 3 of N, O and S′ substituted with one or more R_{1-1c}”, and the “3- to 10-membered heterocyclyl having 1, 2 or 3 heteroatoms selected from 1, 2 or 3 of N, O and S′ substituted with one or more R₁₋₃ₑ” is 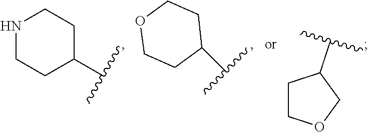 [SMILES: *C1CCNCC1 ; *C1CCOCC1 ; *C1CCOC1]
(4) in ring A, the “5- to 10-membered heterocyclyl” in the “5- to 10-membered heterocyclyl having 1, 2 or 3 heteroatoms selected from 1, 2 or 3 of N, O and S” is 2,3-dihydrobenzofuranyl;
(5) in R₁₋₁ₐ, R_{1-1b}, R_{1-1c}, R_{1-1d}, R₁₋₁ₑ, R₁₋₃ₐ, R_{1-3b}, R_{1-3c}, R_{1-3d}, R₁₋₃ₑ, R_{1-3f}, R_{1-3g}, R₂₋₁, R₃, R₄, and R₄₋₁, the “halogen” in each of the “halogen” and the “C₁₋₆ alkyl substituted with one or more halogens” is fluorine; and
(6) in R₁, R₁₋₂ₐ, R_{1-2b}, R_{1-2c}, R_{1-2d}, R₁₋₂ₑ, R_{1-2f}, R_{1-2g}, R₁₋₃ₐ, R_{1-3b}, R_{1-3c}, R_{1-3d}, R₁₋₃ₑ, R_{1-3f}, R_{1-3g}, R₁₋₄ₐ, R_{1-4b}, R_{1-4c}, and ring A, the “5- to 10-membered heteroaryl” in each of the “5- to 10-membered heteroaryl having 1, 2 or 3 heteroatoms selected from 1, 2 or 3 of N, O and S”, the “5- to 10-membered heteroaryl having 1, 2 or 3 heteroatoms selected from 1, 2 or 3 of N, O and S′ substituted with one or more R₁₋₁ₑ”, and the “5- to 10-membered heteroaryl having 1, 2 or 3 heteroatoms selected from 1, 2 or 3 of N, O and S′ substituted with one or more R_{1-3g}” is independently benzofuranyl.

10. The fused ring compound represented by formula I or the pharmaceutically acceptable salt thereof according to claim 1, wherein the fused ring compound represented by formula I or the pharmaceutically acceptable salt thereof satisfies one or more of the following conditions:
(1) R₁ is 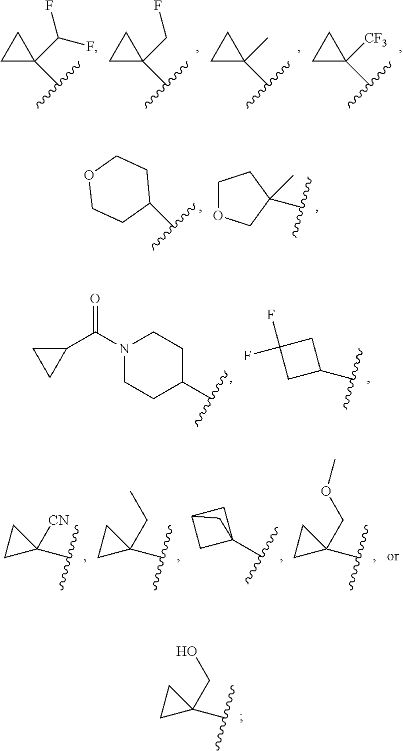
(2) R₂ is hydrogen, methyl, ethyl, —CD₃, isopropyl, —CH₂CF₃, 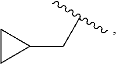 [SMILES: *CC1CC1]
cyclopropyl, 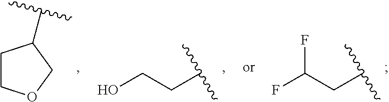
(3) R₃ is methyl;
(4) R₄ is 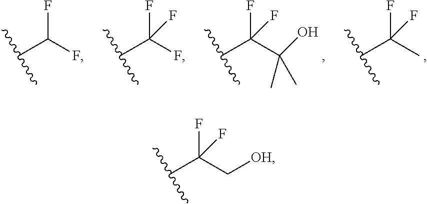
—F, —CH₃, —NH₂, 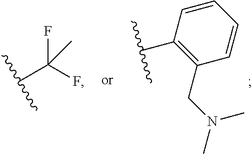
and
(5) ring A is phenyl, benzofuranyl, 2,3-dihydrobenzofuranyl, or thienyl.

11. The fused ring compound represented by formula I or the pharmaceutically acceptable salt thereof according to claim 10, wherein the fused ring compound represented by formula I or the pharmaceutically acceptable salt thereof satisfies one or more of the following conditions:
(1) R₁ is 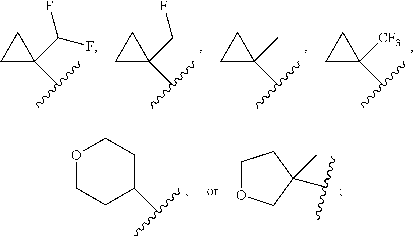
(2) R₂ is methyl, ethyl, or —CD₃;
(3) R₄ is 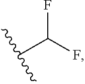 [SMILES: *C(F)F]
—F, —CH₃, 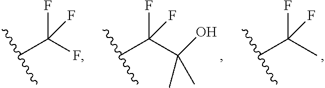
—NH₂, 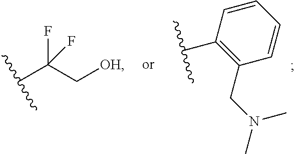 [SMILES: *C(F)(F)CO ; "or" ; *c1ccccc1CN(C)C]
and
(4) ring A is phenyl,

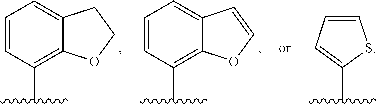

12. The fused ring compound represented by formula I or the pharmaceutically acceptable salt thereof according to claim 1, wherein the fused ring compound represented by formula I or the pharmaceutically acceptable salt thereof satisfies one or more of the following conditions:
(1) -L-R₂ is —OCH₃, —OCH₂CH₃, —OCD₃, —SO₂CH₃, —OCH₂CF₃, 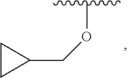 [SMILES: *OCC1CC1]
—SCH₃, —N(CH₃)(CH₃), —NH₂, 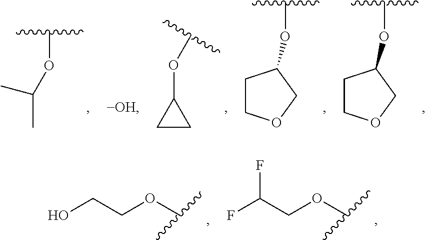
or —SO₂CH₂CH₃; and

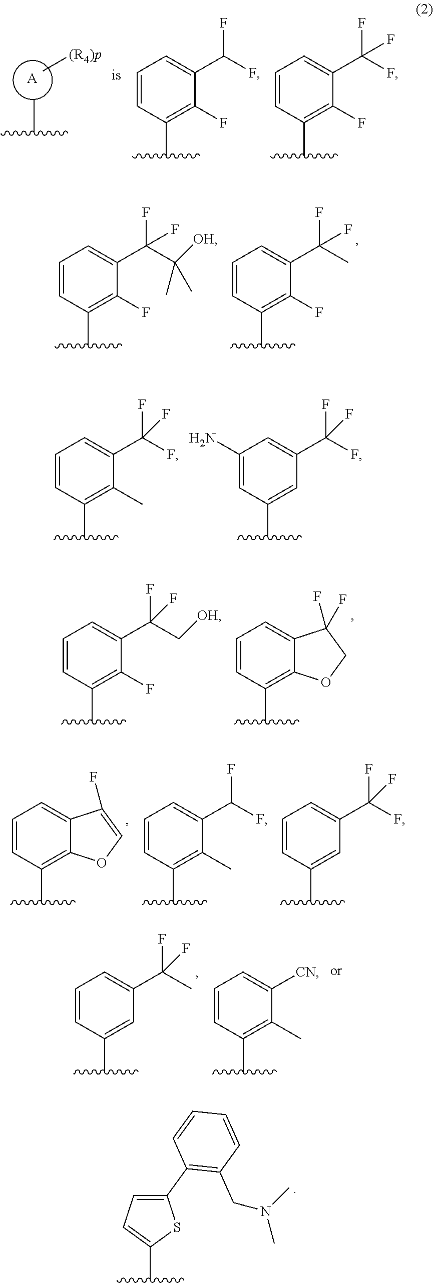

13. The fused ring compound represented by formula I or the pharmaceutically acceptable salt thereof according to claim 12, wherein the fused ring compound represented by formula I or the pharmaceutically acceptable salt thereof satisfies one or more of the following conditions:
(1) -L-R₂ is —OCH₃, —OCH₂CH₃, —OCD₃, or —SCH₃; and

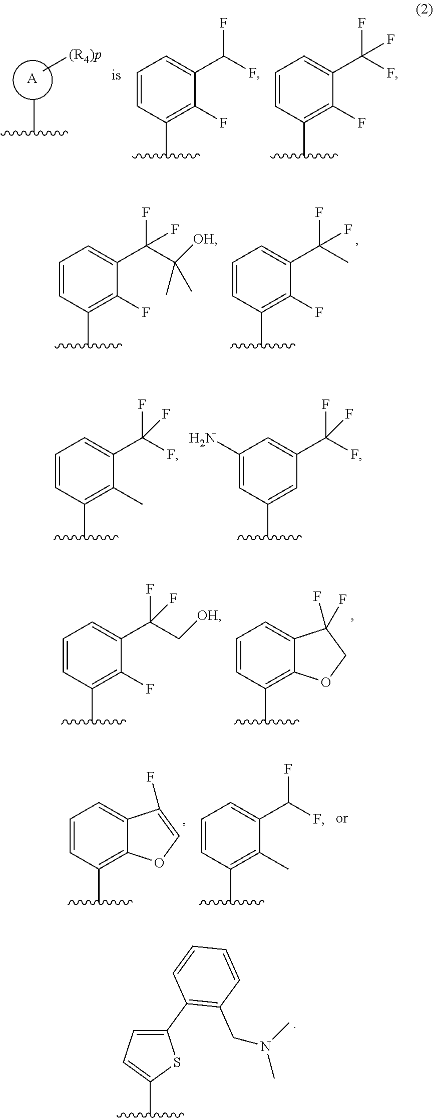

14. The fused ring compound represented by formula I or the pharmaceutically acceptable salt thereof according to claim 1, wherein the fused ring compound represented by formula I is any one of the following compounds:

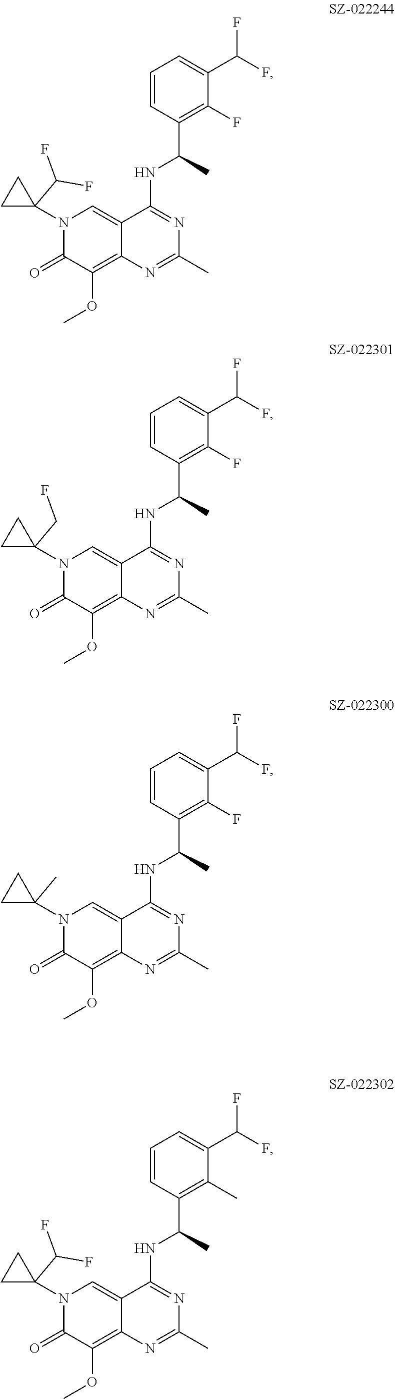 [SMILES: COc1c(=O)n(C2(C(F)F)CC2)cc2c(N[C@H](C)c3cccc(C(F)F)c3F)nc(C)nc12 ; COc1c(=O)n(C2(CF)CC2)cc2c(N[C@H](C)c3cccc(C(F)F)c3F)nc(C)nc12 ; COc1c(=O)n(C2(C)CC2)cc2c(N[C@H](C)c3cccc(C(F)F)c3F)nc(C)nc12 ; COc1c(=O)n(C2(C(F)F)CC2)cc2c(N[C@H](C)c3cccc(C(F)F)c3C)nc(C)nc12]

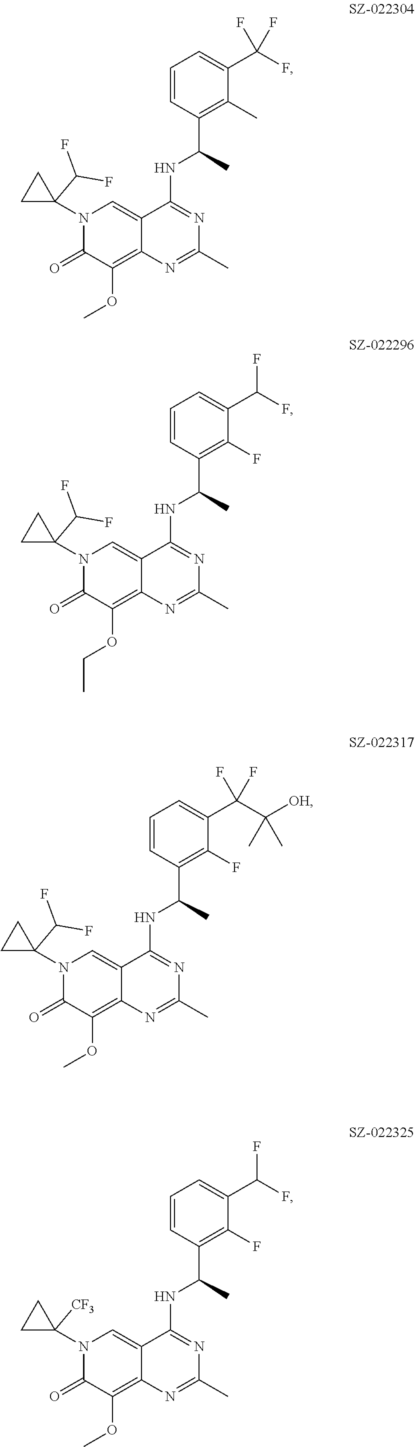 [SMILES: COc1c(=O)n(C2(C(F)F)CC2)cc2c(N[C@H](C)c3cccc(C(F)(F)F)c3C)nc(C)nc12 ; CCOc1c(=O)n(C2(C(F)F)CC2)cc2c(N[C@H](C)c3cccc(C(F)F)c3F)nc(C)nc12 ; COc1c(=O)n(C2(C(F)F)CC2)cc2c(N[C@H](C)c3cccc(C(F)(F)C(C)(C)O)c3F)nc(C)nc12 ; COc1c(=O)n(C2(C(F)(F)F)CC2)cc2c(N[C@H](C)c3cccc(C(F)F)c3F)nc(C)nc12]

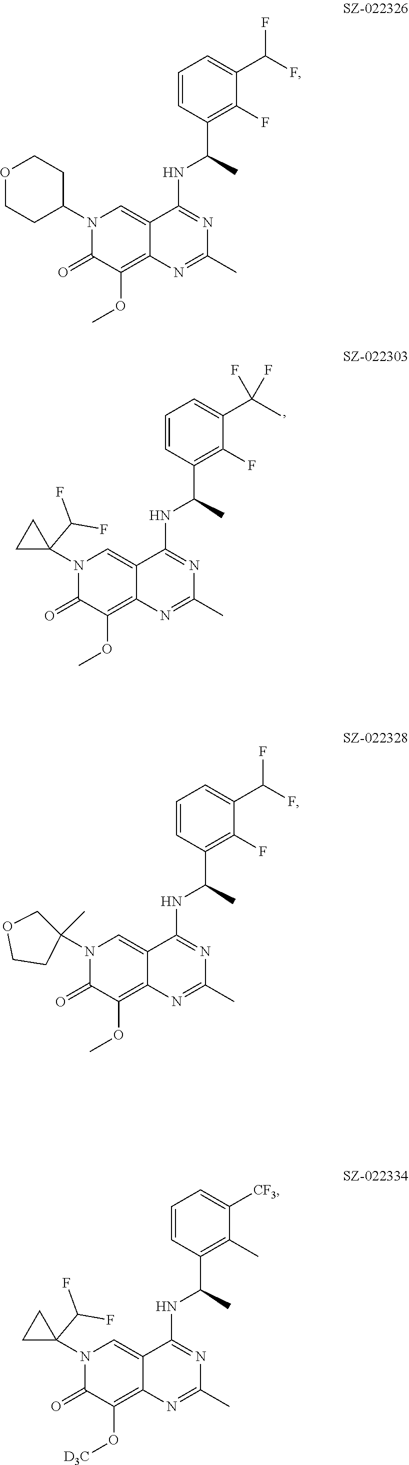 [SMILES: COc1c(=O)n(C2CCOCC2)cc2c(N[C@H](C)c3cccc(C(F)F)c3F)nc(C)nc12 ; COc1c(=O)n(C2(C(F)F)CC2)cc2c(N[C@H](C)c3cccc(C(C)(F)F)c3F)nc(C)nc12 ; COc1c(=O)n(C2(C)CCOC2)cc2c(N[C@H](C)c3cccc(C(F)F)c3F)nc(C)nc12 ; [2H]C([2H])([2H])Oc1c(=O)n(C2(C(F)F)CC2)cc2c(N[C@H](C)c3cccc(C(F)(F)F)c3C)nc(C)nc12]

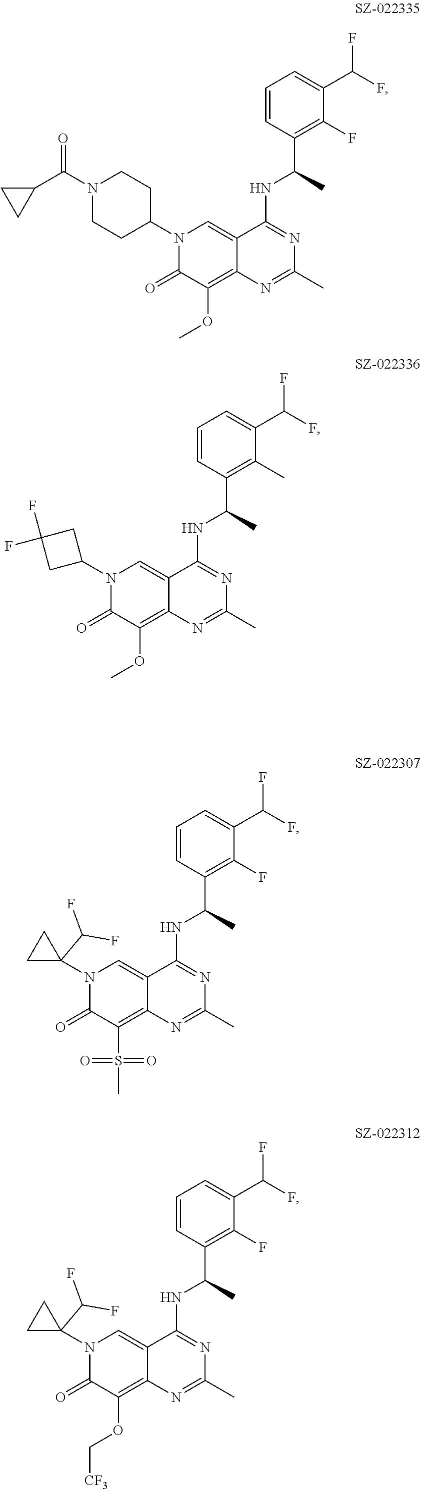 [SMILES: COc1c(=O)n(C2CCN(C(=O)C3CC3)CC2)cc2c(N[C@H](C)c3cccc(C(F)F)c3F)nc(C)nc12 ; COc1c(=O)n(C2CC(F)(F)C2)cc2c(N[C@H](C)c3cccc(C(F)F)c3C)nc(C)nc12 ; Cc1nc(N[C@H](C)c2cccc(C(F)F)c2F)c2cn(C3(C(F)F)CC3)c(=O)c(S(C)(=O)=O)c2n1 ; Cc1nc(N[C@H](C)c2cccc(C(F)F)c2F)c2cn(C3(C(F)F)CC3)c(=O)c(OCC(F)(F)F)c2n1]

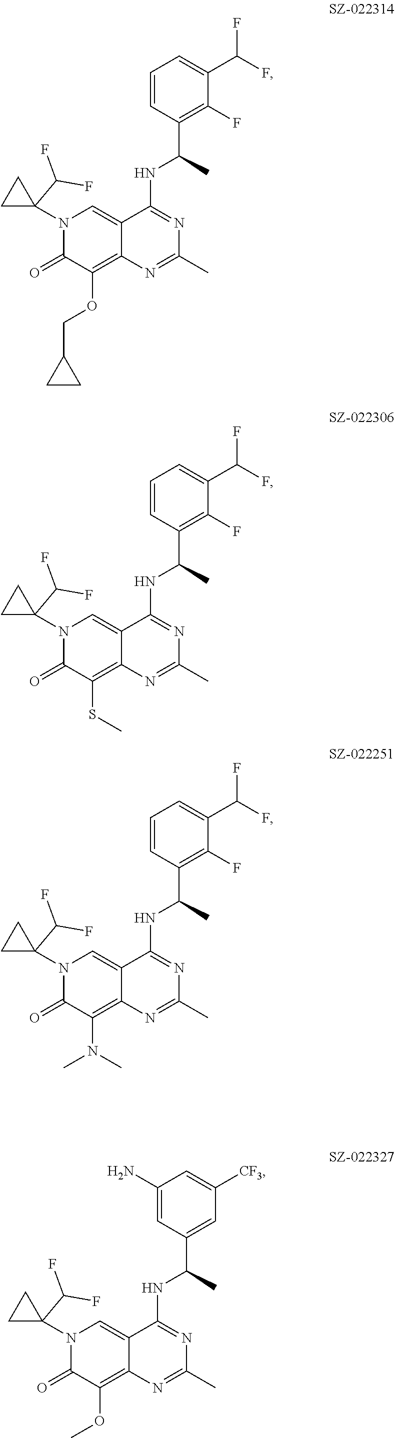 [SMILES: Cc1nc(N[C@H](C)c2cccc(C(F)F)c2F)c2cn(C3(C(F)F)CC3)c(=O)c(OCC3CC3)c2n1 ; CSc1c(=O)n(C2(C(F)F)CC2)cc2c(N[C@H](C)c3cccc(C(F)F)c3F)nc(C)nc12 ; Cc1nc(N[C@H](C)c2cccc(C(F)F)c2F)c2cn(C3(C(F)F)CC3)c(=O)c(N(C)C)c2n1 ; COc1c(=O)n(C2(C(F)F)CC2)cc2c(N[C@H](C)c3cc(N)cc(C(F)(F)F)c3)nc(C)nc12]

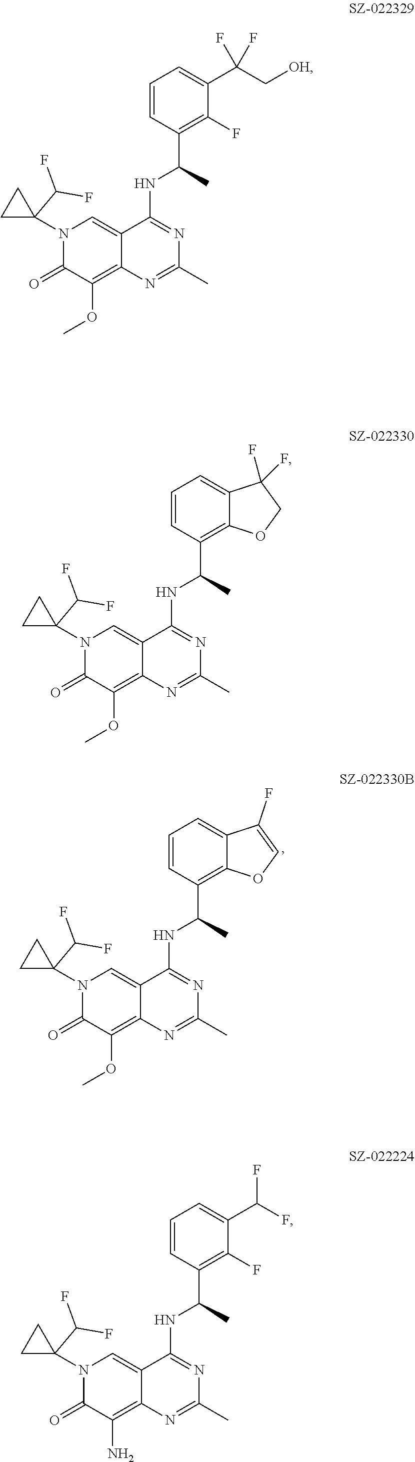 [SMILES: COc1c(=O)n(C2(C(F)F)CC2)cc2c(N[C@H](C)c3cccc(C(F)(F)CO)c3F)nc(C)nc12 ; COc1c(=O)n(C2(C(F)F)CC2)cc2c(N[C@H](C)c3cccc4c3OCC4(F)F)nc(C)nc12 ; COc1c(=O)n(C2(C(F)F)CC2)cc2c(N[C@H](C)c3cccc4c(F)coc34)nc(C)nc12 ; Cc1nc(N[C@H](C)c2cccc(C(F)F)c2F)c2cn(C3(C(F)F)CC3)c(=O)c(N)c2n1]

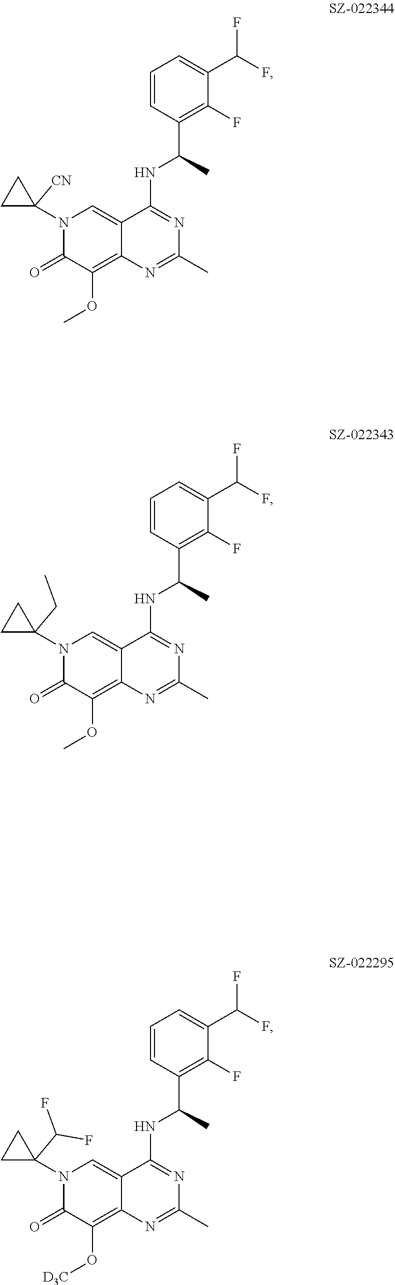 [SMILES: COc1c(=O)n(C2(C#N)CC2)cc2c(N[C@H](C)c3cccc(C(F)F)c3F)nc(C)nc12 ; CCC1(n2cc3c(N[C@H](C)c4cccc(C(F)F)c4F)nc(C)nc3c(OC)c2=O)CC1 ; [2H]C([2H])([2H])Oc1c(=O)n(C2(C(F)F)CC2)cc2c(N[C@H](C)c3cccc(C(F)F)c3F)nc(C)nc12]

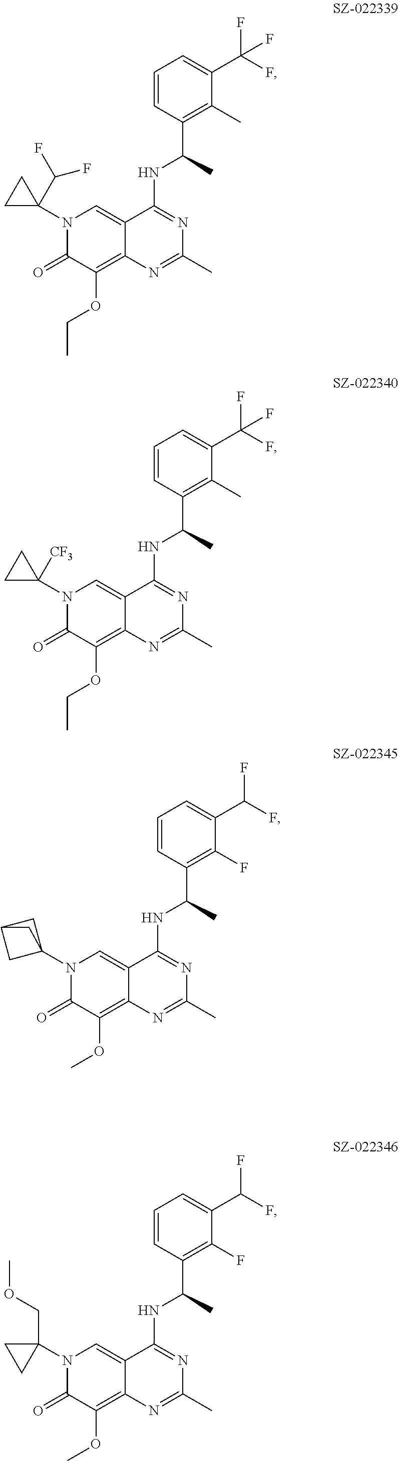 [SMILES: CCOc1c(=O)n(C2(C(F)F)CC2)cc2c(N[C@H](C)c3cccc(C(F)(F)F)c3C)nc(C)nc12 ; CCOc1c(=O)n(C2(C(F)(F)F)CC2)cc2c(N[C@H](C)c3cccc(C(F)(F)F)c3C)nc(C)nc12 ; COc1c(=O)n(C23CC(C2)C3)cc2c(N[C@H](C)c3cccc(C(F)F)c3F)nc(C)nc12 ; COCC1(n2cc3c(N[C@H](C)c4cccc(C(F)F)c4F)nc(C)nc3c(OC)c2=O)CC1]

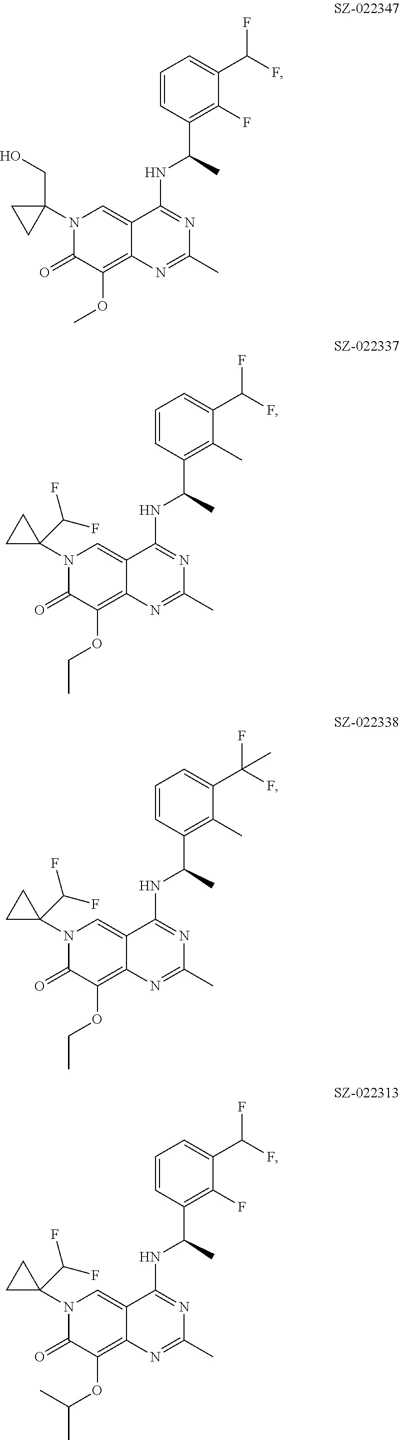 [SMILES: COc1c(=O)n(C2(CO)CC2)cc2c(N[C@H](C)c3cccc(C(F)F)c3F)nc(C)nc12 ; CCOc1c(=O)n(C2(C(F)F)CC2)cc2c(N[C@H](C)c3cccc(C(F)F)c3C)nc(C)nc12 ; CCOc1c(=O)n(C2(C(F)F)CC2)cc2c(N[C@H](C)c3cccc(C(C)(F)F)c3C)nc(C)nc12 ; Cc1nc(N[C@H](C)c2cccc(C(F)F)c2F)c2cn(C3(C(F)F)CC3)c(=O)c(OC(C)C)c2n1]

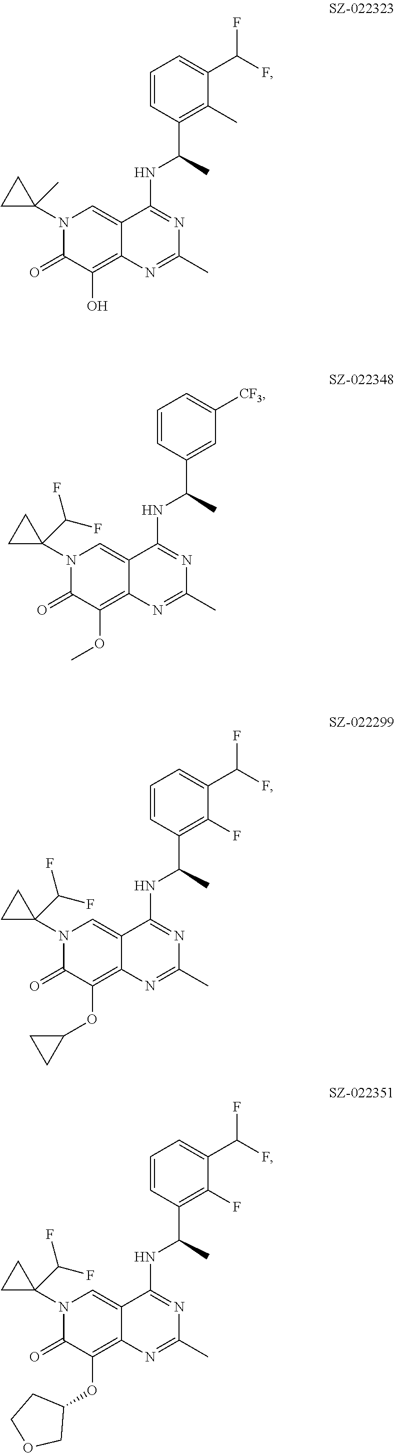 [SMILES: Cc1nc(N[C@H](C)c2cccc(C(F)F)c2C)c2cn(C3(C)CC3)c(=O)c(O)c2n1 ; COc1c(=O)n(C2(C(F)F)CC2)cc2c(N[C@H](C)c3cccc(C(F)(F)F)c3)nc(C)nc12 ; Cc1nc(N[C@H](C)c2cccc(C(F)F)c2F)c2cn(C3(C(F)F)CC3)c(=O)c(OC3CC3)c2n1 ; Cc1nc(N[C@H](C)c2cccc(C(F)F)c2F)c2cn(C3(C(F)F)CC3)c(=O)c(O[C@H]3CCOC3)c2n1]

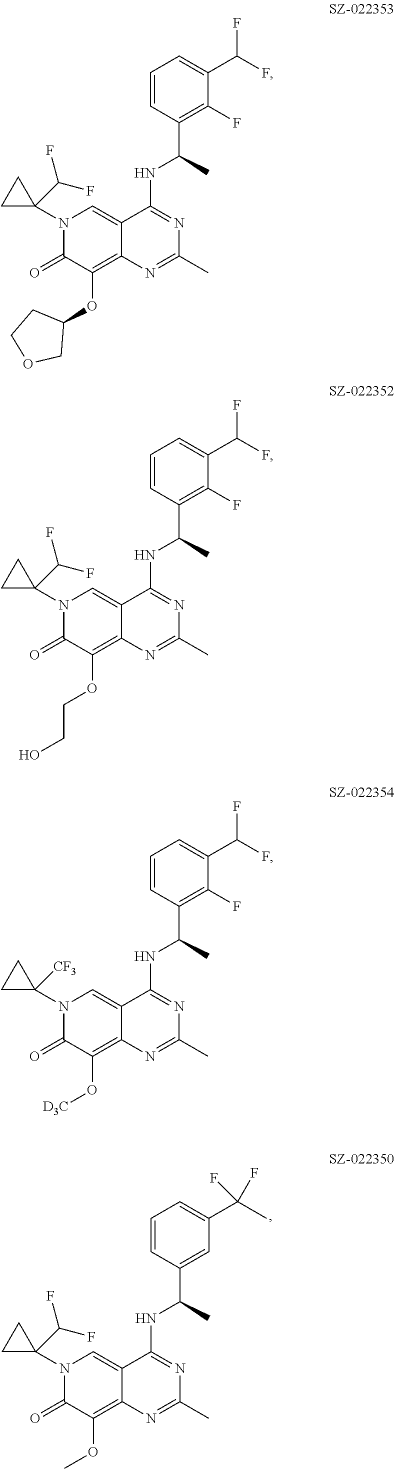 [SMILES: Cc1nc(N[C@H](C)c2cccc(C(F)F)c2F)c2cn(C3(C(F)F)CC3)c(=O)c(O[C@@H]3CCOC3)c2n1 ; Cc1nc(N[C@H](C)c2cccc(C(F)F)c2F)c2cn(C3(C(F)F)CC3)c(=O)c(OCCO)c2n1 ; [2H]C([2H])([2H])Oc1c(=O)n(C2(C(F)(F)F)CC2)cc2c(N[C@H](C)c3cccc(C(F)F)c3F)nc(C)nc12 ; COc1c(=O)n(C2(C(F)F)CC2)cc2c(N[C@H](C)c3cccc(C(C)(F)F)c3)nc(C)nc12]

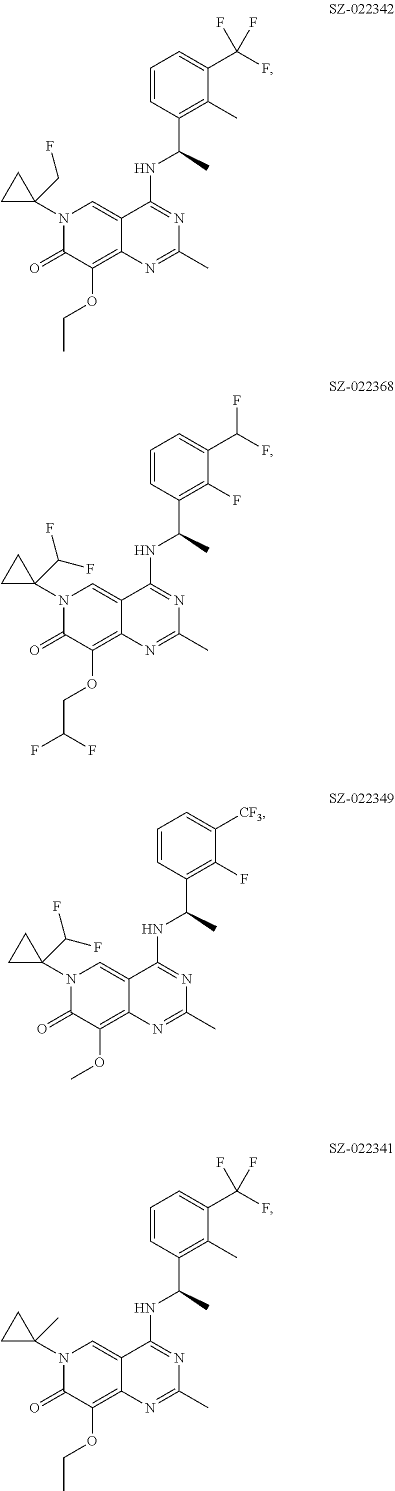 [SMILES: CCOc1c(=O)n(C2(CF)CC2)cc2c(N[C@H](C)c3cccc(C(F)(F)F)c3C)nc(C)nc12 ; Cc1nc(N[C@H](C)c2cccc(C(F)F)c2F)c2cn(C3(C(F)F)CC3)c(=O)c(OCC(F)F)c2n1 ; COc1c(=O)n(C2(C(F)F)CC2)cc2c(N[C@H](C)c3cccc(C(F)(F)F)c3F)nc(C)nc12 ; CCOc1c(=O)n(C2(C)CC2)cc2c(N[C@H](C)c3cccc(C(F)(F)F)c3C)nc(C)nc12]

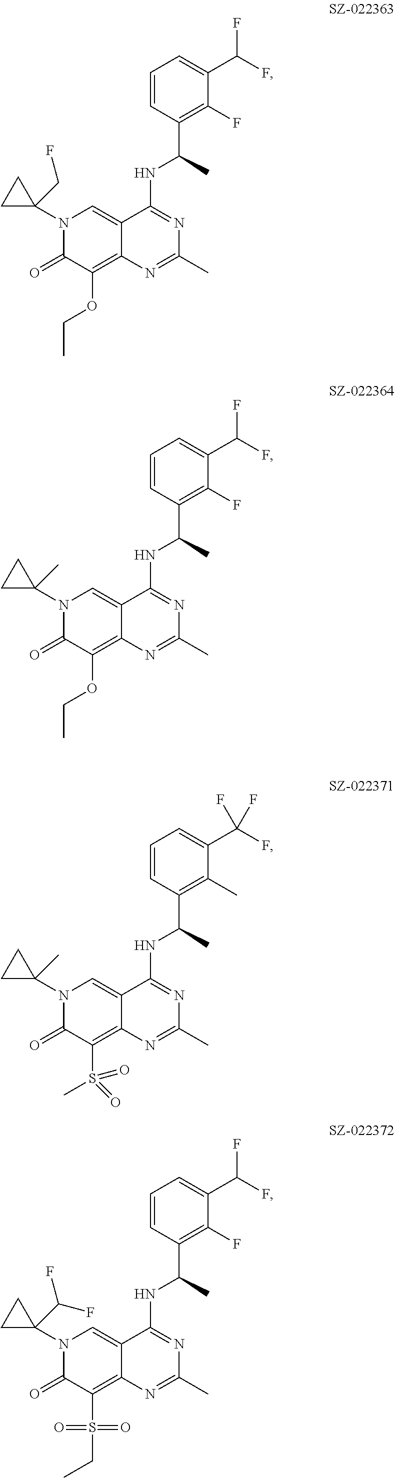 [SMILES: CCOc1c(=O)n(C2(CF)CC2)cc2c(N[C@H](C)c3cccc(C(F)F)c3F)nc(C)nc12 ; CCOc1c(=O)n(C2(C)CC2)cc2c(N[C@H](C)c3cccc(C(F)F)c3F)nc(C)nc12 ; Cc1nc(N[C@H](C)c2cccc(C(F)(F)F)c2C)c2cn(C3(C)CC3)c(=O)c(S(C)(=O)=O)c2n1 ; CCS(=O)(=O)c1c(=O)n(C2(C(F)F)CC2)cc2c(N[C@H](C)c3cccc(C(F)F)c3F)nc(C)nc12]

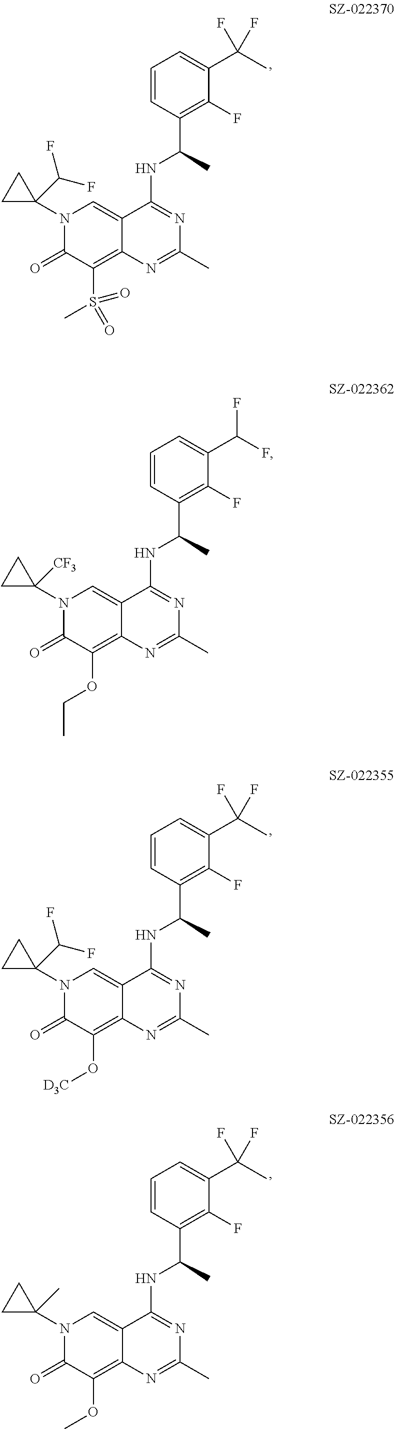 [SMILES: Cc1nc(N[C@H](C)c2cccc(C(C)(F)F)c2F)c2cn(C3(C(F)F)CC3)c(=O)c(S(C)(=O)=O)c2n1 ; CCOc1c(=O)n(C2(C(F)(F)F)CC2)cc2c(N[C@H](C)c3cccc(C(F)F)c3F)nc(C)nc12 ; [2H]C([2H])([2H])Oc1c(=O)n(C2(C(F)F)CC2)cc2c(N[C@H](C)c3cccc(C(C)(F)F)c3F)nc(C)nc12 ; COc1c(=O)n(C2(C)CC2)cc2c(N[C@H](C)c3cccc(C(C)(F)F)c3F)nc(C)nc12]

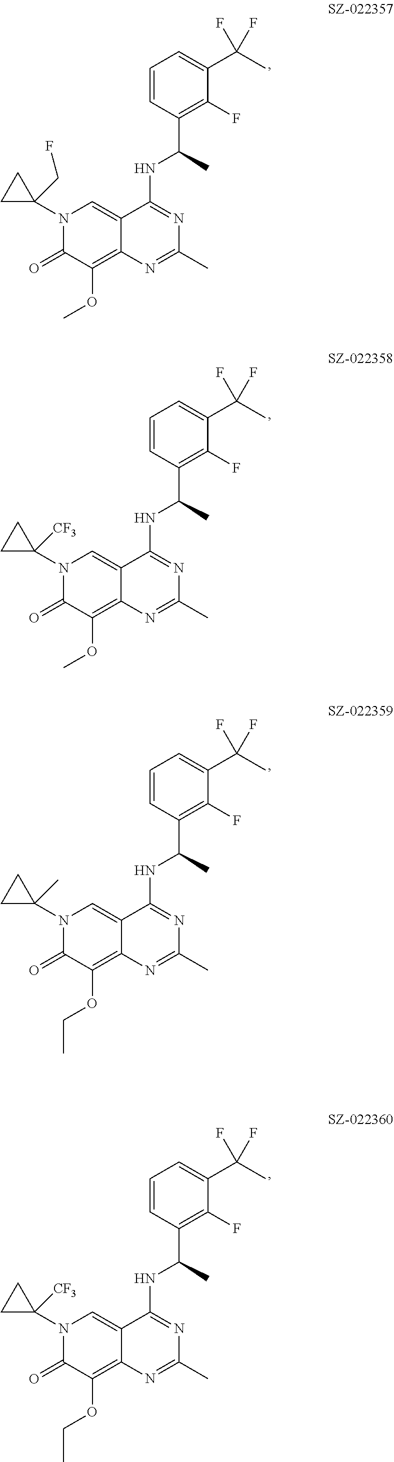 [SMILES: COc1c(=O)n(C2(CF)CC2)cc2c(N[C@H](C)c3cccc(C(C)(F)F)c3F)nc(C)nc12 ; COc1c(=O)n(C2(C(F)(F)F)CC2)cc2c(N[C@H](C)c3cccc(C(C)(F)F)c3F)nc(C)nc12 ; CCOc1c(=O)n(C2(C)CC2)cc2c(N[C@H](C)c3cccc(C(C)(F)F)c3F)nc(C)nc12 ; CCOc1c(=O)n(C2(C(F)(F)F)CC2)cc2c(N[C@H](C)c3cccc(C(C)(F)F)c3F)nc(C)nc12]

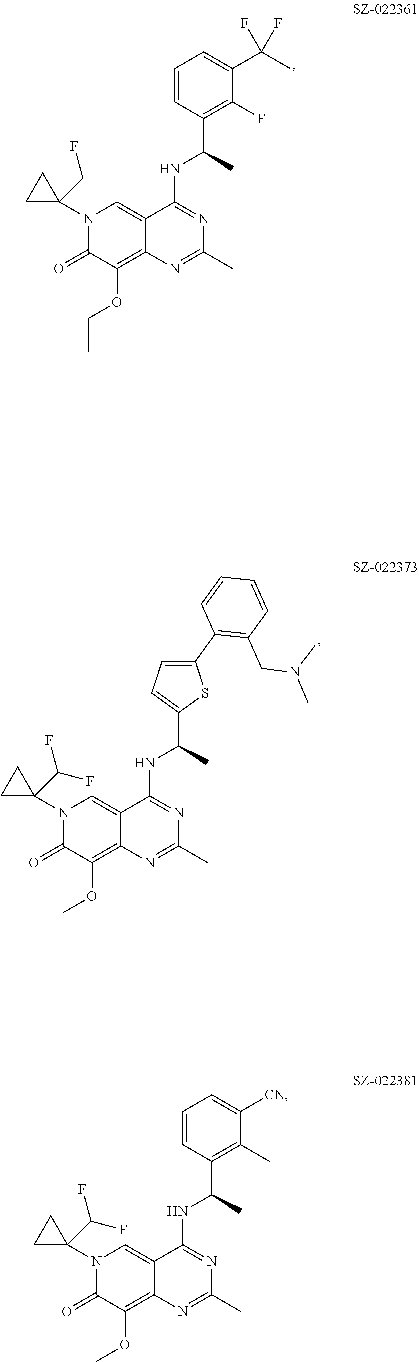 [SMILES: CCOc1c(=O)n(C2(CF)CC2)cc2c(N[C@H](C)c3cccc(C(C)(F)F)c3F)nc(C)nc12 ; COc1c(=O)n(C2(C(F)F)CC2)cc2c(N[C@H](C)c3ccc(-c4ccccc4CN(C)C)s3)nc(C)nc12 ; COc1c(=O)n(C2(C(F)F)CC2)cc2c(N[C@H](C)c3cccc(C#N)c3C)nc(C)nc12]

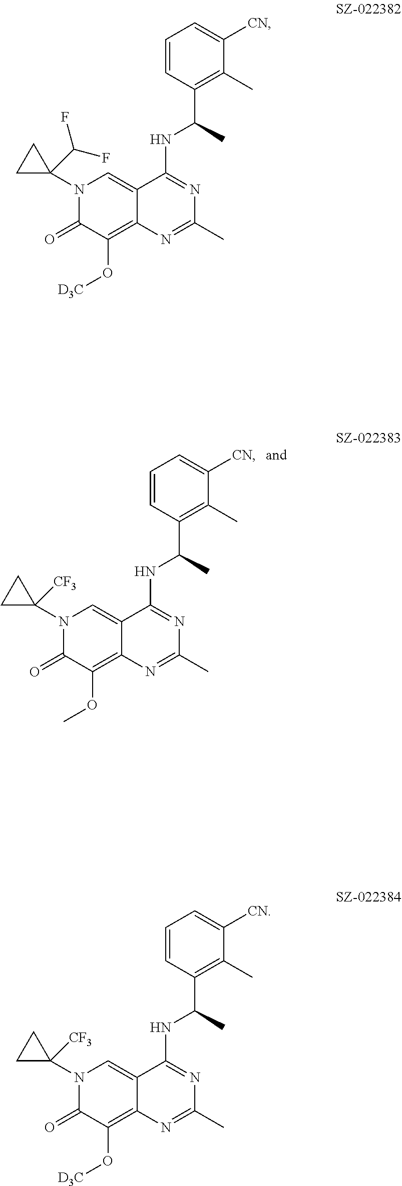 [SMILES: [2H]C([2H])([2H])Oc1c(=O)n(C2(C(F)F)CC2)cc2c(N[C@H](C)c3cccc(C#N)c3C)nc(C)nc12 ; COc1c(=O)n(C2(C(F)(F)F)CC2)cc2c(N[C@H](C)c3cccc(C#N)c3C)nc(C)nc12 ; [2H]C([2H])([2H])Oc1c(=O)n(C2(C(F)(F)F)CC2)cc2c(N[C@H](C)c3cccc(C#N)c3C)nc(C)nc12]

15. A pharmaceutical composition, comprising:
(1) the fused ring compound represented by formula I or the pharmaceutically acceptable salt thereof according to claim 1, and
(2) a pharmaceutically acceptable auxiliary material.

16. A method for inhibiting SOS1 in a subject in need thereof, comprising administering a therapeutically effective amount of the fused ring compound represented by formula I or the pharmaceutically acceptable salt thereof according to claim 1 to the subject.

17. The method according to claim 16, wherein the method satisfies one or more of the following conditions:
(1) a disease mediated by the SOS1 is lung cancer, pancreatic cancer, pancreatic ductal carcinoma, colon cancer, rectal cancer, appendiceal cancer, esophageal squamous carcinoma, head and neck squamous carcinoma, breast cancer, or other solid tumors; and
(2) the method is used in a mammalian organism; and/or the method is used in vitro, mainly for experimental purposes.

18. A method for preventing and/or treating a disease in a subject in need thereof, comprising administering a therapeutically effective amount of the fused ring compound represented by formula I or the pharmaceutically acceptable salt thereof according to claim 1 to the subject, wherein the disease is one or more of the following diseases: lung cancer, pancreatic cancer, pancreatic ductal carcinoma, colon cancer, rectal cancer, appendiceal cancer, esophageal carcinoma, head and neck squamous carcinoma, breast cancer, and other solid tumors.

19. The fused ring compound represented by formula I or the pharmaceutically acceptable salt thereof according to claim 1, wherein R₂ is C₁₋₆ alkyl or C₁₋₆ alkyl substituted with one or more R₂₋₁.

20. The fused ring compound represented by formula I or the pharmaceutically acceptable salt thereof according to claim 8, wherein the fused ring compound represented by formula I or the pharmaceutically acceptable salt thereof satisfies one or more of the following conditions:
(1) in R₁, R₁₋₁ₐ, R_{1-1b}, R_{1-1c}, R_{1-1d}, R₁₋₁ₑ, R₁₋₂ₐ, R_{1-2b}, R_{1-2c}, R_{1-2d}, R₁₋₂ₑ, R_{1-2f}, R_{1-2g}, R₁₋₃ₐ, R_{1-3b}, R_{1-3c}, R_{1-3d}, R₁₋₃ₑ, R_{1-3f}, R_{1-3g}, R₁₋₄ₐ, R_{1-4b}, R_{1-4c}, R₂, and R₂₋₁, the “C₃₋₁₀ cycloalkyl” in each of the “C₃₋₁₀ cycloalkyl”, the “C₃₋₁₀ cycloalkyl substituted with one or more R_{1-1b}”, the “C₃₋₁₀ cycloalkyl substituted with one or more R_{1-3b}”, and the “C₃₋₁₀ cycloalkyl substituted with one or more R_{1-3d}” is 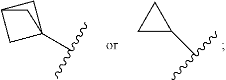
(2) in ring A, the “5- to 10-membered heterocyclyl” in the “5- to 10-membered heterocyclyl having 1, 2 or 3 heteroatoms selected from 1, 2 or 3 of N, O and S” is 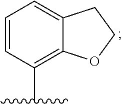 [SMILES: *c1cccc2c1OCC2]
and
(3) in R₁, R₁₋₂ₐ, R_{1-2b}, R_{1-2c}, R_{1-2d}, R₁₋₂ₑ, R_{1-2f}, R_{1-2g}, R₁₋₃ₐ, R_{1-3b}, R_{1-3c}, R_{1-3d}, R₁₋₃ₑ, R_{1-3f}, R_{1-3g}, R₁₋₄ₐ, R_{1-4b}, R_{1-4c}, and ring A, the “5- to 10-membered heteroaryl” in each of the “5- to 10-membered heteroaryl having 1, 2 or 3 heteroatoms selected from 1, 2 or 3 of N, O and S”, the “5- to 10-membered heteroaryl having 1, 2 or 3 heteroatoms selected from 1, 2 or 3 of N, O and S′ substituted with one or more R₁₋₁ₑ”, and the “5- to 10-membered heteroaryl having 1, 2 or 3 heteroatoms selected from 1, 2 or 3 of N, O and S′ substituted with one or more R_{1-3g}” is independently 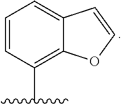 [SMILES: *c1cccc2ccoc12]
